(19) United States
(12) Patent Application Publication
(10) Pub. No.: US 2026/0141980 A1
(43) Pub. Date: May 21, 2026

(54) SYSTEMS AND METHODS FOR DESIGNING AND USING OLIGONUCLEOTIDE PROBES

(71) Applicant: Singular Genomics Systems, Inc., San Diego, CA (US)

(72) Inventors: Kenneth Howard GOUIN, III, Oceanside, CA (US); Michael Lawson, Pasadena, CA (US); Andrew Pawlowski, San Diego, CA (US)

(21) Appl. No.: 19/249,642

(22) Filed: Jun. 25, 2025

Related U.S. Application Data
(60) Provisional application No. 63/664,466, filed on Jun. 26, 2024.

(51) Int. Cl. G16B 25/20; C12Q 1/6834; C12Q 1/6855; G16B 30/10

(52) U.S. Cl. CPC G16B 25/20; C12Q 1/6834; C12Q 1/6855; G16B 30/10; C12Q 2600/16

(57) ABSTRACT

Disclosed herein, inter alia, are oligonucleotide probes for immuno-oncology target molecules and methods of use thereof.

### CROSS-REFERENCES TO RELATED APPLICATIONS

This application claims the benefit of U.S. Provisional Application No. 63/664,466, filed Jun. 26, 2024, which is incorporated herein by reference in its entirety and for all purposes.

### SEQUENCE LISTING

The instant application contains a Sequence Listing which has been submitted electronically in XML format and is hereby incorporated by reference in its entirety. Said XML copy, created on Jun. 20, 2025, is named 051385-625001US.xml, and is 13,679,352 bytes in size.

### BACKGROUND

While all cells within the human body contain substantially identical genetic material, the activation of specific genes varies among different cell types. These variations in gene expression are fundamental to both normal and pathological biological functions. Detecting and quantifying these gene expression patterns in normal versus diseased cells can be instrumental in identifying new targets for drug development and diagnostics. Disclosed herein, inter alia, are solutions to these and other problems in the art.

### BRIEF SUMMARY

In an aspect is provided an oligonucleotide probe including a sequence at least 80% homologous to SEQ ID NO:1 to SEQ ID NO:11418. In another aspect is provided an oligonucleotide probe including a sequence at least 80% homologous to SEQ ID NO:11428 to SEQ ID NO:15539. In an aspect is provided a method of detecting a nucleic acid molecule. In embodiments, the nucleic acid molecule is in a cell or tissue (e.g., in situ).

In another aspect is provided a computer-implemented method designing an oligonucleotide probe for a target gene. In embodiments, the method includes generating, by a processor, a plurality of probe sequences.

### BRIEF DESCRIPTION OF THE DRAWINGS

FIG. 1. Illustration of a code probe. A code probe is a single-stranded oligonucleotide that includes a first code-foot (e.g., 3′ code-foot) and a second code-foot (e.g., 5′ code-foot) on either end and is linked together via a linking oligonucleotide sequence. Each respective code-foot serves two purposes, binding to a specific target sequence, and following circularization, each code-foot may be detected to provide an identifier for the target sequence. The linking oligonucleotide sequence may include one or more primer binding sequences (e.g., an amplification and/or sequencing primer binding sequence).

FIG. 2. Determining off-target hits. An off-target hit must have ≥3 bp aligned on both sides of the ligation junction in order to be considered an off-target. Shown in FIG. 2, an off-target code probe is identified, and should be discarded from the optimized probe set.

FIG. 3. Example workflow for generating circularizable probes, referred to as padlock probes (PLPs) that each include a first hybridization sequence (code-foot 1) and a second hybridization sequence (code-foot 2). For a particular gene of interest, all possible foot-pairs (i.e., code-foot 1 and code-foot 2) are tiled across each exon for a target gene. A first filtering process removes any probes not having the desired GC content and removes probes where the foot-pair includes shared sequences (e.g., sharing a sequence ≤8 nucleotides). A second filtering step subjects the remaining potential code probes to additional filtering. For example, the pool was further refined by eliminating any code probes that overlapped with known transcriptome sequences. Oligonucleotides that had overlaps with each other were also removed. The remaining code probes were filtered to remove any code feet that had a melting temperature of less than (or equal) to 42° C. for off-targets; and further filtered to ensure any remaining code-feet included a melting temperature of greater (or equal) to 50° C. for on target sequences. Since the hybridization sequences function as identifying sequences, a degree of uniqueness to each sequence to facilitate multiplex determination is advantageous. For example, the optimized PLPs included a Hamming distance of greater (or equal) to 3 in the code foot, and any code feet not meeting these criteria were discarded.

FIG. 4 depicts an example system that may execute techniques and methods presented herein.

### DETAILED DESCRIPTION

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. In embodiments, a first template polynucleotide and a second template polynucleotide of an overlapping cluster are not substantially complementary (e.g., are at least 50%, 75%, 90%, or more non-complementary to each other).

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein or enzyme (e.g., a DNA polymerase).

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides.

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment, the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

In some embodiments, a nucleic acid (e.g., oligonucleotide probe) includes a molecular identifier or a molecular barcode. As used herein, the term “molecular barcode” (which may be referred to as a “tag”, a “barcode”, an “index” a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads comprising the barcode can be identified as originating from a single sample polynucleotide molecule on the basis of the barcode alone. In other embodiments, individual barcode sequences may be used more than once, but adapters comprising the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the index includes about 1 to about 10 nucleotides. In embodiments, the index includes about 3, 4, 5, 6, 7, 8, 9, or about 10 nucleotides. In embodiments, the index includes about 3 nucleotides. In embodiments, the index includes about 5 nucleotides. In embodiments, the index includes about 7 nucleotides. In embodiments, the index includes about 10 nucleotides. In embodiments, the index includes about 6 to about 10 nucleotides.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “template polynucleotide” or “template nucleic acid” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports in the form of discrete particles may be referred to herein as “beads,” which alone does not imply or require any particular shape. A bead can be non-spherical in shape. A solid support may further comprise a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate comprises a surface (e.g., a surface of a flow cell, a surface of a tube, a surface of a chip), for example a metal surface (e.g. steel, gold, silver, aluminum, silicon and copper). In some embodiments a substrate (e.g., a substrate surface) is coated and/or comprises functional groups and/or inert materials. In certain embodiments a substrate comprises a bead, a chip, a capillary, a plate, a membrane, a wafer (e.g., silicon wafers), a comb, or a pin for example. In some embodiments a substrate comprises a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In some embodiments a substrate comprises a magnetic material (e.g., iron, nickel, cobalt, platinum, aluminum, and the like).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions. In embodiments, a sequencing cycle incorporates one modified nucleotide into a primer hybridized to a template.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 1, 2, 5, 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acid. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex, which comprises a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by any suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments, a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments, solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution based primers can be used.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The term “nucleobase” or “base” as used herein refers to a purine or pyrimidine compound, or a derivative thereof, that may be a constituent of nucleic acid (i.e., DNA or RNA, or a derivative thereof). In embodiments, the nucleobase is a divalent purine or pyrimidine, or derivative thereof. In embodiments, the nucleobase is a monovalent purine or pyrimidine, or derivative thereof. In embodiments, the base is a derivative of a naturally occurring DNA or RNA base (e.g., a base analogue). In embodiments, the base is a hybridizing base. In embodiments, the base hybridizes to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is adenine, guanine, uracil, cytosine, thymine, hypoxanthine, xanthine, theobromine, caffeine, uric acid, or isoguanine, which may be optionally substituted or modified. In embodiments, the base is adenine, guanine, hypoxanthine, xanthine, theobromine, caffeine, uric acid, or isoguanine, which may be optionally substituted or modified.

“Synthetic” agents refer to non-naturally occurring agents, such as enzymes or nucleotides. The term “synthetic sequence” as used herein refers to a modified nucleic acid sequence such as those constructed by synthetic methods. In embodiments, a synthetic sequence is artificial or engineered, or derived from or contains an artificial or engineered nucleic acid content (e.g., non-natural or not wild type). For example, a polynucleotide sequence that is inserted or ligated to a target sequence (e.g., genomic DNA) may be referred to as a synthetic sequence. A polynucleotide adapter, as used herein, may be considered as providing a synthetic sequence.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (e.g., excitation wavelength, emission wavelength, emission intensity, FWHM (full width at half maximum peak height), or fluorescence lifetime) or a unique nucleic acid sequence characteristic.

As used herein, “capable of hybridizing” is used in accordance with its ordinary meaning in the art and refers to two oligonucleotides that, under suitable conditions, can form a duplex (e.g., Watson-Crick pairing) which includes a double-stranded portion of nucleic acid. Such conditions, known in the art and described herein, depend upon, for example, the nature of the nucleotide sequence, temperature, and buffer conditions. The stringency of hybridization can be influenced by various parameters, including degree of identity and/or complementarity between the polynucleotides (or any target sequences within the polynucleotides) to be hybridized; melting point of the polynucleotides and/or target sequences to be hybridized, referred to as “Tm”; parameters such as salts, buffers, pH, temperature, GC % content of the polynucleotide and primers, and/or time. Typically, hybridization is favored in lower temperatures and/or increased salt concentrations, as well as reduced concentrations of organic solvents. Some exemplary conditions suitable for hybridization include incubation of the polynucleotides to be hybridized in solutions having sodium salts, such as NaCl, sodium citrate and/or sodium phosphate. In some embodiments, hybridization or wash solutions can include about 10-75% formamide and/or about 0.01-0.7% sodium dodecyl sulfate (SDS). In some embodiments, a hybridization solution can be a stringent hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. In embodiments, the hybridization or washing solution can include BSA (bovine serum albumin). In embodiments, hybridization or washing can be conducted at a temperature range of about 20-25° C., or about 25-30° C., or about 30-35° C., or about 35-40° C., or about 40-45° C., or about 45-50° C., or about 50-55° C., or higher. In embodiments, hybridization or washing can be conducted for a time range of about 1-10 minutes, or about 10-20 minutes, or about 20-30 minutes, or about 30-40 minutes, or about 40-50 minutes, or about 50-60 minutes, or longer. In some embodiments, hybridization or wash conditions can be conducted at a pH range of about 5-10, or about pH 6-9, or about pH 6.5-8, or about pH 6.5-7.

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula,

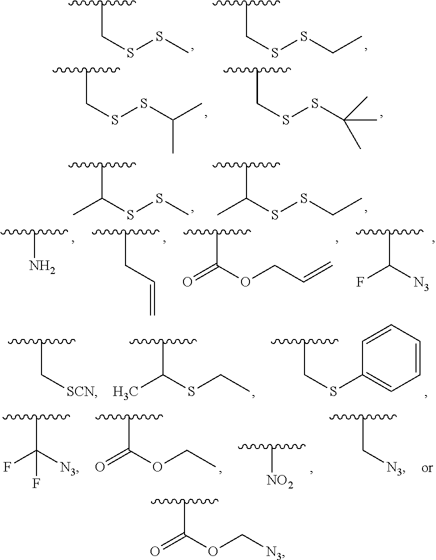

wherein the 3′ oxygen of the nucleotide is not shown in the formulae above. The term “allyl” as described herein refers to an unsubstituted methylene attached to a vinyl group (i.e., —CH═CH₂), e.g.,

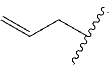 [SMILES: *CC=C]

In embodiments, the reversible terminator moiety is

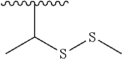 [SMILES: *C(C)SSC]

as described in U.S. Pat. No. 10,738,072, which is incorporated herein by reference for all purposes. For example, a nucleotide including a reversible terminator moiety may be represented by the formula:

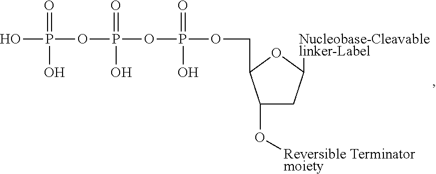

where the nucleobase is adenine or adenine analogue, thymine or thymine analogue, guanine or guanine analogue, or cytosine or cytosine analogue.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

The term “sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4X™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide or solid support as described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO™ Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

As used herein, the term “predicted melting temperature” or “melting temperature” or “Tm” refers to a computationally estimated temperature at which 50% of an oligonucleotide is expected to dissociate from its complementary strand under hybridization conditions (e.g., oligonucleotide concentration, ionic strength (e.g., sodium or magnesium ion concentration), pH, and/or solvent environment). The predicted melting temperature is calculated using thermodynamic models that account for sequence-specific hybridization stability, including base pairing and nearest-neighbor stacking interactions.

As used herein, the term “weak base” refers to refers to adenine (A) and thymine (T) bases as these nitrogenous bases form two hydrogen bond pairs and thus weaker bonds compared to the guanine (G) or cytosine (C) bases pairs.

As used herein, the term “strong base” refers to guanine (G) or cytosine (C) bases as these nitrogenous bases form three hydrogen bond pairs and thus stronger bonds compared to the adenine (A) and thymine (T) base pairs.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

The hardware and systems used to implement the various illustrative logics, logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In embodiments, the functions of the systems described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The term “computing device” is used herein to refer to an electronic device equipped with at least a processor. Examples of computing devices may include system or device described herein, mobile devices (e.g., cellular telephones, wearable devices, smartphones, smartwatches, web-pads, tablet computers, Internet enabled cellular telephones, Wi-Fi® enabled electronic devices, personal data assistants (PDAs), laptop computers, etc.), personal computers, and server computing devices. In various embodiments, computing devices may be configured with memory and/or storage as well as networking capabilities, such as network transceiver(s) and antenna(s) configured to establish a wide area network (WAN) connection (e.g., a cellular network connection, etc.) and/or a local area network (LAN) connection (e.g., a wired/wireless connection to the Internet via a Wi-Fi® router, etc.). In embodiments, the computing device is a mobile device, such as a cellular telephone, wearable device, or smartphone (e.g., iPhone, Android, Blackberry, Palm, Symbian, or Windows).

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

As used herein, the term “data string” refers to a sequence of characters derived from a biological or computational data set, such as nucleotide sequences representing candidate probe segments. Each data string may comprise alphanumeric characters, symbols, or encoded representations corresponding to nucleotides or other sequence-based identifiers. The term “collection,” when used in reference to data strings, refers to one or more data strings grouped, organized, or processed together. A collection may comprise data strings derived from a single data set or from two or more distinct data sets. In one example, a collection of data strings may include candidate oligonucleotide sequences generated from exonic regions of a target gene. In another example, a collection may include sequences derived from multiple target genes or reference genomes. As used herein, a “plurality of data strings” refers to two or more data strings. Each data string may correspond to a candidate probe sequence comprising a first segment and a second segment, each segment having a length between 15 and 20 nucleotides, as described in the methods herein.

A [0070]s used herein, a “subsequence”, “substring”, “prefix” or “suffix” of a string represents a subset of characters, letters, words, etc., of a longer list of characters, letters, words, etc., (i.e., the longer list being the sequence or string) wherein the order of the elements is preserved. A “prefix” typically refers to a subset of characters, letters, numbers, etc. found at the beginning of a sequence or string, whereas a “suffix” typically refers to a subset of characters, letters, numbers, etc. found at the end of a string. Substrings are also known as subwords or factors of a sequence or string.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided an oligonucleotide probe including a sequence at least 80% homologous to SEQ ID NO:1 to SEQ ID NO:11418.

In another aspect is provided an oligonucleotide probe including a sequence at least 80% homologous to SEQ ID NO:11428 to SEQ ID NO:15539. In embodiments, the oligonucleotide probe includes a sequence 90% homologous to SEQ ID NO:11428 to SEQ ID NO:15539. In embodiments, the oligonucleotide probe includes a sequence selected from SEQ ID NO:11428 to SEQ ID NO:15539. In embodiments, the oligonucleotide probe includes first sequence selected from SEQ ID NO:11428 to SEQ ID NO:13483 and a second sequence selected from SEQ ID NO:13484 to SEQ ID NO:15539. In embodiments, the oligonucleotide probe includes a primer binding sequence, or complement thereof, selected from the following: SEQ ID NO:15540, SEQ ID NO:15541, SEQ ID NO:15542, SEQ ID NO:15543, SEQ ID NO:15544, or SEQ ID NO:15545.

In an aspect is provided a plurality of oligonucleotide probes, wherein each oligonucleotide probe includes a first end, a backbone sequence, and a second end, wherein the first end includes a sequence selected from SEQ ID NO:11428 to SEQ ID NO:13483 and the second end includes a sequence selected from SEQ ID NO:13484 to SEQ ID NO:15539. In embodiments, the oligonucleotide probe is in a cell or tissue. In embodiments, the cell or tissue further includes a concatemer including multiple complementary copies of the oligonucleotide probe. In embodiments, the cell or tissue is immobilized to a solid support.

A probe is a molecule designed to recognize (and bind or hybridize to) another molecule, e.g., a target analyte, another probe molecule, etc. As used herein, the term “probe” may refer either to a chemical/physical probe molecule (e.g., a nucleic acid probe molecule) or to its representation in a computer-readable, digital format (e.g., as a string of characters representing the sequence of bases in a nucleic acid probe molecule). In embodiments, the oligonucleotide probe is a linear nucleic acid molecule. Exemplary probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes (e.g., including 1 to 4 ribonucleotides near the ligation junction). The specific probe or probe set design can vary. In some embodiments, the probes, such as a padlock probe or a probe set that comprises a padlock probe, contain one or more barcodes in the backbone sequence. In some embodiments, one or more barcodes are indicative of a sequence in the analyte nucleic acid, such as a single nucleotide (e.g., SNPs or point mutations), a dinucleotide sequence, a short sequence of about 5 nucleotides in length, or a sequence of any suitable length.

In embodiments, the oligonucleotide probe includes a first binding sequence and a second binding sequence. In embodiments, the first sequence is at least 80% homologous to a sequence selected from SEQ ID NO:11428 to SEQ ID NO:13483. In embodiments, the first sequence is 80% homologous to a sequence selected from SEQ ID NO:11428 to SEQ ID NO:13483. In embodiments, the first sequence is at least 90% homologous to a sequence selected from SEQ ID NO:11428 to SEQ ID NO:13483. In embodiments, the first sequence is 90% homologous to a sequence selected from SEQ ID NO:11428 to SEQ ID NO:13483. In embodiments, the first sequence is a sequence selected from SEQ ID NO:11428 to SEQ ID NO:13483. In embodiments, the second sequence is at least 80% homologous to a sequence selected from SEQ ID NO:13484 to SEQ ID NO:15538. In embodiments, the second sequence is 80% homologous to a sequence selected from SEQ ID NO:13484 to SEQ ID NO:15538. In embodiments, the second sequence is at least 90% homologous to a sequence selected from SEQ ID NO:13484 to SEQ ID NO:15538. In embodiments, the second sequence is 90% homologous to a sequence selected from SEQ ID NO:13484 to SEQ ID NO:15538. In embodiments, the oligonucleotide probe includes a primer binding sequence, or complement thereof, selected from the following sequences: SEQ ID NO:15540, SEQ ID NO: 15541, SEQ ID NO: 15542, SEQ ID NO: 15543, SEQ ID NO: 15544, or SEQ ID NO: 15545.

In embodiments, the oligonucleotide probe includes SEQ ID NO:5710 to SEQ ID NO:11418. In embodiments, the oligonucleotide probe includes SEQ ID NO:1 to SEQ ID NO:5709. In embodiments, the oligonucleotide probe includes a primer binding sequence, or complement thereof, selected from the following sequences: SEQ ID NO:11419, SEQ ID NO:11420, SEQ ID NO:11421, SEQ ID NO:11422, SEQ ID NO:11423, SEQ ID NO:11424, SEQ ID NO:11425, or SEQ ID NO:11426.

In an aspect is provided a plurality of nucleic acid molecules, wherein each nucleic acid molecule comprises a first end, a backbone sequence, and a second end, wherein the first end includes a sequence selected from SEQ ID NO:11428 to SEQ ID NO:13483. In embodiments, the backbone sequence includes a sequencing primer binding sequence. In embodiments, the backbone includes a sequencing primer sequence. In embodiments, the backbone sequence includes a sequence selected from: SEQ ID NO:15540, SEQ ID NO: 15541, SEQ ID NO: 15542, SEQ ID NO: 15543, SEQ ID NO: 15544, and SEQ ID NO: 15545. In embodiments, the second end includes a sequence selected from SEQ ID NO:13484 to SEQ ID NO:15538. In embodiments, the backbone sequence includes a barcode sequence.

In an aspect is provided a plurality of nucleic acid molecules, wherein each nucleic acid molecule comprises a first end, a backbone sequence, and a second end, wherein the first end includes a sequence selected from SEQ ID NO:5710 to SEQ ID NO:11418. In embodiments, the backbone sequence includes a sequencing primer binding sequence. In embodiments, the backbone includes a sequencing primer sequence. In embodiments, the backbone sequence includes a sequence selected from: SEQ ID NO:11419, SEQ ID NO:11420, SEQ ID NO:11421, SEQ ID NO:11422, SEQ ID NO:11423, SEQ ID NO:11424, SEQ ID NO:11425, and SEQ ID NO:11426. In embodiments, the second end includes a sequence selected from SEQ ID NO:1 to SEQ ID NO:5709.

In embodiments, the oligonucleotide probe is about 50 to about 500 nucleotides in length. In embodiments, the oligonucleotide probe is about 50 to about 300 nucleotides in length. In embodiments, the oligonucleotide probe is about 80 to about 300 nucleotides in length. In embodiments, the oligonucleotide probe is about 50 to about 150 nucleotides in length. In embodiments, the oligonucleotide probe is about or more than about 40, 50, 75, 100, 125, 150, 175, 200, 250, 300, 350, 400, or 500 nucleotides in length. In embodiments, the oligonucleotide probe is less than about 50, 75, 100, 125, 150, 175, 200, 250, 300, 350, 400, or 500 nucleotides in length. In embodiments, each probe oligonucleotide (e.g., each probe oligonucleotide of a plurality of probe oligonucleotides) includes a primer binding sequence (i.e., a sequence complementary to a primer, such as an amplification or sequencing primer). In embodiments, the oligonucleotide probe is about 80 to about 90 nucleotides in length. In embodiments, the oligonucleotide probe is 80 to 100 nucleotides in length. In embodiments, the oligonucleotide probe is 80, 81, 82, 83, 84, 85, 86, 87, 88, 89, or 90 nucleotides. In embodiments, the oligonucleotide probe is 80 nucleotides. In embodiments, the oligonucleotide probe is 81 nucleotides. In embodiments, the oligonucleotide probe is 82 nucleotides. In embodiments, the oligonucleotide probe is 83 nucleotides. In embodiments, the oligonucleotide probe is 84 nucleotides. In embodiments, the oligonucleotide probe is 85 nucleotides. In embodiments, the oligonucleotide probe is 86 nucleotides. In embodiments, the oligonucleotide probe is 87 nucleotides. In embodiments, the oligonucleotide probe is 88 nucleotides. In embodiments, the oligonucleotide probe is 89 nucleotides. In embodiments, the oligonucleotide probe is 90 nucleotides.

In embodiments, the oligonucleotide probe includes a target hybridization sequence (i.e., a sequence complementary to a sequence of the target nucleic acid molecule). In embodiments, the target hybridization sequence includes 5 to 25 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 15 to 30 nucleotides in length. In embodiments, the target hybridization sequence (e.g., the first and/or second target hybridization sequence) is greater than 30 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is 15 nucleotides in length. In embodiments, the target hybridization sequence is 20 nucleotides in length. In embodiments, the target hybridization sequence is 25 nucleotides in length. In embodiments, the target hybridization sequence is about 5, about 10, about 15, about 20, about 25, about 30, or about 35 nucleotides in length. In embodiments, the target hybridization sequence is about 5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the target hybridization sequence of each oligonucleotide primer is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence includes about 45% to 65% GC content (i.e., the percentage of nucleobases that are either guanine or cytosine). In embodiments, the target hybridization sequence does not include 4 or more guanine or cytosine nucleobases. In embodiments, the target hybridization sequence is designed according to the methods and systems described herein.

In embodiments, the oligonucleotide does not include five consecutive weak bases. In embodiments, the oligonucleotide does not include five consecutive strong bases. In embodiments, the oligonucleotide does not include secondary structure. In embodiments, the oligonucleotide does not include 2, 3, 4, 5, 6, 7, 8, 9, or 10 consecutive weak bases. In embodiments, the oligonucleotide does not include 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases or 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases and does not include 2, 3, 4, 5, 6, 7, 8, 9, or 10 consecutive strong bases.

In embodiments, the oligonucleotide probe includes a sequence at least 80%, 85%, 90%, 95%, 98%, 99%, or 100% identical to: SEQ ID NO:13484, SEQ ID NO:13485, SEQ ID NO:13486, SEQ ID NO:13487, SEQ ID NO:13488, SEQ ID NO:13489, SEQ ID NO:13490, SEQ ID NO:13491, SEQ ID NO:13492, SEQ ID NO:13493, SEQ ID NO:13494, SEQ ID NO:13495, SEQ ID NO:13496, SEQ ID NO:13497, SEQ ID NO:13498, SEQ ID NO:13499, SEQ ID NO:13500, SEQ ID NO:13501, SEQ ID NO:13502, SEQ ID NO:13503, SEQ ID NO:13504, SEQ ID NO:13505, SEQ ID NO:13506, SEQ ID NO:13507, SEQ ID NO:13508, SEQ ID NO:13509, SEQ ID NO:13510, SEQ ID NO:13511, SEQ ID NO:13512, SEQ ID NO:13513, SEQ ID NO:13514, SEQ ID NO:13515, SEQ ID NO:13516, SEQ ID NO:13517, SEQ ID NO:13518, SEQ ID NO:13519, SEQ ID NO:13520, SEQ ID NO:13521, SEQ ID NO:13522, SEQ ID NO:13523, SEQ ID NO:13524, SEQ ID NO:13525, SEQ ID NO:13526, SEQ ID NO:13527, SEQ ID NO:13528, SEQ ID NO:13529, SEQ ID NO:13530, SEQ ID NO:13531, SEQ ID NO:13532, SEQ ID NO:13533, SEQ ID NO:13534, SEQ ID NO:13535, SEQ ID NO:13536, SEQ ID NO:13537, SEQ ID NO:13538, SEQ ID NO:13539, SEQ ID NO:13540, SEQ ID NO:13541, SEQ ID NO:13542, SEQ ID NO:13543, SEQ ID NO:13544, SEQ ID NO:13545, SEQ ID NO:13546, SEQ ID NO:13547, SEQ ID NO:13548, SEQ ID NO:13549, SEQ ID NO:13550, SEQ ID NO:13551, SEQ ID NO:13552, SEQ ID NO:13553, SEQ ID NO:13554, SEQ ID NO:13555, SEQ ID NO:13556, SEQ ID NO:13557, SEQ ID NO:13558, SEQ ID NO:13559, SEQ ID NO:13560, SEQ ID NO:13561, SEQ ID NO:13562, SEQ ID NO:13563, SEQ ID NO:13564, SEQ ID NO:13565, SEQ ID NO:13566, SEQ ID NO:13567, SEQ ID NO:13568, SEQ ID NO:13569, SEQ ID NO:13570, SEQ ID NO:13571, SEQ ID NO:13572, SEQ ID NO:13573, SEQ ID NO:13574, SEQ ID NO:13575, SEQ ID NO:13576, SEQ ID NO:13577, SEQ ID NO:13578, SEQ ID NO:13579, SEQ ID NO:13580, SEQ ID NO:13581, SEQ ID NO:13582, SEQ ID NO:13583, SEQ ID NO:13584, SEQ ID NO:13585, SEQ ID NO:13586, SEQ ID NO:13587, SEQ ID NO:13588, SEQ ID NO:13589, SEQ ID NO:13590, SEQ ID NO:13591, SEQ ID NO:13592, SEQ ID NO:13593, SEQ ID NO:13594, SEQ ID NO:13595, SEQ ID NO:13596, SEQ ID NO:13597, SEQ ID NO:13598, SEQ ID NO:13599, SEQ ID NO:13600, SEQ ID NO:13601, SEQ ID NO:13602, SEQ ID NO:13603, SEQ ID NO:13604, SEQ ID NO:13605, SEQ ID NO:13606, SEQ ID NO:13607, SEQ ID NO:13608, SEQ ID NO:13609, SEQ ID NO:13610, SEQ ID NO:13611, SEQ ID NO:13612, SEQ ID NO:13613, SEQ ID NO:13614, SEQ ID NO:13615, SEQ ID NO:13616, SEQ ID NO:13617, SEQ ID NO:13618, SEQ ID NO:13619, SEQ ID NO:13620, SEQ ID NO:13621, SEQ ID NO:13622, SEQ ID NO:13623, SEQ ID NO:13624, SEQ ID NO:13625, SEQ ID NO:13626, SEQ ID NO:13627, SEQ ID NO:13628, SEQ ID NO:13629, SEQ ID NO:13630, SEQ ID NO:13631, SEQ ID NO:13632, SEQ ID NO:13633, SEQ ID NO:13634, SEQ ID NO:13635, SEQ ID NO:13636, SEQ ID NO:13637, SEQ ID NO:13638, SEQ ID NO:13639, SEQ ID NO:13640, SEQ ID NO:13641, SEQ ID NO:13642, SEQ ID NO:13643, SEQ ID NO:13644, SEQ ID NO:13645, SEQ ID NO:13646, SEQ ID NO:13647, SEQ ID NO:13648, SEQ ID NO:13649, SEQ ID NO:13650, SEQ ID NO:13651, SEQ ID NO:13652, SEQ ID NO:13653, SEQ ID NO:13654, SEQ ID NO:13655, SEQ ID NO:13656, SEQ ID NO:13657, SEQ ID NO:13658, SEQ ID NO:13659, SEQ ID NO:13660, SEQ ID NO:13661, SEQ ID NO:13662, SEQ ID NO:13663, SEQ ID NO:13664, SEQ ID NO:13665, SEQ ID NO:13666, SEQ ID NO:13667, SEQ ID NO:13668, SEQ ID NO:13669, SEQ ID NO:13670, SEQ ID NO:13671, SEQ ID NO:13672, SEQ ID NO:13673, SEQ ID NO:13674, SEQ ID NO:13675, SEQ ID NO:13676, SEQ ID NO:13677, SEQ ID NO:13678, SEQ ID NO:13679, SEQ ID NO:13680, SEQ ID NO:13681, SEQ ID NO:13682, SEQ ID NO:13683, SEQ ID NO:13684, SEQ ID NO:13685, SEQ ID NO:13686, SEQ ID NO:13687, SEQ ID NO:13688, SEQ ID NO:13689, SEQ ID NO:13690, SEQ ID NO:13691, SEQ ID NO:13692, SEQ ID NO:13693, SEQ ID NO:13694, SEQ ID NO:13695, SEQ ID NO:13696, SEQ ID NO:13697, SEQ ID NO:13698, SEQ ID NO:13699, SEQ ID NO:13700, SEQ ID NO:13701, SEQ ID NO:13702, SEQ ID NO:13703, SEQ ID NO:13704, SEQ ID NO:13705, SEQ ID NO:13706, SEQ ID NO:13707, SEQ ID NO:13708, SEQ ID NO:13709, SEQ ID NO:13710, SEQ ID NO:13711, SEQ ID NO:13712, SEQ ID NO:13713, SEQ ID NO:13714, SEQ ID NO:13715, SEQ ID NO:13716, SEQ ID NO:13717, SEQ ID NO:13718, SEQ ID NO:13719, SEQ ID NO:13720, SEQ ID NO:13721, SEQ ID NO:13722, SEQ ID NO:13723, SEQ ID NO:13724, SEQ ID NO:13725, SEQ ID NO:13726, SEQ ID NO:13727, SEQ ID NO:13728, SEQ ID NO:13729, SEQ ID NO:13730, SEQ ID NO:13731, SEQ ID NO:13732, SEQ ID NO:13733, SEQ ID NO:13734, SEQ ID NO:13735, SEQ ID NO:13736, SEQ ID NO:13737, SEQ ID NO:13738, SEQ ID NO:13739, SEQ ID NO:13740, SEQ ID NO:13741, SEQ ID NO:13742, SEQ ID NO:13743, SEQ ID NO:13744, SEQ ID NO:13745, SEQ ID NO:13746, SEQ ID NO:13747, SEQ ID NO:13748, SEQ ID NO:13749, SEQ ID NO:13750, SEQ ID NO:13751, SEQ ID NO:13752, SEQ ID NO:13753, SEQ ID NO:13754, SEQ ID NO:13755, SEQ ID NO:13756, SEQ ID NO:13757, SEQ ID NO:13758, SEQ ID NO:13759, SEQ ID NO:13760, SEQ ID NO:13761, SEQ ID NO:13762, SEQ ID NO:13763, SEQ ID NO:13764, SEQ ID NO:13765, SEQ ID NO:13766, SEQ ID NO:13767, SEQ ID NO:13768, SEQ ID NO:13769, SEQ ID NO:13770, SEQ ID NO:13771, SEQ ID NO:13772, SEQ ID NO:13773, SEQ ID NO:13774, SEQ ID NO:13775, SEQ ID NO:13776, SEQ ID NO:13777, SEQ ID NO:13778, SEQ ID NO:13779, SEQ ID NO:13780, SEQ ID NO:13781, SEQ ID NO:13782, SEQ ID NO:13783, SEQ ID NO:13784, SEQ ID NO:13785, SEQ ID NO:13786, SEQ ID NO:13787, SEQ ID NO:13788, SEQ ID NO:13789, SEQ ID NO:13790, SEQ ID NO:13791, SEQ ID NO:13792, SEQ ID NO:13793, SEQ ID NO:13794, SEQ ID NO:13795, SEQ ID NO:13796, SEQ ID NO:13797, SEQ ID NO:13798, SEQ ID NO:13799, SEQ ID NO:13800, SEQ ID NO:13801, SEQ ID NO:13802, SEQ ID NO:13803, SEQ ID NO:13804, SEQ ID NO:13805, SEQ ID NO:13806, SEQ ID NO:13807, SEQ ID NO:13808, SEQ ID NO:13809, SEQ ID NO:13810, SEQ ID NO:13811, SEQ ID NO:13812, SEQ ID NO:13813, SEQ ID NO:13814, SEQ ID NO:13815, SEQ ID NO:13816, SEQ ID NO:13817, SEQ ID NO:13818, SEQ ID NO:13819, SEQ ID NO:13820, SEQ ID NO:13821, SEQ ID NO:13822, SEQ ID NO:13823, SEQ ID NO:13824, SEQ ID NO:13825, SEQ ID NO:13826, SEQ ID NO:13827, SEQ ID NO:13828, SEQ ID NO:13829, SEQ ID NO:13830, SEQ ID NO:13831, SEQ ID NO:13832, SEQ ID NO:13833, SEQ ID NO:13834, SEQ ID NO:13835, SEQ ID NO:13836, SEQ ID NO:13837, SEQ ID NO:13838, SEQ ID NO:13839, SEQ ID NO:13840, SEQ ID NO:13841, SEQ ID NO:13842, SEQ ID NO:13843, SEQ ID NO:13844, SEQ ID NO:13845, SEQ ID NO:13846, SEQ ID NO:13847, SEQ ID NO:13848, SEQ ID NO:13849, SEQ ID NO:13850, SEQ ID NO:13851, SEQ ID NO:13852, SEQ ID NO:13853, SEQ ID NO:13854, SEQ ID NO:13855, SEQ ID NO:13856, SEQ ID NO:13857, SEQ ID NO:13858, SEQ ID NO:13859, SEQ ID NO:13860, SEQ ID NO:13861, SEQ ID NO:13862, SEQ ID NO:13863, SEQ ID NO:13864, SEQ ID NO:13865, SEQ ID NO:13866, SEQ ID NO:13867, SEQ ID NO:13868, SEQ ID NO:13869, SEQ ID NO:13870, SEQ ID NO:13871, SEQ ID NO:13872, SEQ ID NO:13873, SEQ ID NO:13874, SEQ ID NO:13875, SEQ ID NO:13876, SEQ ID NO:13877, SEQ ID NO:13878, SEQ ID NO:13879, SEQ ID NO:13880, SEQ ID NO:13881, SEQ ID NO:13882, SEQ ID NO:13883, SEQ ID NO:13884, SEQ ID NO:13885, SEQ ID NO:13886, SEQ ID NO:13887, SEQ ID NO:13888, SEQ ID NO:13889, SEQ ID NO:13890, SEQ ID NO:13891, SEQ ID NO:13892, SEQ ID NO:13893, SEQ ID NO:13894, SEQ ID NO:13895, SEQ ID NO:13896, SEQ ID NO:13897, SEQ ID NO:13898, SEQ ID NO:13899, SEQ ID NO:13900, SEQ ID NO:13901, SEQ ID NO:13902, SEQ ID NO:13903, SEQ ID NO:13904, SEQ ID NO:13905, SEQ ID NO:13906, SEQ ID NO:13907, SEQ ID NO:13908, SEQ ID NO:13909, SEQ ID NO:13910, SEQ ID NO:13911, SEQ ID NO:13912, SEQ ID NO:13913, SEQ ID NO:13914, SEQ ID NO:13915, SEQ ID NO:13916, SEQ ID NO:13917, SEQ ID NO:13918, SEQ ID NO:13919, SEQ ID NO:13920, SEQ ID NO:13921, SEQ ID NO:13922, SEQ ID NO:13923, SEQ ID NO:13924, SEQ ID NO:13925, SEQ ID NO:13926, SEQ ID NO:13927, SEQ ID NO:13928, SEQ ID NO:13929, SEQ ID NO:13930, SEQ ID NO:13931, SEQ ID NO:13932, SEQ ID NO:13933, SEQ ID NO:13934, SEQ ID NO:13935, SEQ ID NO:13936, SEQ ID NO:13937, SEQ ID NO:13938, SEQ ID NO:13939, SEQ ID NO:13940, SEQ ID NO:13941, SEQ ID NO:13942, SEQ ID NO:13943, SEQ ID NO:13944, SEQ ID NO:13945, SEQ ID NO:13946, SEQ ID NO:13947, SEQ ID NO:13948, SEQ ID NO:13949, SEQ ID NO:13950, SEQ ID NO:13951, SEQ ID NO:13952, SEQ ID NO:13953, SEQ ID NO:13954, SEQ ID NO:13955, SEQ ID NO:13956, SEQ ID NO:13957, SEQ ID NO:13958, SEQ ID NO:13959, SEQ ID NO:13960, SEQ ID NO:13961, SEQ ID NO:13962, SEQ ID NO:13963, SEQ ID NO:13964, SEQ ID NO:13965, SEQ ID NO:13966, SEQ ID NO:13967, SEQ ID NO:13968, SEQ ID NO:13969, SEQ ID NO:13970, SEQ ID NO:13971, SEQ ID NO:13972, SEQ ID NO:13973, SEQ ID NO:13974, SEQ ID NO:13975, SEQ ID NO:13976, SEQ ID NO:13977, SEQ ID NO:13978, SEQ ID NO:13979, SEQ ID NO:13980, SEQ ID NO:13981, SEQ ID NO:13982, SEQ ID NO:13983, SEQ ID NO:13984, SEQ ID NO:13985, SEQ ID NO:13986, SEQ ID NO:13987, SEQ ID NO:13988, SEQ ID NO:13989, SEQ ID NO:13990, SEQ ID NO:13991, SEQ ID NO:13992, SEQ ID NO:13993, SEQ ID NO:13994, SEQ ID NO:13995, SEQ ID NO:13996, SEQ ID NO:13997, SEQ ID NO:13998, SEQ ID NO:13999, SEQ ID NO:14000, SEQ ID NO:14001, SEQ ID NO:14002, SEQ ID NO:14003, SEQ ID NO:14004, SEQ ID NO:14005, SEQ ID NO:14006, SEQ ID NO:14007, SEQ ID NO:14008, SEQ ID NO:14009, SEQ ID NO:14010, SEQ ID NO:14011, SEQ ID NO:14012, SEQ ID NO:14013, SEQ ID NO:14014, SEQ ID NO:14015, SEQ ID NO:14016, SEQ ID NO:14017, SEQ ID NO:14018, SEQ ID NO:14019, SEQ ID NO:14020, SEQ ID NO:14021, SEQ ID NO:14022, SEQ ID NO:14023, SEQ ID NO:14024, SEQ ID NO:14025, SEQ ID NO:14026, SEQ ID NO:14027, SEQ ID NO:14028, SEQ ID NO:14029, SEQ ID NO:14030, SEQ ID NO:14031, SEQ ID NO:14032, SEQ ID NO:14033, SEQ ID NO:14034, SEQ ID NO:14035, SEQ ID NO:14036, SEQ ID NO:14037, SEQ ID NO:14038, SEQ ID NO:14039, SEQ ID NO:14040, SEQ ID NO:14041, SEQ ID NO:14042, SEQ ID NO:14043, SEQ ID NO:14044, SEQ ID NO:14045, SEQ ID NO:14046, SEQ ID NO:14047, SEQ ID NO:14048, SEQ ID NO:14049, SEQ ID NO:14050, SEQ ID NO:14051, SEQ ID NO:14052, SEQ ID NO:14053, SEQ ID NO:14054, SEQ ID NO:14055, SEQ ID NO:14056, SEQ ID NO:14057, SEQ ID NO:14058, SEQ ID NO:14059, SEQ ID NO:14060, SEQ ID NO:14061, SEQ ID NO:14062, SEQ ID NO:14063, SEQ ID NO:14064, SEQ ID NO:14065, SEQ ID NO:14066, SEQ ID NO:14067, SEQ ID NO:14068, SEQ ID NO:14069, SEQ ID NO:14070, SEQ ID NO:14071, SEQ ID NO:14072, SEQ ID NO:14073, SEQ ID NO:14074, SEQ ID NO:14075, SEQ ID NO:14076, SEQ ID NO:14077, SEQ ID NO:14078, SEQ ID NO:14079, SEQ ID NO:14080, SEQ ID NO:14081, SEQ ID NO:14082, SEQ ID NO:14083, SEQ ID NO:14084, SEQ ID NO:14085, SEQ ID NO:14086, SEQ ID NO:14087, SEQ ID NO:14088, SEQ ID NO:14089, SEQ ID NO:14090, SEQ ID NO:14091, SEQ ID NO:14092, SEQ ID NO:14093, SEQ ID NO:14094, SEQ ID NO:14095, SEQ ID NO:14096, SEQ ID NO:14097, SEQ ID NO:14098, SEQ ID NO:14099, SEQ ID NO:14100, SEQ ID NO:14101, SEQ ID NO:14102, SEQ ID NO:14103, SEQ ID NO:14104, SEQ ID NO:14105, SEQ ID NO:14106, SEQ ID NO:14107, SEQ ID NO:14108, SEQ ID NO:14109, SEQ ID NO:14110, SEQ ID NO:14111, SEQ ID NO:14112, SEQ ID NO:14113, SEQ ID NO:14114, SEQ ID NO:14115, SEQ ID NO:14116, SEQ ID NO:14117, SEQ ID NO:14118, SEQ ID NO:14119, SEQ ID NO:14120, SEQ ID NO:14121, SEQ ID NO:14122, SEQ ID NO:14123, SEQ ID NO:14124, SEQ ID NO:14125, SEQ ID NO:14126, SEQ ID NO:14127, SEQ ID NO:14128, SEQ ID NO:14129, SEQ ID NO:14130, SEQ ID NO:14131, SEQ ID NO:14132, SEQ ID NO:14133, SEQ ID NO:14134, SEQ ID NO:14135, SEQ ID NO:14136, SEQ ID NO:14137, SEQ ID NO:14138, SEQ ID NO:14139, SEQ ID NO:14140, SEQ ID NO:14141, SEQ ID NO:14142, SEQ ID NO:14143, SEQ ID NO:14144, SEQ ID NO:14145, SEQ ID NO:14146, SEQ ID NO:14147, SEQ ID NO:14148, SEQ ID NO:14149, SEQ ID NO:14150, SEQ ID NO:14151, SEQ ID NO:14152, SEQ ID NO:14153, SEQ ID NO:14154, SEQ ID NO:14155, SEQ ID NO:14156, SEQ ID NO:14157, SEQ ID NO:14158, SEQ ID NO:14159, SEQ ID NO:14160, SEQ ID NO:14161, SEQ ID NO:14162, SEQ ID NO:14163, SEQ ID NO:14164, SEQ ID NO:14165, SEQ ID NO:14166, SEQ ID NO:14167, SEQ ID NO:14168, SEQ ID NO:14169, SEQ ID NO:14170, SEQ ID NO:14171, SEQ ID NO:14172, SEQ ID NO:14173, SEQ ID NO:14174, SEQ ID NO:14175, SEQ ID NO:14176, SEQ ID NO:14177, SEQ ID NO:14178, SEQ ID NO:14179, SEQ ID NO:14180, SEQ ID NO:14181, SEQ ID NO:14182, SEQ ID NO:14183, SEQ ID NO:14184, SEQ ID NO:14185, SEQ ID NO:14186, SEQ ID NO:14187, SEQ ID NO:14188, SEQ ID NO:14189, SEQ ID NO:14190, SEQ ID NO:14191, SEQ ID NO:14192, SEQ ID NO:14193, SEQ ID NO:14194, SEQ ID NO:14195, SEQ ID NO:14196, SEQ ID NO:14197, SEQ ID NO:14198, SEQ ID NO:14199, SEQ ID NO:14200, SEQ ID NO:14201, SEQ ID NO:14202, SEQ ID NO:14203, SEQ ID NO:14204, SEQ ID NO:14205, SEQ ID NO:14206, SEQ ID NO:14207, SEQ ID NO:14208, SEQ ID NO:14209, SEQ ID NO:14210, SEQ ID NO:14211, SEQ ID NO:14212, SEQ ID NO:14213, SEQ ID NO:14214, SEQ ID NO:14215, SEQ ID NO:14216, SEQ ID NO:14217, SEQ ID NO:14218, SEQ ID NO:14219, SEQ ID NO:14220, SEQ ID NO:14221, SEQ ID NO:14222, SEQ ID NO:14223, SEQ ID NO:14224, SEQ ID NO:14225, SEQ ID NO:14226, SEQ ID NO:14227, SEQ ID NO:14228, SEQ ID NO:14229, SEQ ID NO:14230, SEQ ID NO:14231, SEQ ID NO:14232, SEQ ID NO:14233, SEQ ID NO:14234, SEQ ID NO:14235, SEQ ID NO:14236, SEQ ID NO:14237, SEQ ID NO:14238, SEQ ID NO:14239, SEQ ID NO:14240, SEQ ID NO:14241, SEQ ID NO:14242, SEQ ID NO:14243, SEQ ID NO:14244, SEQ ID NO:14245, SEQ ID NO:14246, SEQ ID NO:14247, SEQ ID NO:14248, SEQ ID NO:14249, SEQ ID NO:14250, SEQ ID NO:14251, SEQ ID NO:14252, SEQ ID NO:14253, SEQ ID NO:14254, SEQ ID NO:14255, SEQ ID NO:14256, SEQ ID NO:14257, SEQ ID NO:14258, SEQ ID NO:14259, SEQ ID NO:14260, SEQ ID NO:14261, SEQ ID NO:14262, SEQ ID NO:14263, SEQ ID NO:14264, SEQ ID NO:14265, SEQ ID NO:14266, SEQ ID NO:14267, SEQ ID NO:14268, SEQ ID NO:14269, SEQ ID NO:14270, SEQ ID NO:14271, SEQ ID NO:14272, SEQ ID NO:14273, SEQ ID NO:14274, SEQ ID NO:14275, SEQ ID NO:14276, SEQ ID NO:14277, SEQ ID NO:14278, SEQ ID NO:14279, SEQ ID NO:14280, SEQ ID NO:14281, SEQ ID NO:14282, SEQ ID NO:14283, SEQ ID NO:14284, SEQ ID NO:14285, SEQ ID NO:14286, SEQ ID NO:14287, SEQ ID NO:14288, SEQ ID NO:14289, SEQ ID NO:14290, SEQ ID NO:14291, SEQ ID NO:14292, SEQ ID NO:14293, SEQ ID NO:14294, SEQ ID NO:14295, SEQ ID NO:14296, SEQ ID NO:14297, SEQ ID NO:14298, SEQ ID NO:14299, SEQ ID NO:14300, SEQ ID NO:14301, SEQ ID NO:14302, SEQ ID NO:14303, SEQ ID NO:14304, SEQ ID NO:14305, SEQ ID NO:14306, SEQ ID NO:14307, SEQ ID NO:14308, SEQ ID NO:14309, SEQ ID NO:14310, SEQ ID NO:14311, SEQ ID NO:14312, SEQ ID NO:14313, SEQ ID NO:14314, SEQ ID NO:14315, SEQ ID NO:14316, SEQ ID NO:14317, SEQ ID NO:14318, SEQ ID NO:14319, SEQ ID NO:14320, SEQ ID NO:14321, SEQ ID NO:14322, SEQ ID NO:14323, SEQ ID NO:14324, SEQ ID NO:14325, SEQ ID NO:14326, SEQ ID NO:14327, SEQ ID NO:14328, SEQ ID NO:14329, SEQ ID NO:14330, SEQ ID NO:14331, SEQ ID NO:14332, SEQ ID NO:14333, SEQ ID NO:14334, SEQ ID NO:14335, SEQ ID NO:14336, SEQ ID NO:14337, SEQ ID NO:14338, SEQ ID NO:14339, SEQ ID NO:14340, SEQ ID NO:14341, SEQ ID NO:14342, SEQ ID NO:14343, SEQ ID NO:14344, SEQ ID NO:14345, SEQ ID NO:14346, SEQ ID NO:14347, SEQ ID NO:14348, SEQ ID NO:14349, SEQ ID NO:14350, SEQ ID NO:14351, SEQ ID NO:14352, SEQ ID NO:14353, SEQ ID NO:14354, SEQ ID NO:14355, SEQ ID NO:14356, SEQ ID NO:14357, SEQ ID NO:14358, SEQ ID NO:14359, SEQ ID NO:14360, SEQ ID NO:14361, SEQ ID NO:14362, SEQ ID NO:14363, SEQ ID NO:14364, SEQ ID NO:14365, SEQ ID NO:14366, SEQ ID NO:14367, SEQ ID NO:14368, SEQ ID NO:14369, SEQ ID NO:14370, SEQ ID NO:14371, SEQ ID NO:14372, SEQ ID NO:14373, SEQ ID NO:14374, SEQ ID NO:14375, SEQ ID NO:14376, SEQ ID NO:14377, SEQ ID NO:14378, SEQ ID NO:14379, SEQ ID NO:14380, SEQ ID NO:14381, SEQ ID NO:14382, SEQ ID NO:14383, SEQ ID NO:14384, SEQ ID NO:14385, SEQ ID NO:14386, SEQ ID NO:14387, SEQ ID NO:14388, SEQ ID NO:14389, SEQ ID NO:14390, SEQ ID NO:14391, SEQ ID NO:14392, SEQ ID NO:14393, SEQ ID NO:14394, SEQ ID NO:14395, SEQ ID NO:14396, SEQ ID NO:14397, SEQ ID NO:14398, SEQ ID NO:14399, SEQ ID NO:14400, SEQ ID NO:14401, SEQ ID NO:14402, SEQ ID NO:14403, SEQ ID NO:14404, SEQ ID NO:14405, SEQ ID NO:14406, SEQ ID NO:14407, SEQ ID NO:14408, SEQ ID NO:14409, SEQ ID NO:14410, SEQ ID NO:14411, SEQ ID NO:14412, SEQ ID NO:14413, SEQ ID NO:14414, SEQ ID NO:14415, SEQ ID NO:14416, SEQ ID NO:14417, SEQ ID NO:14418, SEQ ID NO:14419, SEQ ID NO:14420, SEQ ID NO:14421, SEQ ID NO:14422, SEQ ID NO:14423, SEQ ID NO:14424, SEQ ID NO:14425, SEQ ID NO:14426, SEQ ID NO:14427, SEQ ID NO:14428, SEQ ID NO:14429, SEQ ID NO:14430, SEQ ID NO:14431, SEQ ID NO:14432, SEQ ID NO:14433, SEQ ID NO:14434, SEQ ID NO:14435, SEQ ID NO:14436, SEQ ID NO:14437, SEQ ID NO:14438, SEQ ID NO:14439, SEQ ID NO:14440, SEQ ID NO:14441, SEQ ID NO:14442, SEQ ID NO:14443, SEQ ID NO:14444, SEQ ID NO:14445, SEQ ID NO:14446, SEQ ID NO:14447, SEQ ID NO:14448, SEQ ID NO:14449, SEQ ID NO:14450, SEQ ID NO:14451, SEQ ID NO:14452, SEQ ID NO:14453, SEQ ID NO:14454, SEQ ID NO:14455, SEQ ID NO:14456, SEQ ID NO:14457, SEQ ID NO:14458, SEQ ID NO:14459, SEQ ID NO:14460, SEQ ID NO:14461, SEQ ID NO:14462, SEQ ID NO:14463, SEQ ID NO:14464, SEQ ID NO:14465, SEQ ID NO:14466, SEQ ID NO:14467, SEQ ID NO:14468, SEQ ID NO:14469, SEQ ID NO:14470, SEQ ID NO:14471, SEQ ID NO:14472, SEQ ID NO:14473, SEQ ID NO:14474, SEQ ID NO:14475, SEQ ID NO:14476, SEQ ID NO:14477, SEQ ID NO:14478, SEQ ID NO:14479, SEQ ID NO:14480, SEQ ID NO:14481, SEQ ID NO:14482, SEQ ID NO:14483, SEQ ID NO:14484, SEQ ID NO:14485, SEQ ID NO:14486, SEQ ID NO:14487, SEQ ID NO:14488, SEQ ID NO:14489, SEQ ID NO:14490, SEQ ID NO:14491, SEQ ID NO:14492, SEQ ID NO:14493, SEQ ID NO:14494, SEQ ID NO:14495, SEQ ID NO:14496, SEQ ID NO:14497, SEQ ID NO:14498, SEQ ID NO:14499, SEQ ID NO:14500, SEQ ID NO:14501, SEQ ID NO:14502, SEQ ID NO:14503, SEQ ID NO:14504, SEQ ID NO:14505, SEQ ID NO:14506, SEQ ID NO:14507, SEQ ID NO:14508, SEQ ID NO:14509, SEQ ID NO:14510, SEQ ID NO:14511, SEQ ID NO:14512, SEQ ID NO:14513, SEQ ID NO:14514, SEQ ID NO:14515, SEQ ID NO:14516, SEQ ID NO:14517, SEQ ID NO:14518, SEQ ID NO:14519, SEQ ID NO:14520, SEQ ID NO:14521, SEQ ID NO:14522, SEQ ID NO:14523, SEQ ID NO:14524, SEQ ID NO:14525, SEQ ID NO:14526, SEQ ID NO:14527, SEQ ID NO:14528, SEQ ID NO:14529, SEQ ID NO:14530, SEQ ID NO:14531, SEQ ID NO:14532, SEQ ID NO:14533, SEQ ID NO:14534, SEQ ID NO:14535, SEQ ID NO:14536, SEQ ID NO:14537, SEQ ID NO:14538, SEQ ID NO:14539, SEQ ID NO:14540, SEQ ID NO:14541, SEQ ID NO:14542, SEQ ID NO:14543, SEQ ID NO:14544, SEQ ID NO:14545, SEQ ID NO:14546, SEQ ID NO:14547, SEQ ID NO:14548, SEQ ID NO:14549, SEQ ID NO:14550, SEQ ID NO:14551, SEQ ID NO:14552, SEQ ID NO:14553, SEQ ID NO:14554, SEQ ID NO:14555, SEQ ID NO:14556, SEQ ID NO:14557, SEQ ID NO:14558, SEQ ID NO:14559, SEQ ID NO:14560, SEQ ID NO:14561, SEQ ID NO:14562, SEQ ID NO:14563, SEQ ID NO:14564, SEQ ID NO:14565, SEQ ID NO:14566, SEQ ID NO:14567, SEQ ID NO:14568, SEQ ID NO:14569, SEQ ID NO:14570, SEQ ID NO:14571, SEQ ID NO:14572, SEQ ID NO:14573, SEQ ID NO:14574, SEQ ID NO:14575, SEQ ID NO:14576, SEQ ID NO:14577, SEQ ID NO:14578, SEQ ID NO:14579, SEQ ID NO:14580, SEQ ID NO:14581, SEQ ID NO:14582, SEQ ID NO:14583, SEQ ID NO:14584, SEQ ID NO:14585, SEQ ID NO:14586, SEQ ID NO:14587, SEQ ID NO:14588, SEQ ID NO:14589, SEQ ID NO:14590, SEQ ID NO:14591, SEQ ID NO:14592, SEQ ID NO:14593, SEQ ID NO:14594, SEQ ID NO:14595, SEQ ID NO:14596, SEQ ID NO:14597, SEQ ID NO:14598, SEQ ID NO:14599, SEQ ID NO:14600, SEQ ID NO:14601, SEQ ID NO:14602, SEQ ID NO:14603, SEQ ID NO:14604, SEQ ID NO:14605, SEQ ID NO:14606, SEQ ID NO:14607, SEQ ID NO:14608, SEQ ID NO:14609, SEQ ID NO:14610, SEQ ID NO:14611, SEQ ID NO:14612, SEQ ID NO:14613, SEQ ID NO:14614, SEQ ID NO:14615, SEQ ID NO:14616, SEQ ID NO:14617, SEQ ID NO:14618, SEQ ID NO:14619, SEQ ID NO:14620, SEQ ID NO:14621, SEQ ID NO:14622, SEQ ID NO:14623, SEQ ID NO:14624, SEQ ID NO:14625, SEQ ID NO:14626, SEQ ID NO:14627, SEQ ID NO:14628, SEQ ID NO:14629, SEQ ID NO:14630, SEQ ID NO:14631, SEQ ID NO:14632, SEQ ID NO:14633, SEQ ID NO:14634, SEQ ID NO:14635, SEQ ID NO:14636, SEQ ID NO:14637, SEQ ID NO:14638, SEQ ID NO:14639, SEQ ID NO:14640, SEQ ID NO:14641, SEQ ID NO:14642, SEQ ID NO:14643, SEQ ID NO:14644, SEQ ID NO:14645, SEQ ID NO:14646, SEQ ID NO:14647, SEQ ID NO:14648, SEQ ID NO:14649, SEQ ID NO:14650, SEQ ID NO:14651, SEQ ID NO:14652, SEQ ID NO:14653, SEQ ID NO:14654, SEQ ID NO:14655, SEQ ID NO:14656, SEQ ID NO:14657, SEQ ID NO:14658, SEQ ID NO:14659, SEQ ID NO:14660, SEQ ID NO:14661, SEQ ID NO:14662, SEQ ID NO:14663, SEQ ID NO:14664, SEQ ID NO:14665, SEQ ID NO:14666, SEQ ID NO:14667, SEQ ID NO:14668, SEQ ID NO:14669, SEQ ID NO:14670, SEQ ID NO:14671, SEQ ID NO:14672, SEQ ID NO:14673, SEQ ID NO:14674, SEQ ID NO:14675, SEQ ID NO:14676, SEQ ID NO:14677, SEQ ID NO:14678, SEQ ID NO:14679, SEQ ID NO:14680, SEQ ID NO:14681, SEQ ID NO:14682, SEQ ID NO:14683, SEQ ID NO:14684, SEQ ID NO:14685, SEQ ID NO:14686, SEQ ID NO:14687, SEQ ID NO:14688, SEQ ID NO:14689, SEQ ID NO:14690, SEQ ID NO:14691, SEQ ID NO:14692, SEQ ID NO:14693, SEQ ID NO:14694, SEQ ID NO:14695, SEQ ID NO:14696, SEQ ID NO:14697, SEQ ID NO:14698, SEQ ID NO:14699, SEQ ID NO:14700, SEQ ID NO:14701, SEQ ID NO:14702, SEQ ID NO:14703, SEQ ID NO:14704, SEQ ID NO:14705, SEQ ID NO:14706, SEQ ID NO:14707, SEQ ID NO:14708, SEQ ID NO:14709, SEQ ID NO:14710, SEQ ID NO:14711, SEQ ID NO:14712, SEQ ID NO:14713, SEQ ID NO:14714, SEQ ID NO:14715, SEQ ID NO:14716, SEQ ID NO:14717, SEQ ID NO:14718, SEQ ID NO:14719, SEQ ID NO:14720, SEQ ID NO:14721, SEQ ID NO:14722, SEQ ID NO:14723, SEQ ID NO:14724, SEQ ID NO:14725, SEQ ID NO:14726, SEQ ID NO:14727, SEQ ID NO:14728, SEQ ID NO:14729, SEQ ID NO:14730, SEQ ID NO:14731, SEQ ID NO:14732, SEQ ID NO:14733, SEQ ID NO:14734, SEQ ID NO:14735, SEQ ID NO:14736, SEQ ID NO:14737, SEQ ID NO:14738, SEQ ID NO:14739, SEQ ID NO:14740, SEQ ID NO:14741, SEQ ID NO:14742, SEQ ID NO:14743, SEQ ID NO:14744, SEQ ID NO:14745, SEQ ID NO:14746, SEQ ID NO:14747, SEQ ID NO:14748, SEQ ID NO:14749, SEQ ID NO:14750, SEQ ID NO:14751, SEQ ID NO:14752, SEQ ID NO:14753, SEQ ID NO:14754, SEQ ID NO:14755, SEQ ID NO:14756, SEQ ID NO:14757, SEQ ID NO:14758, SEQ ID NO:14759, SEQ ID NO:14760, SEQ ID NO:14761, SEQ ID NO:14762, SEQ ID NO:14763, SEQ ID NO:14764, SEQ ID NO:14765, SEQ ID NO:14766, SEQ ID NO:14767, SEQ ID NO:14768, SEQ ID NO:14769, SEQ ID NO:14770, SEQ ID NO:14771, SEQ ID NO:14772, SEQ ID NO:14773, SEQ ID NO:14774, SEQ ID NO:14775, SEQ ID NO:14776, SEQ ID NO:14777, SEQ ID NO:14778, SEQ ID NO:14779, SEQ ID NO:14780, SEQ ID NO:14781, SEQ ID NO:14782, SEQ ID NO:14783, SEQ ID NO:14784, SEQ ID NO:14785, SEQ ID NO:14786, SEQ ID NO:14787, SEQ ID NO:14788, SEQ ID NO:14789, SEQ ID NO:14790, SEQ ID NO:14791, SEQ ID NO:14792, SEQ ID NO:14793, SEQ ID NO:14794, SEQ ID NO:14795, SEQ ID NO:14796, SEQ ID NO:14797, SEQ ID NO:14798, SEQ ID NO:14799, SEQ ID NO:14800, SEQ ID NO:14801, SEQ ID NO:14802, SEQ ID NO:14803, SEQ ID NO:14804, SEQ ID NO:14805, SEQ ID NO:14806, SEQ ID NO:14807, SEQ ID NO:14808, SEQ ID NO:14809, SEQ ID NO:14810, SEQ ID NO:14811, SEQ ID NO:14812, SEQ ID NO:14813, SEQ ID NO:14814, SEQ ID NO:14815, SEQ ID NO:14816, SEQ ID NO:14817, SEQ ID NO:14818, SEQ ID NO:14819, SEQ ID NO:14820, SEQ ID NO:14821, SEQ ID NO:14822, SEQ ID NO:14823, SEQ ID NO:14824, SEQ ID NO:14825, SEQ ID NO:14826, SEQ ID NO:14827, SEQ ID NO:14828, SEQ ID NO:14829, SEQ ID NO:14830, SEQ ID NO:14831, SEQ ID NO:14832, SEQ ID NO:14833, SEQ ID NO:14834, SEQ ID NO:14835, SEQ ID NO:14836, SEQ ID NO:14837, SEQ ID NO:14838, SEQ ID NO:14839, SEQ ID NO:14840, SEQ ID NO:14841, SEQ ID NO:14842, SEQ ID NO:14843, SEQ ID NO:14844, SEQ ID NO:14845, SEQ ID NO:14846, SEQ ID NO:14847, SEQ ID NO:14848, SEQ ID NO:14849, SEQ ID NO:14850, SEQ ID NO:14851, SEQ ID NO:14852, SEQ ID NO:14853, SEQ ID NO:14854, SEQ ID NO:14855, SEQ ID NO:14856, SEQ ID NO:14857, SEQ ID NO:14858, SEQ ID NO:14859, SEQ ID NO:14860, SEQ ID NO:14861, SEQ ID NO:14862, SEQ ID NO:14863, SEQ ID NO:14864, SEQ ID NO:14865, SEQ ID NO:14866, SEQ ID NO:14867, SEQ ID NO:14868, SEQ ID NO:14869, SEQ ID NO:14870, SEQ ID NO:14871, SEQ ID NO:14872, SEQ ID NO:14873, SEQ ID NO:14874, SEQ ID NO:14875, SEQ ID NO:14876, SEQ ID NO:14877, SEQ ID NO:14878, SEQ ID NO:14879, SEQ ID NO:14880, SEQ ID NO:14881, SEQ ID NO:14882, SEQ ID NO:14883, SEQ ID NO:14884, SEQ ID NO:14885, SEQ ID NO:14886, SEQ ID NO:14887, SEQ ID NO:14888, SEQ ID NO:14889, SEQ ID NO:14890, SEQ ID NO:14891, SEQ ID NO:14892, SEQ ID NO:14893, SEQ ID NO:14894, SEQ ID NO:14895, SEQ ID NO:14896, SEQ ID NO:14897, SEQ ID NO:14898, SEQ ID NO:14899, SEQ ID NO:14900, SEQ ID NO:14901, SEQ ID NO:14902, SEQ ID NO:14903, SEQ ID NO:14904, SEQ ID NO:14905, SEQ ID NO:14906, SEQ ID NO:14907, SEQ ID NO:14908, SEQ ID NO:14909, SEQ ID NO:14910, SEQ ID NO:14911, SEQ ID NO:14912, SEQ ID NO:14913, SEQ ID NO:14914, SEQ ID NO:14915, SEQ ID NO:14916, SEQ ID NO:14917, SEQ ID NO:14918, SEQ ID NO:14919, SEQ ID NO:14920, SEQ ID NO:14921, SEQ ID NO:14922, SEQ ID NO:14923, SEQ ID NO:14924, SEQ ID NO:14925, SEQ ID NO:14926, SEQ ID NO:14927, SEQ ID NO:14928, SEQ ID NO:14929, SEQ ID NO:14930, SEQ ID NO:14931, SEQ ID NO:14932, SEQ ID NO:14933, SEQ ID NO:14934, SEQ ID NO:14935, SEQ ID NO:14936, SEQ ID NO:14937, SEQ ID NO:14938, SEQ ID NO:14939, SEQ ID NO:14940, SEQ ID NO:14941, SEQ ID NO:14942, SEQ ID NO:14943, SEQ ID NO:14944, SEQ ID NO:14945, SEQ ID NO:14946, SEQ ID NO:14947, SEQ ID NO:14948, SEQ ID NO:14949, SEQ ID NO:14950, SEQ ID NO:14951, SEQ ID NO:14952, SEQ ID NO:14953, SEQ ID NO:14954, SEQ ID NO:14955, SEQ ID NO:14956, SEQ ID NO:14957, SEQ ID NO:14958, SEQ ID NO:14959, SEQ ID NO:14960, SEQ ID NO:14961, SEQ ID NO:14962, SEQ ID NO:14963, SEQ ID NO:14964, SEQ ID NO:14965, SEQ ID NO:14966, SEQ ID NO:14967, SEQ ID NO:14968, SEQ ID NO:14969, SEQ ID NO:14970, SEQ ID NO:14971, SEQ ID NO:14972, SEQ ID NO:14973, SEQ ID NO:14974, SEQ ID NO:14975, SEQ ID NO:14976, SEQ ID NO:14977, SEQ ID NO:14978, SEQ ID NO:14979, SEQ ID NO:14980, SEQ ID NO:14981, SEQ ID NO:14982, SEQ ID NO:14983, SEQ ID NO:14984, SEQ ID NO:14985, SEQ ID NO:14986, SEQ ID NO:14987, SEQ ID NO:14988, SEQ ID NO:14989, SEQ ID NO:14990, SEQ ID NO:14991, SEQ ID NO:14992, SEQ ID NO:14993, SEQ ID NO:14994, SEQ ID NO:14995, SEQ ID NO:14996, SEQ ID NO:14997, SEQ ID NO:14998, SEQ ID NO:14999, SEQ ID NO:15000, SEQ ID NO:15001, SEQ ID NO:15002, SEQ ID NO:15003, SEQ ID NO:15004, SEQ ID NO:15005, SEQ ID NO:15006, SEQ ID NO:15007, SEQ ID NO:15008, SEQ ID NO:15009, SEQ ID NO:15010, SEQ ID NO:15011, SEQ ID NO:15012, SEQ ID NO:15013, SEQ ID NO:15014, SEQ ID NO:15015, SEQ ID NO:15016, SEQ ID NO:15017, SEQ ID NO:15018, SEQ ID NO:15019, SEQ ID NO:15020, SEQ ID NO:15021, SEQ ID NO:15022, SEQ ID NO:15023, SEQ ID NO:15024, SEQ ID NO:15025, SEQ ID NO:15026, SEQ ID NO:15027, SEQ ID NO:15028, SEQ ID NO:15029, SEQ ID NO:15030, SEQ ID NO:15031, SEQ ID NO:15032, SEQ ID NO:15033, SEQ ID NO:15034, SEQ ID NO:15035, SEQ ID NO:15036, SEQ ID NO:15037, SEQ ID NO:15038, SEQ ID NO:15039, SEQ ID NO:15040, SEQ ID NO:15041, SEQ ID NO:15042, SEQ ID NO:15043, SEQ ID NO:15044, SEQ ID NO:15045, SEQ ID NO:15046, SEQ ID NO:15047, SEQ ID NO:15048, SEQ ID NO:15049, SEQ ID NO:15050, SEQ ID NO:15051, SEQ ID NO:15052, SEQ ID NO:15053, SEQ ID NO:15054, SEQ ID NO:15055, SEQ ID NO:15056, SEQ ID NO:15057, SEQ ID NO:15058, SEQ ID NO:15059, SEQ ID NO:15060, SEQ ID NO:15061, SEQ ID NO:15062, SEQ ID NO:15063, SEQ ID NO:15064, SEQ ID NO:15065, SEQ ID NO:15066, SEQ ID NO:15067, SEQ ID NO:15068, SEQ ID NO:15069, SEQ ID NO:15070, SEQ ID NO:15071, SEQ ID NO:15072, SEQ ID NO:15073, SEQ ID NO:15074, SEQ ID NO:15075, SEQ ID NO:15076, SEQ ID NO:15077, SEQ ID NO:15078, SEQ ID NO:15079, SEQ ID NO:15080, SEQ ID NO:15081, SEQ ID NO:15082, SEQ ID NO:15083, SEQ ID NO:15084, SEQ ID NO:15085, SEQ ID NO:15086, SEQ ID NO:15087, SEQ ID NO:15088, SEQ ID NO:15089, SEQ ID NO:15090, SEQ ID NO:15091, SEQ ID NO:15092, SEQ ID NO:15093, SEQ ID NO:15094, SEQ ID NO:15095, SEQ ID NO:15096, SEQ ID NO:15097, SEQ ID NO:15098, SEQ ID NO:15099, SEQ ID NO:15100, SEQ ID NO:15101, SEQ ID NO:15102, SEQ ID NO:15103, SEQ ID NO:15104, SEQ ID NO:15105, SEQ ID NO:15106, SEQ ID NO:15107, SEQ ID NO:15108, SEQ ID NO:15109, SEQ ID NO:15110, SEQ ID NO:15111, SEQ ID NO:15112, SEQ ID NO:15113, SEQ ID NO:15114, SEQ ID NO:15115, SEQ ID NO:15116, SEQ ID NO:15117, SEQ ID NO:15118, SEQ ID NO:15119, SEQ ID NO:15120, SEQ ID NO:15121, SEQ ID NO:15122, SEQ ID NO:15123, SEQ ID NO:15124, SEQ ID NO:15125, SEQ ID NO:15126, SEQ ID NO:15127, SEQ ID NO:15128, SEQ ID NO:15129, SEQ ID NO:15130, SEQ ID NO:15131, SEQ ID NO:15132, SEQ ID NO:15133, SEQ ID NO:15134, SEQ ID NO:15135, SEQ ID NO:15136, SEQ ID NO:15137, SEQ ID NO:15138, SEQ ID NO:15139, SEQ ID NO:15140, SEQ ID NO:15141, SEQ ID NO:15142, SEQ ID NO:15143, SEQ ID NO:15144, SEQ ID NO:15145, SEQ ID NO:15146, SEQ ID NO:15147, SEQ ID NO:15148, SEQ ID NO:15149, SEQ ID NO:15150, SEQ ID NO:15151, SEQ ID NO:15152, SEQ ID NO:15153, SEQ ID NO:15154, SEQ ID NO:15155, SEQ ID NO:15156, SEQ ID NO:15157, SEQ ID NO:15158, SEQ ID NO:15159, SEQ ID NO:15160, SEQ ID NO:15161, SEQ ID NO:15162, SEQ ID NO:15163, SEQ ID NO:15164, SEQ ID NO:15165, SEQ ID NO:15166, SEQ ID NO:15167, SEQ ID NO:15168, SEQ ID NO:15169, SEQ ID NO:15170, SEQ ID NO:15171, SEQ ID NO:15172, SEQ ID NO:15173, SEQ ID NO:15174, SEQ ID NO:15175, SEQ ID NO:15176, SEQ ID NO:15177, SEQ ID NO:15178, SEQ ID NO:15179, SEQ ID NO:15180, SEQ ID NO:15181, SEQ ID NO:15182, SEQ ID NO:15183, SEQ ID NO:15184, SEQ ID NO:15185, SEQ ID NO:15186, SEQ ID NO:15187, SEQ ID NO:15188, SEQ ID NO:15189, SEQ ID NO:15190, SEQ ID NO:15191, SEQ ID NO:15192, SEQ ID NO:15193, SEQ ID NO:15194, SEQ ID NO:15195, SEQ ID NO:15196, SEQ ID NO:15197, SEQ ID NO:15198, SEQ ID NO:15199, SEQ ID NO:15200, SEQ ID NO:15201, SEQ ID NO:15202, SEQ ID NO:15203, SEQ ID NO:15204, SEQ ID NO:15205, SEQ ID NO:15206, SEQ ID NO:15207, SEQ ID NO:15208, SEQ ID NO:15209, SEQ ID NO:15210, SEQ ID NO:15211, SEQ ID NO:15212, SEQ ID NO:15213, SEQ ID NO:15214, SEQ ID NO:15215, SEQ ID NO:15216, SEQ ID NO:15217, SEQ ID NO:15218, SEQ ID NO:15219, SEQ ID NO:15220, SEQ ID NO:15221, SEQ ID NO:15222, SEQ ID NO:15223, SEQ ID NO:15224, SEQ ID NO:15225, SEQ ID NO:15226, SEQ ID NO:15227, SEQ ID NO:15228, SEQ ID NO:15229, SEQ ID NO:15230, SEQ ID NO:15231, SEQ ID NO:15232, SEQ ID NO:15233, SEQ ID NO:15234, SEQ ID NO:15235, SEQ ID NO:15236, SEQ ID NO:15237, SEQ ID NO:15238, SEQ ID NO:15239, SEQ ID NO:15240, SEQ ID NO:15241, SEQ ID NO:15242, SEQ ID NO:15243, SEQ ID NO:15244, SEQ ID NO:15245, SEQ ID NO:15246, SEQ ID NO:15247, SEQ ID NO:15248, SEQ ID NO:15249, SEQ ID NO:15250, SEQ ID NO:15251, SEQ ID NO:15252, SEQ ID NO:15253, SEQ ID NO:15254, SEQ ID NO:15255, SEQ ID NO:15256, SEQ ID NO:15257, SEQ ID NO:15258, SEQ ID NO:15259, SEQ ID NO:15260, SEQ ID NO:15261, SEQ ID NO:15262, SEQ ID NO:15263, SEQ ID NO:15264, SEQ ID NO:15265, SEQ ID NO:15266, SEQ ID NO:15267, SEQ ID NO:15268, SEQ ID NO:15269, SEQ ID NO:15270, SEQ ID NO:15271, SEQ ID NO:15272, SEQ ID NO:15273, SEQ ID NO:15274, SEQ ID NO:15275, SEQ ID NO:15276, SEQ ID NO:15277, SEQ ID NO:15278, SEQ ID NO:15279, SEQ ID NO:15280, SEQ ID NO:15281, SEQ ID NO:15282, SEQ ID NO:15283, SEQ ID NO:15284, SEQ ID NO:15285, SEQ ID NO:15286, SEQ ID NO:15287, SEQ ID NO:15288, SEQ ID NO:15289, SEQ ID NO:15290, SEQ ID NO:15291, SEQ ID NO:15292, SEQ ID NO:15293, SEQ ID NO:15294, SEQ ID NO:15295, SEQ ID NO:15296, SEQ ID NO:15297, SEQ ID NO:15298, SEQ ID NO:15299, SEQ ID NO:15300, SEQ ID NO:15301, SEQ ID NO:15302, SEQ ID NO:15303, SEQ ID NO:15304, SEQ ID NO:15305, SEQ ID NO:15306, SEQ ID NO:15307, SEQ ID NO:15308, SEQ ID NO:15309, SEQ ID NO:15310, SEQ ID NO:15311, SEQ ID NO:15312, SEQ ID NO:15313, SEQ ID NO:15314, SEQ ID NO:15315, SEQ ID NO:15316, SEQ ID NO:15317, SEQ ID NO:15318, SEQ ID NO:15319, SEQ ID NO:15320, SEQ ID NO:15321, SEQ ID NO:15322, SEQ ID NO:15323, SEQ ID NO:15324, SEQ ID NO:15325, SEQ ID NO:15326, SEQ ID NO:15327, SEQ ID NO:15328, SEQ ID NO:15329, SEQ ID NO:15330, SEQ ID NO:15331, SEQ ID NO:15332, SEQ ID NO:15333, SEQ ID NO:15334, SEQ ID NO:15335, SEQ ID NO:15336, SEQ ID NO:15337, SEQ ID NO:15338, SEQ ID NO:15339, SEQ ID NO:15340, SEQ ID NO:15341, SEQ ID NO:15342, SEQ ID NO:15343, SEQ ID NO:15344, SEQ ID NO:15345, SEQ ID NO:15346, SEQ ID NO:15347, SEQ ID NO:15348, SEQ ID NO:15349, SEQ ID NO:15350, SEQ ID NO:15351, SEQ ID NO:15352, SEQ ID NO:15353, SEQ ID NO:15354, SEQ ID NO:15355, SEQ ID NO:15356, SEQ ID NO:15357, SEQ ID NO:15358, SEQ ID NO:15359, SEQ ID NO:15360, SEQ ID NO:15361, SEQ ID NO:15362, SEQ ID NO:15363, SEQ ID NO:15364, SEQ ID NO:15365, SEQ ID NO:15366, SEQ ID NO:15367, SEQ ID NO:15368, SEQ ID NO:15369, SEQ ID NO:15370, SEQ ID NO:15371, SEQ ID NO:15372, SEQ ID NO:15373, SEQ ID NO:15374, SEQ ID NO:15375, SEQ ID NO:15376, SEQ ID NO:15377, SEQ ID NO:15378, SEQ ID NO:15379, SEQ ID NO:15380, SEQ ID NO:15381, SEQ ID NO:15382, SEQ ID NO:15383, SEQ ID NO:15384, SEQ ID NO:15385, SEQ ID NO:15386, SEQ ID NO:15387, SEQ ID NO:15388, SEQ ID NO:15389, SEQ ID NO:15390, SEQ ID NO:15391, SEQ ID NO:15392, SEQ ID NO:15393, SEQ ID NO:15394, SEQ ID NO:15395, SEQ ID NO:15396, SEQ ID NO:15397, SEQ ID NO:15398, SEQ ID NO:15399, SEQ ID NO:15400, SEQ ID NO:15401, SEQ ID NO:15402, SEQ ID NO:15403, SEQ ID NO:15404, SEQ ID NO:15405, SEQ ID NO:15406, SEQ ID NO:15407, SEQ ID NO:15408, SEQ ID NO:15409, SEQ ID NO:15410, SEQ ID NO:15411, SEQ ID NO:15412, SEQ ID NO:15413, SEQ ID NO:15414, SEQ ID NO:15415, SEQ ID NO:15416, SEQ ID NO:15417, SEQ ID NO:15418, SEQ ID NO:15419, SEQ ID NO:15420, SEQ ID NO:15421, SEQ ID NO:15422, SEQ ID NO:15423, SEQ ID NO:15424, SEQ ID NO:15425, SEQ ID NO:15426, SEQ ID NO:15427, SEQ ID NO:15428, SEQ ID NO:15429, SEQ ID NO:15430, SEQ ID NO:15431, SEQ ID NO:15432, SEQ ID NO:15433, SEQ ID NO:15434, SEQ ID NO:15435, SEQ ID NO:15436, SEQ ID NO:15437, SEQ ID NO:15438, SEQ ID NO:15439, SEQ ID NO:15440, SEQ ID NO:15441, SEQ ID NO:15442, SEQ ID NO:15443, SEQ ID NO:15444, SEQ ID NO:15445, SEQ ID NO:15446, SEQ ID NO:15447, SEQ ID NO:15448, SEQ ID NO:15449, SEQ ID NO:15450, SEQ ID NO:15451, SEQ ID NO:15452, SEQ ID NO:15453, SEQ ID NO:15454, SEQ ID NO:15455, SEQ ID NO:15456, SEQ ID NO:15457, SEQ ID NO:15458, SEQ ID NO:15459, SEQ ID NO:15460, SEQ ID NO:15461, SEQ ID NO:15462, SEQ ID NO:15463, SEQ ID NO:15464, SEQ ID NO:15465, SEQ ID NO:15466, SEQ ID NO:15467, SEQ ID NO:15468, SEQ ID NO:15469, SEQ ID NO:15470, SEQ ID NO:15471, SEQ ID NO:15472, SEQ ID NO:15473, SEQ ID NO:15474, SEQ ID NO:15475, SEQ ID NO:15476, SEQ ID NO:15477, SEQ ID NO:15478, SEQ ID NO:15479, SEQ ID NO:15480, SEQ ID NO:15481, SEQ ID NO:15482, SEQ ID NO:15483, SEQ ID NO:15484, SEQ ID NO:15485, SEQ ID NO:15486, SEQ ID NO:15487, SEQ ID NO:15488, SEQ ID NO:15489, SEQ ID NO:15490, SEQ ID NO:15491, SEQ ID NO:15492, SEQ ID NO:15493, SEQ ID NO:15494, SEQ ID NO:15495, SEQ ID NO:15496, SEQ ID NO:15497, SEQ ID NO:15498, SEQ ID NO:15499, SEQ ID NO:15500, SEQ ID NO:15501, SEQ ID NO:15502, SEQ ID NO:15503, SEQ ID NO:15504, SEQ ID NO:15505, SEQ ID NO:15506, SEQ ID NO:15507, SEQ ID NO:15508, SEQ ID NO:15509, SEQ ID NO:15510, SEQ ID NO:15511, SEQ ID NO:15512, SEQ ID NO:15513, SEQ ID NO:15514, SEQ ID NO:15515, SEQ ID NO:15516, SEQ ID NO:15517, SEQ ID NO:15518, SEQ ID NO:15519, SEQ ID NO:15520, SEQ ID NO:15521, SEQ ID NO:15522, SEQ ID NO:15523, SEQ ID NO:15524, SEQ ID NO:15525, SEQ ID NO:15526, SEQ ID NO:15527, SEQ ID NO:15528, SEQ ID NO:15529, SEQ ID NO:15530, SEQ ID NO:15531, SEQ ID NO:15532, SEQ ID NO:15533, SEQ ID NO:15534, SEQ ID NO:15535, SEQ ID NO:15536, SEQ ID NO:15537, SEQ ID NO:15538, or SEQ ID NO:15539.

In embodiments, the oligonucleotide probe includes a sequence at least 80%, 85%, 90%, 95%, 98%, 99%, or 100% identical to: SEQ ID NO:11428, SEQ ID NO:11429, SEQ ID NO:11430, SEQ ID NO:11431, SEQ ID NO:11432, SEQ ID NO:11433, SEQ ID NO:11434, SEQ ID NO:11435, SEQ ID NO:11436, SEQ ID NO:11437, SEQ ID NO:11438, SEQ ID NO:11439, SEQ ID NO:11440, SEQ ID NO:11441, SEQ ID NO:11442, SEQ ID NO:11443, SEQ ID NO:11444, SEQ ID NO:11445, SEQ ID NO:11446, SEQ ID NO:11447, SEQ ID NO:11448, SEQ ID NO:11449, SEQ ID NO:11450, SEQ ID NO:11451, SEQ ID NO:11452, SEQ ID NO:11453, SEQ ID NO:11454, SEQ ID NO:11455, SEQ ID NO:11456, SEQ ID NO:11457, SEQ ID NO:11458, SEQ ID NO:11459, SEQ ID NO:11460, SEQ ID NO:11461, SEQ ID NO:11462, SEQ ID NO:11463, SEQ ID NO:11464, SEQ ID NO:11465, SEQ ID NO:11466, SEQ ID NO:11467, SEQ ID NO:11468, SEQ ID NO:11469, SEQ ID NO:11470, SEQ ID NO:11471, SEQ ID NO:11472, SEQ ID NO:11473, SEQ ID NO:11474, SEQ ID NO:11475, SEQ ID NO:11476, SEQ ID NO:11477, SEQ ID NO:11478, SEQ ID NO:11479, SEQ ID NO:11480, SEQ ID NO:11481, SEQ ID NO:11482, SEQ ID NO:11483, SEQ ID NO:11484, SEQ ID NO:11485, SEQ ID NO:11486, SEQ ID NO:11487, SEQ ID NO:11488, SEQ ID NO:11489, SEQ ID NO:11490, SEQ ID NO:11491, SEQ ID NO:11492, SEQ ID NO:11493, SEQ ID NO:11494, SEQ ID NO:11495, SEQ ID NO:11496, SEQ ID NO:11497, SEQ ID NO:11498, SEQ ID NO:11499, SEQ ID NO:11500, SEQ ID NO:11501, SEQ ID NO:11502, SEQ ID NO:11503, SEQ ID NO:11504, SEQ ID NO:11505, SEQ ID NO:11506, SEQ ID NO:11507, SEQ ID NO:11508, SEQ ID NO:11509, SEQ ID NO:11510, SEQ ID NO:11511, SEQ ID NO:11512, SEQ ID NO:11513, SEQ ID NO:11514, SEQ ID NO:11515, SEQ ID NO:11516, SEQ ID NO:11517, SEQ ID NO:11518, SEQ ID NO:11519, SEQ ID NO:11520, SEQ ID NO:11521, SEQ ID NO:11522, SEQ ID NO:11523, SEQ ID NO:11524, SEQ ID NO:11525, SEQ ID NO:11526, SEQ ID NO:11527, SEQ ID NO:11528, SEQ ID NO:11529, SEQ ID NO:11530, SEQ ID NO:11531, SEQ ID NO:11532, SEQ ID NO:11533, SEQ ID NO:11534, SEQ ID NO:11535, SEQ ID NO:11536, SEQ ID NO:11537, SEQ ID NO:11538, SEQ ID NO:11539, SEQ ID NO:11540, SEQ ID NO:11541, SEQ ID NO:11542, SEQ ID NO:11543, SEQ ID NO:11544, SEQ ID NO:11545, SEQ ID NO:11546, SEQ ID NO:11547, SEQ ID NO:11548, SEQ ID NO:11549, SEQ ID NO:11550, SEQ ID NO:11551, SEQ ID NO:11552, SEQ ID NO:11553, SEQ ID NO:11554, SEQ ID NO:11555, SEQ ID NO:11556, SEQ ID NO:11557, SEQ ID NO:11558, SEQ ID NO:11559, SEQ ID NO:11560, SEQ ID NO:11561, SEQ ID NO:11562, SEQ ID NO:11563, SEQ ID NO:11564, SEQ ID NO:11565, SEQ ID NO:11566, SEQ ID NO:11567, SEQ ID NO:11568, SEQ ID NO:11569, SEQ ID NO:11570, SEQ ID NO:11571, SEQ ID NO:11572, SEQ ID NO:11573, SEQ ID NO:11574, SEQ ID NO:11575, SEQ ID NO:11576, SEQ ID NO:11577, SEQ ID NO:11578, SEQ ID NO:11579, SEQ ID NO:11580, SEQ ID NO:11581, SEQ ID NO:11582, SEQ ID NO:11583, SEQ ID NO:11584, SEQ ID NO:11585, SEQ ID NO:11586, SEQ ID NO:11587, SEQ ID NO:11588, SEQ ID NO:11589, SEQ ID NO:11590, SEQ ID NO:11591, SEQ ID NO:11592, SEQ ID NO:11593, SEQ ID NO:11594, SEQ ID NO:11595, SEQ ID NO:11596, SEQ ID NO:11597, SEQ ID NO:11598, SEQ ID NO:11599, SEQ ID NO:11600, SEQ ID NO:11601, SEQ ID NO:11602, SEQ ID NO:11603, SEQ ID NO:11604, SEQ ID NO:11605, SEQ ID NO:11606, SEQ ID NO:11607, SEQ ID NO:11608, SEQ ID NO:11609, SEQ ID NO:11610, SEQ ID NO:11611, SEQ ID NO:11612, SEQ ID NO:11613, SEQ ID NO:11614, SEQ ID NO:11615, SEQ ID NO:11616, SEQ ID NO:11617, SEQ ID NO:11618, SEQ ID NO:11619, SEQ ID NO:11620, SEQ ID NO:11621, SEQ ID NO:11622, SEQ ID NO:11623, SEQ ID NO:11624, SEQ ID NO:11625, SEQ ID NO:11626, SEQ ID NO:11627, SEQ ID NO:11628, SEQ ID NO:11629, SEQ ID NO:11630, SEQ ID NO:11631, SEQ ID NO:11632, SEQ ID NO:11633, SEQ ID NO:11634, SEQ ID NO:11635, SEQ ID NO:11636, SEQ ID NO:11637, SEQ ID NO:11638, SEQ ID NO:11639, SEQ ID NO:11640, SEQ ID NO:11641, SEQ ID NO:11642, SEQ ID NO:11643, SEQ ID NO:11644, SEQ ID NO:11645, SEQ ID NO:11646, SEQ ID NO:11647, SEQ ID NO:11648, SEQ ID NO:11649, SEQ ID NO:11650, SEQ ID NO:11651, SEQ ID NO:11652, SEQ ID NO:11653, SEQ ID NO:11654, SEQ ID NO:11655, SEQ ID NO:11656, SEQ ID NO:11657, SEQ ID NO:11658, SEQ ID NO:11659, SEQ ID NO:11660, SEQ ID NO:11661, SEQ ID NO:11662, SEQ ID NO:11663, SEQ ID NO:11664, SEQ ID NO:11665, SEQ ID NO:11666, SEQ ID NO:11667, SEQ ID NO:11668, SEQ ID NO:11669, SEQ ID NO:11670, SEQ ID NO:11671, SEQ ID NO:11672, SEQ ID NO:11673, SEQ ID NO:11674, SEQ ID NO:11675, SEQ ID NO:11676, SEQ ID NO:11677, SEQ ID NO:11678, SEQ ID NO:11679, SEQ ID NO:11680, SEQ ID NO:11681, SEQ ID NO:11682, SEQ ID NO:11683, SEQ ID NO:11684, SEQ ID NO:11685, SEQ ID NO:11686, SEQ ID NO:11687, SEQ ID NO:11688, SEQ ID NO:11689, SEQ ID NO:11690, SEQ ID NO:11691, SEQ ID NO:11692, SEQ ID NO:11693, SEQ ID NO:11694, SEQ ID NO:11695, SEQ ID NO:11696, SEQ ID NO:11697, SEQ ID NO:11698, SEQ ID NO:11699, SEQ ID NO:11700, SEQ ID NO:11701, SEQ ID NO:11702, SEQ ID NO:11703, SEQ ID NO:11704, SEQ ID NO:11705, SEQ ID NO:11706, SEQ ID NO:11707, SEQ ID NO:11708, SEQ ID NO:11709, SEQ ID NO:11710, SEQ ID NO:11711, SEQ ID NO:11712, SEQ ID NO:11713, SEQ ID NO:11714, SEQ ID NO:11715, SEQ ID NO:11716, SEQ ID NO:11717, SEQ ID NO:11718, SEQ ID NO:11719, SEQ ID NO:11720, SEQ ID NO:11721, SEQ ID NO:11722, SEQ ID NO:11723, SEQ ID NO:11724, SEQ ID NO:11725, SEQ ID NO:11726, SEQ ID NO:11727, SEQ ID NO:11728, SEQ ID NO:11729, SEQ ID NO:11730, SEQ ID NO:11731, SEQ ID NO:11732, SEQ ID NO:11733, SEQ ID NO:11734, SEQ ID NO:11735, SEQ ID NO:11736, SEQ ID NO:11737, SEQ ID NO:11738, SEQ ID NO:11739, SEQ ID NO:11740, SEQ ID NO:11741, SEQ ID NO:11742, SEQ ID NO:11743, SEQ ID NO:11744, SEQ ID NO:11745, SEQ ID NO:11746, SEQ ID NO:11747, SEQ ID NO:11748, SEQ ID NO:11749, SEQ ID NO:11750, SEQ ID NO:11751, SEQ ID NO:11752, SEQ ID NO:11753, SEQ ID NO:11754, SEQ ID NO:11755, SEQ ID NO:11756, SEQ ID NO:11757, SEQ ID NO:11758, SEQ ID NO:11759, SEQ ID NO:11760, SEQ ID NO:11761, SEQ ID NO:11762, SEQ ID NO:11763, SEQ ID NO:11764, SEQ ID NO:11765, SEQ ID NO:11766, SEQ ID NO:11767, SEQ ID NO:11768, SEQ ID NO:11769, SEQ ID NO:11770, SEQ ID NO:11771, SEQ ID NO:11772, SEQ ID NO:11773, SEQ ID NO:11774, SEQ ID NO:11775, SEQ ID NO:11776, SEQ ID NO:11777, SEQ ID NO:11778, SEQ ID NO:11779, SEQ ID NO:11780, SEQ ID NO:11781, SEQ ID NO:11782, SEQ ID NO:11783, SEQ ID NO:11784, SEQ ID NO:11785, SEQ ID NO:11786, SEQ ID NO:11787, SEQ ID NO:11788, SEQ ID NO:11789, SEQ ID NO:11790, SEQ ID NO:11791, SEQ ID NO:11792, SEQ ID NO:11793, SEQ ID NO:11794, SEQ ID NO:11795, SEQ ID NO:11796, SEQ ID NO:11797, SEQ ID NO:11798, SEQ ID NO:11799, SEQ ID NO:11800, SEQ ID NO:11801, SEQ ID NO:11802, SEQ ID NO:11803, SEQ ID NO:11804, SEQ ID NO:11805, SEQ ID NO:11806, SEQ ID NO:11807, SEQ ID NO:11808, SEQ ID NO:11809, SEQ ID NO:11810, SEQ ID NO:11811, SEQ ID NO:11812, SEQ ID NO:11813, SEQ ID NO:11814, SEQ ID NO:11815, SEQ ID NO:11816, SEQ ID NO:11817, SEQ ID NO:11818, SEQ ID NO:11819, SEQ ID NO:11820, SEQ ID NO:11821, SEQ ID NO:11822, SEQ ID NO:11823, SEQ ID NO:11824, SEQ ID NO:11825, SEQ ID NO:11826, SEQ ID NO:11827, SEQ ID NO:11828, SEQ ID NO:11829, SEQ ID NO:11830, SEQ ID NO:11831, SEQ ID NO:11832, SEQ ID NO:11833, SEQ ID NO:11834, SEQ ID NO:11835, SEQ ID NO:11836, SEQ ID NO:11837, SEQ ID NO:11838, SEQ ID NO:11839, SEQ ID NO:11840, SEQ ID NO:11841, SEQ ID NO:11842, SEQ ID NO:11843, SEQ ID NO:11844, SEQ ID NO:11845, SEQ ID NO:11846, SEQ ID NO:11847, SEQ ID NO:11848, SEQ ID NO:11849, SEQ ID NO:11850, SEQ ID NO:11851, SEQ ID NO:11852, SEQ ID NO:11853, SEQ ID NO:11854, SEQ ID NO:11855, SEQ ID NO:11856, SEQ ID NO:11857, SEQ ID NO:11858, SEQ ID NO:11859, SEQ ID NO:11860, SEQ ID NO:11861, SEQ ID NO:11862, SEQ ID NO:11863, SEQ ID NO:11864, SEQ ID NO:11865, SEQ ID NO:11866, SEQ ID NO:11867, SEQ ID NO:11868, SEQ ID NO:11869, SEQ ID NO:11870, SEQ ID NO:11871, SEQ ID NO:11872, SEQ ID NO:11873, SEQ ID NO:11874, SEQ ID NO:11875, SEQ ID NO:11876, SEQ ID NO:11877, SEQ ID NO:11878, SEQ ID NO:11879, SEQ ID NO:11880, SEQ ID NO:11881, SEQ ID NO:11882, SEQ ID NO:11883, SEQ ID NO:11884, SEQ ID NO:11885, SEQ ID NO:11886, SEQ ID NO:11887, SEQ ID NO:11888, SEQ ID NO:11889, SEQ ID NO:11890, SEQ ID NO:11891, SEQ ID NO:11892, SEQ ID NO:11893, SEQ ID NO:11894, SEQ ID NO:11895, SEQ ID NO:11896, SEQ ID NO:11897, SEQ ID NO:11898, SEQ ID NO:11899, SEQ ID NO:11900, SEQ ID NO:11901, SEQ ID NO:11902, SEQ ID NO:11903, SEQ ID NO:11904, SEQ ID NO:11905, SEQ ID NO:11906, SEQ ID NO:11907, SEQ ID NO:11908, SEQ ID NO:11909, SEQ ID NO:11910, SEQ ID NO:11911, SEQ ID NO:11912, SEQ ID NO:11913, SEQ ID NO:11914, SEQ ID NO:11915, SEQ ID NO:11916, SEQ ID NO:11917, SEQ ID NO:11918, SEQ ID NO:11919, SEQ ID NO:11920, SEQ ID NO:11921, SEQ ID NO:11922, SEQ ID NO:11923, SEQ ID NO:11924, SEQ ID NO:11925, SEQ ID NO:11926, SEQ ID NO:11927, SEQ ID NO:11928, SEQ ID NO:11929, SEQ ID NO:11930, SEQ ID NO:11931, SEQ ID NO:11932, SEQ ID NO:11933, SEQ ID NO:11934, SEQ ID NO:11935, SEQ ID NO:11936, SEQ ID NO:11937, SEQ ID NO:11938, SEQ ID NO:11939, SEQ ID NO:11940, SEQ ID NO:11941, SEQ ID NO:11942, SEQ ID NO:11943, SEQ ID NO:11944, SEQ ID NO:11945, SEQ ID NO:11946, SEQ ID NO:11947, SEQ ID NO:11948, SEQ ID NO:11949, SEQ ID NO:11950, SEQ ID NO:11951, SEQ ID NO:11952, SEQ ID NO:11953, SEQ ID NO:11954, SEQ ID NO:11955, SEQ ID NO:11956, SEQ ID NO:11957, SEQ ID NO:11958, SEQ ID NO:11959, SEQ ID NO:11960, SEQ ID NO:11961, SEQ ID NO:11962, SEQ ID NO:11963, SEQ ID NO:11964, SEQ ID NO:11965, SEQ ID NO:11966, SEQ ID NO:11967, SEQ ID NO:11968, SEQ ID NO:11969, SEQ ID NO:11970, SEQ ID NO:11971, SEQ ID NO:11972, SEQ ID NO:11973, SEQ ID NO:11974, SEQ ID NO:11975, SEQ ID NO:11976, SEQ ID NO:11977, SEQ ID NO:11978, SEQ ID NO:11979, SEQ ID NO:11980, SEQ ID NO:11981, SEQ ID NO:11982, SEQ ID NO:11983, SEQ ID NO:11984, SEQ ID NO:11985, SEQ ID NO:11986, SEQ ID NO:11987, SEQ ID NO:11988, SEQ ID NO:11989, SEQ ID NO:11990, SEQ ID NO:11991, SEQ ID NO:11992, SEQ ID NO:11993, SEQ ID NO:11994, SEQ ID NO:11995, SEQ ID NO:11996, SEQ ID NO:11997, SEQ ID NO:11998, SEQ ID NO:11999, SEQ ID NO:12000, SEQ ID NO:12001, SEQ ID NO:12002, SEQ ID NO:12003, SEQ ID NO:12004, SEQ ID NO:12005, SEQ ID NO:12006, SEQ ID NO:12007, SEQ ID NO:12008, SEQ ID NO:12009, SEQ ID NO:12010, SEQ ID NO:12011, SEQ ID NO:12012, SEQ ID NO:12013, SEQ ID NO:12014, SEQ ID NO:12015, SEQ ID NO:12016, SEQ ID NO:12017, SEQ ID NO:12018, SEQ ID NO:12019, SEQ ID NO:12020, SEQ ID NO:12021, SEQ ID NO:12022, SEQ ID NO:12023, SEQ ID NO:12024, SEQ ID NO:12025, SEQ ID NO:12026, SEQ ID NO:12027, SEQ ID NO:12028, SEQ ID NO:12029, SEQ ID NO:12030, SEQ ID NO:12031, SEQ ID NO:12032, SEQ ID NO:12033, SEQ ID NO:12034, SEQ ID NO:12035, SEQ ID NO:12036, SEQ ID NO:12037, SEQ ID NO:12038, SEQ ID NO:12039, SEQ ID NO:12040, SEQ ID NO:12041, SEQ ID NO:12042, SEQ ID NO:12043, SEQ ID NO:12044, SEQ ID NO:12045, SEQ ID NO:12046, SEQ ID NO:12047, SEQ ID NO:12048, SEQ ID NO:12049, SEQ ID NO:12050, SEQ ID NO:12051, SEQ ID NO:12052, SEQ ID NO:12053, SEQ ID NO:12054, SEQ ID NO:12055, SEQ ID NO:12056, SEQ ID NO:12057, SEQ ID NO:12058, SEQ ID NO:12059, SEQ ID NO:12060, SEQ ID NO:12061, SEQ ID NO:12062, SEQ ID NO:12063, SEQ ID NO:12064, SEQ ID NO:12065, SEQ ID NO:12066, SEQ ID NO:12067, SEQ ID NO:12068, SEQ ID NO:12069, SEQ ID NO:12070, SEQ ID NO:12071, SEQ ID NO:12072, SEQ ID NO:12073, SEQ ID NO:12074, SEQ ID NO:12075, SEQ ID NO:12076, SEQ ID NO:12077, SEQ ID NO:12078, SEQ ID NO:12079, SEQ ID NO:12080, SEQ ID NO:12081, SEQ ID NO:12082, SEQ ID NO:12083, SEQ ID NO:12084, SEQ ID NO:12085, SEQ ID NO:12086, SEQ ID NO:12087, SEQ ID NO:12088, SEQ ID NO:12089, SEQ ID NO:12090, SEQ ID NO:12091, SEQ ID NO:12092, SEQ ID NO:12093, SEQ ID NO:12094, SEQ ID NO:12095, SEQ ID NO:12096, SEQ ID NO:12097, SEQ ID NO:12098, SEQ ID NO:12099, SEQ ID NO:12100, SEQ ID NO:12101, SEQ ID NO:12102, SEQ ID NO:12103, SEQ ID NO:12104, SEQ ID NO:12105, SEQ ID NO:12106, SEQ ID NO:12107, SEQ ID NO:12108, SEQ ID NO:12109, SEQ ID NO:12110, SEQ ID NO:12111, SEQ ID NO:12112, SEQ ID NO:12113, SEQ ID NO:12114, SEQ ID NO:12115, SEQ ID NO:12116, SEQ ID NO:12117, SEQ ID NO:12118, SEQ ID NO:12119, SEQ ID NO:12120, SEQ ID NO:12121, SEQ ID NO:12122, SEQ ID NO:12123, SEQ ID NO:12124, SEQ ID NO:12125, SEQ ID NO:12126, SEQ ID NO:12127, SEQ ID NO:12128, SEQ ID NO:12129, SEQ ID NO:12130, SEQ ID NO:12131, SEQ ID NO:12132, SEQ ID NO:12133, SEQ ID NO:12134, SEQ ID NO:12135, SEQ ID NO:12136, SEQ ID NO:12137, SEQ ID NO:12138, SEQ ID NO:12139, SEQ ID NO:12140, SEQ ID NO:12141, SEQ ID NO:12142, SEQ ID NO:12143, SEQ ID NO:12144, SEQ ID NO:12145, SEQ ID NO:12146, SEQ ID NO:12147, SEQ ID NO:12148, SEQ ID NO:12149, SEQ ID NO:12150, SEQ ID NO:12151, SEQ ID NO:12152, SEQ ID NO:12153, SEQ ID NO:12154, SEQ ID NO:12155, SEQ ID NO:12156, SEQ ID NO:12157, SEQ ID NO:12158, SEQ ID NO:12159, SEQ ID NO:12160, SEQ ID NO:12161, SEQ ID NO:12162, SEQ ID NO:12163, SEQ ID NO:12164, SEQ ID NO:12165, SEQ ID NO:12166, SEQ ID NO:12167, SEQ ID NO:12168, SEQ ID NO:12169, SEQ ID NO:12170, SEQ ID NO:12171, SEQ ID NO:12172, SEQ ID NO:12173, SEQ ID NO:12174, SEQ ID NO:12175, SEQ ID NO:12176, SEQ ID NO:12177, SEQ ID NO:12178, SEQ ID NO:12179, SEQ ID NO:12180, SEQ ID NO:12181, SEQ ID NO:12182, SEQ ID NO:12183, SEQ ID NO:12184, SEQ ID NO:12185, SEQ ID NO:12186, SEQ ID NO:12187, SEQ ID NO:12188, SEQ ID NO:12189, SEQ ID NO:12190, SEQ ID NO:12191, SEQ ID NO:12192, SEQ ID NO:12193, SEQ ID NO:12194, SEQ ID NO:12195, SEQ ID NO:12196, SEQ ID NO:12197, SEQ ID NO:12198, SEQ ID NO:12199, SEQ ID NO:12200, SEQ ID NO:12201, SEQ ID NO:12202, SEQ ID NO:12203, SEQ ID NO:12204, SEQ ID NO:12205, SEQ ID NO:12206, SEQ ID NO:12207, SEQ ID NO:12208, SEQ ID NO:12209, SEQ ID NO:12210, SEQ ID NO:12211, SEQ ID NO:12212, SEQ ID NO:12213, SEQ ID NO:12214, SEQ ID NO:12215, SEQ ID NO:12216, SEQ ID NO:12217, SEQ ID NO:12218, SEQ ID NO:12219, SEQ ID NO:12220, SEQ ID NO:12221, SEQ ID NO:12222, SEQ ID NO:12223, SEQ ID NO:12224, SEQ ID NO:12225, SEQ ID NO:12226, SEQ ID NO:12227, SEQ ID NO:12228, SEQ ID NO:12229, SEQ ID NO:12230, SEQ ID NO:12231, SEQ ID NO:12232, SEQ ID NO:12233, SEQ ID NO:12234, SEQ ID NO:12235, SEQ ID NO:12236, SEQ ID NO:12237, SEQ ID NO:12238, SEQ ID NO:12239, SEQ ID NO:12240, SEQ ID NO:12241, SEQ ID NO:12242, SEQ ID NO:12243, SEQ ID NO:12244, SEQ ID NO:12245, SEQ ID NO:12246, SEQ ID NO:12247, SEQ ID NO:12248, SEQ ID NO:12249, SEQ ID NO:12250, SEQ ID NO:12251, SEQ ID NO:12252, SEQ ID NO:12253, SEQ ID NO:12254, SEQ ID NO:12255, SEQ ID NO:12256, SEQ ID NO:12257, SEQ ID NO:12258, SEQ ID NO:12259, SEQ ID NO:12260, SEQ ID NO:12261, SEQ ID NO:12262, SEQ ID NO:12263, SEQ ID NO:12264, SEQ ID NO:12265, SEQ ID NO:12266, SEQ ID NO:12267, SEQ ID NO:12268, SEQ ID NO:12269, SEQ ID NO:12270, SEQ ID NO:12271, SEQ ID NO:12272, SEQ ID NO:12273, SEQ ID NO:12274, SEQ ID NO:12275, SEQ ID NO:12276, SEQ ID NO:12277, SEQ ID NO:12278, SEQ ID NO:12279, SEQ ID NO:12280, SEQ ID NO:12281, SEQ ID NO:12282, SEQ ID NO:12283, SEQ ID NO:12284, SEQ ID NO:12285, SEQ ID NO:12286, SEQ ID NO:12287, SEQ ID NO:12288, SEQ ID NO:12289, SEQ ID NO:12290, SEQ ID NO:12291, SEQ ID NO:12292, SEQ ID NO:12293, SEQ ID NO:12294, SEQ ID NO:12295, SEQ ID NO:12296, SEQ ID NO:12297, SEQ ID NO:12298, SEQ ID NO:12299, SEQ ID NO:12300, SEQ ID NO:12301, SEQ ID NO:12302, SEQ ID NO:12303, SEQ ID NO:12304, SEQ ID NO:12305, SEQ ID NO:12306, SEQ ID NO:12307, SEQ ID NO:12308, SEQ ID NO:12309, SEQ ID NO:12310, SEQ ID NO:12311, SEQ ID NO:12312, SEQ ID NO:12313, SEQ ID NO:12314, SEQ ID NO:12315, SEQ ID NO:12316, SEQ ID NO:12317, SEQ ID NO:12318, SEQ ID NO:12319, SEQ ID NO:12320, SEQ ID NO:12321, SEQ ID NO:12322, SEQ ID NO:12323, SEQ ID NO:12324, SEQ ID NO:12325, SEQ ID NO:12326, SEQ ID NO:12327, SEQ ID NO:12328, SEQ ID NO:12329, SEQ ID NO:12330, SEQ ID NO:12331, SEQ ID NO:12332, SEQ ID NO:12333, SEQ ID NO:12334, SEQ ID NO:12335, SEQ ID NO:12336, SEQ ID NO:12337, SEQ ID NO:12338, SEQ ID NO:12339, SEQ ID NO:12340, SEQ ID NO:12341, SEQ ID NO:12342, SEQ ID NO:12343, SEQ ID NO:12344, SEQ ID NO:12345, SEQ ID NO:12346, SEQ ID NO:12347, SEQ ID NO:12348, SEQ ID NO:12349, SEQ ID NO:12350, SEQ ID NO:12351, SEQ ID NO:12352, SEQ ID NO:12353, SEQ ID NO:12354, SEQ ID NO:12355, SEQ ID NO:12356, SEQ ID NO:12357, SEQ ID NO:12358, SEQ ID NO:12359, SEQ ID NO:12360, SEQ ID NO:12361, SEQ ID NO:12362, SEQ ID NO:12363, SEQ ID NO:12364, SEQ ID NO:12365, SEQ ID NO:12366, SEQ ID NO:12367, SEQ ID NO:12368, SEQ ID NO:12369, SEQ ID NO:12370, SEQ ID NO:12371, SEQ ID NO:12372, SEQ ID NO:12373, SEQ ID NO:12374, SEQ ID NO:12375, SEQ ID NO:12376, SEQ ID NO:12377, SEQ ID NO:12378, SEQ ID NO:12379, SEQ ID NO:12380, SEQ ID NO:12381, SEQ ID NO:12382, SEQ ID NO:12383, SEQ ID NO:12384, SEQ ID NO:12385, SEQ ID NO:12386, SEQ ID NO:12387, SEQ ID NO:12388, SEQ ID NO:12389, SEQ ID NO:12390, SEQ ID NO:12391, SEQ ID NO:12392, SEQ ID NO:12393, SEQ ID NO:12394, SEQ ID NO:12395, SEQ ID NO:12396, SEQ ID NO:12397, SEQ ID NO:12398, SEQ ID NO:12399, SEQ ID NO:12400, SEQ ID NO:12401, SEQ ID NO:12402, SEQ ID NO:12403, SEQ ID NO:12404, SEQ ID NO:12405, SEQ ID NO:12406, SEQ ID NO:12407, SEQ ID NO:12408, SEQ ID NO:12409, SEQ ID NO:12410, SEQ ID NO:12411, SEQ ID NO:12412, SEQ ID NO:12413, SEQ ID NO:12414, SEQ ID NO:12415, SEQ ID NO:12416, SEQ ID NO:12417, SEQ ID NO:12418, SEQ ID NO:12419, SEQ ID NO:12420, SEQ ID NO:12421, SEQ ID NO:12422, SEQ ID NO:12423, SEQ ID NO:12424, SEQ ID NO:12425, SEQ ID NO:12426, SEQ ID NO:12427, SEQ ID NO:12428, SEQ ID NO:12429, SEQ ID NO:12430, SEQ ID NO:12431, SEQ ID NO:12432, SEQ ID NO:12433, SEQ ID NO:12434, SEQ ID NO:12435, SEQ ID NO:12436, SEQ ID NO:12437, SEQ ID NO:12438, SEQ ID NO:12439, SEQ ID NO:12440, SEQ ID NO:12441, SEQ ID NO:12442, SEQ ID NO:12443, SEQ ID NO:12444, SEQ ID NO:12445, SEQ ID NO:12446, SEQ ID NO:12447, SEQ ID NO:12448, SEQ ID NO:12449, SEQ ID NO:12450, SEQ ID NO:12451, SEQ ID NO:12452, SEQ ID NO:12453, SEQ ID NO:12454, SEQ ID NO:12455, SEQ ID NO:12456, SEQ ID NO:12457, SEQ ID NO:12458, SEQ ID NO:12459, SEQ ID NO:12460, SEQ ID NO:12461, SEQ ID NO:12462, SEQ ID NO:12463, SEQ ID NO:12464, SEQ ID NO:12465, SEQ ID NO:12466, SEQ ID NO:12467, SEQ ID NO:12468, SEQ ID NO:12469, SEQ ID NO:12470, SEQ ID NO:12471, SEQ ID NO:12472, SEQ ID NO:12473, SEQ ID NO:12474, SEQ ID NO:12475, SEQ ID NO:12476, SEQ ID NO:12477, SEQ ID NO:12478, SEQ ID NO:12479, SEQ ID NO:12480, SEQ ID NO:12481, SEQ ID NO:12482, SEQ ID NO:12483, SEQ ID NO:12484, SEQ ID NO:12485, SEQ ID NO:12486, SEQ ID NO:12487, SEQ ID NO:12488, SEQ ID NO:12489, SEQ ID NO:12490, SEQ ID NO:12491, SEQ ID NO:12492, SEQ ID NO:12493, SEQ ID NO:12494, SEQ ID NO:12495, SEQ ID NO:12496, SEQ ID NO:12497, SEQ ID NO:12498, SEQ ID NO:12499, SEQ ID NO:12500, SEQ ID NO:12501, SEQ ID NO:12502, SEQ ID NO:12503, SEQ ID NO:12504, SEQ ID NO:12505, SEQ ID NO:12506, SEQ ID NO:12507, SEQ ID NO:12508, SEQ ID NO:12509, SEQ ID NO:12510, SEQ ID NO:12511, SEQ ID NO:12512, SEQ ID NO:12513, SEQ ID NO:12514, SEQ ID NO:12515, SEQ ID NO:12516, SEQ ID NO:12517, SEQ ID NO:12518, SEQ ID NO:12519, SEQ ID NO:12520, SEQ ID NO:12521, SEQ ID NO:12522, SEQ ID NO:12523, SEQ ID NO:12524, SEQ ID NO:12525, SEQ ID NO:12526, SEQ ID NO:12527, SEQ ID NO:12528, SEQ ID NO:12529, SEQ ID NO:12530, SEQ ID NO:12531, SEQ ID NO:12532, SEQ ID NO:12533, SEQ ID NO:12534, SEQ ID NO:12535, SEQ ID NO:12536, SEQ ID NO:12537, SEQ ID NO:12538, SEQ ID NO:12539, SEQ ID NO:12540, SEQ ID NO:12541, SEQ ID NO:12542, SEQ ID NO:12543, SEQ ID NO:12544, SEQ ID NO:12545, SEQ ID NO:12546, SEQ ID NO:12547, SEQ ID NO:12548, SEQ ID NO:12549, SEQ ID NO:12550, SEQ ID NO:12551, SEQ ID NO:12552, SEQ ID NO:12553, SEQ ID NO:12554, SEQ ID NO:12555, SEQ ID NO:12556, SEQ ID NO:12557, SEQ ID NO:12558, SEQ ID NO:12559, SEQ ID NO:12560, SEQ ID NO:12561, SEQ ID NO:12562, SEQ ID NO:12563, SEQ ID NO:12564, SEQ ID NO:12565, SEQ ID NO:12566, SEQ ID NO:12567, SEQ ID NO:12568, SEQ ID NO:12569, SEQ ID NO:12570, SEQ ID NO:12571, SEQ ID NO:12572, SEQ ID NO:12573, SEQ ID NO:12574, SEQ ID NO:12575, SEQ ID NO:12576, SEQ ID NO:12577, SEQ ID NO:12578, SEQ ID NO:12579, SEQ ID NO:12580, SEQ ID NO:12581, SEQ ID NO:12582, SEQ ID NO:12583, SEQ ID NO:12584, SEQ ID NO:12585, SEQ ID NO:12586, SEQ ID NO:12587, SEQ ID NO:12588, SEQ ID NO:12589, SEQ ID NO:12590, SEQ ID NO:12591, SEQ ID NO:12592, SEQ ID NO:12593, SEQ ID NO:12594, SEQ ID NO:12595, SEQ ID NO:12596, SEQ ID NO:12597, SEQ ID NO:12598, SEQ ID NO:12599, SEQ ID NO:12600, SEQ ID NO:12601, SEQ ID NO:12602, SEQ ID NO:12603, SEQ ID NO:12604, SEQ ID NO:12605, SEQ ID NO:12606, SEQ ID NO:12607, SEQ ID NO:12608, SEQ ID NO:12609, SEQ ID NO:12610, SEQ ID NO:12611, SEQ ID NO:12612, SEQ ID NO:12613, SEQ ID NO:12614, SEQ ID NO:12615, SEQ ID NO:12616, SEQ ID NO:12617, SEQ ID NO:12618, SEQ ID NO:12619, SEQ ID NO:12620, SEQ ID NO:12621, SEQ ID NO:12622, SEQ ID NO:12623, SEQ ID NO:12624, SEQ ID NO:12625, SEQ ID NO:12626, SEQ ID NO:12627, SEQ ID NO:12628, SEQ ID NO:12629, SEQ ID NO:12630, SEQ ID NO:12631, SEQ ID NO:12632, SEQ ID NO:12633, SEQ ID NO:12634, SEQ ID NO:12635, SEQ ID NO:12636, SEQ ID NO:12637, SEQ ID NO:12638, SEQ ID NO:12639, SEQ ID NO:12640, SEQ ID NO:12641, SEQ ID NO:12642, SEQ ID NO:12643, SEQ ID NO:12644, SEQ ID NO:12645, SEQ ID NO:12646, SEQ ID NO:12647, SEQ ID NO:12648, SEQ ID NO:12649, SEQ ID NO:12650, SEQ ID NO:12651, SEQ ID NO:12652, SEQ ID NO:12653, SEQ ID NO:12654, SEQ ID NO:12655, SEQ ID NO:12656, SEQ ID NO:12657, SEQ ID NO:12658, SEQ ID NO:12659, SEQ ID NO:12660, SEQ ID NO:12661, SEQ ID NO:12662, SEQ ID NO:12663, SEQ ID NO:12664, SEQ ID NO:12665, SEQ ID NO:12666, SEQ ID NO:12667, SEQ ID NO:12668, SEQ ID NO:12669, SEQ ID NO:12670, SEQ ID NO:12671, SEQ ID NO:12672, SEQ ID NO:12673, SEQ ID NO:12674, SEQ ID NO:12675, SEQ ID NO:12676, SEQ ID NO:12677, SEQ ID NO:12678, SEQ ID NO:12679, SEQ ID NO:12680, SEQ ID NO:12681, SEQ ID NO:12682, SEQ ID NO:12683, SEQ ID NO:12684, SEQ ID NO:12685, SEQ ID NO:12686, SEQ ID NO:12687, SEQ ID NO:12688, SEQ ID NO:12689, SEQ ID NO:12690, SEQ ID NO:12691, SEQ ID NO:12692, SEQ ID NO:12693, SEQ ID NO:12694, SEQ ID NO:12695, SEQ ID NO:12696, SEQ ID NO:12697, SEQ ID NO:12698, SEQ ID NO:12699, SEQ ID NO:12700, SEQ ID NO:12701, SEQ ID NO:12702, SEQ ID NO:12703, SEQ ID NO:12704, SEQ ID NO:12705, SEQ ID NO:12706, SEQ ID NO:12707, SEQ ID NO:12708, SEQ ID NO:12709, SEQ ID NO:12710, SEQ ID NO:12711, SEQ ID NO:12712, SEQ ID NO:12713, SEQ ID NO:12714, SEQ ID NO:12715, SEQ ID NO:12716, SEQ ID NO:12717, SEQ ID NO:12718, SEQ ID NO:12719, SEQ ID NO:12720, SEQ ID NO:12721, SEQ ID NO:12722, SEQ ID NO:12723, SEQ ID NO:12724, SEQ ID NO:12725, SEQ ID NO:12726, SEQ ID NO:12727, SEQ ID NO:12728, SEQ ID NO:12729, SEQ ID NO:12730, SEQ ID NO:12731, SEQ ID NO:12732, SEQ ID NO:12733, SEQ ID NO:12734, SEQ ID NO:12735, SEQ ID NO:12736, SEQ ID NO:12737, SEQ ID NO:12738, SEQ ID NO:12739, SEQ ID NO:12740, SEQ ID NO:12741, SEQ ID NO:12742, SEQ ID NO:12743, SEQ ID NO:12744, SEQ ID NO:12745, SEQ ID NO:12746, SEQ ID NO:12747, SEQ ID NO:12748, SEQ ID NO:12749, SEQ ID NO:12750, SEQ ID NO:12751, SEQ ID NO:12752, SEQ ID NO:12753, SEQ ID NO:12754, SEQ ID NO:12755, SEQ ID NO:12756, SEQ ID NO:12757, SEQ ID NO:12758, SEQ ID NO:12759, SEQ ID NO:12760, SEQ ID NO:12761, SEQ ID NO:12762, SEQ ID NO:12763, SEQ ID NO:12764, SEQ ID NO:12765, SEQ ID NO:12766, SEQ ID NO:12767, SEQ ID NO:12768, SEQ ID NO:12769, SEQ ID NO:12770, SEQ ID NO:12771, SEQ ID NO:12772, SEQ ID NO:12773, SEQ ID NO:12774, SEQ ID NO:12775, SEQ ID NO:12776, SEQ ID NO:12777, SEQ ID NO:12778, SEQ ID NO:12779, SEQ ID NO:12780, SEQ ID NO:12781, SEQ ID NO:12782, SEQ ID NO:12783, SEQ ID NO:12784, SEQ ID NO:12785, SEQ ID NO:12786, SEQ ID NO:12787, SEQ ID NO:12788, SEQ ID NO:12789, SEQ ID NO:12790, SEQ ID NO:12791, SEQ ID NO:12792, SEQ ID NO:12793, SEQ ID NO:12794, SEQ ID NO:12795, SEQ ID NO:12796, SEQ ID NO:12797, SEQ ID NO:12798, SEQ ID NO:12799, SEQ ID NO:12800, SEQ ID NO:12801, SEQ ID NO:12802, SEQ ID NO:12803, SEQ ID NO:12804, SEQ ID NO:12805, SEQ ID NO:12806, SEQ ID NO:12807, SEQ ID NO:12808, SEQ ID NO:12809, SEQ ID NO:12810, SEQ ID NO:12811, SEQ ID NO:12812, SEQ ID NO:12813, SEQ ID NO:12814, SEQ ID NO:12815, SEQ ID NO:12816, SEQ ID NO:12817, SEQ ID NO:12818, SEQ ID NO:12819, SEQ ID NO:12820, SEQ ID NO:12821, SEQ ID NO:12822, SEQ ID NO:12823, SEQ ID NO:12824, SEQ ID NO:12825, SEQ ID NO:12826, SEQ ID NO:12827, SEQ ID NO:12828, SEQ ID NO:12829, SEQ ID NO:12830, SEQ ID NO:12831, SEQ ID NO:12832, SEQ ID NO:12833, SEQ ID NO:12834, SEQ ID NO:12835, SEQ ID NO:12836, SEQ ID NO:12837, SEQ ID NO:12838, SEQ ID NO:12839, SEQ ID NO:12840, SEQ ID NO:12841, SEQ ID NO:12842, SEQ ID NO:12843, SEQ ID NO:12844, SEQ ID NO:12845, SEQ ID NO:12846, SEQ ID NO:12847, SEQ ID NO:12848, SEQ ID NO:12849, SEQ ID NO:12850, SEQ ID NO:12851, SEQ ID NO:12852, SEQ ID NO:12853, SEQ ID NO:12854, SEQ ID NO:12855, SEQ ID NO:12856, SEQ ID NO:12857, SEQ ID NO:12858, SEQ ID NO:12859, SEQ ID NO:12860, SEQ ID NO:12861, SEQ ID NO:12862, SEQ ID NO:12863, SEQ ID NO:12864, SEQ ID NO:12865, SEQ ID NO:12866, SEQ ID NO:12867, SEQ ID NO:12868, SEQ ID NO:12869, SEQ ID NO:12870, SEQ ID NO:12871, SEQ ID NO:12872, SEQ ID NO:12873, SEQ ID NO:12874, SEQ ID NO:12875, SEQ ID NO:12876, SEQ ID NO:12877, SEQ ID NO:12878, SEQ ID NO:12879, SEQ ID NO:12880, SEQ ID NO:12881, SEQ ID NO:12882, SEQ ID NO:12883, SEQ ID NO:12884, SEQ ID NO:12885, SEQ ID NO:12886, SEQ ID NO:12887, SEQ ID NO:12888, SEQ ID NO:12889, SEQ ID NO:12890, SEQ ID NO:12891, SEQ ID NO:12892, SEQ ID NO:12893, SEQ ID NO:12894, SEQ ID NO:12895, SEQ ID NO:12896, SEQ ID NO:12897, SEQ ID NO:12898, SEQ ID NO:12899, SEQ ID NO:12900, SEQ ID NO:12901, SEQ ID NO:12902, SEQ ID NO:12903, SEQ ID NO:12904, SEQ ID NO:12905, SEQ ID NO:12906, SEQ ID NO:12907, SEQ ID NO:12908, SEQ ID NO:12909, SEQ ID NO:12910, SEQ ID NO:12911, SEQ ID NO:12912, SEQ ID NO:12913, SEQ ID NO:12914, SEQ ID NO:12915, SEQ ID NO:12916, SEQ ID NO:12917, SEQ ID NO:12918, SEQ ID NO:12919, SEQ ID NO:12920, SEQ ID NO:12921, SEQ ID NO:12922, SEQ ID NO:12923, SEQ ID NO:12924, SEQ ID NO:12925, SEQ ID NO:12926, SEQ ID NO:12927, SEQ ID NO:12928, SEQ ID NO:12929, SEQ ID NO:12930, SEQ ID NO:12931, SEQ ID NO:12932, SEQ ID NO:12933, SEQ ID NO:12934, SEQ ID NO:12935, SEQ ID NO:12936, SEQ ID NO:12937, SEQ ID NO:12938, SEQ ID NO:12939, SEQ ID NO:12940, SEQ ID NO:12941, SEQ ID NO:12942, SEQ ID NO:12943, SEQ ID NO:12944, SEQ ID NO:12945, SEQ ID NO:12946, SEQ ID NO:12947, SEQ ID NO:12948, SEQ ID NO:12949, SEQ ID NO:12950, SEQ ID NO:12951, SEQ ID NO:12952, SEQ ID NO:12953, SEQ ID NO:12954, SEQ ID NO:12955, SEQ ID NO:12956, SEQ ID NO:12957, SEQ ID NO:12958, SEQ ID NO:12959, SEQ ID NO:12960, SEQ ID NO:12961, SEQ ID NO:12962, SEQ ID NO:12963, SEQ ID NO:12964, SEQ ID NO:12965, SEQ ID NO:12966, SEQ ID NO:12967, SEQ ID NO:12968, SEQ ID NO:12969, SEQ ID NO:12970, SEQ ID NO:12971, SEQ ID NO:12972, SEQ ID NO:12973, SEQ ID NO:12974, SEQ ID NO:12975, SEQ ID NO:12976, SEQ ID NO:12977, SEQ ID NO:12978, SEQ ID NO:12979, SEQ ID NO:12980, SEQ ID NO:12981, SEQ ID NO:12982, SEQ ID NO:12983, SEQ ID NO:12984, SEQ ID NO:12985, SEQ ID NO:12986, SEQ ID NO:12987, SEQ ID NO:12988, SEQ ID NO:12989, SEQ ID NO:12990, SEQ ID NO:12991, SEQ ID NO:12992, SEQ ID NO:12993, SEQ ID NO:12994, SEQ ID NO:12995, SEQ ID NO:12996, SEQ ID NO:12997, SEQ ID NO:12998, SEQ ID NO:12999, SEQ ID NO:13000, SEQ ID NO:13001, SEQ ID NO:13002, SEQ ID NO:13003, SEQ ID NO:13004, SEQ ID NO:13005, SEQ ID NO:13006, SEQ ID NO:13007, SEQ ID NO:13008, SEQ ID NO:13009, SEQ ID NO:13010, SEQ ID NO:13011, SEQ ID NO:13012, SEQ ID NO:13013, SEQ ID NO:13014, SEQ ID NO:13015, SEQ ID NO:13016, SEQ ID NO:13017, SEQ ID NO:13018, SEQ ID NO:13019, SEQ ID NO:13020, SEQ ID NO:13021, SEQ ID NO:13022, SEQ ID NO:13023, SEQ ID NO:13024, SEQ ID NO:13025, SEQ ID NO:13026, SEQ ID NO:13027, SEQ ID NO:13028, SEQ ID NO:13029, SEQ ID NO:13030, SEQ ID NO:13031, SEQ ID NO:13032, SEQ ID NO:13033, SEQ ID NO:13034, SEQ ID NO:13035, SEQ ID NO:13036, SEQ ID NO:13037, SEQ ID NO:13038, SEQ ID NO:13039, SEQ ID NO:13040, SEQ ID NO:13041, SEQ ID NO:13042, SEQ ID NO:13043, SEQ ID NO:13044, SEQ ID NO:13045, SEQ ID NO:13046, SEQ ID NO:13047, SEQ ID NO:13048, SEQ ID NO:13049, SEQ ID NO:13050, SEQ ID NO:13051, SEQ ID NO:13052, SEQ ID NO:13053, SEQ ID NO:13054, SEQ ID NO:13055, SEQ ID NO:13056, SEQ ID NO:13057, SEQ ID NO:13058, SEQ ID NO:13059, SEQ ID NO:13060, SEQ ID NO:13061, SEQ ID NO:13062, SEQ ID NO:13063, SEQ ID NO:13064, SEQ ID NO:13065, SEQ ID NO:13066, SEQ ID NO:13067, SEQ ID NO:13068, SEQ ID NO:13069, SEQ ID NO:13070, SEQ ID NO:13071, SEQ ID NO:13072, SEQ ID NO:13073, SEQ ID NO:13074, SEQ ID NO:13075, SEQ ID NO:13076, SEQ ID NO:13077, SEQ ID NO:13078, SEQ ID NO:13079, SEQ ID NO:13080, SEQ ID NO:13081, SEQ ID NO:13082, SEQ ID NO:13083, SEQ ID NO:13084, SEQ ID NO:13085, SEQ ID NO:13086, SEQ ID NO:13087, SEQ ID NO:13088, SEQ ID NO:13089, SEQ ID NO:13090, SEQ ID NO:13091, SEQ ID NO:13092, SEQ ID NO:13093, SEQ ID NO:13094, SEQ ID NO:13095, SEQ ID NO:13096, SEQ ID NO:13097, SEQ ID NO:13098, SEQ ID NO:13099, SEQ ID NO:13100, SEQ ID NO:13101, SEQ ID NO:13102, SEQ ID NO:13103, SEQ ID NO:13104, SEQ ID NO:13105, SEQ ID NO:13106, SEQ ID NO:13107, SEQ ID NO:13108, SEQ ID NO:13109, SEQ ID NO:13110, SEQ ID NO:13111, SEQ ID NO:13112, SEQ ID NO:13113, SEQ ID NO:13114, SEQ ID NO:13115, SEQ ID NO:13116, SEQ ID NO:13117, SEQ ID NO:13118, SEQ ID NO:13119, SEQ ID NO:13120, SEQ ID NO:13121, SEQ ID NO:13122, SEQ ID NO:13123, SEQ ID NO:13124, SEQ ID NO:13125, SEQ ID NO:13126, SEQ ID NO:13127, SEQ ID NO:13128, SEQ ID NO:13129, SEQ ID NO:13130, SEQ ID NO:13131, SEQ ID NO:13132, SEQ ID NO:13133, SEQ ID NO:13134, SEQ ID NO:13135, SEQ ID NO:13136, SEQ ID NO:13137, SEQ ID NO:13138, SEQ ID NO:13139, SEQ ID NO:13140, SEQ ID NO:13141, SEQ ID NO:13142, SEQ ID NO:13143, SEQ ID NO:13144, SEQ ID NO:13145, SEQ ID NO:13146, SEQ ID NO:13147, SEQ ID NO:13148, SEQ ID NO:13149, SEQ ID NO:13150, SEQ ID NO:13151, SEQ ID NO:13152, SEQ ID NO:13153, SEQ ID NO:13154, SEQ ID NO:13155, SEQ ID NO:13156, SEQ ID NO:13157, SEQ ID NO:13158, SEQ ID NO:13159, SEQ ID NO:13160, SEQ ID NO:13161, SEQ ID NO:13162, SEQ ID NO:13163, SEQ ID NO:13164, SEQ ID NO:13165, SEQ ID NO:13166, SEQ ID NO:13167, SEQ ID NO:13168, SEQ ID NO:13169, SEQ ID NO:13170, SEQ ID NO:13171, SEQ ID NO:13172, SEQ ID NO:13173, SEQ ID NO:13174, SEQ ID NO:13175, SEQ ID NO:13176, SEQ ID NO:13177, SEQ ID NO:13178, SEQ ID NO:13179, SEQ ID NO:13180, SEQ ID NO:13181, SEQ ID NO:13182, SEQ ID NO:13183, SEQ ID NO:13184, SEQ ID NO:13185, SEQ ID NO:13186, SEQ ID NO:13187, SEQ ID NO:13188, SEQ ID NO:13189, SEQ ID NO:13190, SEQ ID NO:13191, SEQ ID NO:13192, SEQ ID NO:13193, SEQ ID NO:13194, SEQ ID NO:13195, SEQ ID NO:13196, SEQ ID NO:13197, SEQ ID NO:13198, SEQ ID NO:13199, SEQ ID NO:13200, SEQ ID NO:13201, SEQ ID NO:13202, SEQ ID NO:13203, SEQ ID NO:13204, SEQ ID NO:13205, SEQ ID NO:13206, SEQ ID NO:13207, SEQ ID NO:13208, SEQ ID NO:13209, SEQ ID NO:13210, SEQ ID NO:13211, SEQ ID NO:13212, SEQ ID NO:13213, SEQ ID NO:13214, SEQ ID NO:13215, SEQ ID NO:13216, SEQ ID NO:13217, SEQ ID NO:13218, SEQ ID NO:13219, SEQ ID NO:13220, SEQ ID NO:13221, SEQ ID NO:13222, SEQ ID NO:13223, SEQ ID NO:13224, SEQ ID NO:13225, SEQ ID NO:13226, SEQ ID NO:13227, SEQ ID NO:13228, SEQ ID NO:13229, SEQ ID NO:13230, SEQ ID NO:13231, SEQ ID NO:13232, SEQ ID NO:13233, SEQ ID NO:13234, SEQ ID NO:13235, SEQ ID NO:13236, SEQ ID NO:13237, SEQ ID NO:13238, SEQ ID NO:13239, SEQ ID NO:13240, SEQ ID NO:13241, SEQ ID NO:13242, SEQ ID NO:13243, SEQ ID NO:13244, SEQ ID NO:13245, SEQ ID NO:13246, SEQ ID NO:13247, SEQ ID NO:13248, SEQ ID NO:13249, SEQ ID NO:13250, SEQ ID NO:13251, SEQ ID NO:13252, SEQ ID NO:13253, SEQ ID NO:13254, SEQ ID NO:13255, SEQ ID NO:13256, SEQ ID NO:13257, SEQ ID NO:13258, SEQ ID NO:13259, SEQ ID NO:13260, SEQ ID NO:13261, SEQ ID NO:13262, SEQ ID NO:13263, SEQ ID NO:13264, SEQ ID NO:13265, SEQ ID NO:13266, SEQ ID NO:13267, SEQ ID NO:13268, SEQ ID NO:13269, SEQ ID NO:13270, SEQ ID NO:13271, SEQ ID NO:13272, SEQ ID NO:13273, SEQ ID NO:13274, SEQ ID NO:13275, SEQ ID NO:13276, SEQ ID NO:13277, SEQ ID NO:13278, SEQ ID NO:13279, SEQ ID NO:13280, SEQ ID NO:13281, SEQ ID NO:13282, SEQ ID NO:13283, SEQ ID NO:13284, SEQ ID NO:13285, SEQ ID NO:13286, SEQ ID NO:13287, SEQ ID NO:13288, SEQ ID NO:13289, SEQ ID NO:13290, SEQ ID NO:13291, SEQ ID NO:13292, SEQ ID NO:13293, SEQ ID NO:13294, SEQ ID NO:13295, SEQ ID NO:13296, SEQ ID NO:13297, SEQ ID NO:13298, SEQ ID NO:13299, SEQ ID NO:13300, SEQ ID NO:13301, SEQ ID NO:13302, SEQ ID NO:13303, SEQ ID NO:13304, SEQ ID NO:13305, SEQ ID NO:13306, SEQ ID NO:13307, SEQ ID NO:13308, SEQ ID NO:13309, SEQ ID NO:13310, SEQ ID NO:13311, SEQ ID NO:13312, SEQ ID NO:13313, SEQ ID NO:13314, SEQ ID NO:13315, SEQ ID NO:13316, SEQ ID NO:13317, SEQ ID NO:13318, SEQ ID NO:13319, SEQ ID NO:13320, SEQ ID NO:13321, SEQ ID NO:13322, SEQ ID NO:13323, SEQ ID NO:13324, SEQ ID NO:13325, SEQ ID NO:13326, SEQ ID NO:13327, SEQ ID NO:13328, SEQ ID NO:13329, SEQ ID NO:13330, SEQ ID NO:13331, SEQ ID NO:13332, SEQ ID NO:13333, SEQ ID NO:13334, SEQ ID NO:13335, SEQ ID NO:13336, SEQ ID NO:13337, SEQ ID NO:13338, SEQ ID NO:13339, SEQ ID NO:13340, SEQ ID NO:13341, SEQ ID NO:13342, SEQ ID NO:13343, SEQ ID NO:13344, SEQ ID NO:13345, SEQ ID NO:13346, SEQ ID NO:13347, SEQ ID NO:13348, SEQ ID NO:13349, SEQ ID NO:13350, SEQ ID NO:13351, SEQ ID NO:13352, SEQ ID NO:13353, SEQ ID NO:13354, SEQ ID NO:13355, SEQ ID NO:13356, SEQ ID NO:13357, SEQ ID NO:13358, SEQ ID NO:13359, SEQ ID NO:13360, SEQ ID NO:13361, SEQ ID NO:13362, SEQ ID NO:13363, SEQ ID NO:13364, SEQ ID NO:13365, SEQ ID NO:13366, SEQ ID NO:13367, SEQ ID NO:13368, SEQ ID NO:13369, SEQ ID NO:13370, SEQ ID NO:13371, SEQ ID NO:13372, SEQ ID NO:13373, SEQ ID NO:13374, SEQ ID NO:13375, SEQ ID NO:13376, SEQ ID NO:13377, SEQ ID NO:13378, SEQ ID NO:13379, SEQ ID NO:13380, SEQ ID NO:13381, SEQ ID NO:13382, SEQ ID NO:13383, SEQ ID NO:13384, SEQ ID NO:13385, SEQ ID NO:13386, SEQ ID NO:13387, SEQ ID NO:13388, SEQ ID NO:13389, SEQ ID NO:13390, SEQ ID NO:13391, SEQ ID NO:13392, SEQ ID NO:13393, SEQ ID NO:13394, SEQ ID NO:13395, SEQ ID NO:13396, SEQ ID NO:13397, SEQ ID NO:13398, SEQ ID NO:13399, SEQ ID NO:13400, SEQ ID NO:13401, SEQ ID NO:13402, SEQ ID NO:13403, SEQ ID NO:13404, SEQ ID NO:13405, SEQ ID NO:13406, SEQ ID NO:13407, SEQ ID NO:13408, SEQ ID NO:13409, SEQ ID NO:13410, SEQ ID NO:13411, SEQ ID NO:13412, SEQ ID NO:13413, SEQ ID NO:13414, SEQ ID NO:13415, SEQ ID NO:13416, SEQ ID NO:13417, SEQ ID NO:13418, SEQ ID NO:13419, SEQ ID NO:13420, SEQ ID NO:13421, SEQ ID NO:13422, SEQ ID NO:13423, SEQ ID NO:13424, SEQ ID NO:13425, SEQ ID NO:13426, SEQ ID NO:13427, SEQ ID NO:13428, SEQ ID NO:13429, SEQ ID NO:13430, SEQ ID NO:13431, SEQ ID NO:13432, SEQ ID NO:13433, SEQ ID NO:13434, SEQ ID NO:13435, SEQ ID NO:13436, SEQ ID NO:13437, SEQ ID NO:13438, SEQ ID NO:13439, SEQ ID NO:13440, SEQ ID NO:13441, SEQ ID NO:13442, SEQ ID NO:13443, SEQ ID NO:13444, SEQ ID NO:13445, SEQ ID NO:13446, SEQ ID NO:13447, SEQ ID NO:13448, SEQ ID NO:13449, SEQ ID NO:13450, SEQ ID NO:13451, SEQ ID NO:13452, SEQ ID NO:13453, SEQ ID NO:13454, SEQ ID NO:13455, SEQ ID NO:13456, SEQ ID NO:13457, SEQ ID NO:13458, SEQ ID NO:13459, SEQ ID NO:13460, SEQ ID NO:13461, SEQ ID NO:13462, SEQ ID NO:13463, SEQ ID NO:13464, SEQ ID NO:13465, SEQ ID NO:13466, SEQ ID NO:13467, SEQ ID NO:13468, SEQ ID NO:13469, SEQ ID NO:13470, SEQ ID NO:13471, SEQ ID NO:13472, SEQ ID NO:13473, SEQ ID NO:13474, SEQ ID NO:13475, SEQ ID NO:13476, SEQ ID NO:13477, SEQ ID NO:13478, SEQ ID NO:13479, SEQ ID NO:13480, SEQ ID NO:13481, SEQ ID NO:13482, or SEQ ID NO:13483.

In embodiments, the probe includes SEQ ID NO:13484 and SEQ ID NO:11428. In embodiments, the probe includes SEQ ID NO:13485 and SEQ ID NO:11429. In embodiments, the probe includes SEQ ID NO:13486 and SEQ ID NO:11430. In embodiments, the probe includes SEQ ID NO:13487 and SEQ ID NO:11431. In embodiments, the probe includes SEQ ID NO:13488 and SEQ ID NO:11432. In embodiments, the probe includes SEQ ID NO:13489 and SEQ ID NO:11433. In embodiments, the probe includes SEQ ID NO:13490 and SEQ ID NO:11434. In embodiments, the probe includes SEQ ID NO:13491 and SEQ ID NO:11435. In embodiments, the probe includes SEQ ID NO:13492 and SEQ ID NO:11436. In embodiments, the probe includes SEQ ID NO:13493 and SEQ ID NO:11437. In embodiments, the probe includes SEQ ID NO:13494 and SEQ ID NO:11438. In embodiments, the probe includes SEQ ID NO:13495 and SEQ ID NO:11439. In embodiments, the probe includes SEQ ID NO:13496 and SEQ ID NO:11440. In embodiments, the probe includes SEQ ID NO:13497 and SEQ ID NO:11441. In embodiments, the probe includes SEQ ID NO:13498 and SEQ ID NO:11442. In embodiments, the probe includes SEQ ID NO:13499 and SEQ ID NO:11443. In embodiments, the probe includes SEQ ID NO:13500 and SEQ ID NO:11444. In embodiments, the probe includes SEQ ID NO:13501 and SEQ ID NO:11445. In embodiments, the probe includes SEQ ID NO:13502 and SEQ ID NO:11446. In embodiments, the probe includes SEQ ID NO:13503 and SEQ ID NO:11447. In embodiments, the probe includes SEQ ID NO:13504 and SEQ ID NO:11448. In embodiments, the probe includes SEQ ID NO:13505 and SEQ ID NO:11449. In embodiments, the probe includes SEQ ID NO:13506 and SEQ ID NO:11450. In embodiments, the probe includes SEQ ID NO:13507 and SEQ ID NO:11451. In embodiments, the probe includes SEQ ID NO:13508 and SEQ ID NO:11452. In embodiments, the probe includes SEQ ID NO:13509 and SEQ ID NO:11453. In embodiments, the probe includes SEQ ID NO:13510 and SEQ ID NO:11454. In embodiments, the probe includes SEQ ID NO:13511 and SEQ ID NO:11455. In embodiments, the probe includes SEQ ID NO:13512 and SEQ ID NO:11456. In embodiments, the probe includes SEQ ID NO:13513 and SEQ ID NO:11457. In embodiments, the probe includes SEQ ID NO:13514 and SEQ ID NO:11458. In embodiments, the probe includes SEQ ID NO:13515 and SEQ ID NO:11459. In embodiments, the probe includes SEQ ID NO:13516 and SEQ ID NO:11460. In embodiments, the probe includes SEQ ID NO:13517 and SEQ ID NO:11461. In embodiments, the probe includes SEQ ID NO:13518 and SEQ ID NO:11462. In embodiments, the probe includes SEQ ID NO:13519 and SEQ ID NO:11463. In embodiments, the probe includes SEQ ID NO:13520 and SEQ ID NO:11464. In embodiments, the probe includes SEQ ID NO:13521 and SEQ ID NO:11465. In embodiments, the probe includes SEQ ID NO:13522 and SEQ ID NO:11466. In embodiments, the probe includes SEQ ID NO:13523 and SEQ ID NO:11467. In embodiments, the probe includes SEQ ID NO:13524 and SEQ ID NO:11468. In embodiments, the probe includes SEQ ID NO:13525 and SEQ ID NO:11469. In embodiments, the probe includes SEQ ID NO:13526 and SEQ ID NO:11470. In embodiments, the probe includes SEQ ID NO:13527 and SEQ ID NO:11471. In embodiments, the probe includes SEQ ID NO:13528 and SEQ ID NO:11472. In embodiments, the probe includes SEQ ID NO:13529 and SEQ ID NO:11473. In embodiments, the probe includes SEQ ID NO:13530 and SEQ ID NO:11474. In embodiments, the probe includes SEQ ID NO:13531 and SEQ ID NO:11475. In embodiments, the probe includes SEQ ID NO:13532 and SEQ ID NO:11476. In embodiments, the probe includes SEQ ID NO:13533 and SEQ ID NO:11477. In embodiments, the probe includes SEQ ID NO:13534 and SEQ ID NO:11478. In embodiments, the probe includes SEQ ID NO:13535 and SEQ ID NO:11479. In embodiments, the probe includes SEQ ID NO:13536 and SEQ ID NO:11480. In embodiments, the probe includes SEQ ID NO:13537 and SEQ ID NO:11481. In embodiments, the probe includes SEQ ID NO:13538 and SEQ ID NO:11482. In embodiments, the probe includes SEQ ID NO:13539 and SEQ ID NO:11483. In embodiments, the probe includes SEQ ID NO:13540 and SEQ ID NO:11484. In embodiments, the probe includes SEQ ID NO:13541 and SEQ ID NO:11485. In embodiments, the probe includes SEQ ID NO:13542 and SEQ ID NO:11486. In embodiments, the probe includes SEQ ID NO:13543 and SEQ ID NO:11487. In embodiments, the probe includes SEQ ID NO:13544 and SEQ ID NO:11488. In embodiments, the probe includes SEQ ID NO:13545 and SEQ ID NO:11489. In embodiments, the probe includes SEQ ID NO:13546 and SEQ ID NO:11490. In embodiments, the probe includes SEQ ID NO:13547 and SEQ ID NO:11491. In embodiments, the probe includes SEQ ID NO:13548 and SEQ ID NO:11492. In embodiments, the probe includes SEQ ID NO:13549 and SEQ ID NO:11493. In embodiments, the probe includes SEQ ID NO:13550 and SEQ ID NO:11494. In embodiments, the probe includes SEQ ID NO:13551 and SEQ ID NO:11495. In embodiments, the probe includes SEQ ID NO:13552 and SEQ ID NO:11496. In embodiments, the probe includes SEQ ID NO:13553 and SEQ ID NO:11497. In embodiments, the probe includes SEQ ID NO:13554 and SEQ ID NO:11498. In embodiments, the probe includes SEQ ID NO:13555 and SEQ ID NO:11499. In embodiments, the probe includes SEQ ID NO:13556 and SEQ ID NO:11500. In embodiments, the probe includes SEQ ID NO:13557 and SEQ ID NO:11501. In embodiments, the probe includes SEQ ID NO:13558 and SEQ ID NO:11502. In embodiments, the probe includes SEQ ID NO:13559 and SEQ ID NO:11503. In embodiments, the probe includes SEQ ID NO:13560 and SEQ ID NO:11504. In embodiments, the probe includes SEQ ID NO:13561 and SEQ ID NO:11505. In embodiments, the probe includes SEQ ID NO:13562 and SEQ ID NO:11506. In embodiments, the probe includes SEQ ID NO:13563 and SEQ ID NO:11507. In embodiments, the probe includes SEQ ID NO:13564 and SEQ ID NO:11508. In embodiments, the probe includes SEQ ID NO:13565 and SEQ ID NO:11509. In embodiments, the probe includes SEQ ID NO:13566 and SEQ ID NO:11510. In embodiments, the probe includes SEQ ID NO:13567 and SEQ ID NO:11511. In embodiments, the probe includes SEQ ID NO:13568 and SEQ ID NO:11512. In embodiments, the probe includes SEQ ID NO:13569 and SEQ ID NO:11513. In embodiments, the probe includes SEQ ID NO:13570 and SEQ ID NO:11514. In embodiments, the probe includes SEQ ID NO:13571 and SEQ ID NO:11515. In embodiments, the probe includes SEQ ID NO:13572 and SEQ ID NO:11516. In embodiments, the probe includes SEQ ID NO:13573 and SEQ ID NO:11517. In embodiments, the probe includes SEQ ID NO:13574 and SEQ ID NO:11518. In embodiments, the probe includes SEQ ID NO:13575 and SEQ ID NO:11519. In embodiments, the probe includes SEQ ID NO:13576 and SEQ ID NO:11520. In embodiments, the probe includes SEQ ID NO:13577 and SEQ ID NO:11521. In embodiments, the probe includes SEQ ID NO:13578 and SEQ ID NO:11522. In embodiments, the probe includes SEQ ID NO:13579 and SEQ ID NO:11523. In embodiments, the probe includes SEQ ID NO:13580 and SEQ ID NO:11524. In embodiments, the probe includes SEQ ID NO:13581 and SEQ ID NO:11525. In embodiments, the probe includes SEQ ID NO:13582 and SEQ ID NO:11526. In embodiments, the probe includes SEQ ID NO:13583 and SEQ ID NO:11527. In embodiments, the probe includes SEQ ID NO:13584 and SEQ ID NO:11528. In embodiments, the probe includes SEQ ID NO:13585 and SEQ ID NO:11529. In embodiments, the probe includes SEQ ID NO:13586 and SEQ ID NO:11530. In embodiments, the probe includes SEQ ID NO:13587 and SEQ ID NO:11531. In embodiments, the probe includes SEQ ID NO:13588 and SEQ ID NO:11532. In embodiments, the probe includes SEQ ID NO:13589 and SEQ ID NO:11533. In embodiments, the probe includes SEQ ID NO:13590 and SEQ ID NO:11534. In embodiments, the probe includes SEQ ID NO:13591 and SEQ ID NO:11535. In embodiments, the probe includes SEQ ID NO:13592 and SEQ ID NO:11536. In embodiments, the probe includes SEQ ID NO:13593 and SEQ ID NO:11537. In embodiments, the probe includes SEQ ID NO:13594 and SEQ ID NO:11538. In embodiments, the probe includes SEQ ID NO:13595 and SEQ ID NO:11539. In embodiments, the probe includes SEQ ID NO:13596 and SEQ ID NO:11540. In embodiments, the probe includes SEQ ID NO:13597 and SEQ ID NO:11541. In embodiments, the probe includes SEQ ID NO:13598 and SEQ ID NO:11542. In embodiments, the probe includes SEQ ID NO:13599 and SEQ ID NO:11543. In embodiments, the probe includes SEQ ID NO:13600 and SEQ ID NO:11544. In embodiments, the probe includes SEQ ID NO:13601 and SEQ ID NO:11545. In embodiments, the probe includes SEQ ID NO:13602 and SEQ ID NO:11546. In embodiments, the probe includes SEQ ID NO:13603 and SEQ ID NO:11547. In embodiments, the probe includes SEQ ID NO:13604 and SEQ ID NO:11548. In embodiments, the probe includes SEQ ID NO:13605 and SEQ ID NO:11549. In embodiments, the probe includes SEQ ID NO:13606 and SEQ ID NO:11550. In embodiments, the probe includes SEQ ID NO:13607 and SEQ ID NO:11551. In embodiments, the probe includes SEQ ID NO:13608 and SEQ ID NO:11552. In embodiments, the probe includes SEQ ID NO:13609 and SEQ ID NO:11553. In embodiments, the probe includes SEQ ID NO:13610 and SEQ ID NO:11554. In embodiments, the probe includes SEQ ID NO:13611 and SEQ ID NO:11555. In embodiments, the probe includes SEQ ID NO:13612 and SEQ ID NO:11556. In embodiments, the probe includes SEQ ID NO:13613 and SEQ ID NO:11557. In embodiments, the probe includes SEQ ID NO:13614 and SEQ ID NO:11558. In embodiments, the probe includes SEQ ID NO:13615 and SEQ ID NO:11559. In embodiments, the probe includes SEQ ID NO:13616 and SEQ ID NO:11560. In embodiments, the probe includes SEQ ID NO:13617 and SEQ ID NO:11561. In embodiments, the probe includes SEQ ID NO:13618 and SEQ ID NO:11562. In embodiments, the probe includes SEQ ID NO:13619 and SEQ ID NO:11563. In embodiments, the probe includes SEQ ID NO:13620 and SEQ ID NO:11564. In embodiments, the probe includes SEQ ID NO:13621 and SEQ ID NO:11565. In embodiments, the probe includes SEQ ID NO:13622 and SEQ ID NO:11566. In embodiments, the probe includes SEQ ID NO:13623 and SEQ ID NO:11567. In embodiments, the probe includes SEQ ID NO:13624 and SEQ ID NO:11568. In embodiments, the probe includes SEQ ID NO:13625 and SEQ ID NO:11569. In embodiments, the probe includes SEQ ID NO:13626 and SEQ ID NO:11570. In embodiments, the probe includes SEQ ID NO:13627 and SEQ ID NO:11571. In embodiments, the probe includes SEQ ID NO:13628 and SEQ ID NO:11572. In embodiments, the probe includes SEQ ID NO:13629 and SEQ ID NO:11573. In embodiments, the probe includes SEQ ID NO:13630 and SEQ ID NO:11574. In embodiments, the probe includes SEQ ID NO:13631 and SEQ ID NO:11575. In embodiments, the probe includes SEQ ID NO:13632 and SEQ ID NO:11576. In embodiments, the probe includes SEQ ID NO:13633 and SEQ ID NO:11577. In embodiments, the probe includes SEQ ID NO:13634 and SEQ ID NO:11578. In embodiments, the probe includes SEQ ID NO:13635 and SEQ ID NO:11579. In embodiments, the probe includes SEQ ID NO:13636 and SEQ ID NO:11580. In embodiments, the probe includes SEQ ID NO:13637 and SEQ ID NO:11581. In embodiments, the probe includes SEQ ID NO:13638 and SEQ ID NO:11582. In embodiments, the probe includes SEQ ID NO:13639 and SEQ ID NO:11583. In embodiments, the probe includes SEQ ID NO:13640 and SEQ ID NO:11584. In embodiments, the probe includes SEQ ID NO:13641 and SEQ ID NO:11585. In embodiments, the probe includes SEQ ID NO:13642 and SEQ ID NO:11586. In embodiments, the probe includes SEQ ID NO:13643 and SEQ ID NO:11587. In embodiments, the probe includes SEQ ID NO:13644 and SEQ ID NO:11588. In embodiments, the probe includes SEQ ID NO:13645 and SEQ ID NO:11589. In embodiments, the probe includes SEQ ID NO:13646 and SEQ ID NO:11590. In embodiments, the probe includes SEQ ID NO:13647 and SEQ ID NO:11591. In embodiments, the probe includes SEQ ID NO:13648 and SEQ ID NO:11592. In embodiments, the probe includes SEQ ID NO:13649 and SEQ ID NO:11593. In embodiments, the probe includes SEQ ID NO:13650 and SEQ ID NO:11594. In embodiments, the probe includes SEQ ID NO:13651 and SEQ ID NO:11595. In embodiments, the probe includes SEQ ID NO:13652 and SEQ ID NO:11596. In embodiments, the probe includes SEQ ID NO:13653 and SEQ ID NO:11597. In embodiments, the probe includes SEQ ID NO:13654 and SEQ ID NO:11598. In embodiments, the probe includes SEQ ID NO:13655 and SEQ ID NO:11599. In embodiments, the probe includes SEQ ID NO:13656 and SEQ ID NO:11600. In embodiments, the probe includes SEQ ID NO:13657 and SEQ ID NO:11601. In embodiments, the probe includes SEQ ID NO:13658 and SEQ ID NO:11602. In embodiments, the probe includes SEQ ID NO:13659 and SEQ ID NO:11603. In embodiments, the probe includes SEQ ID NO:13660 and SEQ ID NO:11604. In embodiments, the probe includes SEQ ID NO:13661 and SEQ ID NO:11605. In embodiments, the probe includes SEQ ID NO:13662 and SEQ ID NO:11606. In embodiments, the probe includes SEQ ID NO:13663 and SEQ ID NO:11607. In embodiments, the probe includes SEQ ID NO:13664 and SEQ ID NO:11608. In embodiments, the probe includes SEQ ID NO:13665 and SEQ ID NO:11609. In embodiments, the probe includes SEQ ID NO:13666 and SEQ ID NO:11610. In embodiments, the probe includes SEQ ID NO:13667 and SEQ ID NO:11611. In embodiments, the probe includes SEQ ID NO:13668 and SEQ ID NO:11612. In embodiments, the probe includes SEQ ID NO:13669 and SEQ ID NO:11613. In embodiments, the probe includes SEQ ID NO:13670 and SEQ ID NO:11614. In embodiments, the probe includes SEQ ID NO:13671 and SEQ ID NO:11615. In embodiments, the probe includes SEQ ID NO:13672 and SEQ ID NO:11616. In embodiments, the probe includes SEQ ID NO:13673 and SEQ ID NO:11617. In embodiments, the probe includes SEQ ID NO:13674 and SEQ ID NO:11618. In embodiments, the probe includes SEQ ID NO:13675 and SEQ ID NO:11619. In embodiments, the probe includes SEQ ID NO:13676 and SEQ ID NO:11620. In embodiments, the probe includes SEQ ID NO:13677 and SEQ ID NO:11621. In embodiments, the probe includes SEQ ID NO:13678 and SEQ ID NO:11622. In embodiments, the probe includes SEQ ID NO:13679 and SEQ ID NO:11623. In embodiments, the probe includes SEQ ID NO:13680 and SEQ ID NO:11624. In embodiments, the probe includes SEQ ID NO:13681 and SEQ ID NO:11625. In embodiments, the probe includes SEQ ID NO:13682 and SEQ ID NO:11626. In embodiments, the probe includes SEQ ID NO:13683 and SEQ ID NO:11627. In embodiments, the probe includes SEQ ID NO:13684 and SEQ ID NO:11628. In embodiments, the probe includes SEQ ID NO:13685 and SEQ ID NO:11629. In embodiments, the probe includes SEQ ID NO:13686 and SEQ ID NO:11630. In embodiments, the probe includes SEQ ID NO:13687 and SEQ ID NO:11631. In embodiments, the probe includes SEQ ID NO:13688 and SEQ ID NO:11632. In embodiments, the probe includes SEQ ID NO:13689 and SEQ ID NO:11633. In embodiments, the probe includes SEQ ID NO:13690 and SEQ ID NO:11634. In embodiments, the probe includes SEQ ID NO:13691 and SEQ ID NO:11635. In embodiments, the probe includes SEQ ID NO:13692 and SEQ ID NO:11636. In embodiments, the probe includes SEQ ID NO:13693 and SEQ ID NO:11637. In embodiments, the probe includes SEQ ID NO:13694 and SEQ ID NO:11638. In embodiments, the probe includes SEQ ID NO:13695 and SEQ ID NO:11639. In embodiments, the probe includes SEQ ID NO:13696 and SEQ ID NO:11640. In embodiments, the probe includes SEQ ID NO:13697 and SEQ ID NO:11641. In embodiments, the probe includes SEQ ID NO:13698 and SEQ ID NO:11642. In embodiments, the probe includes SEQ ID NO:13699 and SEQ ID NO:11643. In embodiments, the probe includes SEQ ID NO:13700 and SEQ ID NO:11644. In embodiments, the probe includes SEQ ID NO:13701 and SEQ ID NO:11645. In embodiments, the probe includes SEQ ID NO:13702 and SEQ ID NO:11646. In embodiments, the probe includes SEQ ID NO:13703 and SEQ ID NO:11647. In embodiments, the probe includes SEQ ID NO:13704 and SEQ ID NO:11648. In embodiments, the probe includes SEQ ID NO:13705 and SEQ ID NO:11649. In embodiments, the probe includes SEQ ID NO:13706 and SEQ ID NO:11650. In embodiments, the probe includes SEQ ID NO:13707 and SEQ ID NO:11651. In embodiments, the probe includes SEQ ID NO:13708 and SEQ ID NO:11652. In embodiments, the probe includes SEQ ID NO:13709 and SEQ ID NO:11653. In embodiments, the probe includes SEQ ID NO:13710 and SEQ ID NO:11654. In embodiments, the probe includes SEQ ID NO:13711 and SEQ ID NO:11655. In embodiments, the probe includes SEQ ID NO:13712 and SEQ ID NO:11656. In embodiments, the probe includes SEQ ID NO:13713 and SEQ ID NO:11657. In embodiments, the probe includes SEQ ID NO:13714 and SEQ ID NO:11658. In embodiments, the probe includes SEQ ID NO:13715 and SEQ ID NO:11659. In embodiments, the probe includes SEQ ID NO:13716 and SEQ ID NO:11660. In embodiments, the probe includes SEQ ID NO:13717 and SEQ ID NO:11661. In embodiments, the probe includes SEQ ID NO:13718 and SEQ ID NO:11662. In embodiments, the probe includes SEQ ID NO:13719 and SEQ ID NO:11663. In embodiments, the probe includes SEQ ID NO:13720 and SEQ ID NO:11664. In embodiments, the probe includes SEQ ID NO:13721 and SEQ ID NO:11665. In embodiments, the probe includes SEQ ID NO:13722 and SEQ ID NO:11666. In embodiments, the probe includes SEQ ID NO:13723 and SEQ ID NO:11667. In embodiments, the probe includes SEQ ID NO:13724 and SEQ ID NO:11668. In embodiments, the probe includes SEQ ID NO:13725 and SEQ ID NO:11669. In embodiments, the probe includes SEQ ID NO:13726 and SEQ ID NO:11670. In embodiments, the probe includes SEQ ID NO:13727 and SEQ ID NO:11671. In embodiments, the probe includes SEQ ID NO:13728 and SEQ ID NO:11672. In embodiments, the probe includes SEQ ID NO:13729 and SEQ ID NO:11673. In embodiments, the probe includes SEQ ID NO:13730 and SEQ ID NO:11674. In embodiments, the probe includes SEQ ID NO:13731 and SEQ ID NO:11675. In embodiments, the probe includes SEQ ID NO:13732 and SEQ ID NO:11676. In embodiments, the probe includes SEQ ID NO:13733 and SEQ ID NO:11677. In embodiments, the probe includes SEQ ID NO:13734 and SEQ ID NO:11678. In embodiments, the probe includes SEQ ID NO:13735 and SEQ ID NO:11679. In embodiments, the probe includes SEQ ID NO:13736 and SEQ ID NO:11680. In embodiments, the probe includes SEQ ID NO:13737 and SEQ ID NO:11681. In embodiments, the probe includes SEQ ID NO:13738 and SEQ ID NO:11682. In embodiments, the probe includes SEQ ID NO:13739 and SEQ ID NO:11683. In embodiments, the probe includes SEQ ID NO:13740 and SEQ ID NO:11684. In embodiments, the probe includes SEQ ID NO:13741 and SEQ ID NO:11685. In embodiments, the probe includes SEQ ID NO:13742 and SEQ ID NO:11686. In embodiments, the probe includes SEQ ID NO:13743 and SEQ ID NO:11687. In embodiments, the probe includes SEQ ID NO:13744 and SEQ ID NO:11688. In embodiments, the probe includes SEQ ID NO:13745 and SEQ ID NO:11689. In embodiments, the probe includes SEQ ID NO:13746 and SEQ ID NO:11690. In embodiments, the probe includes SEQ ID NO:13747 and SEQ ID NO:11691. In embodiments, the probe includes SEQ ID NO:13748 and SEQ ID NO:11692. In embodiments, the probe includes SEQ ID NO:13749 and SEQ ID NO:11693. In embodiments, the probe includes SEQ ID NO:13750 and SEQ ID NO:11694. In embodiments, the probe includes SEQ ID NO:13751 and SEQ ID NO:11695. In embodiments, the probe includes SEQ ID NO:13752 and SEQ ID NO:11696. In embodiments, the probe includes SEQ ID NO:13753 and SEQ ID NO:11697. In embodiments, the probe includes SEQ ID NO:13754 and SEQ ID NO:11698. In embodiments, the probe includes SEQ ID NO:13755 and SEQ ID NO:11699. In embodiments, the probe includes SEQ ID NO:13756 and SEQ ID NO:11700. In embodiments, the probe includes SEQ ID NO:13757 and SEQ ID NO:11701. In embodiments, the probe includes SEQ ID NO:13758 and SEQ ID NO:11702. In embodiments, the probe includes SEQ ID NO:13759 and SEQ ID NO:11703. In embodiments, the probe includes SEQ ID NO:13760 and SEQ ID NO:11704. In embodiments, the probe includes SEQ ID NO:13761 and SEQ ID NO:11705. In embodiments, the probe includes SEQ ID NO:13762 and SEQ ID NO:11706. In embodiments, the probe includes SEQ ID NO:13763 and SEQ ID NO:11707. In embodiments, the probe includes SEQ ID NO:13764 and SEQ ID NO:11708. In embodiments, the probe includes SEQ ID NO:13765 and SEQ ID NO:11709. In embodiments, the probe includes SEQ ID NO:13766 and SEQ ID NO:11710. In embodiments, the probe includes SEQ ID NO:13767 and SEQ ID NO:11711. In embodiments, the probe includes SEQ ID NO:13768 and SEQ ID NO:11712. In embodiments, the probe includes SEQ ID NO:13769 and SEQ ID NO:11713. In embodiments, the probe includes SEQ ID NO:13770 and SEQ ID NO:11714. In embodiments, the probe includes SEQ ID NO:13771 and SEQ ID NO:11715. In embodiments, the probe includes SEQ ID NO:13772 and SEQ ID NO:11716. In embodiments, the probe includes SEQ ID NO:13773 and SEQ ID NO:11717. In embodiments, the probe includes SEQ ID NO:13774 and SEQ ID NO:11718. In embodiments, the probe includes SEQ ID NO:13775 and SEQ ID NO:11719. In embodiments, the probe includes SEQ ID NO:13776 and SEQ ID NO:11720. In embodiments, the probe includes SEQ ID NO:13777 and SEQ ID NO:11721. In embodiments, the probe includes SEQ ID NO:13778 and SEQ ID NO:11722. In embodiments, the probe includes SEQ ID NO:13779 and SEQ ID NO:11723. In embodiments, the probe includes SEQ ID NO:13780 and SEQ ID NO:11724. In embodiments, the probe includes SEQ ID NO:13781 and SEQ ID NO:11725. In embodiments, the probe includes SEQ ID NO:13782 and SEQ ID NO:11726. In embodiments, the probe includes SEQ ID NO:13783 and SEQ ID NO:11727. In embodiments, the probe includes SEQ ID NO:13784 and SEQ ID NO:11728. In embodiments, the probe includes SEQ ID NO:13785 and SEQ ID NO:11729. In embodiments, the probe includes SEQ ID NO:13786 and SEQ ID NO:11730. In embodiments, the probe includes SEQ ID NO:13787 and SEQ ID NO:11731. In embodiments, the probe includes SEQ ID NO:13788 and SEQ ID NO:11732. In embodiments, the probe includes SEQ ID NO:13789 and SEQ ID NO:11733. In embodiments, the probe includes SEQ ID NO:13790 and SEQ ID NO:11734. In embodiments, the probe includes SEQ ID NO:13791 and SEQ ID NO:11735. In embodiments, the probe includes SEQ ID NO:13792 and SEQ ID NO:11736. In embodiments, the probe includes SEQ ID NO:13793 and SEQ ID NO:11737. In embodiments, the probe includes SEQ ID NO:13794 and SEQ ID NO:11738. In embodiments, the probe includes SEQ ID NO:13795 and SEQ ID NO:11739. In embodiments, the probe includes SEQ ID NO:13796 and SEQ ID NO:11740. In embodiments, the probe includes SEQ ID NO:13797 and SEQ ID NO:11741. In embodiments, the probe includes SEQ ID NO:13798 and SEQ ID NO:11742. In embodiments, the probe includes SEQ ID NO:13799 and SEQ ID NO:11743. In embodiments, the probe includes SEQ ID NO:13800 and SEQ ID NO:11744. In embodiments, the probe includes SEQ ID NO:13801 and SEQ ID NO:11745. In embodiments, the probe includes SEQ ID NO:13802 and SEQ ID NO:11746. In embodiments, the probe includes SEQ ID NO:13803 and SEQ ID NO:11747. In embodiments, the probe includes SEQ ID NO:13804 and SEQ ID NO:11748. In embodiments, the probe includes SEQ ID NO:13805 and SEQ ID NO:11749. In embodiments, the probe includes SEQ ID NO:13806 and SEQ ID NO:11750. In embodiments, the probe includes SEQ ID NO:13807 and SEQ ID NO:11751. In embodiments, the probe includes SEQ ID NO:13808 and SEQ ID NO:11752. In embodiments, the probe includes SEQ ID NO:13809 and SEQ ID NO:11753. In embodiments, the probe includes SEQ ID NO:13810 and SEQ ID NO:11754. In embodiments, the probe includes SEQ ID NO:13811 and SEQ ID NO:11755. In embodiments, the probe includes SEQ ID NO:13812 and SEQ ID NO:11756. In embodiments, the probe includes SEQ ID NO:13813 and SEQ ID NO:11757. In embodiments, the probe includes SEQ ID NO:13814 and SEQ ID NO:11758. In embodiments, the probe includes SEQ ID NO:13815 and SEQ ID NO:11759. In embodiments, the probe includes SEQ ID NO:13816 and SEQ ID NO:11760. In embodiments, the probe includes SEQ ID NO:13817 and SEQ ID NO:11761. In embodiments, the probe includes SEQ ID NO:13818 and SEQ ID NO:11762. In embodiments, the probe includes SEQ ID NO:13819 and SEQ ID NO:11763. In embodiments, the probe includes SEQ ID NO:13820 and SEQ ID NO:11764. In embodiments, the probe includes SEQ ID NO:13821 and SEQ ID NO:11765. In embodiments, the probe includes SEQ ID NO:13822 and SEQ ID NO:11766. In embodiments, the probe includes SEQ ID NO:13823 and SEQ ID NO:11767. In embodiments, the probe includes SEQ ID NO:13824 and SEQ ID NO:11768. In embodiments, the probe includes SEQ ID NO:13825 and SEQ ID NO:11769. In embodiments, the probe includes SEQ ID NO:13826 and SEQ ID NO:11770. In embodiments, the probe includes SEQ ID NO:13827 and SEQ ID NO:11771. In embodiments, the probe includes SEQ ID NO:13828 and SEQ ID NO:11772. In embodiments, the probe includes SEQ ID NO:13829 and SEQ ID NO:11773. In embodiments, the probe includes SEQ ID NO:13830 and SEQ ID NO:11774. In embodiments, the probe includes SEQ ID NO:13831 and SEQ ID NO:11775. In embodiments, the probe includes SEQ ID NO:13832 and SEQ ID NO:11776. In embodiments, the probe includes SEQ ID NO:13833 and SEQ ID NO:11777. In embodiments, the probe includes SEQ ID NO:13834 and SEQ ID NO:11778. In embodiments, the probe includes SEQ ID NO:13835 and SEQ ID NO:11779. In embodiments, the probe includes SEQ ID NO:13836 and SEQ ID NO:11780. In embodiments, the probe includes SEQ ID NO:13837 and SEQ ID NO:11781. In embodiments, the probe includes SEQ ID NO:13838 and SEQ ID NO:11782. In embodiments, the probe includes SEQ ID NO:13839 and SEQ ID NO:11783. In embodiments, the probe includes SEQ ID NO:13840 and SEQ ID NO:11784. In embodiments, the probe includes SEQ ID NO:13841 and SEQ ID NO:11785. In embodiments, the probe includes SEQ ID NO:13842 and SEQ ID NO:11786. In embodiments, the probe includes SEQ ID NO:13843 and SEQ ID NO:11787. In embodiments, the probe includes SEQ ID NO:13844 and SEQ ID NO:11788. In embodiments, the probe includes SEQ ID NO:13845 and SEQ ID NO:11789. In embodiments, the probe includes SEQ ID NO:13846 and SEQ ID NO:11790. In embodiments, the probe includes SEQ ID NO:13847 and SEQ ID NO:11791. In embodiments, the probe includes SEQ ID NO:13848 and SEQ ID NO:11792. In embodiments, the probe includes SEQ ID NO:13849 and SEQ ID NO:11793. In embodiments, the probe includes SEQ ID NO:13850 and SEQ ID NO:11794. In embodiments, the probe includes SEQ ID NO:13851 and SEQ ID NO:11795. In embodiments, the probe includes SEQ ID NO:13852 and SEQ ID NO:11796. In embodiments, the probe includes SEQ ID NO:13853 and SEQ ID NO:11797. In embodiments, the probe includes SEQ ID NO:13854 and SEQ ID NO:11798. In embodiments, the probe includes SEQ ID NO:13855 and SEQ ID NO:11799. In embodiments, the probe includes SEQ ID NO:13856 and SEQ ID NO:11800. In embodiments, the probe includes SEQ ID NO:13857 and SEQ ID NO:11801. In embodiments, the probe includes SEQ ID NO:13858 and SEQ ID NO:11802. In embodiments, the probe includes SEQ ID NO:13859 and SEQ ID NO:11803. In embodiments, the probe includes SEQ ID NO:13860 and SEQ ID NO:11804. In embodiments, the probe includes SEQ ID NO:13861 and SEQ ID NO:11805. In embodiments, the probe includes SEQ ID NO:13862 and SEQ ID NO:11806. In embodiments, the probe includes SEQ ID NO:13863 and SEQ ID NO:11807. In embodiments, the probe includes SEQ ID NO:13864 and SEQ ID NO:11808. In embodiments, the probe includes SEQ ID NO:13865 and SEQ ID NO:11809. In embodiments, the probe includes SEQ ID NO:13866 and SEQ ID NO:11810. In embodiments, the probe includes SEQ ID NO:13867 and SEQ ID NO:11811. In embodiments, the probe includes SEQ ID NO:13868 and SEQ ID NO:11812. In embodiments, the probe includes SEQ ID NO:13869 and SEQ ID NO:11813. In embodiments, the probe includes SEQ ID NO:13870 and SEQ ID NO:11814. In embodiments, the probe includes SEQ ID NO:13871 and SEQ ID NO:11815. In embodiments, the probe includes SEQ ID NO:13872 and SEQ ID NO:11816. In embodiments, the probe includes SEQ ID NO:13873 and SEQ ID NO:11817. In embodiments, the probe includes SEQ ID NO:13874 and SEQ ID NO:11818. In embodiments, the probe includes SEQ ID NO:13875 and SEQ ID NO:11819. In embodiments, the probe includes SEQ ID NO:13876 and SEQ ID NO:11820. In embodiments, the probe includes SEQ ID NO:13877 and SEQ ID NO:11821. In embodiments, the probe includes SEQ ID NO:13878 and SEQ ID NO:11822. In embodiments, the probe includes SEQ ID NO:13879 and SEQ ID NO:11823. In embodiments, the probe includes SEQ ID NO:13880 and SEQ ID NO:11824. In embodiments, the probe includes SEQ ID NO:13881 and SEQ ID NO:11825. In embodiments, the probe includes SEQ ID NO:13882 and SEQ ID NO:11826. In embodiments, the probe includes SEQ ID NO:13883 and SEQ ID NO:11827. In embodiments, the probe includes SEQ ID NO:13884 and SEQ ID NO:11828. In embodiments, the probe includes SEQ ID NO:13885 and SEQ ID NO:11829. In embodiments, the probe includes SEQ ID NO:13886 and SEQ ID NO:11830. In embodiments, the probe includes SEQ ID NO:13887 and SEQ ID NO:11831. In embodiments, the probe includes SEQ ID NO:13888 and SEQ ID NO:11832. In embodiments, the probe includes SEQ ID NO:13889 and SEQ ID NO:11833. In embodiments, the probe includes SEQ ID NO:13890 and SEQ ID NO:11834. In embodiments, the probe includes SEQ ID NO:13891 and SEQ ID NO:11835. In embodiments, the probe includes SEQ ID NO:13892 and SEQ ID NO:11836. In embodiments, the probe includes SEQ ID NO:13893 and SEQ ID NO:11837. In embodiments, the probe includes SEQ ID NO:13894 and SEQ ID NO:11838. In embodiments, the probe includes SEQ ID NO:13895 and SEQ ID NO:11839. In embodiments, the probe includes SEQ ID NO:13896 and SEQ ID NO:11840. In embodiments, the probe includes SEQ ID NO:13897 and SEQ ID NO:11841. In embodiments, the probe includes SEQ ID NO:13898 and SEQ ID NO:11842. In embodiments, the probe includes SEQ ID NO:13899 and SEQ ID NO:11843. In embodiments, the probe includes SEQ ID NO:13900 and SEQ ID NO:11844. In embodiments, the probe includes SEQ ID NO:13901 and SEQ ID NO:11845. In embodiments, the probe includes SEQ ID NO:13902 and SEQ ID NO:11846. In embodiments, the probe includes SEQ ID NO:13903 and SEQ ID NO:11847. In embodiments, the probe includes SEQ ID NO:13904 and SEQ ID NO:11848. In embodiments, the probe includes SEQ ID NO:13905 and SEQ ID NO:11849. In embodiments, the probe includes SEQ ID NO:13906 and SEQ ID NO:11850. In embodiments, the probe includes SEQ ID NO:13907 and SEQ ID NO:11851. In embodiments, the probe includes SEQ ID NO:13908 and SEQ ID NO:11852. In embodiments, the probe includes SEQ ID NO:13909 and SEQ ID NO:11853. In embodiments, the probe includes SEQ ID NO:13910 and SEQ ID NO:11854. In embodiments, the probe includes SEQ ID NO:13911 and SEQ ID NO:11855. In embodiments, the probe includes SEQ ID NO:13912 and SEQ ID NO:11856. In embodiments, the probe includes SEQ ID NO:13913 and SEQ ID NO:11857. In embodiments, the probe includes SEQ ID NO:13914 and SEQ ID NO:11858. In embodiments, the probe includes SEQ ID NO:13915 and SEQ ID NO:11859. In embodiments, the probe includes SEQ ID NO:13916 and SEQ ID NO:11860. In embodiments, the probe includes SEQ ID NO:13917 and SEQ ID NO:11861. In embodiments, the probe includes SEQ ID NO:13918 and SEQ ID NO:11862. In embodiments, the probe includes SEQ ID NO:13919 and SEQ ID NO:11863. In embodiments, the probe includes SEQ ID NO:13920 and SEQ ID NO:11864. In embodiments, the probe includes SEQ ID NO:13921 and SEQ ID NO:11865. In embodiments, the probe includes SEQ ID NO:13922 and SEQ ID NO:11866. In embodiments, the probe includes SEQ ID NO:13923 and SEQ ID NO:11867. In embodiments, the probe includes SEQ ID NO:13924 and SEQ ID NO:11868. In embodiments, the probe includes SEQ ID NO:13925 and SEQ ID NO:11869. In embodiments, the probe includes SEQ ID NO:13926 and SEQ ID NO:11870. In embodiments, the probe includes SEQ ID NO:13927 and SEQ ID NO:11871. In embodiments, the probe includes SEQ ID NO:13928 and SEQ ID NO:11872. In embodiments, the probe includes SEQ ID NO:13929 and SEQ ID NO:11873. In embodiments, the probe includes SEQ ID NO:13930 and SEQ ID NO:11874. In embodiments, the probe includes SEQ ID NO:13931 and SEQ ID NO:11875. In embodiments, the probe includes SEQ ID NO:13932 and SEQ ID NO:11876. In embodiments, the probe includes SEQ ID NO:13933 and SEQ ID NO:11877. In embodiments, the probe includes SEQ ID NO:13934 and SEQ ID NO:11878. In embodiments, the probe includes SEQ ID NO:13935 and SEQ ID NO:11879. In embodiments, the probe includes SEQ ID NO:13936 and SEQ ID NO:11880. In embodiments, the probe includes SEQ ID NO:13937 and SEQ ID NO:11881. In embodiments, the probe includes SEQ ID NO:13938 and SEQ ID NO:11882. In embodiments, the probe includes SEQ ID NO:13939 and SEQ ID NO:11883. In embodiments, the probe includes SEQ ID NO:13940 and SEQ ID NO:11884. In embodiments, the probe includes SEQ ID NO:13941 and SEQ ID NO:11885. In embodiments, the probe includes SEQ ID NO:13942 and SEQ ID NO:11886. In embodiments, the probe includes SEQ ID NO:13943 and SEQ ID NO:11887. In embodiments, the probe includes SEQ ID NO:13944 and SEQ ID NO:11888. In embodiments, the probe includes SEQ ID NO:13945 and SEQ ID NO:11889. In embodiments, the probe includes SEQ ID NO:13946 and SEQ ID NO:11890. In embodiments, the probe includes SEQ ID NO:13947 and SEQ ID NO:11891. In embodiments, the probe includes SEQ ID NO:13948 and SEQ ID NO:11892. In embodiments, the probe includes SEQ ID NO:13949 and SEQ ID NO:11893. In embodiments, the probe includes SEQ ID NO:13950 and SEQ ID NO:11894. In embodiments, the probe includes SEQ ID NO:13951 and SEQ ID NO:11895. In embodiments, the probe includes SEQ ID NO:13952 and SEQ ID NO:11896. In embodiments, the probe includes SEQ ID NO:13953 and SEQ ID NO:11897. In embodiments, the probe includes SEQ ID NO:13954 and SEQ ID NO:11898. In embodiments, the probe includes SEQ ID NO:13955 and SEQ ID NO:11899. In embodiments, the probe includes SEQ ID NO:13956 and SEQ ID NO:11900. In embodiments, the probe includes SEQ ID NO:13957 and SEQ ID NO:11901. In embodiments, the probe includes SEQ ID NO:13958 and SEQ ID NO:11902. In embodiments, the probe includes SEQ ID NO:13959 and SEQ ID NO:11903. In embodiments, the probe includes SEQ ID NO:13960 and SEQ ID NO:11904. In embodiments, the probe includes SEQ ID NO:13961 and SEQ ID NO:11905. In embodiments, the probe includes SEQ ID NO:13962 and SEQ ID NO:11906. In embodiments, the probe includes SEQ ID NO:13963 and SEQ ID NO:11907. In embodiments, the probe includes SEQ ID NO:13964 and SEQ ID NO:11908. In embodiments, the probe includes SEQ ID NO:13965 and SEQ ID NO:11909. In embodiments, the probe includes SEQ ID NO:13966 and SEQ ID NO:11910. In embodiments, the probe includes SEQ ID NO:13967 and SEQ ID NO:11911. In embodiments, the probe includes SEQ ID NO:13968 and SEQ ID NO:11912. In embodiments, the probe includes SEQ ID NO:13969 and SEQ ID NO:11913. In embodiments, the probe includes SEQ ID NO:13970 and SEQ ID NO:11914. In embodiments, the probe includes SEQ ID NO:13971 and SEQ ID NO:11915. In embodiments, the probe includes SEQ ID NO:13972 and SEQ ID NO:11916. In embodiments, the probe includes SEQ ID NO:13973 and SEQ ID NO:11917. In embodiments, the probe includes SEQ ID NO:13974 and SEQ ID NO:11918. In embodiments, the probe includes SEQ ID NO:13975 and SEQ ID NO:11919. In embodiments, the probe includes SEQ ID NO:13976 and SEQ ID NO:11920. In embodiments, the probe includes SEQ ID NO:13977 and SEQ ID NO:11921. In embodiments, the probe includes SEQ ID NO:13978 and SEQ ID NO:11922. In embodiments, the probe includes SEQ ID NO:13979 and SEQ ID NO:11923. In embodiments, the probe includes SEQ ID NO:13980 and SEQ ID NO:11924. In embodiments, the probe includes SEQ ID NO:13981 and SEQ ID NO:11925. In embodiments, the probe includes SEQ ID NO:13982 and SEQ ID NO:11926. In embodiments, the probe includes SEQ ID NO:13983 and SEQ ID NO:11927. In embodiments, the probe includes SEQ ID NO:13984 and SEQ ID NO:11928. In embodiments, the probe includes SEQ ID NO:13985 and SEQ ID NO:11929. In embodiments, the probe includes SEQ ID NO:13986 and SEQ ID NO:11930. In embodiments, the probe includes SEQ ID NO:13987 and SEQ ID NO:11931. In embodiments, the probe includes SEQ ID NO:13988 and SEQ ID NO:11932. In embodiments, the probe includes SEQ ID NO:13989 and SEQ ID NO:11933. In embodiments, the probe includes SEQ ID NO:13990 and SEQ ID NO:11934. In embodiments, the probe includes SEQ ID NO:13991 and SEQ ID NO:11935. In embodiments, the probe includes SEQ ID NO:13992 and SEQ ID NO:11936. In embodiments, the probe includes SEQ ID NO:13993 and SEQ ID NO:11937. In embodiments, the probe includes SEQ ID NO:13994 and SEQ ID NO:11938. In embodiments, the probe includes SEQ ID NO:13995 and SEQ ID NO:11939. In embodiments, the probe includes SEQ ID NO:13996 and SEQ ID NO:11940. In embodiments, the probe includes SEQ ID NO:13997 and SEQ ID NO:11941. In embodiments, the probe includes SEQ ID NO:13998 and SEQ ID NO:11942. In embodiments, the probe includes SEQ ID NO:13999 and SEQ ID NO:11943. In embodiments, the probe includes SEQ ID NO:14000 and SEQ ID NO:11944. In embodiments, the probe includes SEQ ID NO:14001 and SEQ ID NO:11945. In embodiments, the probe includes SEQ ID NO:14002 and SEQ ID NO:11946. In embodiments, the probe includes SEQ ID NO:14003 and SEQ ID NO:11947. In embodiments, the probe includes SEQ ID NO:14004 and SEQ ID NO:11948. In embodiments, the probe includes SEQ ID NO:14005 and SEQ ID NO:11949. In embodiments, the probe includes SEQ ID NO:14006 and SEQ ID NO:11950. In embodiments, the probe includes SEQ ID NO:14007 and SEQ ID NO:11951. In embodiments, the probe includes SEQ ID NO:14008 and SEQ ID NO:11952. In embodiments, the probe includes SEQ ID NO:14009 and SEQ ID NO:11953. In embodiments, the probe includes SEQ ID NO:14010 and SEQ ID NO:11954. In embodiments, the probe includes SEQ ID NO:14011 and SEQ ID NO:11955. In embodiments, the probe includes SEQ ID NO:14012 and SEQ ID NO:11956. In embodiments, the probe includes SEQ ID NO:14013 and SEQ ID NO:11957. In embodiments, the probe includes SEQ ID NO:14014 and SEQ ID NO:11958. In embodiments, the probe includes SEQ ID NO:14015 and SEQ ID NO:11959. In embodiments, the probe includes SEQ ID NO:14016 and SEQ ID NO:11960. In embodiments, the probe includes SEQ ID NO:14017 and SEQ ID NO:11961. In embodiments, the probe includes SEQ ID NO:14018 and SEQ ID NO:11962. In embodiments, the probe includes SEQ ID NO:14019 and SEQ ID NO:11963. In embodiments, the probe includes SEQ ID NO:14020 and SEQ ID NO:11964. In embodiments, the probe includes SEQ ID NO:14021 and SEQ ID NO:11965. In embodiments, the probe includes SEQ ID NO:14022 and SEQ ID NO:11966. In embodiments, the probe includes SEQ ID NO:14023 and SEQ ID NO:11967. In embodiments, the probe includes SEQ ID NO:14024 and SEQ ID NO:11968. In embodiments, the probe includes SEQ ID NO:14025 and SEQ ID NO:11969. In embodiments, the probe includes SEQ ID NO:14026 and SEQ ID NO:11970. In embodiments, the probe includes SEQ ID NO:14027 and SEQ ID NO:11971. In embodiments, the probe includes SEQ ID NO:14028 and SEQ ID NO:11972. In embodiments, the probe includes SEQ ID NO:14029 and SEQ ID NO:11973. In embodiments, the probe includes SEQ ID NO:14030 and SEQ ID NO:11974. In embodiments, the probe includes SEQ ID NO:14031 and SEQ ID NO:11975. In embodiments, the probe includes SEQ ID NO:14032 and SEQ ID NO:11976. In embodiments, the probe includes SEQ ID NO:14033 and SEQ ID NO:11977. In embodiments, the probe includes SEQ ID NO:14034 and SEQ ID NO:11978. In embodiments, the probe includes SEQ ID NO:14035 and SEQ ID NO:11979. In embodiments, the probe includes SEQ ID NO:14036 and SEQ ID NO:11980. In embodiments, the probe includes SEQ ID NO:14037 and SEQ ID NO:11981. In embodiments, the probe includes SEQ ID NO:14038 and SEQ ID NO:11982. In embodiments, the probe includes SEQ ID NO:14039 and SEQ ID NO:11983. In embodiments, the probe includes SEQ ID NO:14040 and SEQ ID NO:11984. In embodiments, the probe includes SEQ ID NO:14041 and SEQ ID NO:11985. In embodiments, the probe includes SEQ ID NO:14042 and SEQ ID NO:11986. In embodiments, the probe includes SEQ ID NO:14043 and SEQ ID NO:11987. In embodiments, the probe includes SEQ ID NO:14044 and SEQ ID NO:11988. In embodiments, the probe includes SEQ ID NO:14045 and SEQ ID NO:11989. In embodiments, the probe includes SEQ ID NO:14046 and SEQ ID NO:11990. In embodiments, the probe includes SEQ ID NO:14047 and SEQ ID NO:11991. In embodiments, the probe includes SEQ ID NO:14048 and SEQ ID NO:11992. In embodiments, the probe includes SEQ ID NO:14049 and SEQ ID NO:11993. In embodiments, the probe includes SEQ ID NO:14050 and SEQ ID NO:11994. In embodiments, the probe includes SEQ ID NO:14051 and SEQ ID NO:11995. In embodiments, the probe includes SEQ ID NO:14052 and SEQ ID NO:11996. In embodiments, the probe includes SEQ ID NO:14053 and SEQ ID NO:11997. In embodiments, the probe includes SEQ ID NO:14054 and SEQ ID NO:11998. In embodiments, the probe includes SEQ ID NO:14055 and SEQ ID NO:11999. In embodiments, the probe includes SEQ ID NO:14056 and SEQ ID NO:12000. In embodiments, the probe includes SEQ ID NO:14057 and SEQ ID NO:12001. In embodiments, the probe includes SEQ ID NO:14058 and SEQ ID NO:12002. In embodiments, the probe includes SEQ ID NO:14059 and SEQ ID NO:12003. In embodiments, the probe includes SEQ ID NO:14060 and SEQ ID NO:12004. In embodiments, the probe includes SEQ ID NO:14061 and SEQ ID NO:12005. In embodiments, the probe includes SEQ ID NO:14062 and SEQ ID NO:12006. In embodiments, the probe includes SEQ ID NO:14063 and SEQ ID NO:12007. In embodiments, the probe includes SEQ ID NO:14064 and SEQ ID NO:12008. In embodiments, the probe includes SEQ ID NO:14065 and SEQ ID NO:12009. In embodiments, the probe includes SEQ ID NO:14066 and SEQ ID NO:12010. In embodiments, the probe includes SEQ ID NO:14067 and SEQ ID NO:12011. In embodiments, the probe includes SEQ ID NO:14068 and SEQ ID NO:12012. In embodiments, the probe includes SEQ ID NO:14069 and SEQ ID NO:12013. In embodiments, the probe includes SEQ ID NO:14070 and SEQ ID NO:12014. In embodiments, the probe includes SEQ ID NO:14071 and SEQ ID NO:12015. In embodiments, the probe includes SEQ ID NO:14072 and SEQ ID NO:12016. In embodiments, the probe includes SEQ ID NO:14073 and SEQ ID NO:12017. In embodiments, the probe includes SEQ ID NO:14074 and SEQ ID NO:12018. In embodiments, the probe includes SEQ ID NO:14075 and SEQ ID NO:12019. In embodiments, the probe includes SEQ ID NO:14076 and SEQ ID NO:12020. In embodiments, the probe includes SEQ ID NO:14077 and SEQ ID NO:12021. In embodiments, the probe includes SEQ ID NO:14078 and SEQ ID NO:12022. In embodiments, the probe includes SEQ ID NO:14079 and SEQ ID NO:12023. In embodiments, the probe includes SEQ ID NO:14080 and SEQ ID NO:12024. In embodiments, the probe includes SEQ ID NO:14081 and SEQ ID NO:12025. In embodiments, the probe includes SEQ ID NO:14082 and SEQ ID NO:12026. In embodiments, the probe includes SEQ ID NO:14083 and SEQ ID NO:12027. In embodiments, the probe includes SEQ ID NO:14084 and SEQ ID NO:12028. In embodiments, the probe includes SEQ ID NO:14085 and SEQ ID NO:12029. In embodiments, the probe includes SEQ ID NO:14086 and SEQ ID NO:12030. In embodiments, the probe includes SEQ ID NO:14087 and SEQ ID NO:12031. In embodiments, the probe includes SEQ ID NO:14088 and SEQ ID NO:12032. In embodiments, the probe includes SEQ ID NO:14089 and SEQ ID NO:12033. In embodiments, the probe includes SEQ ID NO:14090 and SEQ ID NO:12034. In embodiments, the probe includes SEQ ID NO:14091 and SEQ ID NO:12035. In embodiments, the probe includes SEQ ID NO:14092 and SEQ ID NO:12036. In embodiments, the probe includes SEQ ID NO:14093 and SEQ ID NO:12037. In embodiments, the probe includes SEQ ID NO:14094 and SEQ ID NO:12038. In embodiments, the probe includes SEQ ID NO:14095 and SEQ ID NO:12039. In embodiments, the probe includes SEQ ID NO:14096 and SEQ ID NO:12040. In embodiments, the probe includes SEQ ID NO:14097 and SEQ ID NO:12041. In embodiments, the probe includes SEQ ID NO:14098 and SEQ ID NO:12042. In embodiments, the probe includes SEQ ID NO:14099 and SEQ ID NO:12043. In embodiments, the probe includes SEQ ID NO:14100 and SEQ ID NO:12044. In embodiments, the probe includes SEQ ID NO:14101 and SEQ ID NO:12045. In embodiments, the probe includes SEQ ID NO:14102 and SEQ ID NO:12046. In embodiments, the probe includes SEQ ID NO:14103 and SEQ ID NO:12047. In embodiments, the probe includes SEQ ID NO:14104 and SEQ ID NO:12048. In embodiments, the probe includes SEQ ID NO:14105 and SEQ ID NO:12049. In embodiments, the probe includes SEQ ID NO:14106 and SEQ ID NO:12050. In embodiments, the probe includes SEQ ID NO:14107 and SEQ ID NO:12051. In embodiments, the probe includes SEQ ID NO:14108 and SEQ ID NO:12052. In embodiments, the probe includes SEQ ID NO:14109 and SEQ ID NO:12053. In embodiments, the probe includes SEQ ID NO:14110 and SEQ ID NO:12054. In embodiments, the probe includes SEQ ID NO:14111 and SEQ ID NO:12055. In embodiments, the probe includes SEQ ID NO:14112 and SEQ ID NO:12056. In embodiments, the probe includes SEQ ID NO:14113 and SEQ ID NO:12057. In embodiments, the probe includes SEQ ID NO:14114 and SEQ ID NO:12058. In embodiments, the probe includes SEQ ID NO:14115 and SEQ ID NO:12059. In embodiments, the probe includes SEQ ID NO:14116 and SEQ ID NO:12060. In embodiments, the probe includes SEQ ID NO:14117 and SEQ ID NO:12061. In embodiments, the probe includes SEQ ID NO:14118 and SEQ ID NO:12062. In embodiments, the probe includes SEQ ID NO:14119 and SEQ ID NO:12063. In embodiments, the probe includes SEQ ID NO:14120 and SEQ ID NO:12064. In embodiments, the probe includes SEQ ID NO:14121 and SEQ ID NO:12065. In embodiments, the probe includes SEQ ID NO:14122 and SEQ ID NO:12066. In embodiments, the probe includes SEQ ID NO:14123 and SEQ ID NO:12067. In embodiments, the probe includes SEQ ID NO:14124 and SEQ ID NO:12068. In embodiments, the probe includes SEQ ID NO:14125 and SEQ ID NO:12069. In embodiments, the probe includes SEQ ID NO:14126 and SEQ ID NO:12070. In embodiments, the probe includes SEQ ID NO:14127 and SEQ ID NO:12071. In embodiments, the probe includes SEQ ID NO:14128 and SEQ ID NO:12072. In embodiments, the probe includes SEQ ID NO:14129 and SEQ ID NO:12073. In embodiments, the probe includes SEQ ID NO:14130 and SEQ ID NO:12074. In embodiments, the probe includes SEQ ID NO:14131 and SEQ ID NO:12075. In embodiments, the probe includes SEQ ID NO:14132 and SEQ ID NO:12076. In embodiments, the probe includes SEQ ID NO:14133 and SEQ ID NO:12077. In embodiments, the probe includes SEQ ID NO:14134 and SEQ ID NO:12078. In embodiments, the probe includes SEQ ID NO:14135 and SEQ ID NO:12079. In embodiments, the probe includes SEQ ID NO:14136 and SEQ ID NO:12080. In embodiments, the probe includes SEQ ID NO:14137 and SEQ ID NO:12081. In embodiments, the probe includes SEQ ID NO:14138 and SEQ ID NO:12082. In embodiments, the probe includes SEQ ID NO:14139 and SEQ ID NO:12083. In embodiments, the probe includes SEQ ID NO:14140 and SEQ ID NO:12084. In embodiments, the probe includes SEQ ID NO:14141 and SEQ ID NO:12085. In embodiments, the probe includes SEQ ID NO:14142 and SEQ ID NO:12086. In embodiments, the probe includes SEQ ID NO:14143 and SEQ ID NO:12087. In embodiments, the probe includes SEQ ID NO:14144 and SEQ ID NO:12088. In embodiments, the probe includes SEQ ID NO:14145 and SEQ ID NO:12089. In embodiments, the probe includes SEQ ID NO:14146 and SEQ ID NO:12090. In embodiments, the probe includes SEQ ID NO:14147 and SEQ ID NO:12091. In embodiments, the probe includes SEQ ID NO:14148 and SEQ ID NO:12092. In embodiments, the probe includes SEQ ID NO:14149 and SEQ ID NO:12093. In embodiments, the probe includes SEQ ID NO:14150 and SEQ ID NO:12094. In embodiments, the probe includes SEQ ID NO:14151 and SEQ ID NO:12095. In embodiments, the probe includes SEQ ID NO:14152 and SEQ ID NO:12096. In embodiments, the probe includes SEQ ID NO:14153 and SEQ ID NO:12097. In embodiments, the probe includes SEQ ID NO:14154 and SEQ ID NO:12098. In embodiments, the probe includes SEQ ID NO:14155 and SEQ ID NO:12099. In embodiments, the probe includes SEQ ID NO:14156 and SEQ ID NO:12100. In embodiments, the probe includes SEQ ID NO:14157 and SEQ ID NO:12101. In embodiments, the probe includes SEQ ID NO:14158 and SEQ ID NO:12102. In embodiments, the probe includes SEQ ID NO:14159 and SEQ ID NO:12103. In embodiments, the probe includes SEQ ID NO:14160 and SEQ ID NO:12104. In embodiments, the probe includes SEQ ID NO:14161 and SEQ ID NO:12105. In embodiments, the probe includes SEQ ID NO:14162 and SEQ ID NO:12106. In embodiments, the probe includes SEQ ID NO:14163 and SEQ ID NO:12107. In embodiments, the probe includes SEQ ID NO:14164 and SEQ ID NO:12108. In embodiments, the probe includes SEQ ID NO:14165 and SEQ ID NO:12109. In embodiments, the probe includes SEQ ID NO:14166 and SEQ ID NO:12110. In embodiments, the probe includes SEQ ID NO:14167 and SEQ ID NO:12111. In embodiments, the probe includes SEQ ID NO:14168 and SEQ ID NO:12112. In embodiments, the probe includes SEQ ID NO:14169 and SEQ ID NO:12113. In embodiments, the probe includes SEQ ID NO:14170 and SEQ ID NO:12114. In embodiments, the probe includes SEQ ID NO:14171 and SEQ ID NO:12115. In embodiments, the probe includes SEQ ID NO:14172 and SEQ ID NO:12116. In embodiments, the probe includes SEQ ID NO:14173 and SEQ ID NO:12117. In embodiments, the probe includes SEQ ID NO:14174 and SEQ ID NO:12118. In embodiments, the probe includes SEQ ID NO:14175 and SEQ ID NO:12119. In embodiments, the probe includes SEQ ID NO:14176 and SEQ ID NO:12120. In embodiments, the probe includes SEQ ID NO:14177 and SEQ ID NO:12121. In embodiments, the probe includes SEQ ID NO:14178 and SEQ ID NO:12122. In embodiments, the probe includes SEQ ID NO:14179 and SEQ ID NO:12123. In embodiments, the probe includes SEQ ID NO:14180 and SEQ ID NO:12124. In embodiments, the probe includes SEQ ID NO:14181 and SEQ ID NO:12125. In embodiments, the probe includes SEQ ID NO:14182 and SEQ ID NO:12126. In embodiments, the probe includes SEQ ID NO:14183 and SEQ ID NO:12127. In embodiments, the probe includes SEQ ID NO:14184 and SEQ ID NO:12128. In embodiments, the probe includes SEQ ID NO:14185 and SEQ ID NO:12129. In embodiments, the probe includes SEQ ID NO:14186 and SEQ ID NO:12130. In embodiments, the probe includes SEQ ID NO:14187 and SEQ ID NO:12131. In embodiments, the probe includes SEQ ID NO:14188 and SEQ ID NO:12132. In embodiments, the probe includes SEQ ID NO:14189 and SEQ ID NO:12133. In embodiments, the probe includes SEQ ID NO:14190 and SEQ ID NO:12134. In embodiments, the probe includes SEQ ID NO:14191 and SEQ ID NO:12135. In embodiments, the probe includes SEQ ID NO:14192 and SEQ ID NO:12136. In embodiments, the probe includes SEQ ID NO:14193 and SEQ ID NO:12137. In embodiments, the probe includes SEQ ID NO:14194 and SEQ ID NO:12138. In embodiments, the probe includes SEQ ID NO:14195 and SEQ ID NO:12139. In embodiments, the probe includes SEQ ID NO:14196 and SEQ ID NO:12140. In embodiments, the probe includes SEQ ID NO:14197 and SEQ ID NO:12141. In embodiments, the probe includes SEQ ID NO:14198 and SEQ ID NO:12142. In embodiments, the probe includes SEQ ID NO:14199 and SEQ ID NO:12143. In embodiments, the probe includes SEQ ID NO:14200 and SEQ ID NO:12144. In embodiments, the probe includes SEQ ID NO:14201 and SEQ ID NO:12145. In embodiments, the probe includes SEQ ID NO:14202 and SEQ ID NO:12146. In embodiments, the probe includes SEQ ID NO:14203 and SEQ ID NO:12147. In embodiments, the probe includes SEQ ID NO:14204 and SEQ ID NO:12148. In embodiments, the probe includes SEQ ID NO:14205 and SEQ ID NO:12149. In embodiments, the probe includes SEQ ID NO:14206 and SEQ ID NO:12150. In embodiments, the probe includes SEQ ID NO:14207 and SEQ ID NO:12151. In embodiments, the probe includes SEQ ID NO:14208 and SEQ ID NO:12152. In embodiments, the probe includes SEQ ID NO:14209 and SEQ ID NO:12153. In embodiments, the probe includes SEQ ID NO:14210 and SEQ ID NO:12154. In embodiments, the probe includes SEQ ID NO:14211 and SEQ ID NO:12155. In embodiments, the probe includes SEQ ID NO:14212 and SEQ ID NO:12156. In embodiments, the probe includes SEQ ID NO:14213 and SEQ ID NO:12157. In embodiments, the probe includes SEQ ID NO:14214 and SEQ ID NO:12158. In embodiments, the probe includes SEQ ID NO:14215 and SEQ ID NO:12159. In embodiments, the probe includes SEQ ID NO:14216 and SEQ ID NO:12160. In embodiments, the probe includes SEQ ID NO:14217 and SEQ ID NO:12161. In embodiments, the probe includes SEQ ID NO:14218 and SEQ ID NO:12162. In embodiments, the probe includes SEQ ID NO:14219 and SEQ ID NO:12163. In embodiments, the probe includes SEQ ID NO:14220 and SEQ ID NO:12164. In embodiments, the probe includes SEQ ID NO:14221 and SEQ ID NO:12165. In embodiments, the probe includes SEQ ID NO:14222 and SEQ ID NO:12166. In embodiments, the probe includes SEQ ID NO:14223 and SEQ ID NO:12167. In embodiments, the probe includes SEQ ID NO:14224 and SEQ ID NO:12168. In embodiments, the probe includes SEQ ID NO:14225 and SEQ ID NO:12169. In embodiments, the probe includes SEQ ID NO:14226 and SEQ ID NO:12170. In embodiments, the probe includes SEQ ID NO:14227 and SEQ ID NO:12171. In embodiments, the probe includes SEQ ID NO:14228 and SEQ ID NO:12172. In embodiments, the probe includes SEQ ID NO:14229 and SEQ ID NO:12173. In embodiments, the probe includes SEQ ID NO:14230 and SEQ ID NO:12174. In embodiments, the probe includes SEQ ID NO:14231 and SEQ ID NO:12175. In embodiments, the probe includes SEQ ID NO:14232 and SEQ ID NO:12176. In embodiments, the probe includes SEQ ID NO:14233 and SEQ ID NO:12177. In embodiments, the probe includes SEQ ID NO:14234 and SEQ ID NO:12178. In embodiments, the probe includes SEQ ID NO:14235 and SEQ ID NO:12179. In embodiments, the probe includes SEQ ID NO:14236 and SEQ ID NO:12180. In embodiments, the probe includes SEQ ID NO:14237 and SEQ ID NO:12181. In embodiments, the probe includes SEQ ID NO:14238 and SEQ ID NO:12182. In embodiments, the probe includes SEQ ID NO:14239 and SEQ ID NO:12183. In embodiments, the probe includes SEQ ID NO:14240 and SEQ ID NO:12184. In embodiments, the probe includes SEQ ID NO:14241 and SEQ ID NO:12185. In embodiments, the probe includes SEQ ID NO:14242 and SEQ ID NO:12186. In embodiments, the probe includes SEQ ID NO:14243 and SEQ ID NO:12187. In embodiments, the probe includes SEQ ID NO:14244 and SEQ ID NO:12188. In embodiments, the probe includes SEQ ID NO:14245 and SEQ ID NO:12189. In embodiments, the probe includes SEQ ID NO:14246 and SEQ ID NO:12190. In embodiments, the probe includes SEQ ID NO:14247 and SEQ ID NO:12191. In embodiments, the probe includes SEQ ID NO:14248 and SEQ ID NO:12192. In embodiments, the probe includes SEQ ID NO:14249 and SEQ ID NO:12193. In embodiments, the probe includes SEQ ID NO:14250 and SEQ ID NO:12194. In embodiments, the probe includes SEQ ID NO:14251 and SEQ ID NO:12195. In embodiments, the probe includes SEQ ID NO:14252 and SEQ ID NO:12196. In embodiments, the probe includes SEQ ID NO:14253 and SEQ ID NO:12197. In embodiments, the probe includes SEQ ID NO:14254 and SEQ ID NO:12198. In embodiments, the probe includes SEQ ID NO:14255 and SEQ ID NO:12199. In embodiments, the probe includes SEQ ID NO:14256 and SEQ ID NO:12200. In embodiments, the probe includes SEQ ID NO:14257 and SEQ ID NO:12201. In embodiments, the probe includes SEQ ID NO:14258 and SEQ ID NO:12202. In embodiments, the probe includes SEQ ID NO:14259 and SEQ ID NO:12203. In embodiments, the probe includes SEQ ID NO:14260 and SEQ ID NO:12204. In embodiments, the probe includes SEQ ID NO:14261 and SEQ ID NO:12205. In embodiments, the probe includes SEQ ID NO:14262 and SEQ ID NO:12206. In embodiments, the probe includes SEQ ID NO:14263 and SEQ ID NO:12207. In embodiments, the probe includes SEQ ID NO:14264 and SEQ ID NO:12208. In embodiments, the probe includes SEQ ID NO:14265 and SEQ ID NO:12209. In embodiments, the probe includes SEQ ID NO:14266 and SEQ ID NO:12210. In embodiments, the probe includes SEQ ID NO:14267 and SEQ ID NO:12211. In embodiments, the probe includes SEQ ID NO:14268 and SEQ ID NO:12212. In embodiments, the probe includes SEQ ID NO:14269 and SEQ ID NO:12213. In embodiments, the probe includes SEQ ID NO:14270 and SEQ ID NO:12214. In embodiments, the probe includes SEQ ID NO:14271 and SEQ ID NO:12215. In embodiments, the probe includes SEQ ID NO:14272 and SEQ ID NO:12216. In embodiments, the probe includes SEQ ID NO:14273 and SEQ ID NO:12217. In embodiments, the probe includes SEQ ID NO:14274 and SEQ ID NO:12218. In embodiments, the probe includes SEQ ID NO:14275 and SEQ ID NO:12219. In embodiments, the probe includes SEQ ID NO:14276 and SEQ ID NO:12220. In embodiments, the probe includes SEQ ID NO:14277 and SEQ ID NO:12221. In embodiments, the probe includes SEQ ID NO:14278 and SEQ ID NO:12222. In embodiments, the probe includes SEQ ID NO:14279 and SEQ ID NO:12223. In embodiments, the probe includes SEQ ID NO:14280 and SEQ ID NO:12224. In embodiments, the probe includes SEQ ID NO:14281 and SEQ ID NO:12225. In embodiments, the probe includes SEQ ID NO:14282 and SEQ ID NO:12226. In embodiments, the probe includes SEQ ID NO:14283 and SEQ ID NO:12227. In embodiments, the probe includes SEQ ID NO:14284 and SEQ ID NO:12228. In embodiments, the probe includes SEQ ID NO:14285 and SEQ ID NO:12229. In embodiments, the probe includes SEQ ID NO:14286 and SEQ ID NO:12230. In embodiments, the probe includes SEQ ID NO:14287 and SEQ ID NO:12231. In embodiments, the probe includes SEQ ID NO:14288 and SEQ ID NO:12232. In embodiments, the probe includes SEQ ID NO:14289 and SEQ ID NO:12233. In embodiments, the probe includes SEQ ID NO:14290 and SEQ ID NO:12234. In embodiments, the probe includes SEQ ID NO:14291 and SEQ ID NO:12235. In embodiments, the probe includes SEQ ID NO:14292 and SEQ ID NO:12236. In embodiments, the probe includes SEQ ID NO:14293 and SEQ ID NO:12237. In embodiments, the probe includes SEQ ID NO:14294 and SEQ ID NO:12238. In embodiments, the probe includes SEQ ID NO:14295 and SEQ ID NO:12239. In embodiments, the probe includes SEQ ID NO:14296 and SEQ ID NO:12240. In embodiments, the probe includes SEQ ID NO:14297 and SEQ ID NO:12241. In embodiments, the probe includes SEQ ID NO:14298 and SEQ ID NO:12242. In embodiments, the probe includes SEQ ID NO:14299 and SEQ ID NO:12243. In embodiments, the probe includes SEQ ID NO:14300 and SEQ ID NO:12244. In embodiments, the probe includes SEQ ID NO:14301 and SEQ ID NO:12245. In embodiments, the probe includes SEQ ID NO:14302 and SEQ ID NO:12246. In embodiments, the probe includes SEQ ID NO:14303 and SEQ ID NO:12247. In embodiments, the probe includes SEQ ID NO:14304 and SEQ ID NO:12248. In embodiments, the probe includes SEQ ID NO:14305 and SEQ ID NO:12249. In embodiments, the probe includes SEQ ID NO:14306 and SEQ ID NO:12250. In embodiments, the probe includes SEQ ID NO:14307 and SEQ ID NO:12251. In embodiments, the probe includes SEQ ID NO:14308 and SEQ ID NO:12252. In embodiments, the probe includes SEQ ID NO:14309 and SEQ ID NO:12253. In embodiments, the probe includes SEQ ID NO:14310 and SEQ ID NO:12254. In embodiments, the probe includes SEQ ID NO:14311 and SEQ ID NO:12255. In embodiments, the probe includes SEQ ID NO:14312 and SEQ ID NO:12256. In embodiments, the probe includes SEQ ID NO:14313 and SEQ ID NO:12257. In embodiments, the probe includes SEQ ID NO:14314 and SEQ ID NO:12258. In embodiments, the probe includes SEQ ID NO:14315 and SEQ ID NO:12259. In embodiments, the probe includes SEQ ID NO:14316 and SEQ ID NO:12260. In embodiments, the probe includes SEQ ID NO:14317 and SEQ ID NO:12261. In embodiments, the probe includes SEQ ID NO:14318 and SEQ ID NO:12262. In embodiments, the probe includes SEQ ID NO:14319 and SEQ ID NO:12263. In embodiments, the probe includes SEQ ID NO:14320 and SEQ ID NO:12264. In embodiments, the probe includes SEQ ID NO:14321 and SEQ ID NO:12265. In embodiments, the probe includes SEQ ID NO:14322 and SEQ ID NO:12266. In embodiments, the probe includes SEQ ID NO:14323 and SEQ ID NO:12267. In embodiments, the probe includes SEQ ID NO:14324 and SEQ ID NO:12268. In embodiments, the probe includes SEQ ID NO:14325 and SEQ ID NO:12269. In embodiments, the probe includes SEQ ID NO:14326 and SEQ ID NO:12270. In embodiments, the probe includes SEQ ID NO:14327 and SEQ ID NO:12271. In embodiments, the probe includes SEQ ID NO:14328 and SEQ ID NO:12272. In embodiments, the probe includes SEQ ID NO:14329 and SEQ ID NO:12273. In embodiments, the probe includes SEQ ID NO:14330 and SEQ ID NO:12274. In embodiments, the probe includes SEQ ID NO:14331 and SEQ ID NO:12275. In embodiments, the probe includes SEQ ID NO:14332 and SEQ ID NO:12276. In embodiments, the probe includes SEQ ID NO:14333 and SEQ ID NO:12277. In embodiments, the probe includes SEQ ID NO:14334 and SEQ ID NO:12278. In embodiments, the probe includes SEQ ID NO:14335 and SEQ ID NO:12279. In embodiments, the probe includes SEQ ID NO:14336 and SEQ ID NO:12280. In embodiments, the probe includes SEQ ID NO:14337 and SEQ ID NO:12281. In embodiments, the probe includes SEQ ID NO:14338 and SEQ ID NO:12282. In embodiments, the probe includes SEQ ID NO:14339 and SEQ ID NO:12283. In embodiments, the probe includes SEQ ID NO:14340 and SEQ ID NO:12284. In embodiments, the probe includes SEQ ID NO:14341 and SEQ ID NO:12285. In embodiments, the probe includes SEQ ID NO:14342 and SEQ ID NO:12286. In embodiments, the probe includes SEQ ID NO:14343 and SEQ ID NO:12287. In embodiments, the probe includes SEQ ID NO:14344 and SEQ ID NO:12288. In embodiments, the probe includes SEQ ID NO:14345 and SEQ ID NO:12289. In embodiments, the probe includes SEQ ID NO:14346 and SEQ ID NO:12290. In embodiments, the probe includes SEQ ID NO:14347 and SEQ ID NO:12291. In embodiments, the probe includes SEQ ID NO:14348 and SEQ ID NO:12292. In embodiments, the probe includes SEQ ID NO:14349 and SEQ ID NO:12293. In embodiments, the probe includes SEQ ID NO:14350 and SEQ ID NO:12294. In embodiments, the probe includes SEQ ID NO:14351 and SEQ ID NO:12295. In embodiments, the probe includes SEQ ID NO:14352 and SEQ ID NO:12296. In embodiments, the probe includes SEQ ID NO:14353 and SEQ ID NO:12297. In embodiments, the probe includes SEQ ID NO:14354 and SEQ ID NO:12298. In embodiments, the probe includes SEQ ID NO:14355 and SEQ ID NO:12299. In embodiments, the probe includes SEQ ID NO:14356 and SEQ ID NO:12300. In embodiments, the probe includes SEQ ID NO:14357 and SEQ ID NO:12301. In embodiments, the probe includes SEQ ID NO:14358 and SEQ ID NO:12302. In embodiments, the probe includes SEQ ID NO:14359 and SEQ ID NO:12303. In embodiments, the probe includes SEQ ID NO:14360 and SEQ ID NO:12304. In embodiments, the probe includes SEQ ID NO:14361 and SEQ ID NO:12305. In embodiments, the probe includes SEQ ID NO:14362 and SEQ ID NO:12306. In embodiments, the probe includes SEQ ID NO:14363 and SEQ ID NO:12307. In embodiments, the probe includes SEQ ID NO:14364 and SEQ ID NO:12308. In embodiments, the probe includes SEQ ID NO:14365 and SEQ ID NO:12309. In embodiments, the probe includes SEQ ID NO:14366 and SEQ ID NO:12310. In embodiments, the probe includes SEQ ID NO:14367 and SEQ ID NO:12311. In embodiments, the probe includes SEQ ID NO:14368 and SEQ ID NO:12312. In embodiments, the probe includes SEQ ID NO:14369 and SEQ ID NO:12313. In embodiments, the probe includes SEQ ID NO:14370 and SEQ ID NO:12314. In embodiments, the probe includes SEQ ID NO:14371 and SEQ ID NO:12315. In embodiments, the probe includes SEQ ID NO:14372 and SEQ ID NO:12316. In embodiments, the probe includes SEQ ID NO:14373 and SEQ ID NO:12317. In embodiments, the probe includes SEQ ID NO:14374 and SEQ ID NO:12318. In embodiments, the probe includes SEQ ID NO:14375 and SEQ ID NO:12319. In embodiments, the probe includes SEQ ID NO:14376 and SEQ ID NO:12320. In embodiments, the probe includes SEQ ID NO:14377 and SEQ ID NO:12321. In embodiments, the probe includes SEQ ID NO:14378 and SEQ ID NO:12322. In embodiments, the probe includes SEQ ID NO:14379 and SEQ ID NO:12323. In embodiments, the probe includes SEQ ID NO:14380 and SEQ ID NO:12324. In embodiments, the probe includes SEQ ID NO:14381 and SEQ ID NO:12325. In embodiments, the probe includes SEQ ID NO:14382 and SEQ ID NO:12326. In embodiments, the probe includes SEQ ID NO:14383 and SEQ ID NO:12327. In embodiments, the probe includes SEQ ID NO:14384 and SEQ ID NO:12328. In embodiments, the probe includes SEQ ID NO:14385 and SEQ ID NO:12329. In embodiments, the probe includes SEQ ID NO:14386 and SEQ ID NO:12330. In embodiments, the probe includes SEQ ID NO:14387 and SEQ ID NO:12331. In embodiments, the probe includes SEQ ID NO:14388 and SEQ ID NO:12332. In embodiments, the probe includes SEQ ID NO:14389 and SEQ ID NO:12333. In embodiments, the probe includes SEQ ID NO:14390 and SEQ ID NO:12334. In embodiments, the probe includes SEQ ID NO:14391 and SEQ ID NO:12335. In embodiments, the probe includes SEQ ID NO:14392 and SEQ ID NO:12336. In embodiments, the probe includes SEQ ID NO:14393 and SEQ ID NO:12337. In embodiments, the probe includes SEQ ID NO:14394 and SEQ ID NO:12338. In embodiments, the probe includes SEQ ID NO:14395 and SEQ ID NO:12339. In embodiments, the probe includes SEQ ID NO:14396 and SEQ ID NO:12340. In embodiments, the probe includes SEQ ID NO:14397 and SEQ ID NO:12341. In embodiments, the probe includes SEQ ID NO:14398 and SEQ ID NO:12342. In embodiments, the probe includes SEQ ID NO:14399 and SEQ ID NO:12343. In embodiments, the probe includes SEQ ID NO:14400 and SEQ ID NO:12344. In embodiments, the probe includes SEQ ID NO:14401 and SEQ ID NO:12345. In embodiments, the probe includes SEQ ID NO:14402 and SEQ ID NO:12346. In embodiments, the probe includes SEQ ID NO:14403 and SEQ ID NO:12347. In embodiments, the probe includes SEQ ID NO:14404 and SEQ ID NO:12348. In embodiments, the probe includes SEQ ID NO:14405 and SEQ ID NO:12349. In embodiments, the probe includes SEQ ID NO:14406 and SEQ ID NO:12350. In embodiments, the probe includes SEQ ID NO:14407 and SEQ ID NO:12351. In embodiments, the probe includes SEQ ID NO:14408 and SEQ ID NO:12352. In embodiments, the probe includes SEQ ID NO:14409 and SEQ ID NO:12353. In embodiments, the probe includes SEQ ID NO:14410 and SEQ ID NO:12354. In embodiments, the probe includes SEQ ID NO:14411 and SEQ ID NO:12355. In embodiments, the probe includes SEQ ID NO:14412 and SEQ ID NO:12356. In embodiments, the probe includes SEQ ID NO:14413 and SEQ ID NO:12357. In embodiments, the probe includes SEQ ID NO:14414 and SEQ ID NO:12358. In embodiments, the probe includes SEQ ID NO:14415 and SEQ ID NO:12359. In embodiments, the probe includes SEQ ID NO:14416 and SEQ ID NO:12360. In embodiments, the probe includes SEQ ID NO:14417 and SEQ ID NO:12361. In embodiments, the probe includes SEQ ID NO:14418 and SEQ ID NO:12362. In embodiments, the probe includes SEQ ID NO:14419 and SEQ ID NO:12363. In embodiments, the probe includes SEQ ID NO:14420 and SEQ ID NO:12364. In embodiments, the probe includes SEQ ID NO:14421 and SEQ ID NO:12365. In embodiments, the probe includes SEQ ID NO:14422 and SEQ ID NO:12366. In embodiments, the probe includes SEQ ID NO:14423 and SEQ ID NO:12367. In embodiments, the probe includes SEQ ID NO:14424 and SEQ ID NO:12368. In embodiments, the probe includes SEQ ID NO:14425 and SEQ ID NO:12369. In embodiments, the probe includes SEQ ID NO:14426 and SEQ ID NO:12370. In embodiments, the probe includes SEQ ID NO:14427 and SEQ ID NO:12371. In embodiments, the probe includes SEQ ID NO:14428 and SEQ ID NO:12372. In embodiments, the probe includes SEQ ID NO:14429 and SEQ ID NO:12373. In embodiments, the probe includes SEQ ID NO:14430 and SEQ ID NO:12374. In embodiments, the probe includes SEQ ID NO:14431 and SEQ ID NO:12375. In embodiments, the probe includes SEQ ID NO:14432 and SEQ ID NO:12376. In embodiments, the probe includes SEQ ID NO:14433 and SEQ ID NO:12377. In embodiments, the probe includes SEQ ID NO:14434 and SEQ ID NO:12378. In embodiments, the probe includes SEQ ID NO:14435 and SEQ ID NO:12379. In embodiments, the probe includes SEQ ID NO:14436 and SEQ ID NO:12380. In embodiments, the probe includes SEQ ID NO:14437 and SEQ ID NO:12381. In embodiments, the probe includes SEQ ID NO:14438 and SEQ ID NO:12382. In embodiments, the probe includes SEQ ID NO:14439 and SEQ ID NO:12383. In embodiments, the probe includes SEQ ID NO:14440 and SEQ ID NO:12384. In embodiments, the probe includes SEQ ID NO:14441 and SEQ ID NO:12385. In embodiments, the probe includes SEQ ID NO:14442 and SEQ ID NO:12386. In embodiments, the probe includes SEQ ID NO:14443 and SEQ ID NO:12387. In embodiments, the probe includes SEQ ID NO:14444 and SEQ ID NO:12388. In embodiments, the probe includes SEQ ID NO:14445 and SEQ ID NO:12389. In embodiments, the probe includes SEQ ID NO:14446 and SEQ ID NO:12390. In embodiments, the probe includes SEQ ID NO:14447 and SEQ ID NO:12391. In embodiments, the probe includes SEQ ID NO:14448 and SEQ ID NO:12392. In embodiments, the probe includes SEQ ID NO:14449 and SEQ ID NO:12393. In embodiments, the probe includes SEQ ID NO:14450 and SEQ ID NO:12394. In embodiments, the probe includes SEQ ID NO:14451 and SEQ ID NO:12395. In embodiments, the probe includes SEQ ID NO:14452 and SEQ ID NO:12396. In embodiments, the probe includes SEQ ID NO:14453 and SEQ ID NO:12397. In embodiments, the probe includes SEQ ID NO:14454 and SEQ ID NO:12398. In embodiments, the probe includes SEQ ID NO:14455 and SEQ ID NO:12399. In embodiments, the probe includes SEQ ID NO:14456 and SEQ ID NO:12400. In embodiments, the probe includes SEQ ID NO:14457 and SEQ ID NO:12401. In embodiments, the probe includes SEQ ID NO:14458 and SEQ ID NO:12402. In embodiments, the probe includes SEQ ID NO:14459 and SEQ ID NO:12403. In embodiments, the probe includes SEQ ID NO:14460 and SEQ ID NO:12404. In embodiments, the probe includes SEQ ID NO:14461 and SEQ ID NO:12405. In embodiments, the probe includes SEQ ID NO:14462 and SEQ ID NO:12406. In embodiments, the probe includes SEQ ID NO:14463 and SEQ ID NO:12407. In embodiments, the probe includes SEQ ID NO:14464 and SEQ ID NO:12408. In embodiments, the probe includes SEQ ID NO:14465 and SEQ ID NO:12409. In embodiments, the probe includes SEQ ID NO:14466 and SEQ ID NO:12410. In embodiments, the probe includes SEQ ID NO:14467 and SEQ ID NO:12411. In embodiments, the probe includes SEQ ID NO:14468 and SEQ ID NO:12412. In embodiments, the probe includes SEQ ID NO:14469 and SEQ ID NO:12413. In embodiments, the probe includes SEQ ID NO:14470 and SEQ ID NO:12414. In embodiments, the probe includes SEQ ID NO:14471 and SEQ ID NO:12415. In embodiments, the probe includes SEQ ID NO:14472 and SEQ ID NO:12416. In embodiments, the probe includes SEQ ID NO:14473 and SEQ ID NO:12417. In embodiments, the probe includes SEQ ID NO:14474 and SEQ ID NO:12418. In embodiments, the probe includes SEQ ID NO:14475 and SEQ ID NO:12419. In embodiments, the probe includes SEQ ID NO:14476 and SEQ ID NO:12420. In embodiments, the probe includes SEQ ID NO:14477 and SEQ ID NO:12421. In embodiments, the probe includes SEQ ID NO:14478 and SEQ ID NO:12422. In embodiments, the probe includes SEQ ID NO:14479 and SEQ ID NO:12423. In embodiments, the probe includes SEQ ID NO:14480 and SEQ ID NO:12424. In embodiments, the probe includes SEQ ID NO:14481 and SEQ ID NO:12425. In embodiments, the probe includes SEQ ID NO:14482 and SEQ ID NO:12426. In embodiments, the probe includes SEQ ID NO:14483 and SEQ ID NO:12427. In embodiments, the probe includes SEQ ID NO:14484 and SEQ ID NO:12428. In embodiments, the probe includes SEQ ID NO:14485 and SEQ ID NO:12429. In embodiments, the probe includes SEQ ID NO:14486 and SEQ ID NO:12430. In embodiments, the probe includes SEQ ID NO:14487 and SEQ ID NO:12431. In embodiments, the probe includes SEQ ID NO:14488 and SEQ ID NO:12432. In embodiments, the probe includes SEQ ID NO:14489 and SEQ ID NO:12433. In embodiments, the probe includes SEQ ID NO:14490 and SEQ ID NO:12434. In embodiments, the probe includes SEQ ID NO:14491 and SEQ ID NO:12435. In embodiments, the probe includes SEQ ID NO:14492 and SEQ ID NO:12436. In embodiments, the probe includes SEQ ID NO:14493 and SEQ ID NO:12437. In embodiments, the probe includes SEQ ID NO:14494 and SEQ ID NO:12438. In embodiments, the probe includes SEQ ID NO:14495 and SEQ ID NO:12439. In embodiments, the probe includes SEQ ID NO:14496 and SEQ ID NO:12440. In embodiments, the probe includes SEQ ID NO:14497 and SEQ ID NO:12441. In embodiments, the probe includes SEQ ID NO:14498 and SEQ ID NO:12442. In embodiments, the probe includes SEQ ID NO:14499 and SEQ ID NO:12443. In embodiments, the probe includes SEQ ID NO:14500 and SEQ ID NO:12444. In embodiments, the probe includes SEQ ID NO:14501 and SEQ ID NO:12445. In embodiments, the probe includes SEQ ID NO:14502 and SEQ ID NO:12446. In embodiments, the probe includes SEQ ID NO:14503 and SEQ ID NO:12447. In embodiments, the probe includes SEQ ID NO:14504 and SEQ ID NO:12448. In embodiments, the probe includes SEQ ID NO:14505 and SEQ ID NO:12449. In embodiments, the probe includes SEQ ID NO:14506 and SEQ ID NO:12450. In embodiments, the probe includes SEQ ID NO:14507 and SEQ ID NO:12451. In embodiments, the probe includes SEQ ID NO:14508 and SEQ ID NO:12452. In embodiments, the probe includes SEQ ID NO:14509 and SEQ ID NO:12453. In embodiments, the probe includes SEQ ID NO:14510 and SEQ ID NO:12454. In embodiments, the probe includes SEQ ID NO:14511 and SEQ ID NO:12455. In embodiments, the probe includes SEQ ID NO:14512 and SEQ ID NO:12456. In embodiments, the probe includes SEQ ID NO:14513 and SEQ ID NO:12457. In embodiments, the probe includes SEQ ID NO:14514 and SEQ ID NO:12458. In embodiments, the probe includes SEQ ID NO:14515 and SEQ ID NO:12459. In embodiments, the probe includes SEQ ID NO:14516 and SEQ ID NO:12460. In embodiments, the probe includes SEQ ID NO:14517 and SEQ ID NO:12461. In embodiments, the probe includes SEQ ID NO:14518 and SEQ ID NO:12462. In embodiments, the probe includes SEQ ID NO:14519 and SEQ ID NO:12463. In embodiments, the probe includes SEQ ID NO:14520 and SEQ ID NO:12464. In embodiments, the probe includes SEQ ID NO:14521 and SEQ ID NO:12465. In embodiments, the probe includes SEQ ID NO:14522 and SEQ ID NO:12466. In embodiments, the probe includes SEQ ID NO:14523 and SEQ ID NO:12467. In embodiments, the probe includes SEQ ID NO:14524 and SEQ ID NO:12468. In embodiments, the probe includes SEQ ID NO:14525 and SEQ ID NO:12469. In embodiments, the probe includes SEQ ID NO:14526 and SEQ ID NO:12470. In embodiments, the probe includes SEQ ID NO:14527 and SEQ ID NO:12471. In embodiments, the probe includes SEQ ID NO:14528 and SEQ ID NO:12472. In embodiments, the probe includes SEQ ID NO:14529 and SEQ ID NO:12473. In embodiments, the probe includes SEQ ID NO:14530 and SEQ ID NO:12474. In embodiments, the probe includes SEQ ID NO:14531 and SEQ ID NO:12475. In embodiments, the probe includes SEQ ID NO:14532 and SEQ ID NO:12476. In embodiments, the probe includes SEQ ID NO:14533 and SEQ ID NO:12477. In embodiments, the probe includes SEQ ID NO:14534 and SEQ ID NO:12478. In embodiments, the probe includes SEQ ID NO:14535 and SEQ ID NO:12479. In embodiments, the probe includes SEQ ID NO:14536 and SEQ ID NO:12480. In embodiments, the probe includes SEQ ID NO:14537 and SEQ ID NO:12481. In embodiments, the probe includes SEQ ID NO:14538 and SEQ ID NO:12482. In embodiments, the probe includes SEQ ID NO:14539 and SEQ ID NO:12483. In embodiments, the probe includes SEQ ID NO:14540 and SEQ ID NO:12484. In embodiments, the probe includes SEQ ID NO:14541 and SEQ ID NO:12485. In embodiments, the probe includes SEQ ID NO:14542 and SEQ ID NO:12486. In embodiments, the probe includes SEQ ID NO:14543 and SEQ ID NO:12487. In embodiments, the probe includes SEQ ID NO:14544 and SEQ ID NO:12488. In embodiments, the probe includes SEQ ID NO:14545 and SEQ ID NO:12489. In embodiments, the probe includes SEQ ID NO:14546 and SEQ ID NO:12490. In embodiments, the probe includes SEQ ID NO:14547 and SEQ ID NO:12491. In embodiments, the probe includes SEQ ID NO:14548 and SEQ ID NO:12492. In embodiments, the probe includes SEQ ID NO:14549 and SEQ ID NO:12493. In embodiments, the probe includes SEQ ID NO:14550 and SEQ ID NO:12494. In embodiments, the probe includes SEQ ID NO:14551 and SEQ ID NO:12495. In embodiments, the probe includes SEQ ID NO:14552 and SEQ ID NO:12496. In embodiments, the probe includes SEQ ID NO:14553 and SEQ ID NO:12497. In embodiments, the probe includes SEQ ID NO:14554 and SEQ ID NO:12498. In embodiments, the probe includes SEQ ID NO:14555 and SEQ ID NO:12499. In embodiments, the probe includes SEQ ID NO:14556 and SEQ ID NO:12500. In embodiments, the probe includes SEQ ID NO:14557 and SEQ ID NO:12501. In embodiments, the probe includes SEQ ID NO:14558 and SEQ ID NO:12502. In embodiments, the probe includes SEQ ID NO:14559 and SEQ ID NO:12503. In embodiments, the probe includes SEQ ID NO:14560 and SEQ ID NO:12504. In embodiments, the probe includes SEQ ID NO:14561 and SEQ ID NO:12505. In embodiments, the probe includes SEQ ID NO:14562 and SEQ ID NO:12506. In embodiments, the probe includes SEQ ID NO:14563 and SEQ ID NO:12507. In embodiments, the probe includes SEQ ID NO:14564 and SEQ ID NO:12508. In embodiments, the probe includes SEQ ID NO:14565 and SEQ ID NO:12509. In embodiments, the probe includes SEQ ID NO:14566 and SEQ ID NO:12510. In embodiments, the probe includes SEQ ID NO:14567 and SEQ ID NO:12511. In embodiments, the probe includes SEQ ID NO:14568 and SEQ ID NO:12512. In embodiments, the probe includes SEQ ID NO:14569 and SEQ ID NO:12513. In embodiments, the probe includes SEQ ID NO:14570 and SEQ ID NO:12514. In embodiments, the probe includes SEQ ID NO:14571 and SEQ ID NO:12515. In embodiments, the probe includes SEQ ID NO:14572 and SEQ ID NO:12516. In embodiments, the probe includes SEQ ID NO:14573 and SEQ ID NO:12517. In embodiments, the probe includes SEQ ID NO:14574 and SEQ ID NO:12518. In embodiments, the probe includes SEQ ID NO:14575 and SEQ ID NO:12519. In embodiments, the probe includes SEQ ID NO:14576 and SEQ ID NO:12520. In embodiments, the probe includes SEQ ID NO:14577 and SEQ ID NO:12521. In embodiments, the probe includes SEQ ID NO:14578 and SEQ ID NO:12522. In embodiments, the probe includes SEQ ID NO:14579 and SEQ ID NO:12523. In embodiments, the probe includes SEQ ID NO:14580 and SEQ ID NO:12524. In embodiments, the probe includes SEQ ID NO:14581 and SEQ ID NO:12525. In embodiments, the probe includes SEQ ID NO:14582 and SEQ ID NO:12526. In embodiments, the probe includes SEQ ID NO:14583 and SEQ ID NO:12527. In embodiments, the probe includes SEQ ID NO:14584 and SEQ ID NO:12528. In embodiments, the probe includes SEQ ID NO:14585 and SEQ ID NO:12529. In embodiments, the probe includes SEQ ID NO:14586 and SEQ ID NO:12530. In embodiments, the probe includes SEQ ID NO:14587 and SEQ ID NO:12531. In embodiments, the probe includes SEQ ID NO:14588 and SEQ ID NO:12532. In embodiments, the probe includes SEQ ID NO:14589 and SEQ ID NO:12533. In embodiments, the probe includes SEQ ID NO:14590 and SEQ ID NO:12534. In embodiments, the probe includes SEQ ID NO:14591 and SEQ ID NO:12535. In embodiments, the probe includes SEQ ID NO:14592 and SEQ ID NO:12536. In embodiments, the probe includes SEQ ID NO:14593 and SEQ ID NO:12537. In embodiments, the probe includes SEQ ID NO:14594 and SEQ ID NO:12538. In embodiments, the probe includes SEQ ID NO:14595 and SEQ ID NO:12539. In embodiments, the probe includes SEQ ID NO:14596 and SEQ ID NO:12540. In embodiments, the probe includes SEQ ID NO:14597 and SEQ ID NO:12541. In embodiments, the probe includes SEQ ID NO:14598 and SEQ ID NO:12542. In embodiments, the probe includes SEQ ID NO:14599 and SEQ ID NO:12543. In embodiments, the probe includes SEQ ID NO:14600 and SEQ ID NO:12544. In embodiments, the probe includes SEQ ID NO:14601 and SEQ ID NO:12545. In embodiments, the probe includes SEQ ID NO:14602 and SEQ ID NO:12546. In embodiments, the probe includes SEQ ID NO:14603 and SEQ ID NO:12547. In embodiments, the probe includes SEQ ID NO:14604 and SEQ ID NO:12548. In embodiments, the probe includes SEQ ID NO:14605 and SEQ ID NO:12549. In embodiments, the probe includes SEQ ID NO:14606 and SEQ ID NO:12550. In embodiments, the probe includes SEQ ID NO:14607 and SEQ ID NO:12551. In embodiments, the probe includes SEQ ID NO:14608 and SEQ ID NO:12552. In embodiments, the probe includes SEQ ID NO:14609 and SEQ ID NO:12553. In embodiments, the probe includes SEQ ID NO:14610 and SEQ ID NO:12554. In embodiments, the probe includes SEQ ID NO:14611 and SEQ ID NO:12555. In embodiments, the probe includes SEQ ID NO:14612 and SEQ ID NO:12556. In embodiments, the probe includes SEQ ID NO:14613 and SEQ ID NO:12557. In embodiments, the probe includes SEQ ID NO:14614 and SEQ ID NO:12558. In embodiments, the probe includes SEQ ID NO:14615 and SEQ ID NO:12559. In embodiments, the probe includes SEQ ID NO:14616 and SEQ ID NO:12560. In embodiments, the probe includes SEQ ID NO:14617 and SEQ ID NO:12561. In embodiments, the probe includes SEQ ID NO:14618 and SEQ ID NO:12562. In embodiments, the probe includes SEQ ID NO:14619 and SEQ ID NO:12563. In embodiments, the probe includes SEQ ID NO:14620 and SEQ ID NO:12564. In embodiments, the probe includes SEQ ID NO:14621 and SEQ ID NO:12565. In embodiments, the probe includes SEQ ID NO:14622 and SEQ ID NO:12566. In embodiments, the probe includes SEQ ID NO:14623 and SEQ ID NO:12567. In embodiments, the probe includes SEQ ID NO:14624 and SEQ ID NO:12568. In embodiments, the probe includes SEQ ID NO:14625 and SEQ ID NO:12569. In embodiments, the probe includes SEQ ID NO:14626 and SEQ ID NO:12570. In embodiments, the probe includes SEQ ID NO:14627 and SEQ ID NO:12571. In embodiments, the probe includes SEQ ID NO:14628 and SEQ ID NO:12572. In embodiments, the probe includes SEQ ID NO:14629 and SEQ ID NO:12573. In embodiments, the probe includes SEQ ID NO:14630 and SEQ ID NO:12574. In embodiments, the probe includes SEQ ID NO:14631 and SEQ ID NO:12575. In embodiments, the probe includes SEQ ID NO:14632 and SEQ ID NO:12576. In embodiments, the probe includes SEQ ID NO:14633 and SEQ ID NO:12577. In embodiments, the probe includes SEQ ID NO:14634 and SEQ ID NO:12578. In embodiments, the probe includes SEQ ID NO:14635 and SEQ ID NO:12579. In embodiments, the probe includes SEQ ID NO:14636 and SEQ ID NO:12580. In embodiments, the probe includes SEQ ID NO:14637 and SEQ ID NO:12581. In embodiments, the probe includes SEQ ID NO:14638 and SEQ ID NO:12582. In embodiments, the probe includes SEQ ID NO:14639 and SEQ ID NO:12583. In embodiments, the probe includes SEQ ID NO:14640 and SEQ ID NO:12584. In embodiments, the probe includes SEQ ID NO:14641 and SEQ ID NO:12585. In embodiments, the probe includes SEQ ID NO:14642 and SEQ ID NO:12586. In embodiments, the probe includes SEQ ID NO:14643 and SEQ ID NO:12587. In embodiments, the probe includes SEQ ID NO:14644 and SEQ ID NO:12588. In embodiments, the probe includes SEQ ID NO:14645 and SEQ ID NO:12589. In embodiments, the probe includes SEQ ID NO:14646 and SEQ ID NO:12590. In embodiments, the probe includes SEQ ID NO:14647 and SEQ ID NO:12591. In embodiments, the probe includes SEQ ID NO:14648 and SEQ ID NO:12592. In embodiments, the probe includes SEQ ID NO:14649 and SEQ ID NO:12593. In embodiments, the probe includes SEQ ID NO:14650 and SEQ ID NO:12594. In embodiments, the probe includes SEQ ID NO:14651 and SEQ ID NO:12595. In embodiments, the probe includes SEQ ID NO:14652 and SEQ ID NO:12596. In embodiments, the probe includes SEQ ID NO:14653 and SEQ ID NO:12597. In embodiments, the probe includes SEQ ID NO:14654 and SEQ ID NO:12598. In embodiments, the probe includes SEQ ID NO:14655 and SEQ ID NO:12599. In embodiments, the probe includes SEQ ID NO:14656 and SEQ ID NO:12600. In embodiments, the probe includes SEQ ID NO:14657 and SEQ ID NO:12601. In embodiments, the probe includes SEQ ID NO:14658 and SEQ ID NO:12602. In embodiments, the probe includes SEQ ID NO:14659 and SEQ ID NO:12603. In embodiments, the probe includes SEQ ID NO:14660 and SEQ ID NO:12604. In embodiments, the probe includes SEQ ID NO:14661 and SEQ ID NO:12605. In embodiments, the probe includes SEQ ID NO:14662 and SEQ ID NO:12606. In embodiments, the probe includes SEQ ID NO:14663 and SEQ ID NO:12607. In embodiments, the probe includes SEQ ID NO:14664 and SEQ ID NO:12608. In embodiments, the probe includes SEQ ID NO:14665 and SEQ ID NO:12609. In embodiments, the probe includes SEQ ID NO:14666 and SEQ ID NO:12610. In embodiments, the probe includes SEQ ID NO:14667 and SEQ ID NO:12611. In embodiments, the probe includes SEQ ID NO:14668 and SEQ ID NO:12612. In embodiments, the probe includes SEQ ID NO:14669 and SEQ ID NO:12613. In embodiments, the probe includes SEQ ID NO:14670 and SEQ ID NO:12614. In embodiments, the probe includes SEQ ID NO:14671 and SEQ ID NO:12615. In embodiments, the probe includes SEQ ID NO:14672 and SEQ ID NO:12616. In embodiments, the probe includes SEQ ID NO:14673 and SEQ ID NO:12617. In embodiments, the probe includes SEQ ID NO:14674 and SEQ ID NO:12618. In embodiments, the probe includes SEQ ID NO:14675 and SEQ ID NO:12619. In embodiments, the probe includes SEQ ID NO:14676 and SEQ ID NO:12620. In embodiments, the probe includes SEQ ID NO:14677 and SEQ ID NO:12621. In embodiments, the probe includes SEQ ID NO:14678 and SEQ ID NO:12622. In embodiments, the probe includes SEQ ID NO:14679 and SEQ ID NO:12623. In embodiments, the probe includes SEQ ID NO:14680 and SEQ ID NO:12624. In embodiments, the probe includes SEQ ID NO:14681 and SEQ ID NO:12625. In embodiments, the probe includes SEQ ID NO:14682 and SEQ ID NO:12626. In embodiments, the probe includes SEQ ID NO:14683 and SEQ ID NO:12627. In embodiments, the probe includes SEQ ID NO:14684 and SEQ ID NO:12628. In embodiments, the probe includes SEQ ID NO:14685 and SEQ ID NO:12629. In embodiments, the probe includes SEQ ID NO:14686 and SEQ ID NO:12630. In embodiments, the probe includes SEQ ID NO:14687 and SEQ ID NO:12631. In embodiments, the probe includes SEQ ID NO:14688 and SEQ ID NO:12632. In embodiments, the probe includes SEQ ID NO:14689 and SEQ ID NO:12633. In embodiments, the probe includes SEQ ID NO:14690 and SEQ ID NO:12634. In embodiments, the probe includes SEQ ID NO:14691 and SEQ ID NO:12635. In embodiments, the probe includes SEQ ID NO:14692 and SEQ ID NO:12636. In embodiments, the probe includes SEQ ID NO:14693 and SEQ ID NO:12637. In embodiments, the probe includes SEQ ID NO:14694 and SEQ ID NO:12638. In embodiments, the probe includes SEQ ID NO:14695 and SEQ ID NO:12639. In embodiments, the probe includes SEQ ID NO:14696 and SEQ ID NO:12640. In embodiments, the probe includes SEQ ID NO:14697 and SEQ ID NO:12641. In embodiments, the probe includes SEQ ID NO:14698 and SEQ ID NO:12642. In embodiments, the probe includes SEQ ID NO:14699 and SEQ ID NO:12643. In embodiments, the probe includes SEQ ID NO:14700 and SEQ ID NO:12644. In embodiments, the probe includes SEQ ID NO:14701 and SEQ ID NO:12645. In embodiments, the probe includes SEQ ID NO:14702 and SEQ ID NO:12646. In embodiments, the probe includes SEQ ID NO:14703 and SEQ ID NO:12647. In embodiments, the probe includes SEQ ID NO:14704 and SEQ ID NO:12648. In embodiments, the probe includes SEQ ID NO:14705 and SEQ ID NO:12649. In embodiments, the probe includes SEQ ID NO:14706 and SEQ ID NO:12650. In embodiments, the probe includes SEQ ID NO:14707 and SEQ ID NO:12651. In embodiments, the probe includes SEQ ID NO:14708 and SEQ ID NO:12652. In embodiments, the probe includes SEQ ID NO:14709 and SEQ ID NO:12653. In embodiments, the probe includes SEQ ID NO:14710 and SEQ ID NO:12654. In embodiments, the probe includes SEQ ID NO:14711 and SEQ ID NO:12655. In embodiments, the probe includes SEQ ID NO:14712 and SEQ ID NO:12656. In embodiments, the probe includes SEQ ID NO:14713 and SEQ ID NO:12657. In embodiments, the probe includes SEQ ID NO:14714 and SEQ ID NO:12658. In embodiments, the probe includes SEQ ID NO:14715 and SEQ ID NO:12659. In embodiments, the probe includes SEQ ID NO:14716 and SEQ ID NO:12660. In embodiments, the probe includes SEQ ID NO:14717 and SEQ ID NO:12661. In embodiments, the probe includes SEQ ID NO:14718 and SEQ ID NO:12662. In embodiments, the probe includes SEQ ID NO:14719 and SEQ ID NO:12663. In embodiments, the probe includes SEQ ID NO:14720 and SEQ ID NO:12664. In embodiments, the probe includes SEQ ID NO:14721 and SEQ ID NO:12665. In embodiments, the probe includes SEQ ID NO:14722 and SEQ ID NO:12666. In embodiments, the probe includes SEQ ID NO:14723 and SEQ ID NO:12667. In embodiments, the probe includes SEQ ID NO:14724 and SEQ ID NO:12668. In embodiments, the probe includes SEQ ID NO:14725 and SEQ ID NO:12669. In embodiments, the probe includes SEQ ID NO:14726 and SEQ ID NO:12670. In embodiments, the probe includes SEQ ID NO:14727 and SEQ ID NO:12671. In embodiments, the probe includes SEQ ID NO:14728 and SEQ ID NO:12672. In embodiments, the probe includes SEQ ID NO:14729 and SEQ ID NO:12673. In embodiments, the probe includes SEQ ID NO:14730 and SEQ ID NO:12674. In embodiments, the probe includes SEQ ID NO:14731 and SEQ ID NO:12675. In embodiments, the probe includes SEQ ID NO:14732 and SEQ ID NO:12676. In embodiments, the probe includes SEQ ID NO:14733 and SEQ ID NO:12677. In embodiments, the probe includes SEQ ID NO:14734 and SEQ ID NO:12678. In embodiments, the probe includes SEQ ID NO:14735 and SEQ ID NO:12679. In embodiments, the probe includes SEQ ID NO:14736 and SEQ ID NO:12680. In embodiments, the probe includes SEQ ID NO:14737 and SEQ ID NO:12681. In embodiments, the probe includes SEQ ID NO:14738 and SEQ ID NO:12682. In embodiments, the probe includes SEQ ID NO:14739 and SEQ ID NO:12683. In embodiments, the probe includes SEQ ID NO:14740 and SEQ ID NO:12684. In embodiments, the probe includes SEQ ID NO:14741 and SEQ ID NO:12685. In embodiments, the probe includes SEQ ID NO:14742 and SEQ ID NO:12686. In embodiments, the probe includes SEQ ID NO:14743 and SEQ ID NO:12687. In embodiments, the probe includes SEQ ID NO:14744 and SEQ ID NO:12688. In embodiments, the probe includes SEQ ID NO:14745 and SEQ ID NO:12689. In embodiments, the probe includes SEQ ID NO:14746 and SEQ ID NO:12690. In embodiments, the probe includes SEQ ID NO:14747 and SEQ ID NO:12691. In embodiments, the probe includes SEQ ID NO:14748 and SEQ ID NO:12692. In embodiments, the probe includes SEQ ID NO:14749 and SEQ ID NO:12693. In embodiments, the probe includes SEQ ID NO:14750 and SEQ ID NO:12694. In embodiments, the probe includes SEQ ID NO:14751 and SEQ ID NO:12695. In embodiments, the probe includes SEQ ID NO:14752 and SEQ ID NO:12696. In embodiments, the probe includes SEQ ID NO:14753 and SEQ ID NO:12697. In embodiments, the probe includes SEQ ID NO:14754 and SEQ ID NO:12698. In embodiments, the probe includes SEQ ID NO:14755 and SEQ ID NO:12699. In embodiments, the probe includes SEQ ID NO:14756 and SEQ ID NO:12700. In embodiments, the probe includes SEQ ID NO:14757 and SEQ ID NO:12701. In embodiments, the probe includes SEQ ID NO:14758 and SEQ ID NO:12702. In embodiments, the probe includes SEQ ID NO:14759 and SEQ ID NO:12703. In embodiments, the probe includes SEQ ID NO:14760 and SEQ ID NO:12704. In embodiments, the probe includes SEQ ID NO:14761 and SEQ ID NO:12705. In embodiments, the probe includes SEQ ID NO:14762 and SEQ ID NO:12706. In embodiments, the probe includes SEQ ID NO:14763 and SEQ ID NO:12707. In embodiments, the probe includes SEQ ID NO:14764 and SEQ ID NO:12708. In embodiments, the probe includes SEQ ID NO:14765 and SEQ ID NO:12709. In embodiments, the probe includes SEQ ID NO:14766 and SEQ ID NO:12710. In embodiments, the probe includes SEQ ID NO:14767 and SEQ ID NO:12711. In embodiments, the probe includes SEQ ID NO:14768 and SEQ ID NO:12712. In embodiments, the probe includes SEQ ID NO:14769 and SEQ ID NO:12713. In embodiments, the probe includes SEQ ID NO:14770 and SEQ ID NO:12714. In embodiments, the probe includes SEQ ID NO:14771 and SEQ ID NO:12715. In embodiments, the probe includes SEQ ID NO:14772 and SEQ ID NO:12716. In embodiments, the probe includes SEQ ID NO:14773 and SEQ ID NO:12717. In embodiments, the probe includes SEQ ID NO:14774 and SEQ ID NO:12718. In embodiments, the probe includes SEQ ID NO:14775 and SEQ ID NO:12719. In embodiments, the probe includes SEQ ID NO:14776 and SEQ ID NO:12720. In embodiments, the probe includes SEQ ID NO:14777 and SEQ ID NO:12721. In embodiments, the probe includes SEQ ID NO:14778 and SEQ ID NO:12722. In embodiments, the probe includes SEQ ID NO:14779 and SEQ ID NO:12723. In embodiments, the probe includes SEQ ID NO:14780 and SEQ ID NO:12724. In embodiments, the probe includes SEQ ID NO:14781 and SEQ ID NO:12725. In embodiments, the probe includes SEQ ID NO:14782 and SEQ ID NO:12726. In embodiments, the probe includes SEQ ID NO:14783 and SEQ ID NO:12727. In embodiments, the probe includes SEQ ID NO:14784 and SEQ ID NO:12728. In embodiments, the probe includes SEQ ID NO:14785 and SEQ ID NO:12729. In embodiments, the probe includes SEQ ID NO:14786 and SEQ ID NO:12730. In embodiments, the probe includes SEQ ID NO:14787 and SEQ ID NO:12731. In embodiments, the probe includes SEQ ID NO:14788 and SEQ ID NO:12732. In embodiments, the probe includes SEQ ID NO:14789 and SEQ ID NO:12733. In embodiments, the probe includes SEQ ID NO:14790 and SEQ ID NO:12734. In embodiments, the probe includes SEQ ID NO:14791 and SEQ ID NO:12735. In embodiments, the probe includes SEQ ID NO:14792 and SEQ ID NO:12736. In embodiments, the probe includes SEQ ID NO:14793 and SEQ ID NO:12737. In embodiments, the probe includes SEQ ID NO:14794 and SEQ ID NO:12738. In embodiments, the probe includes SEQ ID NO:14795 and SEQ ID NO:12739. In embodiments, the probe includes SEQ ID NO:14796 and SEQ ID NO:12740. In embodiments, the probe includes SEQ ID NO:14797 and SEQ ID NO:12741. In embodiments, the probe includes SEQ ID NO:14798 and SEQ ID NO:12742. In embodiments, the probe includes SEQ ID NO:14799 and SEQ ID NO:12743. In embodiments, the probe includes SEQ ID NO:14800 and SEQ ID NO:12744. In embodiments, the probe includes SEQ ID NO:14801 and SEQ ID NO:12745. In embodiments, the probe includes SEQ ID NO:14802 and SEQ ID NO:12746. In embodiments, the probe includes SEQ ID NO:14803 and SEQ ID NO:12747. In embodiments, the probe includes SEQ ID NO:14804 and SEQ ID NO:12748. In embodiments, the probe includes SEQ ID NO:14805 and SEQ ID NO:12749. In embodiments, the probe includes SEQ ID NO:14806 and SEQ ID NO:12750. In embodiments, the probe includes SEQ ID NO:14807 and SEQ ID NO:12751. In embodiments, the probe includes SEQ ID NO:14808 and SEQ ID NO:12752. In embodiments, the probe includes SEQ ID NO:14809 and SEQ ID NO:12753. In embodiments, the probe includes SEQ ID NO:14810 and SEQ ID NO:12754. In embodiments, the probe includes SEQ ID NO:14811 and SEQ ID NO:12755. In embodiments, the probe includes SEQ ID NO:14812 and SEQ ID NO:12756. In embodiments, the probe includes SEQ ID NO:14813 and SEQ ID NO:12757. In embodiments, the probe includes SEQ ID NO:14814 and SEQ ID NO:12758. In embodiments, the probe includes SEQ ID NO:14815 and SEQ ID NO:12759. In embodiments, the probe includes SEQ ID NO:14816 and SEQ ID NO:12760. In embodiments, the probe includes SEQ ID NO:14817 and SEQ ID NO:12761. In embodiments, the probe includes SEQ ID NO:14818 and SEQ ID NO:12762. In embodiments, the probe includes SEQ ID NO:14819 and SEQ ID NO:12763. In embodiments, the probe includes SEQ ID NO:14820 and SEQ ID NO:12764. In embodiments, the probe includes SEQ ID NO:14821 and SEQ ID NO:12765. In embodiments, the probe includes SEQ ID NO:14822 and SEQ ID NO:12766. In embodiments, the probe includes SEQ ID NO:14823 and SEQ ID NO:12767. In embodiments, the probe includes SEQ ID NO:14824 and SEQ ID NO:12768. In embodiments, the probe includes SEQ ID NO:14825 and SEQ ID NO:12769. In embodiments, the probe includes SEQ ID NO:14826 and SEQ ID NO:12770. In embodiments, the probe includes SEQ ID NO:14827 and SEQ ID NO:12771. In embodiments, the probe includes SEQ ID NO:14828 and SEQ ID NO:12772. In embodiments, the probe includes SEQ ID NO:14829 and SEQ ID NO:12773. In embodiments, the probe includes SEQ ID NO:14830 and SEQ ID NO:12774. In embodiments, the probe includes SEQ ID NO:14831 and SEQ ID NO:12775. In embodiments, the probe includes SEQ ID NO:14832 and SEQ ID NO:12776. In embodiments, the probe includes SEQ ID NO:14833 and SEQ ID NO:12777. In embodiments, the probe includes SEQ ID NO:14834 and SEQ ID NO:12778. In embodiments, the probe includes SEQ ID NO:14835 and SEQ ID NO:12779. In embodiments, the probe includes SEQ ID NO:14836 and SEQ ID NO:12780. In embodiments, the probe includes SEQ ID NO:14837 and SEQ ID NO:12781. In embodiments, the probe includes SEQ ID NO:14838 and SEQ ID NO:12782. In embodiments, the probe includes SEQ ID NO:14839 and SEQ ID NO:12783. In embodiments, the probe includes SEQ ID NO:14840 and SEQ ID NO:12784. In embodiments, the probe includes SEQ ID NO:14841 and SEQ ID NO:12785. In embodiments, the probe includes SEQ ID NO:14842 and SEQ ID NO:12786. In embodiments, the probe includes SEQ ID NO:14843 and SEQ ID NO:12787. In embodiments, the probe includes SEQ ID NO:14844 and SEQ ID NO:12788. In embodiments, the probe includes SEQ ID NO:14845 and SEQ ID NO:12789. In embodiments, the probe includes SEQ ID NO:14846 and SEQ ID NO:12790. In embodiments, the probe includes SEQ ID NO:14847 and SEQ ID NO:12791. In embodiments, the probe includes SEQ ID NO:14848 and SEQ ID NO:12792. In embodiments, the probe includes SEQ ID NO:14849 and SEQ ID NO:12793. In embodiments, the probe includes SEQ ID NO:14850 and SEQ ID NO:12794. In embodiments, the probe includes SEQ ID NO:14851 and SEQ ID NO:12795. In embodiments, the probe includes SEQ ID NO:14852 and SEQ ID NO:12796. In embodiments, the probe includes SEQ ID NO:14853 and SEQ ID NO:12797. In embodiments, the probe includes SEQ ID NO:14854 and SEQ ID NO:12798. In embodiments, the probe includes SEQ ID NO:14855 and SEQ ID NO:12799. In embodiments, the probe includes SEQ ID NO:14856 and SEQ ID NO:12800. In embodiments, the probe includes SEQ ID NO:14857 and SEQ ID NO:12801. In embodiments, the probe includes SEQ ID NO:14858 and SEQ ID NO:12802. In embodiments, the probe includes SEQ ID NO:14859 and SEQ ID NO:12803. In embodiments, the probe includes SEQ ID NO:14860 and SEQ ID NO:12804. In embodiments, the probe includes SEQ ID NO:14861 and SEQ ID NO:12805. In embodiments, the probe includes SEQ ID NO:14862 and SEQ ID NO:12806. In embodiments, the probe includes SEQ ID NO:14863 and SEQ ID NO:12807. In embodiments, the probe includes SEQ ID NO:14864 and SEQ ID NO:12808. In embodiments, the probe includes SEQ ID NO:14865 and SEQ ID NO:12809. In embodiments, the probe includes SEQ ID NO:14866 and SEQ ID NO:12810. In embodiments, the probe includes SEQ ID NO:14867 and SEQ ID NO:12811. In embodiments, the probe includes SEQ ID NO:14868 and SEQ ID NO:12812. In embodiments, the probe includes SEQ ID NO:14869 and SEQ ID NO:12813. In embodiments, the probe includes SEQ ID NO:14870 and SEQ ID NO:12814. In embodiments, the probe includes SEQ ID NO:14871 and SEQ ID NO:12815. In embodiments, the probe includes SEQ ID NO:14872 and SEQ ID NO:12816. In embodiments, the probe includes SEQ ID NO:14873 and SEQ ID NO:12817. In embodiments, the probe includes SEQ ID NO:14874 and SEQ ID NO:12818. In embodiments, the probe includes SEQ ID NO:14875 and SEQ ID NO:12819. In embodiments, the probe includes SEQ ID NO:14876 and SEQ ID NO:12820. In embodiments, the probe includes SEQ ID NO:14877 and SEQ ID NO:12821. In embodiments, the probe includes SEQ ID NO:14878 and SEQ ID NO:12822. In embodiments, the probe includes SEQ ID NO:14879 and SEQ ID NO:12823. In embodiments, the probe includes SEQ ID NO:14880 and SEQ ID NO:12824. In embodiments, the probe includes SEQ ID NO:14881 and SEQ ID NO:12825. In embodiments, the probe includes SEQ ID NO:14882 and SEQ ID NO:12826. In embodiments, the probe includes SEQ ID NO:14883 and SEQ ID NO:12827. In embodiments, the probe includes SEQ ID NO:14884 and SEQ ID NO:12828. In embodiments, the probe includes SEQ ID NO:14885 and SEQ ID NO:12829. In embodiments, the probe includes SEQ ID NO:14886 and SEQ ID NO:12830. In embodiments, the probe includes SEQ ID NO:14887 and SEQ ID NO:12831. In embodiments, the probe includes SEQ ID NO:14888 and SEQ ID NO:12832. In embodiments, the probe includes SEQ ID NO:14889 and SEQ ID NO:12833. In embodiments, the probe includes SEQ ID NO:14890 and SEQ ID NO:12834. In embodiments, the probe includes SEQ ID NO:14891 and SEQ ID NO:12835. In embodiments, the probe includes SEQ ID NO:14892 and SEQ ID NO:12836. In embodiments, the probe includes SEQ ID NO:14893 and SEQ ID NO:12837. In embodiments, the probe includes SEQ ID NO:14894 and SEQ ID NO:12838. In embodiments, the probe includes SEQ ID NO:14895 and SEQ ID NO:12839. In embodiments, the probe includes SEQ ID NO:14896 and SEQ ID NO:12840. In embodiments, the probe includes SEQ ID NO:14897 and SEQ ID NO:12841. In embodiments, the probe includes SEQ ID NO:14898 and SEQ ID NO:12842. In embodiments, the probe includes SEQ ID NO:14899 and SEQ ID NO:12843. In embodiments, the probe includes SEQ ID NO:14900 and SEQ ID NO:12844. In embodiments, the probe includes SEQ ID NO:14901 and SEQ ID NO:12845. In embodiments, the probe includes SEQ ID NO:14902 and SEQ ID NO:12846. In embodiments, the probe includes SEQ ID NO:14903 and SEQ ID NO:12847. In embodiments, the probe includes SEQ ID NO:14904 and SEQ ID NO:12848. In embodiments, the probe includes SEQ ID NO:14905 and SEQ ID NO:12849. In embodiments, the probe includes SEQ ID NO:14906 and SEQ ID NO:12850. In embodiments, the probe includes SEQ ID NO:14907 and SEQ ID NO:12851. In embodiments, the probe includes SEQ ID NO:14908 and SEQ ID NO:12852. In embodiments, the probe includes SEQ ID NO:14909 and SEQ ID NO:12853. In embodiments, the probe includes SEQ ID NO:14910 and SEQ ID NO:12854. In embodiments, the probe includes SEQ ID NO:14911 and SEQ ID NO:12855. In embodiments, the probe includes SEQ ID NO:14912 and SEQ ID NO:12856. In embodiments, the probe includes SEQ ID NO:14913 and SEQ ID NO:12857. In embodiments, the probe includes SEQ ID NO:14914 and SEQ ID NO:12858. In embodiments, the probe includes SEQ ID NO:14915 and SEQ ID NO:12859. In embodiments, the probe includes SEQ ID NO:14916 and SEQ ID NO:12860. In embodiments, the probe includes SEQ ID NO:14917 and SEQ ID NO:12861. In embodiments, the probe includes SEQ ID NO:14918 and SEQ ID NO:12862. In embodiments, the probe includes SEQ ID NO:14919 and SEQ ID NO:12863. In embodiments, the probe includes SEQ ID NO:14920 and SEQ ID NO:12864. In embodiments, the probe includes SEQ ID NO:14921 and SEQ ID NO:12865. In embodiments, the probe includes SEQ ID NO:14922 and SEQ ID NO:12866. In embodiments, the probe includes SEQ ID NO:14923 and SEQ ID NO:12867. In embodiments, the probe includes SEQ ID NO:14924 and SEQ ID NO:12868. In embodiments, the probe includes SEQ ID NO:14925 and SEQ ID NO:12869. In embodiments, the probe includes SEQ ID NO:14926 and SEQ ID NO:12870. In embodiments, the probe includes SEQ ID NO:14927 and SEQ ID NO:12871. In embodiments, the probe includes SEQ ID NO:14928 and SEQ ID NO:12872. In embodiments, the probe includes SEQ ID NO:14929 and SEQ ID NO:12873. In embodiments, the probe includes SEQ ID NO:14930 and SEQ ID NO:12874. In embodiments, the probe includes SEQ ID NO:14931 and SEQ ID NO:12875. In embodiments, the probe includes SEQ ID NO:14932 and SEQ ID NO:12876. In embodiments, the probe includes SEQ ID NO:14933 and SEQ ID NO:12877. In embodiments, the probe includes SEQ ID NO:14934 and SEQ ID NO:12878. In embodiments, the probe includes SEQ ID NO:14935 and SEQ ID NO:12879. In embodiments, the probe includes SEQ ID NO:14936 and SEQ ID NO:12880. In embodiments, the probe includes SEQ ID NO:14937 and SEQ ID NO:12881. In embodiments, the probe includes SEQ ID NO:14938 and SEQ ID NO:12882. In embodiments, the probe includes SEQ ID NO:14939 and SEQ ID NO:12883. In embodiments, the probe includes SEQ ID NO:14940 and SEQ ID NO:12884. In embodiments, the probe includes SEQ ID NO:14941 and SEQ ID NO:12885. In embodiments, the probe includes SEQ ID NO:14942 and SEQ ID NO:12886. In embodiments, the probe includes SEQ ID NO:14943 and SEQ ID NO:12887. In embodiments, the probe includes SEQ ID NO:14944 and SEQ ID NO:12888. In embodiments, the probe includes SEQ ID NO:14945 and SEQ ID NO:12889. In embodiments, the probe includes SEQ ID NO:14946 and SEQ ID NO:12890. In embodiments, the probe includes SEQ ID NO:14947 and SEQ ID NO:12891. In embodiments, the probe includes SEQ ID NO:14948 and SEQ ID NO:12892. In embodiments, the probe includes SEQ ID NO:14949 and SEQ ID NO:12893. In embodiments, the probe includes SEQ ID NO:14950 and SEQ ID NO:12894. In embodiments, the probe includes SEQ ID NO:14951 and SEQ ID NO:12895. In embodiments, the probe includes SEQ ID NO:14952 and SEQ ID NO:12896. In embodiments, the probe includes SEQ ID NO:14953 and SEQ ID NO:12897. In embodiments, the probe includes SEQ ID NO:14954 and SEQ ID NO:12898. In embodiments, the probe includes SEQ ID NO:14955 and SEQ ID NO:12899. In embodiments, the probe includes SEQ ID NO:14956 and SEQ ID NO:12900. In embodiments, the probe includes SEQ ID NO:14957 and SEQ ID NO:12901. In embodiments, the probe includes SEQ ID NO:14958 and SEQ ID NO:12902. In embodiments, the probe includes SEQ ID NO:14959 and SEQ ID NO:12903. In embodiments, the probe includes SEQ ID NO:14960 and SEQ ID NO:12904. In embodiments, the probe includes SEQ ID NO:14961 and SEQ ID NO:12905. In embodiments, the probe includes SEQ ID NO:14962 and SEQ ID NO:12906. In embodiments, the probe includes SEQ ID NO:14963 and SEQ ID NO:12907. In embodiments, the probe includes SEQ ID NO:14964 and SEQ ID NO:12908. In embodiments, the probe includes SEQ ID NO:14965 and SEQ ID NO:12909. In embodiments, the probe includes SEQ ID NO:14966 and SEQ ID NO:12910. In embodiments, the probe includes SEQ ID NO:14967 and SEQ ID NO:12911. In embodiments, the probe includes SEQ ID NO:14968 and SEQ ID NO:12912. In embodiments, the probe includes SEQ ID NO:14969 and SEQ ID NO:12913. In embodiments, the probe includes SEQ ID NO:14970 and SEQ ID NO:12914. In embodiments, the probe includes SEQ ID NO:14971 and SEQ ID NO:12915. In embodiments, the probe includes SEQ ID NO:14972 and SEQ ID NO:12916. In embodiments, the probe includes SEQ ID NO:14973 and SEQ ID NO:12917. In embodiments, the probe includes SEQ ID NO:14974 and SEQ ID NO:12918. In embodiments, the probe includes SEQ ID NO:14975 and SEQ ID NO:12919. In embodiments, the probe includes SEQ ID NO:14976 and SEQ ID NO:12920. In embodiments, the probe includes SEQ ID NO:14977 and SEQ ID NO:12921. In embodiments, the probe includes SEQ ID NO:14978 and SEQ ID NO:12922. In embodiments, the probe includes SEQ ID NO:14979 and SEQ ID NO:12923. In embodiments, the probe includes SEQ ID NO:14980 and SEQ ID NO:12924. In embodiments, the probe includes SEQ ID NO:14981 and SEQ ID NO:12925. In embodiments, the probe includes SEQ ID NO:14982 and SEQ ID NO:12926. In embodiments, the probe includes SEQ ID NO:14983 and SEQ ID NO:12927. In embodiments, the probe includes SEQ ID NO:14984 and SEQ ID NO:12928. In embodiments, the probe includes SEQ ID NO:14985 and SEQ ID NO:12929. In embodiments, the probe includes SEQ ID NO:14986 and SEQ ID NO:12930. In embodiments, the probe includes SEQ ID NO:14987 and SEQ ID NO:12931. In embodiments, the probe includes SEQ ID NO:14988 and SEQ ID NO:12932. In embodiments, the probe includes SEQ ID NO:14989 and SEQ ID NO:12933. In embodiments, the probe includes SEQ ID NO:14990 and SEQ ID NO:12934. In embodiments, the probe includes SEQ ID NO:14991 and SEQ ID NO:12935. In embodiments, the probe includes SEQ ID NO:14992 and SEQ ID NO:12936. In embodiments, the probe includes SEQ ID NO:14993 and SEQ ID NO:12937. In embodiments, the probe includes SEQ ID NO:14994 and SEQ ID NO:12938. In embodiments, the probe includes SEQ ID NO:14995 and SEQ ID NO:12939. In embodiments, the probe includes SEQ ID NO:14996 and SEQ ID NO:12940. In embodiments, the probe includes SEQ ID NO:14997 and SEQ ID NO:12941. In embodiments, the probe includes SEQ ID NO:14998 and SEQ ID NO:12942. In embodiments, the probe includes SEQ ID NO:14999 and SEQ ID NO:12943. In embodiments, the probe includes SEQ ID NO:15000 and SEQ ID NO:12944. In embodiments, the probe includes SEQ ID NO:15001 and SEQ ID NO:12945. In embodiments, the probe includes SEQ ID NO:15002 and SEQ ID NO:12946. In embodiments, the probe includes SEQ ID NO:15003 and SEQ ID NO:12947. In embodiments, the probe includes SEQ ID NO:15004 and SEQ ID NO:12948. In embodiments, the probe includes SEQ ID NO:15005 and SEQ ID NO:12949. In embodiments, the probe includes SEQ ID NO:15006 and SEQ ID NO:12950. In embodiments, the probe includes SEQ ID NO:15007 and SEQ ID NO:12951. In embodiments, the probe includes SEQ ID NO:15008 and SEQ ID NO:12952. In embodiments, the probe includes SEQ ID NO:15009 and SEQ ID NO:12953. In embodiments, the probe includes SEQ ID NO:15010 and SEQ ID NO:12954. In embodiments, the probe includes SEQ ID NO:15011 and SEQ ID NO:12955. In embodiments, the probe includes SEQ ID NO:15012 and SEQ ID NO:12956. In embodiments, the probe includes SEQ ID NO:15013 and SEQ ID NO:12957. In embodiments, the probe includes SEQ ID NO:15014 and SEQ ID NO:12958. In embodiments, the probe includes SEQ ID NO:15015 and SEQ ID NO:12959. In embodiments, the probe includes SEQ ID NO:15016 and SEQ ID NO:12960. In embodiments, the probe includes SEQ ID NO:15017 and SEQ ID NO:12961. In embodiments, the probe includes SEQ ID NO:15018 and SEQ ID NO:12962. In embodiments, the probe includes SEQ ID NO:15019 and SEQ ID NO:12963. In embodiments, the probe includes SEQ ID NO:15020 and SEQ ID NO:12964. In embodiments, the probe includes SEQ ID NO:15021 and SEQ ID NO:12965. In embodiments, the probe includes SEQ ID NO:15022 and SEQ ID NO:12966. In embodiments, the probe includes SEQ ID NO:15023 and SEQ ID NO:12967. In embodiments, the probe includes SEQ ID NO:15024 and SEQ ID NO:12968. In embodiments, the probe includes SEQ ID NO:15025 and SEQ ID NO:12969. In embodiments, the probe includes SEQ ID NO:15026 and SEQ ID NO:12970. In embodiments, the probe includes SEQ ID NO:15027 and SEQ ID NO:12971. In embodiments, the probe includes SEQ ID NO:15028 and SEQ ID NO:12972. In embodiments, the probe includes SEQ ID NO:15029 and SEQ ID NO:12973. In embodiments, the probe includes SEQ ID NO:15030 and SEQ ID NO:12974. In embodiments, the probe includes SEQ ID NO:15031 and SEQ ID NO:12975. In embodiments, the probe includes SEQ ID NO:15032 and SEQ ID NO:12976. In embodiments, the probe includes SEQ ID NO:15033 and SEQ ID NO:12977. In embodiments, the probe includes SEQ ID NO:15034 and SEQ ID NO:12978. In embodiments, the probe includes SEQ ID NO:15035 and SEQ ID NO:12979. In embodiments, the probe includes SEQ ID NO:15036 and SEQ ID NO:12980. In embodiments, the probe includes SEQ ID NO:15037 and SEQ ID NO:12981. In embodiments, the probe includes SEQ ID NO:15038 and SEQ ID NO:12982. In embodiments, the probe includes SEQ ID NO:15039 and SEQ ID NO:12983. In embodiments, the probe includes SEQ ID NO:15040 and SEQ ID NO:12984. In embodiments, the probe includes SEQ ID NO:15041 and SEQ ID NO:12985. In embodiments, the probe includes SEQ ID NO:15042 and SEQ ID NO:12986. In embodiments, the probe includes SEQ ID NO:15043 and SEQ ID NO:12987. In embodiments, the probe includes SEQ ID NO:15044 and SEQ ID NO:12988. In embodiments, the probe includes SEQ ID NO:15045 and SEQ ID NO:12989. In embodiments, the probe includes SEQ ID NO:15046 and SEQ ID NO:12990. In embodiments, the probe includes SEQ ID NO:15047 and SEQ ID NO:12991. In embodiments, the probe includes SEQ ID NO:15048 and SEQ ID NO:12992. In embodiments, the probe includes SEQ ID NO:15049 and SEQ ID NO:12993. In embodiments, the probe includes SEQ ID NO:15050 and SEQ ID NO:12994. In embodiments, the probe includes SEQ ID NO:15051 and SEQ ID NO:12995. In embodiments, the probe includes SEQ ID NO:15052 and SEQ ID NO:12996. In embodiments, the probe includes SEQ ID NO:15053 and SEQ ID NO:12997. In embodiments, the probe includes SEQ ID NO:15054 and SEQ ID NO:12998. In embodiments, the probe includes SEQ ID NO:15055 and SEQ ID NO:12999. In embodiments, the probe includes SEQ ID NO:15056 and SEQ ID NO:13000. In embodiments, the probe includes SEQ ID NO:15057 and SEQ ID NO:13001. In embodiments, the probe includes SEQ ID NO:15058 and SEQ ID NO:13002. In embodiments, the probe includes SEQ ID NO:15059 and SEQ ID NO:13003. In embodiments, the probe includes SEQ ID NO:15060 and SEQ ID NO:13004. In embodiments, the probe includes SEQ ID NO:15061 and SEQ ID NO:13005. In embodiments, the probe includes SEQ ID NO:15062 and SEQ ID NO:13006. In embodiments, the probe includes SEQ ID NO:15063 and SEQ ID NO:13007. In embodiments, the probe includes SEQ ID NO:15064 and SEQ ID NO:13008. In embodiments, the probe includes SEQ ID NO:15065 and SEQ ID NO:13009. In embodiments, the probe includes SEQ ID NO:15066 and SEQ ID NO:13010. In embodiments, the probe includes SEQ ID NO:15067 and SEQ ID NO:13011. In embodiments, the probe includes SEQ ID NO:15068 and SEQ ID NO:13012. In embodiments, the probe includes SEQ ID NO:15069 and SEQ ID NO:13013. In embodiments, the probe includes SEQ ID NO:15070 and SEQ ID NO:13014. In embodiments, the probe includes SEQ ID NO:15071 and SEQ ID NO:13015. In embodiments, the probe includes SEQ ID NO:15072 and SEQ ID NO:13016. In embodiments, the probe includes SEQ ID NO:15073 and SEQ ID NO:13017. In embodiments, the probe includes SEQ ID NO:15074 and SEQ ID NO:13018. In embodiments, the probe includes SEQ ID NO:15075 and SEQ ID NO:13019. In embodiments, the probe includes SEQ ID NO:15076 and SEQ ID NO:13020. In embodiments, the probe includes SEQ ID NO:15077 and SEQ ID NO:13021. In embodiments, the probe includes SEQ ID NO:15078 and SEQ ID NO:13022. In embodiments, the probe includes SEQ ID NO:15079 and SEQ ID NO:13023. In embodiments, the probe includes SEQ ID NO:15080 and SEQ ID NO:13024. In embodiments, the probe includes SEQ ID NO:15081 and SEQ ID NO:13025. In embodiments, the probe includes SEQ ID NO:15082 and SEQ ID NO:13026. In embodiments, the probe includes SEQ ID NO:15083 and SEQ ID NO:13027. In embodiments, the probe includes SEQ ID NO:15084 and SEQ ID NO:13028. In embodiments, the probe includes SEQ ID NO:15085 and SEQ ID NO:13029. In embodiments, the probe includes SEQ ID NO:15086 and SEQ ID NO:13030. In embodiments, the probe includes SEQ ID NO:15087 and SEQ ID NO:13031. In embodiments, the probe includes SEQ ID NO:15088 and SEQ ID NO:13032. In embodiments, the probe includes SEQ ID NO:15089 and SEQ ID NO:13033. In embodiments, the probe includes SEQ ID NO:15090 and SEQ ID NO:13034. In embodiments, the probe includes SEQ ID NO:15091 and SEQ ID NO:13035. In embodiments, the probe includes SEQ ID NO:15092 and SEQ ID NO:13036. In embodiments, the probe includes SEQ ID NO:15093 and SEQ ID NO:13037. In embodiments, the probe includes SEQ ID NO:15094 and SEQ ID NO:13038. In embodiments, the probe includes SEQ ID NO:15095 and SEQ ID NO:13039. In embodiments, the probe includes SEQ ID NO:15096 and SEQ ID NO:13040. In embodiments, the probe includes SEQ ID NO:15097 and SEQ ID NO:13041. In embodiments, the probe includes SEQ ID NO:15098 and SEQ ID NO:13042. In embodiments, the probe includes SEQ ID NO:15099 and SEQ ID NO:13043. In embodiments, the probe includes SEQ ID NO:15100 and SEQ ID NO:13044. In embodiments, the probe includes SEQ ID NO:15101 and SEQ ID NO:13045. In embodiments, the probe includes SEQ ID NO:15102 and SEQ ID NO:13046. In embodiments, the probe includes SEQ ID NO:15103 and SEQ ID NO:13047. In embodiments, the probe includes SEQ ID NO:15104 and SEQ ID NO:13048. In embodiments, the probe includes SEQ ID NO:15105 and SEQ ID NO:13049. In embodiments, the probe includes SEQ ID NO:15106 and SEQ ID NO:13050. In embodiments, the probe includes SEQ ID NO:15107 and SEQ ID NO:13051. In embodiments, the probe includes SEQ ID NO:15108 and SEQ ID NO:13052. In embodiments, the probe includes SEQ ID NO:15109 and SEQ ID NO:13053. In embodiments, the probe includes SEQ ID NO:15110 and SEQ ID NO:13054. In embodiments, the probe includes SEQ ID NO:15111 and SEQ ID NO:13055. In embodiments, the probe includes SEQ ID NO:15112 and SEQ ID NO:13056. In embodiments, the probe includes SEQ ID NO:15113 and SEQ ID NO:13057. In embodiments, the probe includes SEQ ID NO:15114 and SEQ ID NO:13058. In embodiments, the probe includes SEQ ID NO:15115 and SEQ ID NO:13059. In embodiments, the probe includes SEQ ID NO:15116 and SEQ ID NO:13060. In embodiments, the probe includes SEQ ID NO:15117 and SEQ ID NO:13061. In embodiments, the probe includes SEQ ID NO:15118 and SEQ ID NO:13062. In embodiments, the probe includes SEQ ID NO:15119 and SEQ ID NO:13063. In embodiments, the probe includes SEQ ID NO:15120 and SEQ ID NO:13064. In embodiments, the probe includes SEQ ID NO:15121 and SEQ ID NO:13065. In embodiments, the probe includes SEQ ID NO:15122 and SEQ ID NO:13066. In embodiments, the probe includes SEQ ID NO:15123 and SEQ ID NO:13067. In embodiments, the probe includes SEQ ID NO:15124 and SEQ ID NO:13068. In embodiments, the probe includes SEQ ID NO:15125 and SEQ ID NO:13069. In embodiments, the probe includes SEQ ID NO:15126 and SEQ ID NO:13070. In embodiments, the probe includes SEQ ID NO:15127 and SEQ ID NO:13071. In embodiments, the probe includes SEQ ID NO:15128 and SEQ ID NO:13072. In embodiments, the probe includes SEQ ID NO:15129 and SEQ ID NO:13073. In embodiments, the probe includes SEQ ID NO:15130 and SEQ ID NO:13074. In embodiments, the probe includes SEQ ID NO:15131 and SEQ ID NO:13075. In embodiments, the probe includes SEQ ID NO:15132 and SEQ ID NO:13076. In embodiments, the probe includes SEQ ID NO:15133 and SEQ ID NO:13077. In embodiments, the probe includes SEQ ID NO:15134 and SEQ ID NO:13078. In embodiments, the probe includes SEQ ID NO:15135 and SEQ ID NO:13079. In embodiments, the probe includes SEQ ID NO:15136 and SEQ ID NO:13080. In embodiments, the probe includes SEQ ID NO:15137 and SEQ ID NO:13081. In embodiments, the probe includes SEQ ID NO:15138 and SEQ ID NO:13082. In embodiments, the probe includes SEQ ID NO:15139 and SEQ ID NO:13083. In embodiments, the probe includes SEQ ID NO:15140 and SEQ ID NO:13084. In embodiments, the probe includes SEQ ID NO:15141 and SEQ ID NO:13085. In embodiments, the probe includes SEQ ID NO:15142 and SEQ ID NO:13086. In embodiments, the probe includes SEQ ID NO:15143 and SEQ ID NO:13087. In embodiments, the probe includes SEQ ID NO:15144 and SEQ ID NO:13088. In embodiments, the probe includes SEQ ID NO:15145 and SEQ ID NO:13089. In embodiments, the probe includes SEQ ID NO:15146 and SEQ ID NO:13090. In embodiments, the probe includes SEQ ID NO:15147 and SEQ ID NO:13091. In embodiments, the probe includes SEQ ID NO:15148 and SEQ ID NO:13092. In embodiments, the probe includes SEQ ID NO:15149 and SEQ ID NO:13093. In embodiments, the probe includes SEQ ID NO:15150 and SEQ ID NO:13094. In embodiments, the probe includes SEQ ID NO:15151 and SEQ ID NO:13095. In embodiments, the probe includes SEQ ID NO:15152 and SEQ ID NO:13096. In embodiments, the probe includes SEQ ID NO:15153 and SEQ ID NO:13097. In embodiments, the probe includes SEQ ID NO:15154 and SEQ ID NO:13098. In embodiments, the probe includes SEQ ID NO:15155 and SEQ ID NO:13099. In embodiments, the probe includes SEQ ID NO:15156 and SEQ ID NO:13100. In embodiments, the probe includes SEQ ID NO:15157 and SEQ ID NO:13101. In embodiments, the probe includes SEQ ID NO:15158 and SEQ ID NO:13102. In embodiments, the probe includes SEQ ID NO:15159 and SEQ ID NO:13103. In embodiments, the probe includes SEQ ID NO:15160 and SEQ ID NO:13104. In embodiments, the probe includes SEQ ID NO:15161 and SEQ ID NO:13105. In embodiments, the probe includes SEQ ID NO:15162 and SEQ ID NO:13106. In embodiments, the probe includes SEQ ID NO:15163 and SEQ ID NO:13107. In embodiments, the probe includes SEQ ID NO:15164 and SEQ ID NO:13108. In embodiments, the probe includes SEQ ID NO:15165 and SEQ ID NO:13109. In embodiments, the probe includes SEQ ID NO:15166 and SEQ ID NO:13110. In embodiments, the probe includes SEQ ID NO:15167 and SEQ ID NO:13111. In embodiments, the probe includes SEQ ID NO:15168 and SEQ ID NO:13112. In embodiments, the probe includes SEQ ID NO:15169 and SEQ ID NO:13113. In embodiments, the probe includes SEQ ID NO:15170 and SEQ ID NO:13114. In embodiments, the probe includes SEQ ID NO:15171 and SEQ ID NO:13115. In embodiments, the probe includes SEQ ID NO:15172 and SEQ ID NO:13116. In embodiments, the probe includes SEQ ID NO:15173 and SEQ ID NO:13117. In embodiments, the probe includes SEQ ID NO:15174 and SEQ ID NO:13118. In embodiments, the probe includes SEQ ID NO:15175 and SEQ ID NO:13119. In embodiments, the probe includes SEQ ID NO:15176 and SEQ ID NO:13120. In embodiments, the probe includes SEQ ID NO:15177 and SEQ ID NO:13121. In embodiments, the probe includes SEQ ID NO:15178 and SEQ ID NO:13122. In embodiments, the probe includes SEQ ID NO:15179 and SEQ ID NO:13123. In embodiments, the probe includes SEQ ID NO:15180 and SEQ ID NO:13124. In embodiments, the probe includes SEQ ID NO:15181 and SEQ ID NO:13125. In embodiments, the probe includes SEQ ID NO:15182 and SEQ ID NO:13126. In embodiments, the probe includes SEQ ID NO:15183 and SEQ ID NO:13127. In embodiments, the probe includes SEQ ID NO:15184 and SEQ ID NO:13128. In embodiments, the probe includes SEQ ID NO:15185 and SEQ ID NO:13129. In embodiments, the probe includes SEQ ID NO:15186 and SEQ ID NO:13130. In embodiments, the probe includes SEQ ID NO:15187 and SEQ ID NO:13131. In embodiments, the probe includes SEQ ID NO:15188 and SEQ ID NO:13132. In embodiments, the probe includes SEQ ID NO:15189 and SEQ ID NO:13133. In embodiments, the probe includes SEQ ID NO:15190 and SEQ ID NO:13134. In embodiments, the probe includes SEQ ID NO:15191 and SEQ ID NO:13135. In embodiments, the probe includes SEQ ID NO:15192 and SEQ ID NO:13136. In embodiments, the probe includes SEQ ID NO:15193 and SEQ ID NO:13137. In embodiments, the probe includes SEQ ID NO:15194 and SEQ ID NO:13138. In embodiments, the probe includes SEQ ID NO:15195 and SEQ ID NO:13139. In embodiments, the probe includes SEQ ID NO:15196 and SEQ ID NO:13140. In embodiments, the probe includes SEQ ID NO:15197 and SEQ ID NO:13141. In embodiments, the probe includes SEQ ID NO:15198 and SEQ ID NO:13142. In embodiments, the probe includes SEQ ID NO:15199 and SEQ ID NO:13143. In embodiments, the probe includes SEQ ID NO:15200 and SEQ ID NO:13144. In embodiments, the probe includes SEQ ID NO:15201 and SEQ ID NO:13145. In embodiments, the probe includes SEQ ID NO:15202 and SEQ ID NO:13146. In embodiments, the probe includes SEQ ID NO:15203 and SEQ ID NO:13147. In embodiments, the probe includes SEQ ID NO:15204 and SEQ ID NO:13148. In embodiments, the probe includes SEQ ID NO:15205 and SEQ ID NO:13149. In embodiments, the probe includes SEQ ID NO:15206 and SEQ ID NO:13150. In embodiments, the probe includes SEQ ID NO:15207 and SEQ ID NO:13151. In embodiments, the probe includes SEQ ID NO:15208 and SEQ ID NO:13152. In embodiments, the probe includes SEQ ID NO:15209 and SEQ ID NO:13153. In embodiments, the probe includes SEQ ID NO:15210 and SEQ ID NO:13154. In embodiments, the probe includes SEQ ID NO:15211 and SEQ ID NO:13155. In embodiments, the probe includes SEQ ID NO:15212 and SEQ ID NO:13156. In embodiments, the probe includes SEQ ID NO:15213 and SEQ ID NO:13157. In embodiments, the probe includes SEQ ID NO:15214 and SEQ ID NO:13158. In embodiments, the probe includes SEQ ID NO:15215 and SEQ ID NO:13159. In embodiments, the probe includes SEQ ID NO:15216 and SEQ ID NO:13160. In embodiments, the probe includes SEQ ID NO:15217 and SEQ ID NO:13161. In embodiments, the probe includes SEQ ID NO:15218 and SEQ ID NO:13162. In embodiments, the probe includes SEQ ID NO:15219 and SEQ ID NO:13163. In embodiments, the probe includes SEQ ID NO:15220 and SEQ ID NO:13164. In embodiments, the probe includes SEQ ID NO:15221 and SEQ ID NO:13165. In embodiments, the probe includes SEQ ID NO:15222 and SEQ ID NO:13166. In embodiments, the probe includes SEQ ID NO:15223 and SEQ ID NO:13167. In embodiments, the probe includes SEQ ID NO:15224 and SEQ ID NO:13168. In embodiments, the probe includes SEQ ID NO:15225 and SEQ ID NO:13169. In embodiments, the probe includes SEQ ID NO:15226 and SEQ ID NO:13170. In embodiments, the probe includes SEQ ID NO:15227 and SEQ ID NO:13171. In embodiments, the probe includes SEQ ID NO:15228 and SEQ ID NO:13172. In embodiments, the probe includes SEQ ID NO:15229 and SEQ ID NO:13173. In embodiments, the probe includes SEQ ID NO:15230 and SEQ ID NO:13174. In embodiments, the probe includes SEQ ID NO:15231 and SEQ ID NO:13175. In embodiments, the probe includes SEQ ID NO:15232 and SEQ ID NO:13176. In embodiments, the probe includes SEQ ID NO:15233 and SEQ ID NO:13177. In embodiments, the probe includes SEQ ID NO:15234 and SEQ ID NO:13178. In embodiments, the probe includes SEQ ID NO:15235 and SEQ ID NO:13179. In embodiments, the probe includes SEQ ID NO:15236 and SEQ ID NO:13180. In embodiments, the probe includes SEQ ID NO:15237 and SEQ ID NO:13181. In embodiments, the probe includes SEQ ID NO:15238 and SEQ ID NO:13182. In embodiments, the probe includes SEQ ID NO:15239 and SEQ ID NO:13183. In embodiments, the probe includes SEQ ID NO:15240 and SEQ ID NO:13184. In embodiments, the probe includes SEQ ID NO:15241 and SEQ ID NO:13185. In embodiments, the probe includes SEQ ID NO:15242 and SEQ ID NO:13186. In embodiments, the probe includes SEQ ID NO:15243 and SEQ ID NO:13187. In embodiments, the probe includes SEQ ID NO:15244 and SEQ ID NO:13188. In embodiments, the probe includes SEQ ID NO:15245 and SEQ ID NO:13189. In embodiments, the probe includes SEQ ID NO:15246 and SEQ ID NO:13190. In embodiments, the probe includes SEQ ID NO:15247 and SEQ ID NO:13191. In embodiments, the probe includes SEQ ID NO:15248 and SEQ ID NO:13192. In embodiments, the probe includes SEQ ID NO:15249 and SEQ ID NO:13193. In embodiments, the probe includes SEQ ID NO:15250 and SEQ ID NO:13194. In embodiments, the probe includes SEQ ID NO:15251 and SEQ ID NO:13195. In embodiments, the probe includes SEQ ID NO:15252 and SEQ ID NO:13196. In embodiments, the probe includes SEQ ID NO:15253 and SEQ ID NO:13197. In embodiments, the probe includes SEQ ID NO:15254 and SEQ ID NO:13198. In embodiments, the probe includes SEQ ID NO:15255 and SEQ ID NO:13199. In embodiments, the probe includes SEQ ID NO:15256 and SEQ ID NO:13200. In embodiments, the probe includes SEQ ID NO:15257 and SEQ ID NO:13201. In embodiments, the probe includes SEQ ID NO:15258 and SEQ ID NO:13202. In embodiments, the probe includes SEQ ID NO:15259 and SEQ ID NO:13203. In embodiments, the probe includes SEQ ID NO:15260 and SEQ ID NO:13204. In embodiments, the probe includes SEQ ID NO:15261 and SEQ ID NO:13205. In embodiments, the probe includes SEQ ID NO:15262 and SEQ ID NO:13206. In embodiments, the probe includes SEQ ID NO:15263 and SEQ ID NO:13207. In embodiments, the probe includes SEQ ID NO:15264 and SEQ ID NO:13208. In embodiments, the probe includes SEQ ID NO:15265 and SEQ ID NO:13209. In embodiments, the probe includes SEQ ID NO:15266 and SEQ ID NO:13210. In embodiments, the probe includes SEQ ID NO:15267 and SEQ ID NO:13211. In embodiments, the probe includes SEQ ID NO:15268 and SEQ ID NO:13212. In embodiments, the probe includes SEQ ID NO:15269 and SEQ ID NO:13213. In embodiments, the probe includes SEQ ID NO:15270 and SEQ ID NO:13214. In embodiments, the probe includes SEQ ID NO:15271 and SEQ ID NO:13215. In embodiments, the probe includes SEQ ID NO:15272 and SEQ ID NO:13216. In embodiments, the probe includes SEQ ID NO:15273 and SEQ ID NO:13217. In embodiments, the probe includes SEQ ID NO:15274 and SEQ ID NO:13218. In embodiments, the probe includes SEQ ID NO:15275 and SEQ ID NO:13219. In embodiments, the probe includes SEQ ID NO:15276 and SEQ ID NO:13220. In embodiments, the probe includes SEQ ID NO:15277 and SEQ ID NO:13221. In embodiments, the probe includes SEQ ID NO:15278 and SEQ ID NO:13222. In embodiments, the probe includes SEQ ID NO:15279 and SEQ ID NO:13223. In embodiments, the probe includes SEQ ID NO:15280 and SEQ ID NO:13224. In embodiments, the probe includes SEQ ID NO:15281 and SEQ ID NO:13225. In embodiments, the probe includes SEQ ID NO:15282 and SEQ ID NO:13226. In embodiments, the probe includes SEQ ID NO:15283 and SEQ ID NO:13227. In embodiments, the probe includes SEQ ID NO:15284 and SEQ ID NO:13228. In embodiments, the probe includes SEQ ID NO:15285 and SEQ ID NO:13229. In embodiments, the probe includes SEQ ID NO:15286 and SEQ ID NO:13230. In embodiments, the probe includes SEQ ID NO:15287 and SEQ ID NO:13231. In embodiments, the probe includes SEQ ID NO:15288 and SEQ ID NO:13232. In embodiments, the probe includes SEQ ID NO:15289 and SEQ ID NO:13233. In embodiments, the probe includes SEQ ID NO:15290 and SEQ ID NO:13234. In embodiments, the probe includes SEQ ID NO:15291 and SEQ ID NO:13235. In embodiments, the probe includes SEQ ID NO:15292 and SEQ ID NO:13236. In embodiments, the probe includes SEQ ID NO:15293 and SEQ ID NO:13237. In embodiments, the probe includes SEQ ID NO:15294 and SEQ ID NO:13238. In embodiments, the probe includes SEQ ID NO:15295 and SEQ ID NO:13239. In embodiments, the probe includes SEQ ID NO:15296 and SEQ ID NO:13240. In embodiments, the probe includes SEQ ID NO:15297 and SEQ ID NO:13241. In embodiments, the probe includes SEQ ID NO:15298 and SEQ ID NO:13242. In embodiments, the probe includes SEQ ID NO:15299 and SEQ ID NO:13243. In embodiments, the probe includes SEQ ID NO:15300 and SEQ ID NO:13244. In embodiments, the probe includes SEQ ID NO:15301 and SEQ ID NO:13245. In embodiments, the probe includes SEQ ID NO:15302 and SEQ ID NO:13246. In embodiments, the probe includes SEQ ID NO:15303 and SEQ ID NO:13247. In embodiments, the probe includes SEQ ID NO:15304 and SEQ ID NO:13248. In embodiments, the probe includes SEQ ID NO:15305 and SEQ ID NO:13249. In embodiments, the probe includes SEQ ID NO:15306 and SEQ ID NO:13250. In embodiments, the probe includes SEQ ID NO:15307 and SEQ ID NO:13251. In embodiments, the probe includes SEQ ID NO:15308 and SEQ ID NO:13252. In embodiments, the probe includes SEQ ID NO:15309 and SEQ ID NO:13253. In embodiments, the probe includes SEQ ID NO:15310 and SEQ ID NO:13254. In embodiments, the probe includes SEQ ID NO:15311 and SEQ ID NO:13255. In embodiments, the probe includes SEQ ID NO:15312 and SEQ ID NO:13256. In embodiments, the probe includes SEQ ID NO:15313 and SEQ ID NO:13257. In embodiments, the probe includes SEQ ID NO:15314 and SEQ ID NO:13258. In embodiments, the probe includes SEQ ID NO:15315 and SEQ ID NO:13259. In embodiments, the probe includes SEQ ID NO:15316 and SEQ ID NO:13260. In embodiments, the probe includes SEQ ID NO:15317 and SEQ ID NO:13261. In embodiments, the probe includes SEQ ID NO:15318 and SEQ ID NO:13262. In embodiments, the probe includes SEQ ID NO:15319 and SEQ ID NO:13263. In embodiments, the probe includes SEQ ID NO:15320 and SEQ ID NO:13264. In embodiments, the probe includes SEQ ID NO:15321 and SEQ ID NO:13265. In embodiments, the probe includes SEQ ID NO:15322 and SEQ ID NO:13266. In embodiments, the probe includes SEQ ID NO:15323 and SEQ ID NO:13267. In embodiments, the probe includes SEQ ID NO:15324 and SEQ ID NO:13268. In embodiments, the probe includes SEQ ID NO:15325 and SEQ ID NO:13269. In embodiments, the probe includes SEQ ID NO:15326 and SEQ ID NO:13270. In embodiments, the probe includes SEQ ID NO:15327 and SEQ ID NO:13271. In embodiments, the probe includes SEQ ID NO:15328 and SEQ ID NO:13272. In embodiments, the probe includes SEQ ID NO:15329 and SEQ ID NO:13273. In embodiments, the probe includes SEQ ID NO:15330 and SEQ ID NO:13274. In embodiments, the probe includes SEQ ID NO:15331 and SEQ ID NO:13275. In embodiments, the probe includes SEQ ID NO:15332 and SEQ ID NO:13276. In embodiments, the probe includes SEQ ID NO:15333 and SEQ ID NO:13277. In embodiments, the probe includes SEQ ID NO:15334 and SEQ ID NO:13278. In embodiments, the probe includes SEQ ID NO:15335 and SEQ ID NO:13279. In embodiments, the probe includes SEQ ID NO:15336 and SEQ ID NO:13280. In embodiments, the probe includes SEQ ID NO:15337 and SEQ ID NO:13281. In embodiments, the probe includes SEQ ID NO:15338 and SEQ ID NO:13282. In embodiments, the probe includes SEQ ID NO:15339 and SEQ ID NO:13283. In embodiments, the probe includes SEQ ID NO:15340 and SEQ ID NO:13284. In embodiments, the probe includes SEQ ID NO:15341 and SEQ ID NO:13285. In embodiments, the probe includes SEQ ID NO:15342 and SEQ ID NO:13286. In embodiments, the probe includes SEQ ID NO:15343 and SEQ ID NO:13287. In embodiments, the probe includes SEQ ID NO:15344 and SEQ ID NO:13288. In embodiments, the probe includes SEQ ID NO:15345 and SEQ ID NO:13289. In embodiments, the probe includes SEQ ID NO:15346 and SEQ ID NO:13290. In embodiments, the probe includes SEQ ID NO:15347 and SEQ ID NO:13291. In embodiments, the probe includes SEQ ID NO:15348 and SEQ ID NO:13292. In embodiments, the probe includes SEQ ID NO:15349 and SEQ ID NO:13293. In embodiments, the probe includes SEQ ID NO:15350 and SEQ ID NO:13294. In embodiments, the probe includes SEQ ID NO:15351 and SEQ ID NO:13295. In embodiments, the probe includes SEQ ID NO:15352 and SEQ ID NO:13296. In embodiments, the probe includes SEQ ID NO:15353 and SEQ ID NO:13297. In embodiments, the probe includes SEQ ID NO:15354 and SEQ ID NO:13298. In embodiments, the probe includes SEQ ID NO:15355 and SEQ ID NO:13299. In embodiments, the probe includes SEQ ID NO:15356 and SEQ ID NO:13300. In embodiments, the probe includes SEQ ID NO:15357 and SEQ ID NO:13301. In embodiments, the probe includes SEQ ID NO:15358 and SEQ ID NO:13302. In embodiments, the probe includes SEQ ID NO:15359 and SEQ ID NO:13303. In embodiments, the probe includes SEQ ID NO:15360 and SEQ ID NO:13304. In embodiments, the probe includes SEQ ID NO:15361 and SEQ ID NO:13305. In embodiments, the probe includes SEQ ID NO:15362 and SEQ ID NO:13306. In embodiments, the probe includes SEQ ID NO:15363 and SEQ ID NO:13307. In embodiments, the probe includes SEQ ID NO:15364 and SEQ ID NO:13308. In embodiments, the probe includes SEQ ID NO:15365 and SEQ ID NO:13309. In embodiments, the probe includes SEQ ID NO:15366 and SEQ ID NO:13310. In embodiments, the probe includes SEQ ID NO:15367 and SEQ ID NO:13311. In embodiments, the probe includes SEQ ID NO:15368 and SEQ ID NO:13312. In embodiments, the probe includes SEQ ID NO:15369 and SEQ ID NO:13313. In embodiments, the probe includes SEQ ID NO:15370 and SEQ ID NO:13314. In embodiments, the probe includes SEQ ID NO:15371 and SEQ ID NO:13315. In embodiments, the probe includes SEQ ID NO:15372 and SEQ ID NO:13316. In embodiments, the probe includes SEQ ID NO:15373 and SEQ ID NO:13317. In embodiments, the probe includes SEQ ID NO:15374 and SEQ ID NO:13318. In embodiments, the probe includes SEQ ID NO:15375 and SEQ ID NO:13319. In embodiments, the probe includes SEQ ID NO:15376 and SEQ ID NO:13320. In embodiments, the probe includes SEQ ID NO:15377 and SEQ ID NO:13321. In embodiments, the probe includes SEQ ID NO:15378 and SEQ ID NO:13322. In embodiments, the probe includes SEQ ID NO:15379 and SEQ ID NO:13323. In embodiments, the probe includes SEQ ID NO:15380 and SEQ ID NO:13324. In embodiments, the probe includes SEQ ID NO:15381 and SEQ ID NO:13325. In embodiments, the probe includes SEQ ID NO:15382 and SEQ ID NO:13326. In embodiments, the probe includes SEQ ID NO:15383 and SEQ ID NO:13327. In embodiments, the probe includes SEQ ID NO:15384 and SEQ ID NO:13328. In embodiments, the probe includes SEQ ID NO:15385 and SEQ ID NO:13329. In embodiments, the probe includes SEQ ID NO:15386 and SEQ ID NO:13330. In embodiments, the probe includes SEQ ID NO:15387 and SEQ ID NO:13331. In embodiments, the probe includes SEQ ID NO:15388 and SEQ ID NO:13332. In embodiments, the probe includes SEQ ID NO:15389 and SEQ ID NO:13333. In embodiments, the probe includes SEQ ID NO:15390 and SEQ ID NO:13334. In embodiments, the probe includes SEQ ID NO:15391 and SEQ ID NO:13335. In embodiments, the probe includes SEQ ID NO:15392 and SEQ ID NO:13336. In embodiments, the probe includes SEQ ID NO:15393 and SEQ ID NO:13337. In embodiments, the probe includes SEQ ID NO:15394 and SEQ ID NO:13338. In embodiments, the probe includes SEQ ID NO:15395 and SEQ ID NO:13339. In embodiments, the probe includes SEQ ID NO:15396 and SEQ ID NO:13340. In embodiments, the probe includes SEQ ID NO:15397 and SEQ ID NO:13341. In embodiments, the probe includes SEQ ID NO:15398 and SEQ ID NO:13342. In embodiments, the probe includes SEQ ID NO:15399 and SEQ ID NO:13343. In embodiments, the probe includes SEQ ID NO:15400 and SEQ ID NO:13344. In embodiments, the probe includes SEQ ID NO:15401 and SEQ ID NO:13345. In embodiments, the probe includes SEQ ID NO:15402 and SEQ ID NO:13346. In embodiments, the probe includes SEQ ID NO:15403 and SEQ ID NO:13347. In embodiments, the probe includes SEQ ID NO:15404 and SEQ ID NO:13348. In embodiments, the probe includes SEQ ID NO:15405 and SEQ ID NO:13349. In embodiments, the probe includes SEQ ID NO:15406 and SEQ ID NO:13350. In embodiments, the probe includes SEQ ID NO:15407 and SEQ ID NO:13351. In embodiments, the probe includes SEQ ID NO:15408 and SEQ ID NO:13352. In embodiments, the probe includes SEQ ID NO:15409 and SEQ ID NO:13353. In embodiments, the probe includes SEQ ID NO:15410 and SEQ ID NO:13354. In embodiments, the probe includes SEQ ID NO:15411 and SEQ ID NO:13355. In embodiments, the probe includes SEQ ID NO:15412 and SEQ ID NO:13356. In embodiments, the probe includes SEQ ID NO:15413 and SEQ ID NO:13357. In embodiments, the probe includes SEQ ID NO:15414 and SEQ ID NO:13358. In embodiments, the probe includes SEQ ID NO:15415 and SEQ ID NO:13359. In embodiments, the probe includes SEQ ID NO:15416 and SEQ ID NO:13360. In embodiments, the probe includes SEQ ID NO:15417 and SEQ ID NO:13361. In embodiments, the probe includes SEQ ID NO:15418 and SEQ ID NO:13362. In embodiments, the probe includes SEQ ID NO:15419 and SEQ ID NO:13363. In embodiments, the probe includes SEQ ID NO:15420 and SEQ ID NO:13364. In embodiments, the probe includes SEQ ID NO:15421 and SEQ ID NO:13365. In embodiments, the probe includes SEQ ID NO:15422 and SEQ ID NO:13366. In embodiments, the probe includes SEQ ID NO:15423 and SEQ ID NO:13367. In embodiments, the probe includes SEQ ID NO:15424 and SEQ ID NO:13368. In embodiments, the probe includes SEQ ID NO:15425 and SEQ ID NO:13369. In embodiments, the probe includes SEQ ID NO:15426 and SEQ ID NO:13370. In embodiments, the probe includes SEQ ID NO:15427 and SEQ ID NO:13371. In embodiments, the probe includes SEQ ID NO:15428 and SEQ ID NO:13372. In embodiments, the probe includes SEQ ID NO:15429 and SEQ ID NO:13373. In embodiments, the probe includes SEQ ID NO:15430 and SEQ ID NO:13374. In embodiments, the probe includes SEQ ID NO:15431 and SEQ ID NO:13375. In embodiments, the probe includes SEQ ID NO:15432 and SEQ ID NO:13376. In embodiments, the probe includes SEQ ID NO:15433 and SEQ ID NO:13377. In embodiments, the probe includes SEQ ID NO:15434 and SEQ ID NO:13378. In embodiments, the probe includes SEQ ID NO:15435 and SEQ ID NO:13379. In embodiments, the probe includes SEQ ID NO:15436 and SEQ ID NO:13380. In embodiments, the probe includes SEQ ID NO:15437 and SEQ ID NO:13381. In embodiments, the probe includes SEQ ID NO:15438 and SEQ ID NO:13382. In embodiments, the probe includes SEQ ID NO:15439 and SEQ ID NO:13383. In embodiments, the probe includes SEQ ID NO:15440 and SEQ ID NO:13384. In embodiments, the probe includes SEQ ID NO:15441 and SEQ ID NO:13385. In embodiments, the probe includes SEQ ID NO:15442 and SEQ ID NO:13386. In embodiments, the probe includes SEQ ID NO:15443 and SEQ ID NO:13387. In embodiments, the probe includes SEQ ID NO:15444 and SEQ ID NO:13388. In embodiments, the probe includes SEQ ID NO:15445 and SEQ ID NO:13389. In embodiments, the probe includes SEQ ID NO:15446 and SEQ ID NO:13390. In embodiments, the probe includes SEQ ID NO:15447 and SEQ ID NO:13391. In embodiments, the probe includes SEQ ID NO:15448 and SEQ ID NO:13392. In embodiments, the probe includes SEQ ID NO:15449 and SEQ ID NO:13393. In embodiments, the probe includes SEQ ID NO:15450 and SEQ ID NO:13394. In embodiments, the probe includes SEQ ID NO:15451 and SEQ ID NO:13395. In embodiments, the probe includes SEQ ID NO:15452 and SEQ ID NO:13396. In embodiments, the probe includes SEQ ID NO:15453 and SEQ ID NO:13397. In embodiments, the probe includes SEQ ID NO:15454 and SEQ ID NO:13398. In embodiments, the probe includes SEQ ID NO:15455 and SEQ ID NO:13399. In embodiments, the probe includes SEQ ID NO:15456 and SEQ ID NO:13400. In embodiments, the probe includes SEQ ID NO:15457 and SEQ ID NO:13401. In embodiments, the probe includes SEQ ID NO:15458 and SEQ ID NO:13402. In embodiments, the probe includes SEQ ID NO:15459 and SEQ ID NO:13403. In embodiments, the probe includes SEQ ID NO:15460 and SEQ ID NO:13404. In embodiments, the probe includes SEQ ID NO:15461 and SEQ ID NO:13405. In embodiments, the probe includes SEQ ID NO:15462 and SEQ ID NO:13406. In embodiments, the probe includes SEQ ID NO:15463 and SEQ ID NO:13407. In embodiments, the probe includes SEQ ID NO:15464 and SEQ ID NO:13408. In embodiments, the probe includes SEQ ID NO:15465 and SEQ ID NO:13409. In embodiments, the probe includes SEQ ID NO:15466 and SEQ ID NO:13410. In embodiments, the probe includes SEQ ID NO:15467 and SEQ ID NO:13411. In embodiments, the probe includes SEQ ID NO:15468 and SEQ ID NO:13412. In embodiments, the probe includes SEQ ID NO:15469 and SEQ ID NO:13413. In embodiments, the probe includes SEQ ID NO:15470 and SEQ ID NO:13414. In embodiments, the probe includes SEQ ID NO:15471 and SEQ ID NO:13415. In embodiments, the probe includes SEQ ID NO:15472 and SEQ ID NO:13416. In embodiments, the probe includes SEQ ID NO:15473 and SEQ ID NO:13417. In embodiments, the probe includes SEQ ID NO:15474 and SEQ ID NO:13418. In embodiments, the probe includes SEQ ID NO:15475 and SEQ ID NO:13419. In embodiments, the probe includes SEQ ID NO:15476 and SEQ ID NO:13420. In embodiments, the probe includes SEQ ID NO:15477 and SEQ ID NO:13421. In embodiments, the probe includes SEQ ID NO:15478 and SEQ ID NO:13422. In embodiments, the probe includes SEQ ID NO:15479 and SEQ ID NO:13423. In embodiments, the probe includes SEQ ID NO:15480 and SEQ ID NO:13424. In embodiments, the probe includes SEQ ID NO:15481 and SEQ ID NO:13425. In embodiments, the probe includes SEQ ID NO:15482 and SEQ ID NO:13426. In embodiments, the probe includes SEQ ID NO:15483 and SEQ ID NO:13427. In embodiments, the probe includes SEQ ID NO:15484 and SEQ ID NO:13428. In embodiments, the probe includes SEQ ID NO:15485 and SEQ ID NO:13429. In embodiments, the probe includes SEQ ID NO:15486 and SEQ ID NO:13430. In embodiments, the probe includes SEQ ID NO:15487 and SEQ ID NO:13431. In embodiments, the probe includes SEQ ID NO:15488 and SEQ ID NO:13432. In embodiments, the probe includes SEQ ID NO:15489 and SEQ ID NO:13433. In embodiments, the probe includes SEQ ID NO:15490 and SEQ ID NO:13434. In embodiments, the probe includes SEQ ID NO:15491 and SEQ ID NO:13435. In embodiments, the probe includes SEQ ID NO:15492 and SEQ ID NO:13436. In embodiments, the probe includes SEQ ID NO:15493 and SEQ ID NO:13437. In embodiments, the probe includes SEQ ID NO:15494 and SEQ ID NO:13438. In embodiments, the probe includes SEQ ID NO:15495 and SEQ ID NO:13439. In embodiments, the probe includes SEQ ID NO:15496 and SEQ ID NO:13440. In embodiments, the probe includes SEQ ID NO:15497 and SEQ ID NO:13441. In embodiments, the probe includes SEQ ID NO:15498 and SEQ ID NO:13442. In embodiments, the probe includes SEQ ID NO:15499 and SEQ ID NO:13443. In embodiments, the probe includes SEQ ID NO:15500 and SEQ ID NO:13444. In embodiments, the probe includes SEQ ID NO:15501 and SEQ ID NO:13445. In embodiments, the probe includes SEQ ID NO:15502 and SEQ ID NO:13446. In embodiments, the probe includes SEQ ID NO:15503 and SEQ ID NO:13447. In embodiments, the probe includes SEQ ID NO:15504 and SEQ ID NO:13448. In embodiments, the probe includes SEQ ID NO:15505 and SEQ ID NO:13449. In embodiments, the probe includes SEQ ID NO:15506 and SEQ ID NO:13450. In embodiments, the probe includes SEQ ID NO:15507 and SEQ ID NO:13451. In embodiments, the probe includes SEQ ID NO:15508 and SEQ ID NO:13452. In embodiments, the probe includes SEQ ID NO:15509 and SEQ ID NO:13453. In embodiments, the probe includes SEQ ID NO:15510 and SEQ ID NO:13454. In embodiments, the probe includes SEQ ID NO:15511 and SEQ ID NO:13455. In embodiments, the probe includes SEQ ID NO:15512 and SEQ ID NO:13456. In embodiments, the probe includes SEQ ID NO:15513 and SEQ ID NO:13457. In embodiments, the probe includes SEQ ID NO:15514 and SEQ ID NO:13458. In embodiments, the probe includes SEQ ID NO:15515 and SEQ ID NO:13459. In embodiments, the probe includes SEQ ID NO:15516 and SEQ ID NO:13460. In embodiments, the probe includes SEQ ID NO:15517 and SEQ ID NO:13461. In embodiments, the probe includes SEQ ID NO:15518 and SEQ ID NO:13462. In embodiments, the probe includes SEQ ID NO:15519 and SEQ ID NO:13463. In embodiments, the probe includes SEQ ID NO:15520 and SEQ ID NO:13464. In embodiments, the probe includes SEQ ID NO:15521 and SEQ ID NO:13465. In embodiments, the probe includes SEQ ID NO:15522 and SEQ ID NO:13466. In embodiments, the probe includes SEQ ID NO:15523 and SEQ ID NO:13467. In embodiments, the probe includes SEQ ID NO:15524 and SEQ ID NO:13468. In embodiments, the probe includes SEQ ID NO:15525 and SEQ ID NO:13469. In embodiments, the probe includes SEQ ID NO:15526 and SEQ ID NO:13470. In embodiments, the probe includes SEQ ID NO:15527 and SEQ ID NO:13471. In embodiments, the probe includes SEQ ID NO:15528 and SEQ ID NO:13472. In embodiments, the probe includes SEQ ID NO:15529 and SEQ ID NO:13473. In embodiments, the probe includes SEQ ID NO:15530 and SEQ ID NO:13474. In embodiments, the probe includes SEQ ID NO:15531 and SEQ ID NO:13475. In embodiments, the probe includes SEQ ID NO:15532 and SEQ ID NO:13476. In embodiments, the probe includes SEQ ID NO:15533 and SEQ ID NO:13477. In embodiments, the probe includes SEQ ID NO:15534 and SEQ ID NO:13478. In embodiments, the probe includes SEQ ID NO:15535 and SEQ ID NO:13479. In embodiments, the probe includes SEQ ID NO:15536 and SEQ ID NO:13480. In embodiments, the probe includes SEQ ID NO:15537 and SEQ ID NO:13481. In embodiments, the probe includes SEQ ID NO:15538 and SEQ ID NO:13482. In embodiments, the probe includes SEQ ID NO:15539 and SEQ ID NO:13483.

In embodiments, the plurality of probes is ordered into subsets. In embodiments, the probes in a probe subset have a similar melting temperature for binding to their target sites. For example, the Tm may range from about 45° C. to about 65° C., including any Tm within this range such as 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, the subset of oligonucleotide probes are grouped according to their sequencing primer sequence. For example, a first subset includes the sequencing primer sequence SEQ ID NO:15540 and the subset includes 592 oligonucleotide probes. The second subset includes the sequencing primer sequence SEQ ID NO:15541 and the subset includes 636 oligonucleotide probes. In embodiments, the plurality is divided into 7 subsets and grouped according to a sequencing primer sequence. In embodiments, the first subset includes the sequencing primer sequence SEQ ID NO:15540 and the subset includes 382 oligonucleotide probes. In embodiments, the second subset includes the sequencing primer sequence SEQ ID NO:15541 and the subset includes 373 oligonucleotide probes. In embodiments, the third subset includes the sequencing primer sequence SEQ ID NO:15542 and the subset includes 353 oligonucleotide probes. In embodiments, the fourth subset includes the sequencing primer sequence SEQ ID NO:15543 and the subset includes 334 oligonucleotide probes. In embodiments, the fifth subset includes the sequencing primer sequence SEQ ID NO:15544 and the subset includes 318 oligonucleotide probes. In embodiments, the sixth subset includes the sequencing primer sequence SEQ ID NO:15545 and the subset includes 296 oligonucleotide probes. In embodiments, the probes of the first set are capable of binding between 75 and 150 different gene sequences. In embodiments, the probes are capable of binding between 25 and 75 different gene sequences. In embodiments, the probes are capable of binding between 30 and 100 different gene sequences. In embodiments, the probes are capable of binding between 40 and 120 different gene sequences. In embodiments, the probes are capable of binding between 50 and 150 different gene sequences. In embodiments, the probes are capable of binding between 60 and 180 different gene sequences. In embodiments, the probes are capable of binding between 70 and 200 different gene sequences. In embodiments, the probes are capable of binding between 80 and 220 different gene sequences. In embodiments, the probes are capable of binding between 90 and 250 different gene sequences. In embodiments, the probes are capable of binding between 100 and 300 different gene sequences. In embodiments, the probes are capable of binding between 150 and 400 different gene sequences.

In embodiments, the first probe set includes a plurality of oligonucleotide probes, wherein a first subset of the plurality of oligonucleotide probes includes a first sequencing primer binding sequence. A second subset includes a plurality of oligonucleotide probes, wherein each of the oligonucleotide probes includes the second sequencing primer binding sequence. A third subset includes a plurality of oligonucleotide probes, wherein each of the oligonucleotide probes includes the third sequencing primer binding sequence. A fourth subset includes a plurality of oligonucleotide probes, wherein each of the oligonucleotide probes includes the fourth sequencing primer binding sequence. A fifth subset includes a plurality of oligonucleotide probes, wherein each of the oligonucleotide probes includes the fifth sequencing primer binding sequence. A sixth subset includes a plurality of oligonucleotide probes, wherein each of the oligonucleotide probes includes the sixth sequencing primer binding sequence. A seventh subset includes a plurality of oligonucleotide probes, wherein each of the oligonucleotide probes includes the seventh sequencing primer binding sequence. An eighth subset includes a plurality of oligonucleotide probes, wherein each of the oligonucleotide probes includes the eighth sequencing primer binding sequence. A ninth subset includes a plurality of oligonucleotide probes, wherein each of the oligonucleotide probes includes the ninth sequencing primer binding sequence. A tenth subset includes a plurality of oligonucleotide probes, wherein each of the oligonucleotide probes includes the tenth sequencing primer binding sequence. An eleventh subset includes a plurality of oligonucleotide probes, wherein each of the oligonucleotide probes includes the eleventh sequencing primer binding sequence. A twelfth subset includes a plurality of oligonucleotide probes, wherein each of the oligonucleotide probes includes the twelfth sequencing primer binding sequence.

The field of cellular biology has recently been engaged in the intricate task of identifying and quantifying proteins within their native cellular environments. This endeavor, known as in situ protein detection, is pivotal for understanding the complex interplay of biomolecules within cells, thus offering insights into cellular functions, signaling pathways, and the underlying mechanisms of various diseases. Integrating nucleic acid-based methods (e.g., sequencing) with traditional protein detection strategies (e.g., antibody-oligo conjugates) opens new avenues for enhancing sensitivity, specificity, and multiplexing. Antibody-oligo conjugates leverages the specificity of antibodies to target proteins, for example proteins within cells, coupled with the precision and diversity of oligonucleotide sequences for subsequent detection and multiplexing, respectively. Detection includes binding a plurality of different specific binding reagents to a plurality of different targets and subsequently detecting the targets. This enhances the accuracy and reduces the likelihood of false positives in protein detection. Each binding reagent is specific for a particular target, and includes a sequence associated with that specific binding reagent. For example, a first specific binding reagent specific for protein PD-L1 includes an oligonucleotide having the sequence: “AGT.” A second specific binding reagent specific for protein CD15 includes an oligonucleotide having the sequence: “ACG.” When detecting both PD-L1 and CD15 in the same sample, every time the sequence “AGT” is detected, this corresponds to PD-L1 being present. Similarly, when “ACG” is detected, the presence of CD15 is determined. Detection of the oligonucleotide sequences may occur directly. For example, fluorescent probes complementary to the oligonucleotides, may be bound to the Ab-oligo conjugates. Subsequently, the fluorescence emitted by the probes is observed and analyzed under a fluorescence microscope, enabling the detection and localization of specific nucleic acid sequences, typically within the cellular or tissue context. In embodiments, amplification of the oligonucleotide sequence is often pursued to enhance the sensitivity and specificity of molecular probes, such as Fluorescent In Situ Hybridization (FISH) probes. Amplifying an oligonucleotide sequence significantly enhancing the signal-to-noise ratio, markedly increases the intensity of the signal detected from target sequences, making them more discernible against background noise. Amplification of the oligonucleotide sequence is crucial when detecting sequences present in low abundance or alongside other independent readout modalities such as RNA, as it substantially improves the precision and reliability of localization and quantification within native cellular or tissue structure. In embodiments, kits described herein includes antibodies and/or antibody-oligonucleotide (Ab-O) conjugates. In embodiments, the antibodies and/or Ab-O conjugates target CD11c, CD20, CD3e, CD31, CD4, CD45RA, CD56, CD8, HLA-DR, Ki-67, PanCK, PD-1, or PD-L1.

In embodiments, the target hybridization sequence of each probe (e.g., each probe of a plurality of probes) is complementary to different portions of the same target polynucleotide. In embodiments, the target hybridization sequence of each probe (e.g., each probe of a plurality of probes) is complementary to different portions of different target polynucleotides. In embodiments, the target hybridization sequence of each probe is complementary to portions of the same target polynucleotide that are separated by about 10 to about 500 nucleotides. In embodiments, the target hybridization sequence of each probe are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the target hybridization sequence of each probe is complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides. In embodiments, the target hybridization sequence includes one or more modified nucleotide(s). In embodiments, the modified nucleotide includes one or more locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, Zip nucleic acids (ZNAs), or combinations thereof. In embodiments, the first target hybridization sequence includes one or more locked nucleic acids (LNAs), Zip nucleic acids (ZNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleic acids. In embodiments, the target hybridization includes from 5′ to 3′ a plurality (e.g., 2 to 5) of phosphorothioate nucleic acids, followed by a plurality of synthetic nucleotides (e.g., LNAs), and subsequently followed by a plurality (e.g., 2 to 5) of canonical nucleobases. In some embodiments, the target hybridization sequence includes a plurality of canonical nucleobases, wherein the canonical nucleobases terminate (i.e., at the 3′ end) with a deoxyuracil nucleobase (dU).

In embodiments, the target hybridization sequence includes a plurality of LNAs interspersed throughout the polynucleotide. In embodiments, the target hybridization sequence includes a plurality of consecutive LNAs (e.g., 2 to 5 LNAs, 5 to 7 LNAs, or 7 to 10 LNAs) throughout the target hybridization sequence and/or probe sequence. In embodiments, the target hybridization sequence includes a plurality of consecutive LNAs (e.g., 2 to 5 LNAs, 5 to 7 LNAs, or 7 to 10 LNAs) throughout the target hybridization sequence. In embodiments, the target hybridization sequence includes a plurality of consecutive LNAs (e.g., 2 to 5 LNAs, 5 to 7 LNAs, or 7 to 10 LNAs) throughout the probe sequence.

In embodiments, the oligonucleotide probe binds to a target nucleic acid molecule. In embodiments, the nucleic acid molecule includes a gene sequence. In embodiments, the gene sequence is a ABCG2, ACE2, ACKR1, ACKR4, ACTA2, ACTG2, ADIPOQ, AGR3, AKT1, ALDOB, ANGPT2, ANKRD29, ANKRD30A, AQP1, AQP2, AQP3, AQP5, AR, ARG1, ATP6V0D2, BATF, BCL2, BEST2, BEST4, BTLA, C1QA, C1QB, C1QC, CALB1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CEACAM8, CKB, CLDN10, CLEC9A, CLU, COL1A1, CP, CSF1R, CSPG4, CTLA4, CTSL, CTSS, CUBN, CX3CL1, CX3CR1, CXCL10, CXCL11, CXCL12, CXCL13, CXCL9, CXCR3, CXCR4, CXCR5, CXCR6, CYP1A1, DCLK1, DEFA5, DES, DGKG, DMBT1, DMKN, DPT, DST, ECSCR, EGFR, EOMES, ERBB2, ESM1, ESR1, FABP1, FAP, FAS, FASN, FBLN1, FBN1, FCAR, FCGR1A, FCGR3A, FGB, FGFR4, FLNB, FN1, FOXA1, FOXI1, FOXJ1, FOXP3, FSCN1, GATA3, GATM, GIP, GNLY, GPC1, GPR183, GRHL1, GZMA, GZMB, GZMH, GZMK, HAVCR2, HLA-A, HLA-DRA, HOXD8, HSP90AA1, ICA1, ICOSLG, ID2, IDO1, IFNG, IGHA1, IGHD, IGHG1, IGHM, IL10, IL10RA, IL17A, IL1B, IL2RA, IL2RB, IL6, IL7R, INS, IRF1, ITGAM, ITGAX, ITGB2, JCHAIN, KDR, KIT, KLRB1, KLRD1, KLRF1, KLRK1, KRAS, KRT14, KRT23, KRT5, KRT6B, KRT7, KRT8, LAG3, LAMC3, LARS1, LGR5, LPL, LRP2, LTBP2, LUM, LYVE1, MADCAM1, MAPK1, MEDAG, MET, MLN, MLPH, MMP1, MMRN1, MMRN2, MRC1, MS4A1, MUC1, MUC2, MUC5AC, MUC5B, MYH11, MYLK, NCAM1, NCR1, NEUROD1, NEUROG3, NKG7, NOS1, NPHS2, NRXN1, NTS, OLFM4, PDCD1, PDCD1LG2, PDE4A, PDGFRA, PDGFRB, PDK4, PDPN, PECAM1, PGR, PHGR1, PIM1, PLIN1, PODXL, POLR2A, POSTN, POU2F3, PPARG, PRDM1, PRF1, PTGDS, PTGS2, PYY, RARRES1, RBP2, RGS5, RSPO3, S100A9, SCGB1A1, SCGB3A2, SDC1, SELE, SELL, SERPINA3, SFTPD, SH2D6, SLC12A1, SLC12A3, SLC26A4, SLC2A1, SLC4A1, SLC4A9, SLC8A1, SNCA, SNCG, SOX10, SPP1, SST, STAT1, STAT3, STAT4, STC1, TAGLN, TAP1, TAP2, TBX21, TCL1A, TFPI, TGFB1, THY1, TIGIT, TLR2, TLR4, TLR9, TMPRSS2, TNF, TNFRSF17, TNFRSF4, TNFRSF9, TNFSF13B, TNFSF9, TOMM7, TP63, TPD52, UMOD, UPK3B, VCAM1, VCAN, VEGFA, VIM, VWF, or WARS1 gene sequence. In embodiments, the oligonucleotide probe binds to External RNA Controls Consortium (ERCC) molecules. In embodiments, the ERCC molecule is ERCC-00019, ERCC-00024, ERCC-00059, ERCC-00142, or ERCC-00143.

In embodiments, the gene sequence includes a ACKR1, ACKR4, ACTA2, ADIPOQ, ANGPT2, ANKRD29, AQP1, CAVIN2, CCL2, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CEACAM8, CLEC9A, COL1A1, CSF1R, CSPG4, CTLA4, CTSS, CX3CL1, CX3CR1, CXCL10, CXCL11, CXCL13, CXCL9, CXCR3, CXCR4, CXCR5, CXCR6, CYP1A1, DES, DGKG, DPT, EGFR, EOMES, EPCAM, ERBB2, ESM1, FAP, FAS, FASN, FBLN1, FBN1, FCAR, FCGR1A, FCGR3A, FGFR4, FN1, FOXP3, FSCN1, GATA3, GATM, GNLY, GPC1, GPR183, GZMA, GZMB, GZMH, GZMK, HAVCR2, HLA-A, HLA-DRA, HOXD8, ICOSLG, ID2, IDO1, IFNG, IGHA1, IGHD, IGHG1, IGHM, IL10, IL10RA, IL17A, IL1B, IL2RA, IL2RB, IL6, IL7R, IRF1, ITGAM, ITGAX, ITGB2, JCHAIN, KDR, KIT, KLRB1, KLRD1, KLRF1, KLRK1, KRAS, LAG3, LAMC3, LARS1, LPL, LTBP2, LUM, LYVE1, MADCAM1, MAPK1, MET, MKI67, MMP1, MMRN1, MMRN2, MRC1, MS4A1, MYH11, NCAM1, NCR1, NKG7, NRXN1, PDCD1, PDCD1LG2, PDE4A, PDGFRA, PDGFRB, PDK4, PECAM1, PIM1, PLIN1, POSTN, PPARG, PRDM1, PRF1, PTGS2, RARRES1, RGS5, RSPO3, S100A9, SDC1, SELE, SELL, SLC2A1, SNCA, SNCG, SOX10, STAT1, STAT4, STC1, TAGLN, TAP1, TAP2, TBX21, TCL1A, TFPI, TGFB1, THY1, TIGIT, TLR2, TLR4, TLR9, TNF, TNFRSF17, TNFRSF4, TNFRSF9, TNFSF13B, TNFSF9, VCAM1, VCAN, VEGFA, VIM, VWF, or WARS1 gene sequence.

In embodiments, the gene sequence is CCL2, CCR2, CD14, CD19, CD274, CD28, CD3D, CD3E, CD4, CD44, CD8A, CTLA4, FOXP3, HAVCR2, IFNG, IL2RA, IL2RB, IL7R, MS4A1, PDCD1LG2, PDCD1, TIGIT, CD68, CXCL13, EPCAM, MKI67, IGHG1, IGHA1, COL1A1, ACTA2, LYVE1, MADCAM1, PDGFRB, PECAM1, or THY1. In embodiments, the oligonucleotide probe includes SEQ ID NO:11530 and SEQ ID NO:13586. In embodiments, the oligonucleotide probe includes SEQ ID NO:11534 and SEQ ID NO:13590. In embodiments, the oligonucleotide probe includes SEQ ID NO:11540 and SEQ ID NO:13596. In embodiments, the oligonucleotide probe includes SEQ ID NO:11544 and SEQ ID NO:13600. In embodiments, the oligonucleotide probe includes SEQ ID NO:11550 and SEQ ID NO:13606. In embodiments, the oligonucleotide probe includes SEQ ID NO:11553 and SEQ ID NO:13609. In embodiments, the oligonucleotide probe includes SEQ ID NO:11563 and SEQ ID NO:13619. In embodiments, the oligonucleotide probe includes SEQ ID NO:11565 and SEQ ID NO:13621. In embodiments, the oligonucleotide probe includes SEQ ID NO:11572 and SEQ ID NO:13628. In embodiments, the oligonucleotide probe includes SEQ ID NO:11570 and SEQ ID NO:13626. In embodiments, the oligonucleotide probe includes SEQ ID NO:11583 and SEQ ID NO:13639. In embodiments, the oligonucleotide probe includes SEQ ID NO:11590 and SEQ ID NO:13646. In embodiments, the oligonucleotide probe includes SEQ ID NO:11605 and SEQ ID NO:13661. In embodiments, the oligonucleotide probe includes SEQ ID NO:11618 and SEQ ID NO:13674. In embodiments, the oligonucleotide probe includes SEQ ID NO:11626 and SEQ ID NO:13682. In embodiments, the oligonucleotide probe includes SEQ ID NO:11638 and SEQ ID NO:13694. In embodiments, the oligonucleotide probe includes SEQ ID NO:11640 and SEQ ID NO:13696. In embodiments, the oligonucleotide probe includes SEQ ID NO:11644 and SEQ ID NO:13700. In embodiments, the oligonucleotide probe includes SEQ ID NO:11666 and SEQ ID NO:13722. In embodiments, the oligonucleotide probe includes SEQ ID NO:11671 and SEQ ID NO:13727. In embodiments, the oligonucleotide probe includes SEQ ID NO:11673 and SEQ ID NO:13729. In embodiments, the oligonucleotide probe includes SEQ ID NO:11696 and SEQ ID NO:13752. In embodiments, the oligonucleotide probe includes SEQ ID NO:11718 and SEQ ID NO:13774. In embodiments, the oligonucleotide probe includes SEQ ID NO:11741 and SEQ ID NO:13797. In embodiments, the oligonucleotide probe includes SEQ ID NO:11763 and SEQ ID NO:13819. In embodiments, the oligonucleotide probe includes SEQ ID NO:11764 and SEQ ID NO:13820. In embodiments, the oligonucleotide probe includes SEQ ID NO:11727 and SEQ ID NO:13783. In embodiments, the oligonucleotide probe includes SEQ ID NO:11726 and SEQ ID NO:13782. In embodiments, the oligonucleotide probe includes SEQ ID NO:11443 and SEQ ID NO:13499. In embodiments, the oligonucleotide probe includes SEQ ID NO:11430 and SEQ ID NO:13486. In embodiments, the oligonucleotide probe includes SEQ ID NO:11472 and SEQ ID NO:13528. In embodiments, the oligonucleotide probe includes SEQ ID NO:11474 and SEQ ID NO:13530. In embodiments, the oligonucleotide probe includes SEQ ID NO:11490 and SEQ ID NO:13546. In embodiments, the oligonucleotide probe includes SEQ ID NO:11492 and SEQ ID NO:13548. In embodiments, the oligonucleotide probe includes SEQ ID NO:11518 and SEQ ID NO:13574.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell (e.g., a bacterial cell or bacterial spore), a fungal cell (e.g., a fungal spore), a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell.

In another aspect is provided a cell including the oligonucleotide probe as described herein. In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell. In embodiments, the target nucleic acid molecule is in a cell or tissue.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program.

In an aspect is provided a tissue including the oligonucleotide probe as described herein. In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm. In embodiments, the thickness of the tissue section is about 1 μm. In embodiments, the thickness of the tissue section is about 2 μm. In embodiments, the thickness of the tissue section is about 3 μm. In embodiments, the thickness of the tissue section is about 4 μm. In embodiments, the thickness of the tissue section is about 5 μm. In embodiments, the thickness of the tissue section is about 6 μm. In embodiments, the thickness of the tissue section is about 7 μm. In embodiments, the thickness of the tissue section is about 8 μm. In embodiments, the thickness of the tissue section is about 9 μm. In embodiments, the thickness of the tissue section is about 10 μm. In embodiments, the thickness of the tissue section is about 11 μm. In embodiments, the thickness of the tissue section is about 12 μm. In embodiments, the thickness of the tissue section is about 13 μm. In embodiments, the thickness of the tissue section is about 14 μm. In embodiments, the thickness of the tissue section is about 15 μm. In embodiments, the thickness of the tissue section is less than about 10 μm. In embodiments, the thickness of the tissue section is less about 6 μm, 7 μm, 8 μm, 9 μm or 10 μm. In embodiments, the cell or tissue is obtained via Laser capture microdissection (LCM). LCM is a method for isolating in micrometer-scale tissue or even single cells while retaining spatial information to link histology with molecular measurements. In LCM, a region of interest in the tissue section is isolated through laser cutting.

Tissue sections, e.g., tumor tissue samples, may be obtained surgically or using a laparoscope. A tissue section may be a tissue sample obtained from any part of the body to examine it for disease or injury, e.g., presence of cancer tissue or cells, or the extent or characteristics thereof. In particular embodiments, the tissue section includes abdominal tissue, bone, bone marrow, breast tissue, endometrial tissue, kidney tissue, liver tissue, lung or chest tissue, lymph node, nerve tissue, skin, testicular tissue, head or neck tissue, or thyroid tissue. In certain embodiments, the tissue is obtained from brain, breast, skin, bone, joint, skeletal muscle, smooth muscle, red bone marrow, thymus, lymphatic vessel, thoracic duct, spleen, lymph node, nasal cavity, pharynx, larynx, trachea, bronchus, lung, oral cavity, esophagus, liver, stomach, small intestine, large intestine, rectum, anus, spinal cord, nerve, pineal gland, pituitary gland, thyroid gland, thymus, adrenal gland, pancreas, ovary, testis, heart, blood vessel, kidney, uterus, urinary bladder, urethra, prostate gland, penis, prostate, testis, scrotum, ductus deferens, mammary glands, ovary, uterus, vagina, or uterine tube.

In embodiments, the tissue section includes a tissue or a cell. Biological tissue samples suitable for use with the methods and systems described herein generally include any type of tissue samples collected from living or dead subjects, such as, for example, tumor tissue and autopsy samples. Tissue samples may be collected and processed using the methods and systems described herein and subjected to microscopic analysis immediately following processing, or may be preserved and subjected to microscopic analysis at a future time, e.g., after storage for an extended period of time. In some embodiments, the methods described herein may be used to preserve tissue samples in a stable, accessible and fully intact form for future analysis. For example, tissue samples, such as, e.g., human tumor tissue samples, may be processed as described herein and cleared to remove a plurality of cellular components, such as, e.g., lipids, and then stored for future analysis. In some embodiments, the methods and systems described herein may be used to analyze a fresh tissue section. In some embodiments, the methods and systems described herein may be used to analyze a previously-preserved (e.g., previously fixed) or stored tissue section (e.g., tissue sample). For example, in some embodiments a previously-preserved tissue sample that has not been subjected to a sample preparation process described herein may be processed and analyzed as described herein. In particular methods, a tissue sample is frozen prior to being processed as described herein

The probe sequences described herein may be screened to avoid common six-base-cutter restriction enzyme recognition sites to aid in the ease of manipulation for conventional molecular cloning techniques. Selected sequences are additionally subjected to predicted secondary structure analysis, and those with the least secondary structure are chosen for further evaluation. Any program known in the art can be used to predict secondary structure, such as the MFOLD program (Zuker, 2003, Nucleic Acids Res. 31 (13):3406-15; Mathews et al., 1999, J. Mol. Biol. 288:911-940).

In embodiments, the probe oligonucleotide includes one or more modifications, for example the oligonucleotide may include nucleotides modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1):5-23). As used herein, the terms “peptide nucleic acids” (PNAs) refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength.

In an aspect is provided computer program products for use in conjunction with a computer system having a processor and a memory connected to the processor. The computer program products of the disclosure comprise a computer readable storage medium having a computer program mechanism encoded or embedded thereon. The computer program mechanism can be loaded into the memory of the computer and cause the processor to execute the steps of the methods of the disclosure. The methods described herein can preferably be implemented by use of the following computer systems, and according to the following methods. An exemplary computer system suitable for implementation of the methods of this disclosure comprises internal components and being linked to external components. The external components include one or more storage device(s). For example, a storage device can be one or more hard disks which are typically packaged together with the processor and memory. Other external components include user interface device, which can be a monitor and a keyboard, together with pointing device.

In an aspect is provided a device configured to detect multiple targets as described herein. In embodiments, the device includes a microscope. Examples of suitable microscopes include, but are not limited to, the Zeiss Axioscope 5 multichannel microscope (Carl Zeiss Microscopy, LLC), the Olympus BX63 automated microscope (Olympus Scientific Solutions Americas Corp), and the Nikon Eclipse Ti2 microscope (Nikon Instruments, Inc.). In embodiments, the device includes one or more light sources, one or more objective lenses, one or more sample carriers (e.g., sample holders, sample stages, and/or translation stages), one or more tube lenses, one or more image sensors or cameras, one or more processors or controllers, one or more additional optical components (e.g., lenses, mirrors, prisms, beam-splitters, optical filters, colored glass filters, narrowband interference filters, broadband interference filters, dichroic reflectors, diffraction gratings, apertures, shutters, optical fibers, optical waveguides, and/or acousto-optic modulators), or any combination thereof. In embodiments, the device includes a focus mechanism, e.g., an autofocus mechanism. In embodiments, the device is be configured to perform multichannel imaging, e.g., multichannel fluorescence imaging including the use of excitation light at one or more excitation wavelengths, and imaging the emitted fluorescence at two or more different emission wavelengths.

In embodiments, the device includes one or more image sensors (or cameras) that may be the same or may be different, and may include any of a variety of image sensors including but not limited to, photodiode arrays, charge-coupled device (CCD) sensors or cameras, or complementary metal-oxide-semiconductor (CMOS) image sensors or cameras. In embodiments, the one or more image sensors may comprise one-dimensional (linear) or two-dimensional pixel array sensors. In embodiments, the one or more image sensors may comprise monochrome image sensors (e.g., configured to capture greyscale images) or color image sensors (e.g., configured to capture RGB or color images). In embodiments, the one or more image sensors may be used to capture single images, e.g., a single image for each cycle of a plurality of cycles. In embodiments, the one or more image sensors may be used to capture a series of images, e.g., a series of images during each cycle of a plurality of cycles. In embodiments, a series of images may include images (or video frames) that correspond to images captured before, during, and/or after an event, e.g., before, during, and/or after addition of a probe to the sample being imaged. In embodiments, a series of images includes 2 images, 3 images, 4 images, 5 images, 10 images, 20 images, 30 images, 40 images, 50 images, 100 images, 200 images, 300 images, 400 images, 500 images, 1,000 images, or more than 1,000 images.

In embodiments, the device is configured to perform volumetric imaging (or optical sectioning). In embodiments, the imaging includes the acquisition of a plurality (or “stack”) of two-dimensional (2D) images to form a three-dimensional (3D) representation of the sample, where each two-dimensional image is aligned with the other images of the plurality in the sample plane (e.g., the X-Y plane), but is offset from the other two-dimensional images in a direction parallel to the optical axis of the imaging module (e.g., in the Z-direction). In embodiments, the stack of images may be acquired sequentially. In embodiments, the stack of images may be acquired simultaneously.

In an aspect is provided a kit, wherein the kit includes a plurality of probes described herein. In embodiments, the individual components of the kit can be alternatively contained either together in one storage container or separately in two or more storage containers (e.g., separate bottles or vials). In embodiments, the kit includes nucleotides in a buffer. In embodiments, the kit includes a buffer. For example, the sequencing solution and/or the chase solution may include a buffer such as ethanolamine (EA), tris(hydroxymethyl)aminomethane (Tris), glycine, a carbonate salt, a phosphate salt, a borate salt, 2-dimethyalaminomethanol (DMEA), 2-diethyalaminomethanol (DEEA), N,N,N′,N′-tetramethylethylenediamine (TEMED), and N,N,N′,N′-tetraethylethylenediamine (TEEDA), and combinations thereof. For example, the buffer may Tris-HCl (pH 9.2 at 25° C.), ammonium sulfate, MgCl₂, 0.1% Tween® 20, and dNTPs.

In embodiments, the kit includes a first plurality of nucleic acid molecules, wherein each nucleic acid molecule of the first plurality includes a first end, a backbone sequence including a first sequencing primer sequence, and a second end, wherein the first end includes a sequence selected from SEQ ID NO:11428 to SEQ ID NO:13483 and the second end includes a sequence selected from SEQ ID NO:13484 to SEQ ID NO:15539; and a second plurality of nucleic acid molecules, wherein each nucleic acid molecule of the second plurality includes a first end, a backbone sequence including a second sequencing primer sequence, and a second end, wherein the first end includes a sequence selected from SEQ ID NO:11428 to SEQ ID NO:13483 and the second end includes a sequence selected from SEQ ID NO:13484 to SEQ ID NO:15539. In embodiments, the kit includes a third, fourth, fifth, and sixth plurality of nucleic acid molecules, wherein each plurality includes a plurality of nucleic acid molecules with a different sequencing primer sequence (e.g., a sequence provided herein).

In embodiments, the kit is stored for 1 to 90 days. In embodiments, the kit is stored for greater than 90 days. In embodiments, the kit is stored for 1 to 30 days. In embodiments, the kit is stored for 1, 5, 7, 14, 21, 30, 45, 60, 75, 90, or more days. In embodiments, the kit is stored at less than about 25° C. In embodiments, the kit is stored at less than about 5° C. In embodiments, the kit is stored at about 4° C. In embodiments, the kit is stored in the dark (e.g., in the absence of light, such as visible light or UV light). In embodiments, the kit is stored at 2-8° C. In embodiments, the kit is stored for at least 1 day, at least 2 days, at least 3 days, or at least 7 days. In embodiments, the kit is stored for about 1 week, about 2 weeks, about 3 weeks, about 4 weeks, about 5 weeks, about 6 weeks, about 7 weeks, or about 8 weeks. In embodiments, the kit is stored for about 1 month, about 2 months, about 3 months, about 4 months, about 5 months, about 6 months, about 7 months, about 8 months, about 9 months, about 10 months, about 11 months, or about 12 months. In embodiments, the kit is stored at about 2° C.
8° C., about 20° C.-30° C., or about 4° C.-37° C. In embodiments, the kit is stored at about −5° C. to −30° C. and protected from light. In embodiments, the kit is stored at about 2° C.-8° C. and protected from light. In embodiments, the kit is stored at about 20° C.-30° C. and protected from light. In embodiments, the kit is stored at or about 4° C.-37° C. and protected from light. In embodiments, the kit includes a polymerase. In embodiments, the kit includes a ligase.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, Coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In embodiments, the kit is designed for staining tissue samples for imaging and detecting target molecules (e.g., proteins) can be significantly expanded beyond the inclusion of fluorophores. For instance, the kit can include eosin and hematoxylin, which are classic histological stains. Eosin, a red dye, typically stains acidic components of the cell such as cytoplasmic proteins, while hematoxylin, a basic dye, binds to nucleic acids, coloring the cell nucleus blue. This combination is widely used in histopathology for detailed tissue structure visualization. Moreover, the kit can encompass stains such as acridine orange, a nucleic acid-selective fluorescent cationic dye, and Bismarck brown, which is often used for staining backgrounds in histological tissue sections. Carmine, another potential inclusion, is a natural red dye used for staining glycogen, while Coomassie blue is a popular choice for protein staining in gel electrophoresis. Crystal violet, a triarylmethane dye, can be included for staining cell walls and nuclei, and DAPI, a fluorescent stain that binds strongly to A-T rich regions in DNA, is useful in fluorescence microscopy. Ethidium bromide, a fluorescent intercalator, is also a valuable addition for its role in nucleic acid staining, especially in gel electrophoresis. Further, the kit can include acid fuchsine, used in Masson's trichrome stain; Hoechst stains, which are cell-permeable, DNA-specific blue fluorescent dyes; and iodine, commonly used in Gram staining and for staining starch in plant cells. Methyl green and methylene blue, both traditional histological stains, can be included for their affinity towards nucleic acids. Neutral red, a vital stain that accumulates in lysosomes, Nile blue and Nile red, both used for staining lipids, and osmium tetroxide, a heavy metal stain for lipid bilayers in electron microscopy, can be part of the kit. Propidium iodide, a popular red-fluorescent nuclear and chromosome counterstain, along with rhodamine, may be utilized. Safranin, commonly used in Gram staining, can be included for its ability to stain cell components like nuclei, cytoplasm, and cell walls in various colors, enhancing the contrast and detail in tissue imaging.

In embodiments, the kit includes a flow cell, for example a solid support as described herein. In embodiments, the kit includes labeled nucleotides including differently labeled nucleotides, enzymes, buffers, oligonucleotides, and related solvents and solutions. In embodiments, the kit includes one or more oligonucleotide probes (e.g., an oligonucleotide probe as described herein). The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., Circligase™ enzyme, Taq DNA Ligase, HiFi Taq DNA Ligase, T4 ligase, SplintR® ligase, or Ampligase® DNA Ligase). For example, such a kit further includes the following components: (a) reaction buffer for controlling pH and providing an optimized salt composition for a ligation enzyme (e.g., Circligase™ enzyme, Taq DNA Ligase, HiFi Taq DNA Ligase, T4 ligase, SplintR® ligase, or Ampligase® DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a microplate, and reagents for sample preparation and purification, amplification, and/or sequencing (e.g., one or more sequencing reaction mixtures). In embodiments, the kit includes a flow cell, and reagents for sample preparation and purification, amplification, and/or sequencing (e.g., one or more sequencing reaction mixtures). In embodiments, the kit includes reagents for protein detection includes a plurality of specific binding agents linked to an oligonucleotide (e.g., DNA-conjugated antibodies).

In embodiments, the kit further includes a ligase. In embodiments, the kit includes one or more ligases. In embodiments, the kit includes a plurality of ligases. In embodiments, the kit further includes a polymerase. In embodiments, the kit further includes one or more polymerases. In embodiments, the kit includes a plurality of polymerases. In embodiments, the kit includes a ligase and one or more polymerases. In embodiments, the one or more polymerases include a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase. In embodiments, the polymerase is a Thermus thermophilus (Tth) DNA polymerase or mutant thereof. In embodiments, the polymerase is a Reverse Transcription Xenopolymerase (RTX). In embodiments, the polymerase is a mutant M-MLV reverse transcriptase from the Moloney murine leukemia virus.

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site.

In embodiments, kit includes the flow cell assembly as described herein and a flow cell carrier (e.g., a flow cell carrier as described in U.S. Pat. No. 11,747,262, which is incorporated herein by reference for all purposes). In embodiments, the kit includes a sequencing primer, wherein the sequencing primer includes the sequence: SEQ ID NO:11419, SEQ ID NO:11420, SEQ ID NO:11421, SEQ ID NO:11422, SEQ ID NO:11423, SEQ ID NO:11424, SEQ ID NO:11425, or SEQ ID NO:11426. In embodiments, the sequencing primer includes the sequence: SEQ ID NO:15540, SEQ ID NO: 15541, SEQ ID NO: 15542, SEQ ID NO: 15543, SEQ ID NO: 15544, and SEQ ID NO: 15545. In embodiments, the second end includes a sequence selected from SEQ ID NO:13484 to SEQ ID NO:15538. In embodiments, the kit includes a polymerase and a plurality of deoxynucleotides (dNTPs).

In an aspect is provided a solid support comprising one or more tissue sections, wherein the tissues include a plurality of compositions and/or complexes as described herein. In embodiments, the tissue includes liver tissue, kidney tissue, bone tissue, lung tissue, thymus tissue, adrenal tissue, skin tissue, bladder tissue, colon tissue, spleen tissue, or brain tissue. In embodiments, the tissue section is from adrenal glands, appendix, bladder, bones, bone marrow, brain, lung bronchi, diaphragm, ears, esophagus, eyes, fallopian tubes, gallbladder, genitals, heart, hypothalamus, joints, kidneys, large intestine, larynx, liver, lungs, lymph nodes, mammary glands, mesentery, mouth, nasal cavity, nose, ovaries, pancreas, pineal gland, parathyroid glands, pharynx, pituitary gland, prostate, rectum, salivary glands, skeletal muscles, skin, small intestine, spinal cord, spleen, stomach, thymus gland, thyroid, trachea, tongue, ureters, urethra, uterus, vagina, placenta, testes, nails, vas deferens, seminal vesicles, bulbourethral glands, penis, scrotum, parathyroid glands, tonsils, nerves, subcutaneous tissue, olfactory epithelium, or cerebellum.

### III. Methods

In an aspect is provided a method of detecting a nucleic acid molecule. In embodiments, the nucleic acid molecule is in a cell or tissue. In embodiments, the nucleic acid molecule includes a gene sequence. In embodiments, the method includes contacting the nucleic acid molecule with an oligonucleotide probe, wherein the probe includes a sequence provided herein, and binding the oligonucleotide probe to the nucleic acid molecule. In embodiments, the oligonucleotide probe includes a first sequence and a second sequence, wherein the first sequence is as a first end and the second sequence is at a second end. In embodiments, the first sequence selected from SEQ ID NO:11428 to SEQ ID NO:13483 and a second sequence selected from SEQ ID NO:13484 to SEQ ID NO:15539. In embodiments, the oligonucleotide probe includes a primer binding sequence, or complement thereof, selected from the following: SEQ ID NO:15540, SEQ ID NO:15541, SEQ ID NO:15542, SEQ ID NO:15543, SEQ ID NO:15544, or SEQ ID NO:15545. In embodiments, the first sequence is selected from SEQ ID NO:5710 to SEQ ID NO:11418 and the second sequence is selected from SEQ ID NO:1 to SEQ ID NO:5709. In embodiments, the method includes sequencing the first sequence and/or the second sequence. In embodiments, prior to sequencing, the method includes amplifying the oligonucleotide probe. For example, following annealing the oligonucleotide probe to the nucleic acid molecule, the first sequence and the second sequence are ligated together to form a circular polynucleotide. The circular polynucleotide may then be amplified using rolling circle amplification (RCA) to form amplification produces including copies of the first sequence and the second sequence (and/or complements thereof) which may then be sequenced. In embodiments, amplification is performed at a temperature between or between about 20° C. and about 60° C. In embodiments, amplification is performed at a temperature between or between about 30° C. and about 40° C. In some embodiments, the amplification step, such as the rolling circle amplification (RCA) is performed at a temperature between at or about 25° C. and at or about 50° C., e.g., 25° C., 27° C., 29° C., 31° C., 33° C., 35° C., 37° C., 39° C., 41° C., 43° C., 45° C., 47° C., or 49° C.

In embodiments the probe oligonucleotides include a sequence provided herein, or a sequence with 80% or more homology with a sequence described herein. In embodiments, the probe oligonucleotide includes a sequence that is 85%, 90%, 95%, 96%, 97%, 98%, 99%, or 100% identical to a sequence described herein.

In embodiments, the methods of detection are performed in multiplex assays, whereby a plurality of target molecules are detected in the same assay (a single reaction mixture). In embodiments, the plurality of target molecules is detected simultaneously. In embodiments, the plurality of target molecules detected in the same assay is at least 5 different target molecules, at least 10 different target molecules, at least 20 different target molecules, at least 50 different target molecules, at least 75 different target molecules, at least 100 different target molecules, at least 200 different target molecules, at least 500 different target molecules, or at least 750 different target molecules, or at least 1000 different target molecules. In embodiments, the plurality of target molecules detected in the same assay is up to 50 different target molecules, up to 100 different target molecules, up to 150 different target molecules, up to 200 different target molecules, up to 300 different target molecules, up to 500 different target molecules, up to 750 different target molecules, up to 1000 different target molecules, up to 2000 different target molecules, or up to 5000 different target molecules. In embodiments, the plurality of target molecules detected is any range in between the foregoing numbers of different target molecules, such as, but not limited to, from 20 to 50 different target molecules, from 50 to 200 different target molecules, from 100 to 1000 different target molecules, or from 500 to 5000 different target molecules.

In embodiments, the method further includes detecting a target protein molecule. In embodiments, the method includes detecting the target protein molecule in a cell or tissue. In embodiments, the method includes binding a polynucleotide probe to an oligonucleotide, wherein the oligonucleotide is covalently bound to a specific binding agent (e.g., an antibody); detecting the polynucleotide probe, thereby detecting the target molecule. In embodiments, detecting includes serially contacting the oligonucleotides with labeled probes (e.g., labeled oligonucleotides or labeled nucleotides).

In embodiments, the polynucleotide probe includes a fluorescently labeled probe. The phrase “labeled probes” refers to mixture of nucleic acids that are detectably labeled, e.g., fluorescently labeled, such that the presence of the probe, as well as, any target sequence to which the probe is bound can be detected by assessing the presence of the label. In embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety. In embodiments, the method includes binding a polynucleotide probe including a first binding sequence and a second binding sequence to an oligonucleotide, wherein the oligonucleotide is attached to the target molecule (e.g., covalently attached to an antibody, wherein the antibody is specifically bound to the target protein molecule); amplifying the polynucleotide probe to form an amplification product comprising one or more copies of the first binding sequence and the second binding sequence; hybridizing a primer to the amplification product and incorporating a labeled nucleotide into the primer, thereby detecting the target protein molecule. In embodiments, the method includes hybridizing a labeled probe to the amplification product and detecting the labeled probe, thereby detecting the target protein molecule. In embodiments, the method includes detecting CD11c, CD20, CD3e, CD31, CD4, CD45RA, CD56, CD8, HLA-DR, Ki-67, PanCK, PD-1, and/or PD-L1.

In embodiments, prior to contacting a cell or tissue with a probe oligonucleotide, the method further includes immobilizing a cell or tissue section onto a solid support (e.g., a flow cell or well in a microplate). In embodiments, the solid support includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate. In embodiments, the method includes immobilizing a plurality of tissue sections to the solid support (e.g., a flow cell), wherein a tissue in a plurality of tissue sections includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 m sections).

In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

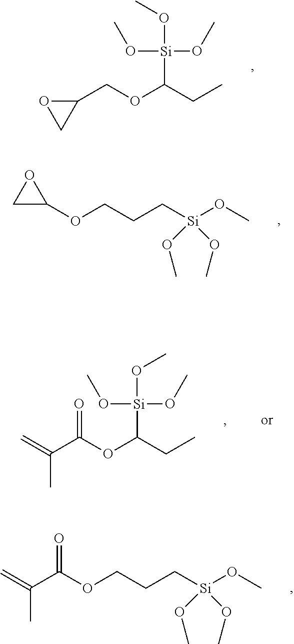

or a copolymer thereof. In embodiments, the polymer layer is an organically-modified ceramic polymer. In embodiments, the polymer includes polymerized monomers of alkoxysilyl polymers, such as

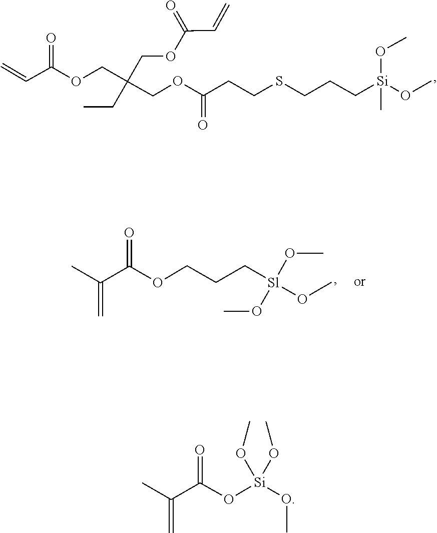

In embodiments, the solid support includes polymerized units of

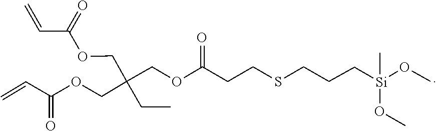 [SMILES: C=CC(=O)OCC(CC)(COC(=O)C=C)COC(=O)CCSCCC[Si](C)(OC)OC]

In embodiments, the solid support includes polymerized units of

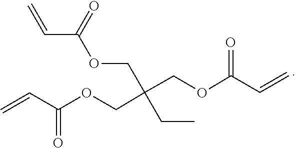 [SMILES: C=CC(=O)OCC(CC)(COC(=O)C=C)COC(=O)C=C]

In embodiments, the solid support includes polymerized unites of

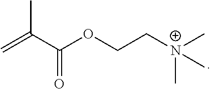

In embodiments, the polymer layer includes one or more ceramic particles, (e.g., silicates, aluminates, and titanates). In embodiments, the polymer layer includes tantalum(V) oxide, titanium dioxide, zinc oxide, and/or iron oxide.

In embodiments, the method includes immobilizing a plurality of tissue sections to the solid support (e.g., a flow cell), wherein a tissue in a plurality of tissue sections includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 m sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the first solid support as described herein (e.g., inverting the punch device); pushing the sample portion out of the punch device using a piston, so that all or a portion thereof of the sample portion is positioned on the first solid support as described herein. In embodiments, the method further includes cutting a sample portion from the biological sample using two or more punch devices such that each punch device contains a different the sample portion; mounting each punch device containing the sample portion onto the first solid support as described herein; pushing the sample portions out of the punch devices using one or more pistons so that the sample portions are positioned onto the solid support as described herein (e.g., a flow cell described herein).

In embodiments, the method further includes permeabilizing the tissue or cell prior to binding the probe to the biomolecule. Methods for permeabilization are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the tissue and/or cell is cleared (e.g., digested) of proteins, lipids, or proteins and lipids. In embodiments, permeabilizing the tissue or cell does not release the biomolecules (e.g., the one or more biomolecules) from within the tissue or cell. For example, after a fixation process (e.g. formaldehyde cross-linking), proteins and nucleic acids are immobilized within the cells of a tissue section, and are therefore not liberated into the environment following permeabilization of the cells. In embodiments, permeabilizing includes contacting the sample (e.g., the cell or tissue) with a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X100™, Tween-20™, or sodium dodecyl sulfate (SDS)), and enzymes (e.g., trypsin, proteases (e.g., proteinase K). In embodiments, the detergent is an anionic detergent (e.g., SDS or N-lauroylsarcosine sodium salt solution). In embodiments, the permeabilization agent includes polyethylene glycol (PEG). In embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, or 16K. In embodiments, the PEG is present at a concentration from about 2% to 25%, from about 4% to about 23%, from about 6% to about 21%, or from about 8% to about 20% (v/v).

Imaging deep into a tissue volume is problematic due to inherently fluorescent molecules present in the tissue or introduced during processing which give rise to autofluorescence that masks fluorescently labelled structures of interest. Typically, autofluorescence decreases image quality by lowering the signal to noise ratio across multiple fluorescence channels and undermines sharp images. Autofluorescence may arise from endogenous fluorescent biomolecules (NADPH, collagen, flavins, tyrosine, and others) or be introduced by the formation of Schiffs bases during fixation with aldehydes (e.g., glutaraldehyde and paraformaldehyde). Additional light scattering is provided by various cellular components, such as ribosomes, nuclei, nucleoli, mitochondria, lipid droplets, membranes, myelin, cytoskeletal components, and extracellular matrix components such as collagen and elastin.

In embodiments, the tissue or cell is cleared using a solvent-based clearing approach. Solvent-based clearing techniques typically includes two steps: 1) dehydration (e.g., contacting the sample with methanol with or without hexane or, tetrahydrofuran (THF) alone) and 2) clearing by refractive index matching to the remaining dehydrated tissue's index (e.g., contacting the tissue sample with methyl salicylate, benzyl alcohol, benzyl benzoate, dichloromethane, or dibenzyl ether). Alternatively, the initial dehydration may be performed using phosphate buffered saline (PBS), detergent, and dimethyl sulfoxide (DMSO). In embodiments, the tissue is cleared by contacting the tissue sample with an aqueous solution containing sucrose, fructose, 2,2′-thiodiethanol (TDE), or formamide.

In embodiments, the tissue or cell is cleared utilizing the 3D imaging of solvent-cleared organs (3DISCO) method as described in ErtUrk Aet al. Nat Protoc. 2012 Nov. 7(11):1983-95, which is incorporated herein by reference. For example, a sample is incubated overnight in 50% v/v tetrahydrofuran/H₂O (THF), followed by incubation for at least one hour 80% THF/H₂O and followed by incubation in a 100% THF solution. This is then followed by contacting the sample with dichloromethane (DCM) and an incubation in dibenzyl ether (DBE) until clear.

In embodiments, the tissue or cell is cleared according to a known technique in the art, for example CLARITY (Chung K., et al. Nature 497, 332-337 (2013)), PACT-PARS (Yang Bet al. Cell 158, 945-958 (2014).), CUBIC (Susaki E. A. et al. Cell 157, 726-739 (2014), 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, e0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

In embodiments, the tissue or cell is contacted with an alkaline solution containing a combination of 2,2′-thiodiethanol (TDE), DMSO, D-sorbitol, and Tris. In embodiments, the tissue section is contacted with an aqueous solution including 20% (vol/vol) DMSO, 40% (vol/vol) TDE, 20% (wt/vol) sorbitol, and 6% (wt/vol, equal to 0.5 M) Tris base. In embodiments, the tissue section is contacted with an aqueous solution including 25% (wt/wt) urea, 25% (wt/wt) N,N,N′,N′-Tetrakis (2-hydroxypropyl) ethylenediamine, and 15% (wt/wt) Triton X-100. In embodiments, the tissue section is contact with an aqueous solution including 9.1 M urea, 22.5% (wt/vol) D-sorbitol, and 5% (wt/vol) Triton X-100. In embodiments, the tissue section is contact with an aqueous solution including 30% (wt/vol) urea, 20% (wt/vol) D-sorbitol, and 5% (wt/vol) glycerol dissolved in DMSO. In embodiments, the tissue or cell is contacted with an aqueous solution according to the protocols described in Shan, Q H., Qin, X Y., Zhou, N. et al. BMC Biol 20, 77 (2022).

In embodiments, the method includes imaging the immobilized tissue section. In embodiments, the method further includes an imaging modality, immunofluorescence (IF), or immunohistochemistry modality (e.g., immunostaining). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 m. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 m, and for image acquisition in the regions of interest, an interval in the range of 2 to 5 m may be used. In embodiments, the tissue sample is stained using immunofluorescence, immunohistochemistry, or hematoxylin and/or eosin (H&E) stain.

To microscopically visualize tissue sections prepared by the subject methods, in some embodiments the tissue section is embedded in a mounting medium. Mounting medium is typically selected based on its suitability for the reagents used to visualize the cellular biomolecules, the refractive index of the tissue section, and the microscopic analysis to be performed. For example, for phase-contrast work, the refractive index of the mounting medium should be different from the refractive index of the specimen, whereas for bright-field work the refractive indexes should be similar. As another example, for epifluorescence work, a mounting medium should be selected that reduces fading, photobleaching or quenching during microscopy or storage. In certain embodiments, a mounting medium or mounting solution may be selected to enhance or increase the optical clarity of the cleared tissue specimen. Nonlimiting examples of suitable mounting media that may be used include glycerol, CC/Mount™, Fluoromount™ Fluoroshield™, ImmunHistoMount™, Vectashield™, Permount™, Acrytol™, CureMount™ FocusClear™, or equivalents thereof.

The biological targets or molecules to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA. For example, following immobilization on the receiving substrate, the sections may be fixed with methanol, permeabilized with 0.025% Triton in PBS solution, and stained with primary antibodies directed against vimentin (fibroblasts) and macrophages, followed by secondary antibody labeling (e.g., Alexa-594 conjugated secondary antibodies). Additional counterstaining may be performed, for example using 4,6-diamidino-2-phenylindole (DAPI) mounting media to counterstain nuclei.

In an aspect is provided a method of imaging a cell or tissue. In embodiments, the method includes: contacting a cell or tissue with a first probe set, wherein the first probe set includes a plurality of probes as described herein; binding each probe to a different target of the cell or tissue; imaging (e.g., obtaining an image) the cell or tissue; contacting the cell or tissue with a second probe set, wherein the second probe set includes a plurality of probes as described herein; binding each probe to a different target of the cell or tissue; and imaging (e.g., obtaining an image) the cell or tissue.

In embodiments, amplifying the circular polynucleotide generates an amplification product including multiple copies of the target sequence, or a complement thereof. In embodiments, the method includes serially cycling through detection cycles to determine the sequence (e.g., the order of the nucleotides) of the target sequence), wherein each detection cycle includes hybridizing, detecting, and removing a fluorescently labelled oligonucleotide. In embodiments, detecting includes sequencing the amplification product (e.g., using a sequencing by synthesis or sequencing by binding process).

In embodiments, forming the circular polynucleotide includes ligating a first end and a second end of the oligonucleotide probe together. In embodiments, ligating includes forming a covalent bond from the first end and the second end. As those of skill in the art appreciate, two nucleotide sequences that that are to ligated together will generally directly abut one another.

In embodiments, the method includes contacting a cell or tissue with a polynucleotide probe and hybridizing a first hybridization sequence of the polynucleotide probe to a first target sequence of the RNA or DNA molecule, and hybridizing a second hybridization sequence of the polynucleotide probe to a second target sequence of the RNA or DNA molecule, ligating the first hybridization sequence and the second hybridization sequence together thereby forming a circular oligonucleotide; extending an amplification primer hybridized to the circular oligonucleotide with a polymerase to generate an extension product comprising the target sequence; and hybridizing a sequencing primer to the extension product and sequencing the target sequence (i.e., the first and/or second hybridization sequence) in the cell or tissue, thereby detecting the RNA or DNA molecule. In embodiments, the nucleic acid molecule is cDNA. In embodiments, the nucleic acid molecule is DNA. In embodiments, the nucleic acid molecule is RNA. In embodiments, the target is an RNA nucleic acid sequence or DNA nucleic acid sequence. In embodiments, the target is an RNA nucleic acid sequence or DNA nucleic acid sequence from the same cell. In embodiments, the target is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments, the target is mRNA, tRNA (transfer RNA), rRNA (ribosomal RNA), or noncoding RNA (such as lncRNA (long noncoding RNA)). In embodiments, the targets are on different regions of the same RNA nucleic acid sequence. In embodiments, the targets are cDNA target nucleic acid sequences and before step i), the RNA nucleic acid sequences are reverse transcribed to generate the cDNA target nucleic acid sequences. In embodiments, reverse transcription of the RNA nucleic acid is performed with a reverse transcriptase, for example, Tth DNA polymerase or mutants thereof. In embodiments, the targets are not reverse transcribed to cDNA, i.e., the oligonucleotide primer is hybridized directly to the target nucleic acid.

In embodiments the target is an RNA transcript. In embodiments the target is a single stranded RNA nucleic acid sequence. In embodiments, the target is an RNA nucleic acid sequence or a DNA nucleic acid sequence (e.g., cDNA). In embodiments, the target is a cDNA target nucleic acid sequence and before step i), the RNA nucleic acid sequence is reverse transcribed to generate the cDNA target nucleic acid sequence. In embodiments, reverse transcription of the RNA nucleic acid is performed with a reverse transcriptase, for example, Tth DNA polymerase or mutants thereof. In embodiments, the target is genomic DNA (gDNA), mitochondrial DNA, chloroplast DNA, episomal DNA, viral DNA, or copy DNA (cDNA). In embodiments, the target is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target is a cancer-associated gene. In embodiments, to minimize amplification errors or bias, the target is not reverse transcribed to generate cDNA.

In embodiments, the polynucleotide probe includes DNA. In embodiments, the polynucleotide probe consists of DNA. In embodiments, the polynucleotide probe is a single-stranded polynucleotide comprising at least one amplification primer binding sequence, at least one sequencing primer binding sequence, or both one amplification primer binding sequence and one sequencing primer binding sequence. In embodiments, the first hybridization sequence, the second hybridization sequence, or both the first and second hybridization sequences of the polynucleotide probe comprise one or more locked nucleic acid (LNA) nucleotides. In embodiments, an amplification primer and a sequencing primer are complementary to different primer binding sequences. In embodiments, the primer binding sequence is complementary to a fluorescent in situ hybridization (FISH) probe. FISH probes may be custom designed using known techniques in the art, see for example Gelali, E., Girelli, G., Matsumoto, M. et al. Nat Commun 10, 1636 (2019).

In embodiments, the method includes circularizing and ligating the complementary sequence to the 5′ end of the polynucleotide probe. In embodiments, circularizing the oligonucleotide primer to generate a circular oligonucleotide includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer. In embodiments, the ligation includes enzymatic ligation. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR® ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof.

In embodiments, amplifying the circular polynucleotide includes hybridizing a primer to the circular polynucleotide and extending the primer with a strand-displacing polymerase. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the method includes contacting the target with an amplification primer and oligonucleotide primer in the same reaction (e.g., simultaneously). In embodiments, the method includes fixing the amplification products (e.g., contacting the amplification product with formalin). In embodiments, amplifying includes incubating the circular polynucleotide with a strand-displacing polymerase for about 15 minutes to about 2 hours. In embodiments, amplifying includes incubating the circular polynucleotide with a strand-displacing polymerase for about 30 minutes to about 60 minutes. In embodiments, amplifying includes binding an amplification primer to the primer binding sequence and extending the amplification primer with a strand-displacing polymerase.

In embodiments, sequencing includes hybridizing a sequencing primer to the amplification product and incorporating one or more labeled nucleotides, and detecting the incorporated one or more labeled nucleotides so as to identify the sequence.

In embodiments, the method includes sequencing the extension products, which includes the target nucleic acid sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, the method includes sequencing the amplification products (e.g., a plurality of different amplification products). In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP or acyclic nucleotide) into the first sequencing primer.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule. In embodiments, each oligonucleotide probe further includes a barcode sequence. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon TK. Error Correction Coding: Mathematical Methods and Algorithms. ed. 1st Wiley: 2005, each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) or acyclic nucleotide triphosphates to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) or acyclic nucleotide triphosphates to prevent further extension of the sequencing read product.

In embodiments, sequencing includes sequencing by synthesis, sequencing by binding, or sequencing by ligation. In embodiments, sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the amplification product. In embodiments, the sequencing primer includes the sequence: SEQ ID NO:11419, SEQ ID NO:11420, SEQ ID NO:11421, SEQ ID NO:11422, SEQ ID NO:11423, SEQ ID NO:11424, SEQ ID NO:11425, or SEQ ID NO:11426.

In embodiments, the method further includes determining the cell morphology (e.g., the cell boundary or cell shape). Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

In embodiments, the cell or tissue is stained (e.g., contacted and/or incubated with a stain agent). Non-limiting examples of stains include histological stains (e.g., hematoxylin and/or eosin) and immunological stains (e.g., fluorescent stains). A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the first fluorescent dye. In embodiments, detecting includes directing an excitation light to the cell or tissue and detecting an emission light from the second fluorescent dye. In embodiments, detecting includes directing an excitation light to the cell or tissue and detecting an emission light from the stain. In embodiments, detecting includes directing an excitation light to the cell or tissue and detecting an emission light from the first fluorescent dye, the second fluorescent dye, and the stain. In embodiments, a cell or tissue can be imaged. In embodiments, the method includes detecting an organelle.

In an aspect is provided a method of detecting a biomolecule in or on a cell or tissue. In embodiments, the method includes immobilizing a cell or tissue including a biomolecule to a solid support; contacting the biomolecule in or on the cell or tissue with an oligonucleotide as described herein, binding a detection agent (e.g., a probe) including a label to the probe oligonucleotide (or an amplification product thereof); detecting the label, thereby detecting the biomolecule. In embodiments, the method includes imaging the tissue section. In embodiments, the detection agent is a biomolecule-specific binding agent. In embodiments, the biomolecule-specific binding agent is a protein-specific binding agent. In embodiments, the biomolecule-specific binding agent is an oligonucleotide-specific binding agent. In embodiments, the biomolecule-specific binding agent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, intracellular organelle, or transcription factor. In embodiments, the intracellular organelle includes actin, carbohydrate, centrosomes and centrioles, chloroplasts (in plant cells and some protists), cytoskeleton, endoplasmic reticulum, endosome, Golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/β-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the detection agent is an oligonucleotide-specific binding agent capable of hybridizing to a target oligonucleotide sequence in a tissue section. In embodiments, the detection agent is an oligonucleotide. In embodiments, the detection agent is an oligonucleotide, wherein the oligonucleotide includes: a) a first region at a 3′ end that is hybridized to a first complementary region of the polynucleotide, and b) a second region at a 5′ end that is hybridized to a second complementary region of the polynucleotide, wherein the second complementary region is 5′ with respect to the first complementary region. In embodiments, the method includes i) circularizing the oligonucleotide agent to generate a circular oligonucleotide and ligating the oligonucleotide-specific binding agent; ii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iii) sequencing the extension product of step (ii). In embodiments, circularizing the oligonucleotide-specific binding agent includes extending the 3′ end of the oligonucleotide-specific binding agent (using a polymerase to incorporate one or more nucleotides) along the target nucleic acid to generate a complementary sequence and ligating the extended 3′ end of the oligonucleotide-specific binding agent to the 5′ end of the oligonucleotide-specific binding agent. In embodiments, the circular oligonucleotide includes a barcode sequence. In embodiments, circularizing in step i) further includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence) prior to ligating the complementary sequence to the 5′ end of the oligonucleotide primer. In embodiments, the oligonucleotide is an oligonucleotide primer.

In another aspect is provided a computer-implemented method designing an oligonucleotide probe for a target gene. In embodiments, the method includes generating, by a processor, a plurality of probe sequences. In embodiments, each probe sequence including a first segment and a second segment, each segment including computationally derived characters corresponding to 15 to 20 nucleotides; selecting from the plurality a first subset of probe sequences wherein each segment is complementary to a sequence of the target gene; and selecting, from the first subset, a second subset of probe sequences including: 45% to 65% guanine and/or cytosine nucleotides; no more than four consecutive guanine or cytosine nucleotides; and a predicted melting temperature of greater than 50° C. when forming a duplex with the sequence of the target gene.

The oligonucleotide probes can also be ranked or selected in the methods based on any mathematical combination of two or more nucleotide identities. For example, and not by way of limitation, probes can be ranked and/or selected based on the percentage or fraction of bases that are either guanine or cytosine (“G+C %”) or, alternatively, based on the percentage or fraction of bases that are either adenine or thymine (“A+T”). In another embodiment, oligonucleotide probes can be ranked or selected according to a differential between the percent or fraction of two or more nucleotide identities, such as the difference between the percent or fraction of bases in a probe that are adenine and the percent or fraction of bases that are cytosine (“A<C %”). In embodiments, oligonucleotide probes are ranked and/or selected to retain the number of G and C bases within a preferred range of 40% to 70% of the binding sequence. In particular, it is already well known in the art that guanine-cytosine base pairs have a higher stability than do adenine-thymine base pairs and, further, that many guanine containing mismatches have a higher stability than do non-guanine containing mismatches (see, e.g., SantaLucia, 1998, Proc. Natl. Acad. Sci. U.S.A. 95:1460-1465). As a result, although the percentage of guanine-cytosine base pairs is therefore somewhat correlated with the perfect match duplex binding energy discussed above, high number of guanine-cytosine base pairs are also correlated with higher levels of cross-hybridization. Preferably the percentage of G-C base pairs is between 0 and 75%, more preferably between 0 and 55%, and still more preferably between 8 and 45%.

In embodiments, the method includes removing from the first subset any probe sequence including a region complementary to a sequence in a transcriptome database. In embodiments, a homology search method such as BLAST (“Basic Local Alignment Search Tool”) and PowerBLAST (see, in particular, Altschul et al., 1990, J. Mol. Biol. 215:403-410; Altschul, 1997, Nucleic Acids Res. 25:3389-3402; and Zhang and Madden, 1997, Genome Res. 7:649-656) are performed against each probe sequence to identify polynucleotides, e.g., in a database of expressed sequences such as the GenBank or the dbEST database, which comprises sequences that are most identical or homologous to each probe's complementary sequence. For example, in embodiments, sequences which are at least 50%, 60%, 70%, 80%, 90%, 95%, or 99% identical to a probe's target sequence are identified using a search algorithm such as BLAST or PowerBLAST according to its default parameters. Preferably the search algorithm is employed using parameters set to detect perfect-match sequences of a seed length of, e.g., 7 to 15 or, more preferably, 7 to 12 bases. Binding energies and binding specificity are then evaluated only for polynucleotide sequences identified in such searches. Any probe sequences that have a match identified in a database may then be discarded.

In embodiments, the sequence of the target gene includes an exon. An exon is a contiguous nucleotide sequence within a gene that is transcribed into precursor messenger RNA (pre-mRNA) and retained following RNA splicing to form mature messenger RNA (mRNA). Exons encode amino acid sequences in protein-coding genes and represent functional units of genetic information that are typically translated into polypeptides. In the context of oligonucleotide probe design, exons are preferred target regions due to their biological relevance, transcriptional stability, and conserved expression across cells and tissues. Targeting exonic sequences enables the detection of expressed gene products and allows for transcript-specific discrimination in applications such as multiplexed gene expression profiling, spatial transcriptomics, and in situ hybridization. In embodiments, candidate probe sequences are computationally tiled across one or more exons of the target gene to ensure complete coverage. The first and second segments of each probe are selected from adjacent or nearby regions within a single exon or across exon-exon junctions, depending on the assay design. By focusing on exonic regions, the resulting probe set is optimized for hybridization to transcribed mRNA while minimizing background signal from intronic or intergenic regions.

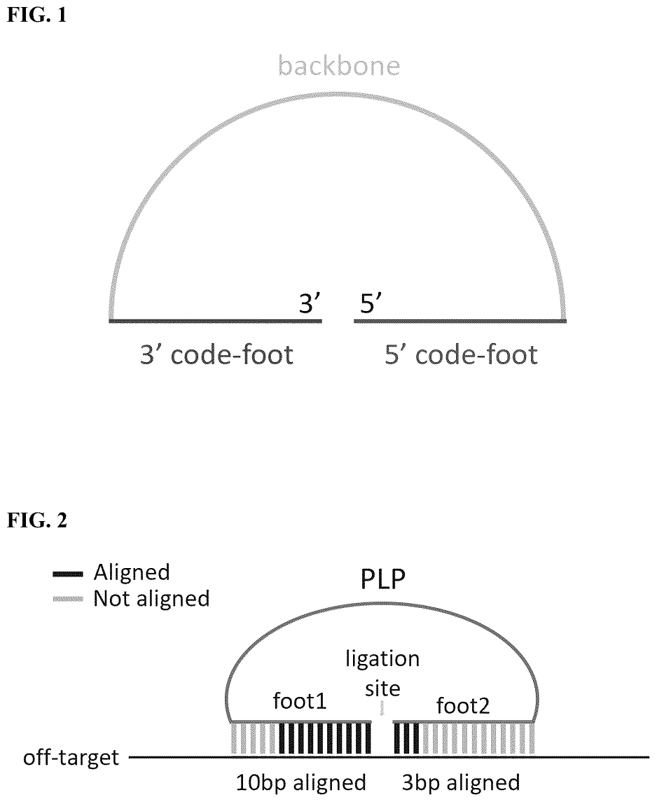

In embodiments, each probe sequence includes a sequence overlap of less than eight nucleotides between the first segment and the second segment of the same probe sequence. The first and second segments correspond to the two hybridization arms of a circularizable oligonucleotide probe (see, for example FIG. 1). Limiting the overlap between these segments ensures that each segment binds to a distinct region of the target sequence, thereby preserving the specificity of hybridization. Overlap of greater than or equal to eight nucleotides may result in undesirable intra-molecular interactions, such as the formation of secondary structures or partial self-annealing, which may interfere with the hybridization, ligation, or amplification steps of the assay. The restriction on segment overlap reduces the likelihood of internal complementarity that could compromise probe performance, and facilitates the design of structurally stable probes suitable for multiplexed applications. In embodiments, the overlap constraint is applied during the computational design process and serves as an exclusion criterion when selecting candidate probes. The constraint supports the generation of probe sequences that retain structural distinction between their hybridization arms and minimizes sequence redundancy within each probe. In embodiments, the method includes removing any probe sequence that overlaps with another probe sequence in the second subset.

In embodiments, the method includes (a) identifying, for each probe sequence in the second subset, one or more off-target alignments, wherein an off-target alignment is a genomic or transcriptomic sequence that aligns with the probe sequence such that at least three contiguous nucleotides align to each of the first and second segments of the probe sequence; and (b) removing from the second subset any probe sequence including a predicted melting temperature of less than or equal to 42° C. when hybridized to any such off-target alignment. In embodiments, the method includes removing from the second subset any probe sequence including a predicted melting temperature of less than or equal to 40° C. (e.g., when bound to an off target).

In embodiments, the method includes (a) comparing, for each of the first and second segments of the probe sequences in the second subset, a Hamming distance to the corresponding segment of all other probe sequences in the second subset; and (b) filtering out any probe sequence in which either the first or second segment has a Hamming distance of less than three compared to any other corresponding segment in the second subset. In embodiments, the Hamming distance is 3. In embodiments, the Hamming distance is 2. In embodiments, the Hamming distance is 4. In embodiments, the Hamming distance is 5. In embodiments, the Hamming distance is 1.

### IV. Systems and Devices

In another aspect is provided a non-transitory computer-readable medium storing instructions that, when executed by a processor, perform a method as described herein. For example, the method includes (a) identifying, for each probe sequence in the second subset, one or more off-target alignments, wherein an off-target alignment is a genomic or transcriptomic sequence that aligns with the probe sequence such that at least three contiguous nucleotides align to each of the first and second segments of the probe sequence; and (b) removing from the second subset any probe sequence including a predicted melting temperature of less than or equal to 42° C. when hybridized to any such off-target alignment. In embodiments, the method includes removing from the second subset any probe sequence including a predicted melting temperature of less than or equal to 40° C. In embodiments, the non-transitory computer-readable medium is a computing device. In embodiments, the computing device is a personal computer system, server computer system, hand-held or laptop device, multiprocessor system, microprocessor-based system, set top box, programmable consumer electronic, network PC, minicomputer system, mainframe computer system, smartphone, or distributed cloud computing environments that include any of the above systems or devices. The computing device can include one or more processors or processing units, a memory architecture that may include RAM and non-volatile memory. The memory architecture may further include removable/non-removable, volatile/non-volatile computer system storage media. Further, the memory architecture may include one or more readers for reading from and writing to a non-removable, non-volatile magnetic media, such as a hard drive, a magnetic disk drive for reading from and writing to a removable, non-volatile magnetic disk, and/or an optical disk drive for reading from or writing to a removable, non-volatile optical disk such as a CD-ROM or DVD-ROM.

In an aspect is provided a system for designing oligonucleotide probes for a target gene. In embodiments, the system includes a memory storing computer-executable instructions; and a processor configured to execute the instructions to: (a) generate a plurality of candidate probe sequences, each comprising a first segment and a second segment of between 15 and 20 nucleotides; (b) identify a first subset of the candidate probe sequences that are complementary to an exon sequence of the target gene; (c) select a second subset from the first subset based on GC content, avoidance of shared sequence regions, and melting temperature thresholds for on-target and off-target hybridization; (d) compare the Hamming distance between the first and second segments of each probe sequence and all others in the second subset; and (e) exclude from the second subset any probe sequence in which either segment has a Hamming distance of less than three compared to a corresponding segment of another probe sequence in the second subset.

In embodiments, the system includes one or more processing units CPU(s) (also referred to as processors), one or more network interfaces, a user interface including a display and an input module, a non-persistent, a persistent memory, and one or more communication buses for interconnecting these components. The one or more communication buses optionally include circuitry (sometimes called a chipset) that interconnects and controls communications between system components. The non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory optionally includes one or more storage devices remotely located from the CPU(s). The persistent memory, and the non-volatile memory device(s) within the non-persistent memory, comprise non-transitory computer readable storage medium. In embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In embodiments, the computing device includes memory in electronic communication with the processor. The memory architecture may include at least one program module implemented as executable instructions that are configured to carry out one or more steps of a method set forth herein. For example, executable instructions may include an operating system, one or more application programs, other program modules, and program data. Generally, program modules may include routines, programs, objects, components, logic, and data structures that perform particular tasks. A computing device can optionally communicate with one or more external devices such as a keyboard, a pointing device (e.g., a mouse), a display, such as a graphical user interface (GUI), or other device that facilitates interaction of a use with the unmanned autonomous vehicle. Similarly, the computing device can communicate with other devices (e.g., via network card, modem, etc.). Such communication can occur via I/O interfaces. In embodiments, the computing system may communicate with one or more networks such as a local area network (LAN), a general wide area network (WAN), and/or a public network (e.g., the Internet) via a suitable network adapter.

In embodiments, the computing device includes software with instructions configured to perform a method as described herein. The software component of the computer system also preferably accepts one or more parameters or ranges of parameters for use in selecting nucleotide probes. Exemplary parameters that can be accepted by the software component include: probe length, maximum distance from the 3′-end or 5′-end of the target sequence, the maximum and/or minimum allowable binding energy scores (including perfect-match and/or cross-hybridization binding energies), upper and/or lower limits of acceptable base composition (e.g., G %, C %, A %, T %, G+C %, A-C %), the longest permissible single base run, and the maximum number of base overlap allowed among different probes. The software component can also accept parameters, such as temperature, salt concentration and target polynucleotide concentration, for use in calculating hybridization binding energies. These values can be input, e.g., directly by a user or, alternatively, can be read by the software component from a file.

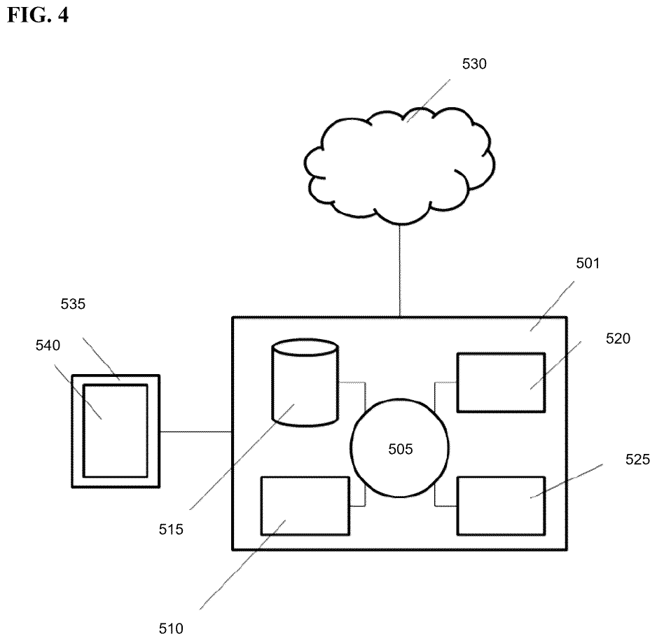

FIG. 4 shows an example computer system that can implement methods provided herein. For example, the present disclosure provides computer systems that are programmed to implement methods of the disclosure. FIG. 4 shows a computer system 501 that is programmed or otherwise configured to analyze a tissue sample including a plurality of cells. The computer system 501 can regulate various aspects of components of the system of the present disclosure. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet. The computer system 501 can communicate with one or more remote computer systems through the network 530. For instance, the computer system 501 can communicate with a remote computer system of a user. Examples of remote computer systems include personal computers (e.g., portable PC), slates, or tablets (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processor adapted to execute the code, or can be compiled during runtime. The code can be supplied in a programming language that can be selected to enable the code to execute in a pre compiled or as-compiled fashion.

Examples of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media (e.g., computer-readable media) include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for tissue sample analysis. Examples of UIs include, without limitation, a graphical user interface (GUI) and web-based user interface. Methods and systems of the present disclosure can be implemented by way of one or more algorithms. An algorithm can be implemented by way of software upon execution by the central processing unit 505.

As another example, the computer storage media may be implemented using magnetic or optical technology. In such implementations, the program modules may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

According to certain embodiments, the above-described data feeds may be stored in databases such as database servers that store master data as well as logging and trace information. The databases may also provide an API and/or API access (e.g., for open source) to the web server for data interchange based on JSON specifications. According to certain embodiments, the database servers may be optimally designed for storing large amounts of data, responding quickly to incoming requests, having a high availability and historizing master data.

Certain embodiments of the present disclosure are described above with reference to block and flow diagrams of systems and methods and/or computer program products according to example embodiments of the present disclosure. It will be understood that one or more blocks of the block diagrams and flow diagrams, and combinations of blocks in the block diagrams and flow diagrams, respectively, may be implemented by computer-executable program instructions. Likewise, some blocks of the block diagrams and flow diagrams may not necessarily need to be performed in the order presented, or may not necessarily need to be performed at all, according to some embodiments of the present disclosure.

These computer-executable program instructions may be loaded onto a general-purpose computer, a special-purpose computer, a processor (e.g., a processor chip, single/multiprocessor architectures, sequential (Von Neumann)/parallel architectures, and specialized circuits, etc.), or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that may direct a computer or other programmable data processing apparatus to function in a particular manner, such that the instructions stored in the computer-readable memory produce an article of manufacture including instruction means that implement one or more functions specified in the flow diagram block or blocks.

As an example, embodiments of the present disclosure may provide for a computer program product, including a computer-usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Various aspects described herein may be implemented using standard programming and/or engineering techniques to produce software, firmware, hardware, and/or any combination thereof to control a computing device to implement the disclosed subject matter. A computer-readable medium may include, for example: a magnetic storage device such as a hard disk, a floppy disk or a magnetic strip; an optical storage device such as a compact disk (CD) or digital versatile disk (DVD); a smart card; and a flash memory device such as a card, stick or key drive, or embedded component. Additionally, it should be appreciated that a carrier wave may be employed to carry computer-readable electronic data including those used in transmitting and receiving electronic data such as streaming video or in accessing a computer network such as the Internet or a local area network (LAN). Of course, a person of ordinary skill in the art will recognize many modifications may be made to this configuration without departing from the scope or spirit of the claimed subject matter.

### Examples

### Example 1. Code-Probe Considerations

The human cell contains about 20,000 to 30,000 genes coding for functional proteins, yet only a small fraction is expressed in any cell at any time. The specific phenotype of a cell is thus derived from the expression of the subset of genes, and studies of cell phenotypes can be performed by gene expression analysis. Messenger RNA (mRNA) is the molecular carrier transporting genetic information from nuclear DNA to the cytoplasmic machinery of protein synthesis. Consequently, mRNA provides an ideal point of investigation in understanding gene functions and correlating their expression to the physiological and pathological phenotype of cells and tissues. For example, analyzing gene expression levels and the biological pathways associated with the genes involved in a cancer, enables one can study the difference between normal cell and cancerous cell pathways to determine the genetic origin of the faulty pathway, thereby identifying potential targets for treating cancer. Furthermore, gene expression analysis facilitates the discovery of biomarkers and gene signatures, which are crucial for diagnosing diseases, monitoring their progression, and predicting responses to therapeutic treatments. In cancer, gene expression profiling aids in developing gene biomarkers and signatures that enhance diagnosis, track disease progression, and predict treatment outcomes, ultimately contributing to personalized medicine approaches.

Traditional methods for analyzing gene expression are essential tools in molecular biology, involving the extraction and quantification of mRNA from cells or tissues. One foundational technique is Northern blotting, where specific RNA molecules are detected within a sample by separating RNA by gel electrophoresis, transferring it to a membrane, and hybridizing it with a labeled complementary probe, with the signal intensity reflecting the presence and quantity of the target RNA. A brute-force approach of capturing gene expression information is simply by isolating regions of interest from within a sample. For example, these regions can then be individually placed in test tubes for RNA extraction and subsequent gene expression profiling. For comprehensive data on the entire transcriptome, RNA sequencing (RNA-Seq) is often employed. In RNA-Seq, RNA is converted to cDNA and used to make a library of nucleic acid molecules, which are then sequenced on next generation sequencing (NGS) platforms like the G4™ or NextSeq 2000™.

Instead of extracting RNA molecules from individual parts (or cells) within a tissue, one can visualize them directly in their original environment. Detecting RNA in situ with spatial surroundings provides critical insights into the spatial organization of gene expression, enhancing our understanding of tissue architecture, cellular interactions, developmental processes, disease mechanisms, and functional genomics. Detecting nucleic acid molecules in cells or tissues enables the observation of spatial heterogeneity within tissues, revealing how gene expression varies across different regions and cell types, and illuminates how local interactions regulate gene expression patterns. Technologically, in situ RNA detection preserves spatial information that is often lost in traditional bulk RNA sequencing, providing a more accurate representation of gene expression patterns. With single-cell resolution, it enables detailed analysis of cellular diversity and heterogeneity, and when combined with other imaging techniques, it offers a multi-dimensional view of cellular function.

An early variant of in situ hybridization (ISH) detection was published in the late 1960s (J. G. Gall, M. L. Pardue, Proc. Natl. Acad. Sci. USA 1969, 63, 378) and has since evolved for visualizing gene expression. One widely used in situ technique is Fluorescence In Situ Hybridization (FISH). FISH utilizes fluorescently labeled probes that hybridize to specific RNA sequences within the tissue. This method allows for the visualization and localization of target RNA molecules with high spatial resolution. FISH is particularly useful for detecting specific gene transcripts and chromosomal abnormalities. However, the number of distinct fluorophores that can be used simultaneously is limited due to spectral overlap. The number of RNA species that can be simultaneously imaged by FISH has been limited. Most experiments stain and image only one RNA species at a time, and even the most advanced multiplexing efforts have only extended this approach to the simultaneous measurement of approximately 10-30 RNA species (Lubeck E, Coskun A F, Zhiyentayev T, Ahmad M, Cai L. Single-cell in situ RNA profiling by sequential hybridization. Nat. Methods. 2014; 11:360-361 and Martin K C, Ephrussi A. mRNA localization: gene expression in the spatial dimension. Cell. 2009; 136:719-730). However, many interesting questions require much higher levels of multiplexing, such as transcriptional definitions of cell phenotyping. Additionally, the resolution of FISH detection, while sufficient for many applications, may not be adequate for detecting very small or low-abundance transcripts, potentially leading to incomplete or biased gene expression data.

Multiplexed, Error-Robust, Fluorescence In Situ Hybridization (MERFISH) (Chen et al. Science. 2015; 348:aaa6090) enables multiplexing by assigning barcodes to different RNA species and then reading out these barcodes through successive rounds of hybridization and imaging on the same sample. NanoString Technologies (now Bruker Spatial Biology) provides a platform for in situ gene expression, the CosMX Spatial Molecular Imager, which utilizes barcoded probes to target thousands of RNA sequences simultaneously, greatly improving multiplexing. 10× Genomics also offers a platform to in situ analysis relying on barcoded probes. In these approaches, each RNA species is often identified not by a single color of fluorescently labeled FISH probe but rather with a unique combination or “barcode” of colors. For example, one RNA molecule might be identified by red-labeled probes alone while another might be identified by the combination of red-, green-, and yellow-labeled probes. The barcoded probes extend the concept used by FISH probes, by replacing the single fluorescent label with a barcode domain. The barcode domain contains consecutive regions that hybridize to unique reporters that are read out over sequential imaging rounds. In practice, a substantial limitation to multiplexing arises from the readout errors that are inherent to FISH. For example, occasionally an RNA molecule that should fluoresce in one imaging round does not accumulate enough fluorescently labeled probe to produce a bright enough signal to be detected. Additionally, stray probes or a bright autofluorescent spots in the cell can sometimes produce a spot bright enough to be called an RNA when it is not actually present.

Barcoded probes typically include both a target sequence binding domain (e.g., 60-80 nucleotides) and a unique barcode domain (e.g., 40-50 nucleotides). The necessity of unique barcodes for each target limits the number of RNA species that can be analyzed simultaneously, constraining multiplexing capacity. Managing these barcodes introduces additional complexity and potential sources of error, as there is a risk of barcode cross-reactivity and misassignment, which can lead to erroneous data. Furthermore, the added barcode sequence may interfere with the binding efficiency or stability of the probe, potentially compromising sensitivity and specificity. For example, if a barcoded probe binds to an off-target sequence due to a mismatch in the target domain or off-target hybridization of the barcode domain, the detection of the barcode could lead to a false positive, mistakenly identifying the target RNA as present. Barcodes can also increase the molecular weight and steric hindrance of probes, potentially affecting their performance in dense or complex tissue environments.

In the context of in situ RNA detection, relying on barcodes for target identification presents several challenges. One significant issue is the limited spatial resolution that can result from insufficient barcode sequence length. If the barcode is too short, it may not generate enough unique sequences to accurately identify and distinguish between different RNA molecules within the complex tissue environment, leading to overestimation of molecular counts due to amplification artifacts. On the other hand, an overly long barcode sequence or an inadequate number of barcode sequences can result in under-sampling, where not all unique RNA molecules are captured, causing underestimation of molecular counts. Additionally, errors during barcode sequence synthesis or sequencing can introduce noise and cause misassignment of barcodes, complicating the spatial mapping of RNA molecules and leading to inaccuracies in gene expression data. This misidentification can be particularly problematic in in situ hybridization, where precise spatial information is crucial for understanding tissue architecture and cellular interactions. Moreover, the additional steps required for decoding and mapping barcode sequences increase the complexity and computational burden of the analysis, making the process more time-consuming and prone to errors.

In contrast, the “barcode-free” probes described herein includes the target sequence binding domain and utilizes sequencing that domain for target identification, offer significant advantages. These probes simplify design and synthesis, reduce production costs, and enhance scalability. Without barcodes, there is less risk of cross-reactivity, and the binding efficiency and stability of the probes are potentially improved, leading to higher sensitivity and specificity. Additionally, without the need for barcode decoding, data processing is streamlined, reducing computational demands. The streamlined data processing reduces computational demands, making “barcode-free” probes a superior choice for comprehensive and efficient in situ gene expression analysis. The uniqueness of these probes enables the detection of a vast array of genes, (e.g., greater than 250 different target sequences) within the same sample without unintended non-specific interactions with other cellular components, particularly genomic DNA which exhibits a natural non-specific affinity for single stranded DNA. Careful design of the sequences is thus required to ensure target specificity and orthogonality to other probes when detecting multiple targets.

The approach described herein significantly enhances specificity and robustness by requiring two hybridization events for detection of a target sequence. As illustrated in FIG. 1, each oligonucleotide probe includes two target hybridization sequences (code-foot 1 and code-foot 2). These sequences bind to adjacent regions of the target RNA. Following successful binding of both code-feet, a ligation step forms a circular polynucleotide, which may be amplified using rolling circle amplification (RCA), and subsequently detected. This dual-binding requirement ensures that if only one code-foot binds to an off-target sequence, the second code-foot will likely fail to bind, preventing the formation of an amplifiable circular probe and thus avoiding false positives. This mechanism significantly enhances the accuracy and specificity of target detection compared to single hybridization events employed in common barcoded probe techniques.

### Example 2. Code-Probe Design

In our current implementation of Code-Probe detection, each oligonucleotide probe includes three basic components as illustrated in FIG. 1. A code probe is a single-stranded oligonucleotide that includes a first code-foot (e.g., 3′ code-foot) and a second code-foot (e.g., 5′ code-foot) on either end and is linked together via a linking oligonucleotide sequence (referred to as a backbone). The linking oligonucleotide sequence may include one or more primer binding sequences (e.g., an amplification and/or sequencing primer binding sequence) and/or a error-correcting barcode which may be sequenced in combination with the code-feet.

In designing our panel of probes for gene expression analysis, we imposed several stringent constraints to ensure specificity and efficiency. Each probe consists of two hybridization sequences, referred to herein as code-feet, that are typically less than 20 bp each. Additionally, each code-foot was constrained to a GC content of 45-65% and prohibited from containing four consecutive guanines (Gs) or cytosines (Cs) to minimize the risk of secondary structures and non-specific binding. The binding of the probe to the target sequence should be tight and highly specific in order to avoid any interference, for example having less than or equal to 8 bp overlap with any backbone. The thermodynamics of base pairing, in fact, determine the strength of binding while the nucleotide combination ensures its specificity as a consequence of the uniqueness of base succession. However, unique sequences endowed with high binding energy are not necessarily good probes if the target sequence is inaccessible due to secondary structures.

Both the sensitivity and specificity of a particular probe depends upon the binding energies, AG, of polynucleotide molecules to the probe. In particular, the hybridization intensity traces a sigmoidal curve which follows the melting curve of the probe, decreasing as the binding energy increases. Specificity, however, is maximum at or slightly above the melting temperature of the probe (i.e., at a binding energy that is equal to or slightly greater than zero). Thus, in embodiments, the sensitivity and/or specificity are determined or predicted from the binding energies, provided additional criteria described herein are met. It is noted that the skilled artisan readily appreciates that the term “binding energy,” as used herein, refers to the difference of the energy of polynucleotide molecules (e.g., a target polynucleotide and a polynucleotide probe) when they are in a bound state (i.e., when they are bound or hybridized to each other) from when they are in an unbound state. This definition is readily expressed mathematically by the formula ΔG=G_{bound}−G_{unbound}. Thus, a polynucleotide that has the “largest” binding energy to a particular probe is one for which the difference between the energies of the bound and unbound polynucleotide is greatest. In particular and as the skilled artisan also readily appreciates, because the energy of polynucleotides in a bound state is ordinarily lower than the energy of the unbound polynucleotides, the binding energy (i.e., AG) will ordinarily be a negative number. Thus, as used herein, the polynucleotide having the “largest” binding energy to a particular probe will, in fact, be the polynucleotide for which AG is the most negative.

Reliable oligonucleotide design is crucial for successful binding and detection, and given the diversity of targets and multiplexing, the design of oligonucleotides requires flexibility in the approach. Thus, a number of oligonucleotide design tools exist, for example PrimerSelect (Plasterer T N., Methods Mol. Biol., 1997, vol. 70 (pg. 291-302)), Primer Express (Applied BiosystemsPrimer Express® Software Version 3.0 Getting Started Guide, 2004), OLIGO 7 (Rychlik W., Methods Mol. Biol., 2007, vol. 402 (pg. 35-60)) and Primer3 (Untergasser, A. et al., Nucleic Acids Res 40, e115 (2012)). Additional online tools, for example the OligoAnalyzer™ Tool provided by Integrated DNA Technologies (accessible at www.sg.idtdna.com/pages/tools/oligoanalyzer) or PrimerROC (Johnston, A. D., Lu, J., Ru, Kl. et al. Sci Rep 9, 209 (2019)) sheds additional insight into the secondary structure of oligonucleotides and the resulting amplification products, as well as the self- and heterodimerization tendencies of each primer set. Primer-BLAST is another web service that supports the selection of effective sequences by considering opportunities for mispriming across an entire genome or transcriptome (Sayers E W et al., Nucleic Acids Res., 2012, vol. 40 (pg. D13-D25)). Additionally, RNAFold, provided with ViennaRNA Package 2.0 (Lorenz R, et al. Algorithms Mol Biol. 2011 Nov. 24; 6:26.) utilizes thermodynamic principles to determine the most stable structure based on the sequence provided, offering insights into the functional properties and cross-interactions. These tools are useful starting points for evaluating oligonucleotide sequences.

A goal of effective oligonucleotide design is to maximize detection and minimize off-target hybridization, without introducing any biases (e.g., skewing the amplification products to over- or under-represent targets). Primer3 allows for the selection of the binding sequence on the basis of melting temperature (Tm), primer length, and 3′-end stability, which was considered when designing each primer set. Calculating the melting temperature and performing thermodynamic modelling for estimating the propensity of oligonucleotides to hybridize with other oligonucleotides or to hybridize at unintended sites in the genome offer an accurate approach for predicting the energetic stability of DNA structures. For example, because of electronic effects of nucleobase stacking, the stability of 5′-CT-3′ hybridized to 3′-GA-5′ is different from that of 5′-CA-3′ hybridized to 3′-GT-5′, despite the base pairings C:G and T:A are the same. It is recommended to perform oligonucleotides analysis with sophisticated modelling capabilities to capture such electronic effects. Additionally, in silico validation of oligonucleotides may be useful. The online software OligoAnalyzer™ Tool provides information on secondary structure and the possibility of self- or heterodimer formation by the oligonucleotides sequence itself by calculating the Gibbs free energy (AG).

The design further stipulated that the feet of each probe (i.e., the hybridization sequences) must have a maximum overlap of 8 bp with each other and with the probe backbone, ensuring structural stability and minimizing non-specific interactions. Probes were specifically targeted to exonic regions, excluding any intronic sequences to focus on mature mRNA transcripts and avoid potential complications from unspliced pre-mRNA. This approach differs from alternative probes described in the literature and commercially available, which includes intronic sequences in their target binding regions.

Spacing between probe binding sequences is another complicated design consideration. Regular spacing (tiling) is the most common approach because it is easy to implement, but it does not ensure optimal positioning of probes. The geometry of folded conformations of a given RNA around the target sequence can impair the binding of a probe. The folding of RNA is a unimolecular process occurring spontaneously in situ, dependent upon the pairing of self-complementary stretches of different regions of an RNA molecule which produces a number of different secondary structures. Consequently, predictions and experimental validation of secondary structures of RNA are useful for selecting target elements.

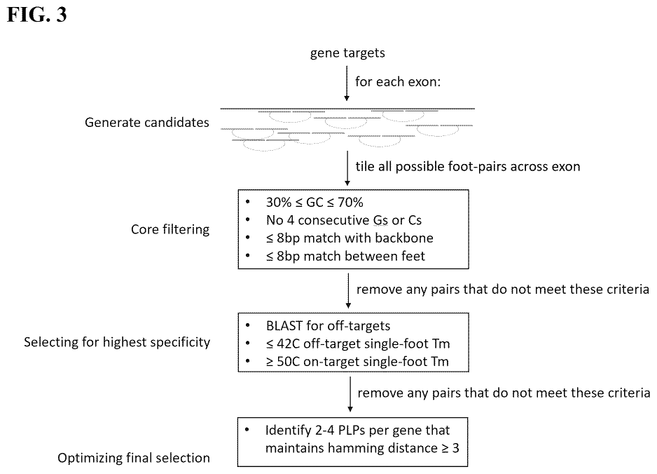

To ensure high specificity, we required that the probes exhibit zero off-target hits, a phenomenon illustrated in FIG. 2. Off-target hits were defined using a refined approach: instead of merely counting base pairs matched in each foot, we used the predicted melting temperature (Tm) of the off-target alignment for each foot and enforced a minimum difference between the maximum off-target Tm and the maximum on-target Tm across the two feet for each probe. The true on-target Tm may be higher, but we set a maximum off-target Tm of 40° C. and an on-target Tm of 50° C. to ensure specificity. Using BLAST against the RefSeq RNA database with an E-value cutoff of 100, we refined our off-target definition to include a 3 bp cross-junction requirement, which potentially leads to false-negatives but ensures rigorous specificity. Additional cross checks against GENCODE, RefSeq, or CHESS databases can also be performed. With these parameters, we can generate at least one probe for all genes in our immuno-oncology panel, optimizing both the specificity and efficiency of our probe design. In embodiments, we generate at least 3 probes per gene.

Each respective code-foot serves two purposes, binding to a specific target sequence, and following circularization, each code-foot may be detected to provide an identifier for the target sequence. The oligonucleotide probes used herein must satisfy two purposes: (1) specifically bind to a target nucleic acid molecule including a target sequence and (2) maintain dissimilarity between all other binding sequences to uniquely identify the target nucleic acid molecule. Regarding the first goal, designing target binding regions with a high melting temperature (Tm) alone is not sufficient. The geometry of folded RNA conformations around the target sequence can hinder probe binding. RNA folding is a spontaneous, unimolecular process, involving the pairing of self-complementary regions of the RNA molecule, which produces various secondary structures. Therefore, accurate predictions of RNA secondary structures are essential for selecting target sequences that are suitable for probe design. Further, to enable identification of unique targets the resulting sequences should be designed to include orthogonality (e.g., ensuring a particular Hamming distance) to increase the accuracy of target identification. The Hamming Distance between two sequences (i.e., the number of nucleotides that must be switched to convert one barcode sequence into another) is a useful concept in understanding the behaviors of these encoding schemes. Increasing the minimum Hamming Distance between used sequences by leaving more possible sequences unassigned will produce even larger reductions in a misidentification rate.

### Example 3. Code-Probes for Immuno-Oncology

It is generally accepted that gene mutations initiate cancer development. Proto-oncogenes, such as Ras, are transcribed into products like receptors, growth factors, transcription factors, and signaling enzymes crucial for cellular proliferation. Gain-of-function mutations in proto-oncogenes result in dominant oncogenes that differ from their proto-oncogenes or are over-expressed. These mutations can occur through point mutations, localized reduplication, or chromosomal translocation, leading to disrupted cellular activity and uncontrolled cell division, ultimately resulting in cancer. Measuring biological activity simultaneously in both the tumor and the immune system is key to predicting who will respond to immunotherapy, rationally design novel immunotherapy treatments, and understand the biological underpinnings of cancer.

The immune response to tumors is a complex, multifactorial interaction that is shaped by the host, the tumor, and the environment. By analyzing immune-oncology (IO) gene expression levels and the biological pathways associated with these genes, researchers can differentiate between normal and cancerous cell pathways, determining the genetic origin of the faulty pathways. This differentiation aids in identifying potential targets for cancer treatment. Moreover, gene expression analysis facilitates class discovery, identifying novel cancer subtypes, and class prediction, assigning tumor samples to pre-defined classes to predict outcomes. This approach allows for the discovery of biomarkers and gene signatures, enabling the diagnosis, progression, and aggressiveness analyses, prognosis, and prediction of therapeutic treatment. It also helps identify patients who would benefit from specific therapies, enhancing our understanding of the disease and its biology.

In this disclosure, we developed a probe panel for immuno-oncology, designed with stringent constraints to ensure high specificity and efficiency. Each probe consists of two code-feet having a GC content of 45-65% and do not contain four consecutive guanines (Gs) or cytosines (Cs), minimizing secondary structures and non-specific binding. The probes target exonic regions, excluding intronic sequences to focus on mature mRNA transcripts. We ensured zero off-target hits by defining off-targets based on predicted melting temperatures (Tm) and requiring a minimum difference between the maximum off-target Tm and the maximum on-target Tm across the two feet of each probe. See FIG. 3 for an example of the workflow. Using these stringent criteria, we achieved a maximum off-target Tm of 40° C. and an on-target Tm of 50° C., allowing us to generate at least one probe for all genes in our immuno-oncology panel. This approach ensures reliable and accurate detection of target gene sequences, crucial for advancing cancer research and therapy.

Suitable gene targets were determined to be universal across different tissue types (e.g., kidney, tonsil, breast, colon, lung). For some genes we purposely designed fewer than 24 probes, this was based on empirical data showing that these genes are extremely abundant in some cell types, leading to optical density issues. For other genes, the natural sequence diversity did not allow us to design 24 probes within our design constraints (e.g., GC content, Hamming Distance). The probes provided herein, for example in the sequence listing and incorporated herein, discloses the 5′ code-foot and the 3′ code-foot useful for targeting particular gene targets: ABCG2, ACE2, ACKR1, ACKR4, ACTA2, ACTG2, ADIPOQ, AGR3, AKT1, ALDOB, ANGPT2, ANKRD29, ANKRD30A, AQP1, AQP2, AQP3, AQP5, AR, ARG1, ATP6V0D2, BATF, BCL2, BEST2, BEST4, BTLA, C1QA, C1QB, C1QC, CALB1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CEACAM8, CKB, CLDN10, CLEC9A, CLU, COL1A1, CP, CSF1R, CSPG4, CTLA4, CTSL, CTSS, CUBN, CX3CL1, CX3CR1, CXCL10, CXCL11, CXCL12, CXCL13, CXCL9, CXCR3, CXCR4, CXCR5, CXCR6, CYP1A1, DCLK1, DEFA5, DES, DGKG, DMBT1, DMKN, DPT, DST, ECSCR, EGFR, EOMES, ERBB2, ERCC-00019, ERCC-00024, ERCC-00059, ERCC-00142, ERCC-00143, ESM1, ESR1, FABP1, FAP, FAS, FASN, FBLN1, FBN1, FCAR, FCGR1A, FCGR3A, FGB, FGFR4, FLNB, FN1, FOXA1, FOXI1, FOXJ1, FOXP3, FSCN1, GATA3, GATM, GIP, GNLY, GPC1, GPR183, GRHL1, GZMA, GZMB, GZMH, GZMK, HAVCR2, HLA-A, HLA-DRA, HOXD8, HSP90AA1, ICA1, ICOSLG, ID2, IDO1, IFNG, IGHA1, IGHD, IGHG1, IGHM, IL10, IL10RA, IL17A, IL1B, IL2RA, IL2RB, IL6, IL7R, INS, IRF1, ITGAM, ITGAX, ITGB2, JCHAIN, KDR, KIT, KLRB1, KLRD1, KLRF1, KLRK1, KRAS, KRT14, KRT23, KRT5, KRT6B, KRT7, KRT8, LAG3, LAMC3, LARS1, LGR5, LPL, LRP2, LTBP2, LUM, LYVE1, MADCAM1, MAPK1, MEDAG, MET, MLN, MLPH, MMP1, MMRN1, MMRN2, MRC1, MS4A1, MUC1, MUC2, MUC5AC, MUC5B, MYH11, MYLK, NCAM1, NCR1, NEUROD1, NEUROG3, NKG7, NOS1, NPHS2, NRXN1, NTS, OLFM4, PDCD1, PDCD1LG2, PDE4A, PDGFRA, PDGFRB, PDK4, PDPN, PECAM1, PGR, PHGR1, PIM1, PLIN1, PODXL, POLR2A, POSTN, POU2F3, PPARG, PRDM1, PRF1, PTGDS, PTGS2, PYY, RARRES1, RBP2, RGS5, RSPO3, S100A9, SCGB1A1, SCGB3A2, SDC1, SELE, SELL, SERPINA3, SFTPD, SH2D6, SLC12A1, SLC12A3, SLC26A4, SLC2A1, SLC4A1, SLC4A9, SLC8A1, SNCA, SNCG, SOX10, SPP1, SST, STAT1, STAT3, STAT4, STC1, TAGLN, TAP1, TAP2, TBX21, TCL1A, TFPI, TGFB1, THY1, TIGIT, TLR2, TLR4, TLR9, TMPRSS2, TNF, TNFRSF17, TNFRSF4, TNFRSF9, TNFSF13B, TNFSF9, TOMM7, TP63, TPD52, UMOD, UPK3B, VCAM1, VCAN, VEGFA, VIM, VWF, and WARS1. The table is formatted such that the 5′ code-foot is paired with the respective 3′ code-foot immediately across in the same horizontal row to form the probe binding sequence. For example, a probe binding sequence may be the concatenation of the two sequences SEQ ID NO:5710 and SEQ ID NO:1, to form 5′-TTGCATTGAGTCCTG GGCAGAAGTTTTGTC-3′ (SEQ ID NO: 11427).

The code probes additionally include a backbone which includes a sequencing primer binding sequence. In this manner, sets of probes may be combined to facilitate multiple rounds of detection. Herein we provide eight orthogonal sequencing primer sequences which may be included in the backbone of the code probe. For example, a first subset of probes may detect ABCG2, ACE2, WARS1, and VCAN wherein each probe includes the SP1 sequence. Following detection of the first subset, a second subset of probes may be used to detect ABCG2, ACE2, C1QC, and CCR5 wherein each probe in the second subset includes the SP5 sequence in the backbone. To enable greater detection, the code-foot sequences for the probe targeting ABCG2 (e.g., SEQ ID NO:1 and SEQ ID NO:5710) in the first subset may be different in the second subset (e.g., SEQ ID NO:17 and SEQ ID NO:5726). In embodiments, the backbone may include a supplemental error corrected barcode (ECB) to further facilitate multiplexing.

SEQUENCING PRIMER TABLE

| Sequencing | Sequencing primer | |
|---|---|---|
| Primer | sequence (5′ to 3′) | |
| SP1 | ACACTCTTTCCCTACAC | SEQ |
| | GACGCTCTTCCGATC | ID |
| | | NO: |
| | | 11419 |
| SP2 | TCGGCGTTGTCTGCTAT | SEQ |
| | CGTTCTTGGCACTCC | ID |
| | | NO: |
| | | 11420 |
| SP3 | CACAACGGGAGCTGTGG | SEQ |
| | AATTGGTTCACCTGG | ID |
| | | NO: |
| | | 11421 |
| SP4 | TGTTGCATCTCCACCCG | SEQ |
| | GATTGAGCCTTCAGC | ID |
| | | NO: |
| | | 11422 |
| SP5 | TGGACTAAGACTCGTCC | SEQ |
| | TCCAGCGGACCTAAG | ID |
| | | NO: |
| | | 11423 |
| SP6 | TACGACACACTCGGGCT | SEQ |
| | CTATGGGCTTCATGG | ID |
| | | NO: |
| | | 11424 |
| SP7 | GTATGATGGTGTTGCGG | SEQ |
| | CTTCTCGCTTAACGC | ID |
| | | NO: |
| | | 11425 |
| SP8 | TCTTGAGTCATTCGCAG | SEQ |
| | GGCATGTGCCAGACC | ID |
| | | NO: |
| | | 11426 |

Controls. The National Institute of Standards and Technology (NIST) developed the External RNA Controls Consortium (ERCC) to derive universal references. The ERCC assembled a sequence library of 176 DNA sequences that could be transcribed into RNA to serve as controls in systems used to measure gene expression. These controls were cataloged as ERCC-00001 through ERCC-00176, and are collectively referred to as ERCC controls. We designed probes to be specific to some ERCC probes, enabling the detection of false-positive ERCC probes.

What is claimed is:

1. A computer-implemented method designing a gene probe for a target gene, comprising:
generating, by a processor, a plurality of probe sequences, each probe sequence comprising a first segment and a second segment, each segment comprising computationally derived characters corresponding to 15 to 20 nucleotides;
selecting from the plurality a first subset of probe sequences wherein each segment is complementary to a sequence of the target gene; and
selecting, from the first subset, a second subset of probe sequences comprising:
45% to 65% guanine and/or cytosine nucleotides;
no more than four consecutive guanine or cytosine nucleotides; and
a predicted melting temperature of greater than 50° C. when forming a duplex with the sequence of the target gene.

2. The computer-implemented method of claim 1, further comprising removing from the first subset any probe sequence comprising a region complementary to a sequence in a genome or transcriptome database.

3. The computer-implemented method of claim 1, wherein the sequence of the target gene comprises an exon.

4. The computer-implemented method of claim 1, wherein each probe sequence comprises a sequence overlap of less than eight nucleotides between the first segment and the second segment of the same probe sequence.

5. The method of claim 1, further comprising removing any probe sequence that overlaps with another probe sequence in the second subset.

6. The method of claim 1, further comprising:
(a) for each probe sequence in the second subset, identifying, using the computer, one or more off-target alignments, wherein an off-target alignment is a genomic or transcriptomic sequence that aligns with the probe sequence such that at least three contiguous nucleotides align to each of the first and second segments of the probe sequence; and (b) computationally removing from the second subset any probe sequence comprising a predicted melting temperature of less than or equal to 42° C. when hybridized to any such off-target alignment.

7. The method of claim 1, further comprising: (a) comparing, using the computer, for each of the first and second segments of the probe sequences in the second subset, a Hamming distance to the corresponding segment of all other probe sequences in the second subset; and (b) computationally filtering out any probe sequence in which either the first or second segment has a Hamming distance of less than three compared to any other corresponding segment in the second subset.

8. A kit comprising:
a first plurality of nucleic acid molecules, wherein each nucleic acid molecule of the first plurality comprises a first end, a backbone sequence comprising a first sequencing primer sequence, and a second end, wherein the first end comprises a sequence selected from SEQ ID NO:11428 to SEQ ID NO:13483 and the second end comprises a sequence selected from SEQ ID NO:13484 to SEQ ID NO:15539; and
a second plurality of nucleic acid molecules, wherein each nucleic acid molecule of the second plurality comprises a first end, a backbone sequence comprising a second sequencing primer sequence, and a second end, wherein the first end comprises a sequence selected from SEQ ID NO:11428 to SEQ ID NO:13483 and the second end comprises a sequence selected from SEQ ID NO:13484 to SEQ ID NO:15539.

9. The kit of claim 8, further comprising a third, fourth, fifth, and sixth plurality of nucleic acid molecules, wherein each plurality comprises a plurality of nucleic acid molecules with a different sequencing primer sequence.

10. A gene probe including a sequence 80% homologous to SEQ ID NO:11428 to SEQ ID NO:15539.

11. The gene probe of claim 10, comprising a sequence 90% homologous to SEQ ID NO:11428 to SEQ ID NO:15539.

12. The gene probe of claim 10, comprising a sequence selected from SEQ ID NO:11428 to SEQ ID NO:15539.

13. The gene probe of claim 10, comprising a first sequence selected from SEQ ID NO:11428 to SEQ ID NO:13483 and a second sequence selected from SEQ ID NO:13484 to SEQ ID NO:15539.

14. The gene probe of claim 10, further comprising a primer binding sequence, or complement thereof, selected from: SEQ ID NO:15540, SEQ ID NO:15541, SEQ ID NO:15542, SEQ ID NO:15543, SEQ ID NO:15544, or SEQ ID NO:15545.

15. A cell or tissue comprising the gene probe of claim 10.

16. The cell of tissue of claim 15, wherein the cell or tissue further comprises a concatemer comprising multiple complementary copies of the gene probe.

17. The cell or tissue of claim 16, wherein the cell or tissue is immobilized to a solid support.

18. A kit comprising the gene probe of claim 10.

19. The kit of claim 18, further comprising a polymerase and a plurality of deoxynucleotides (dNTPs).

20. The kit of claim 18, further comprising a ligase.
